SEQUENCE LISTING (227 sequences)

SEQ ID NO: 1 - protein, 5 aa, synthetic construct
  REGION 1..5: note = Description of sequence: antibody component
TSINN

SEQ ID NO: 2 - protein, 7 aa, synthetic construct
  REGION 1..7: note = Description of sequence: antibody component
IRSNERE

SEQ ID NO: 3 - protein, 16 aa, synthetic construct
  REGION 1..16: note = Description of sequence: antibody component
CATGSNYGGSQGNLIF

SEQ ID NO: 4 - protein, 5 aa, synthetic construct
  REGION 1..5: note = Description of sequence: antibody component
SGHAT

SEQ ID NO: 5 - protein, 6 aa, synthetic construct
  REGION 1..6: note = Description of sequence: antibody component
FQNNGV

SEQ ID NO: 6 - protein, 15 aa, synthetic construct
  REGION 1..15: note = Description of sequence: antibody component
CASSLDYGSSYEQYF

SEQ ID NO: 7 - protein, 134 aa, synthetic construct
  REGION 1..134: note = Description of sequence: antibody component
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATGSNYGGSQGNLIFGKGTKLSVKP

SEQ ID NO: 8 - protein, 134 aa, synthetic construct
  REGION 1..134: note = Description of sequence: antibody component
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSLDYGSSYEQYFGPGTRLTVT

SEQ ID NO: 9 - protein, 275 aa, synthetic construct
  REGION 1..275: note = Description of sequence: antibody component
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATGSNYGGSQGNLIFGKGTKLSVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 10 - protein, 311 aa, synthetic construct
  REGION 1..311: note = Description of sequence: antibody component
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSLDYGSSYEQYFGPGTRLTVTEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 11 - protein, 7 aa, synthetic construct
  REGION 1..7: note = Description of sequence: antibody component
TSDPSYG

SEQ ID NO: 12 - protein, 8 aa, synthetic construct
  REGION 1..8: note = Description of sequence: antibody component
QGSYDQQN

SEQ ID NO: 13 - protein, 16 aa, synthetic construct
  REGION 1..16: note = Description of sequence: antibody component
CAMREGFSGGYNKLIF

SEQ ID NO: 14 - protein, 5 aa, synthetic construct
  REGION 1..5: note = Description of sequence: antibody component
SGHKS

SEQ ID NO: 15 - protein, 6 aa, synthetic construct
  REGION 1..6: note = Description of sequence: antibody component
YYEKEE

SEQ ID NO: 16 - protein, 20 aa, synthetic construct
  REGION 1..20: note = Description of sequence: antibody component
CASSDDTGTTKSSGANVLTF

SEQ ID NO: 17 - protein, 137 aa, synthetic construct
  REGION 1..137: note = Description of sequence: antibody component
MSLSSLLKVVTASLWLGPGIAQKITQTQPGMFVQEKEAVTLDCTYDTSDPSYGLFWYKQPSSGEMIFLIYQGSYDQQNATEGRYSLNFQKARKSANLVISASQLGDSAMYFCAMREGFSGGYNKLIFGAGTRLAVHP

SEQ ID NO: 18 - protein, 138 aa, synthetic construct
  REGION 1..138: note = Description of sequence: antibody component
MGPGLLCWVLLCLLGAGPVDAGVTQSPTHLIKTRGQQVTLRCSPISGHKSVSWYQQVLGQGPQFIFQYYEKEERGRGNFPDRFSARQFPNYSSELNVNALLLGDSALYLCASSDDTGTTKSSGANVLTFGAGSRLTVL

SEQ ID NO: 19 - protein, 278 aa, synthetic construct
  REGION 1..278: note = Description of sequence: antibody component
MSLSSLLKVVTASLWLGPGIAQKITQTQPGMFVQEKEAVTLDCTYDTSDPSYGLFWYKQPSSGEMIFLIYQGSYDQQNATEGRYSLNFQKARKSANLVISASQLGDSAMYFCAMREGFSGGYNKLIFGAGTRLAVHPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 20 - protein, 315 aa, synthetic construct
  REGION 1..315: note = Description of sequence: antibody component
MGPGLLCWVLLCLLGAGPVDAGVTQSPTHLIKTRGQQVTLRCSPISGHKSVSWYQQVLGQGPQFIFQYYEKEERGRGNFPDRFSARQFPNYSSELNVNALLLGDSALYLCASSDDTGTTKSSGANVLTFGAGSRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 21 - protein, 6 aa, synthetic construct
  REGION 1..6: note = Description of sequence: antibody component
NSMFDY

SEQ ID NO: 22 - protein, 7 aa, synthetic construct
  REGION 1..7: note = Description of sequence: antibody component
ISSIKDK

SEQ ID NO: 23 - protein, 14 aa, synthetic construct
  REGION 1..14: note = Description of sequence: antibody component
CAATVGLAGNMLTF

SEQ ID NO: 24 - protein, 5 aa, synthetic construct
  REGION 1..5: note = Description of sequence: antibody component
SGDLS

SEQ ID NO: 25 - protein, 6 aa, synthetic construct
  REGION 1..6: note = Description of sequence: antibody component
YYNGEE

SEQ ID NO: 26 - protein, 17 aa, synthetic construct
  REGION 1..17: note = Description of sequence: antibody component
CASSTPLSGSSTDTQYF

SEQ ID NO: 27 - protein, 139 aa, synthetic construct
  REGION 1..139: note = Description of sequence: antibody component
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAATVGLAGNMLTFGGGTRLMVKP

SEQ ID NO: 28 - protein, 135 aa, synthetic construct
  REGION 1..135: note = Description of sequence: antibody component
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSTPLSGSSTDTQYFGPGTRLTVL

SEQ ID NO: 29 - protein, 280 aa, synthetic construct
  REGION 1..280: note = Description of sequence: antibody component
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAATVGLAGNMLTFGGGTRLMVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 30 - protein, 312 aa, synthetic construct
  REGION 1..312: note = Description of sequence: antibody component
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSTPLSGSSTDTQYFGPGTRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 31 - protein, 6 aa, synthetic construct
  REGION 1..6: note = Description of sequence: antibody component
VSGLRG

SEQ ID NO: 32 - protein, 7 aa, synthetic construct
  REGION 1..7: note = Description of sequence: antibody component
LYSAGEE

SEQ ID NO: 33 - protein, 11 aa, synthetic construct
  REGION 1..11: note = Description of sequence: antibody component
CAVHSNYQLIW

SEQ ID NO: 34 - protein, 5 aa, synthetic construct
  REGION 1..5: note = Description of sequence: antibody component
SGDLS

SEQ ID NO: 35 - protein, 6 aa, synthetic construct
  REGION 1..6: note = Description of sequence: antibody component
YYNGEE

SEQ ID NO: 36 - protein, 17 aa, synthetic construct
  REGION 1..17: note = Description of sequence: antibody component
CASSTPLRTRGTDTQYF

SEQ ID NO: 37 - protein, 129 aa, synthetic construct
  REGION 1..129: note = Description of sequence: antibody component
MEKMLECAFIVLWLQLGWLSGEDQVTQSPEALRLQEGESSSLNCSYTVSGLRGLFWYRQDPGKGPEFLFTLYSAGEEKEKERLKATLTKKESFLHITAPKPEDSATYLCAVHSNYQLIWGAGTKLIIKP

SEQ ID NO: 38 - protein, 135 aa, synthetic construct
  REGION 1..135: note = Description of sequence: antibody component
MGFRLLCCVAFCLLGAGPVDSGVTQTPKHLITATGQRVTLRCSPRSGDLSVYWYQQSLDQGLQFLIHYYNGEERAKGNILERFSAQQFPDLHSELNLSSLELGDSALYFCASSTPLRTRGTDTQYFGPGTRLTVL

SEQ ID NO: 39 - protein, 270 aa, synthetic construct
  REGION 1..270: note = Description of sequence: antibody component
MEKMLECAFIVLWLQLGWLSGEDQVTQSPEALRLQEGESSSLNCSYTVSGLRGLFWYRQDPGKGPEFLFTLYSAGEEKEKERLKATLTKKESFLHITAPKPEDSATYLCAVHSNYQLIWGAGTKLIIKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 40 - protein, 312 aa, synthetic construct
  REGION 1..312: note = Description of sequence: antibody component
MGFRLLCCVAFCLLGAGPVDSGVTQTPKHLITATGQRVTLRCSPRSGDLSVYWYQQSLDQGLQFLIHYYNGEERAKGNILERFSAQQFPDLHSELNLSSLELGDSALYFCASSTPLRTRGTDTQYFGPGTRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 41 - protein, 6 aa, synthetic construct
  REGION 1..6: note = Description of sequence: antibody component
DSSSTY

SEQ ID NO: 42 - protein, 7 aa, synthetic construct
  REGION 1..7: note = Description of sequence: antibody component
IFSNMDM

SEQ ID NO: 43 - protein, 11 aa, synthetic construct
  REGION 1..11: note = Description of sequence: antibody component
CAEGYGGKLIF

SEQ ID NO: 44 - protein, 5 aa, synthetic construct
  REGION 1..5: note = Description of sequence: antibody component
SGHTA

SEQ ID NO: 45 - protein, 6 aa, synthetic construct
  REGION 1..6: note = Description of sequence: antibody component
FQGTGA

SEQ ID NO: 46 - protein, 15 aa, synthetic construct
  REGION 1..15: note = Description of sequence: antibody component
CASSSKTGVVGEQFF

SEQ ID NO: 47 - protein, 130 aa, synthetic construct
  REGION 1..130: note = Description of sequence: antibody component
MKTFAGFSFLFLWLQLDCMSRGEDVEQSLFLSVREGDSSVINCTYTDSSSTYLYWYKQEPGAGLQLLTYIFSNMDMKQDQRLTVLLNKKDKHLSLRIADTQTGDSAIYFCAEGYGGKLIFGQGTELSVKP

SEQ ID NO: 48 - protein, 134 aa, synthetic construct
  REGION 1..134: note = Description of sequence: antibody component
MGTRLLCWAALCLLGADHTGAGVSQTPSNKVTEKGKYVELRCDPISGHTALYWYRQSLGQGPEFLIYFQGTGAADDSGLPNDRFFAVRPEGSVSTLKIQRTERGDSAVYLCASSSKTGVVGEQFFGPGTRLTVL

SEQ ID NO: 49 - protein, 271 aa, synthetic construct
  REGION 1..271: note = Description of sequence: antibody component
MKTFAGFSFLFLWLQLDCMSRGEDVEQSLFLSVREGDSSVINCTYTDSSSTYLYWYKQEPGAGLQLLTYIFSNMDMKQDQRLTVLLNKKDKHLSLRIADTQTGDSAIYFCAEGYGGKLIFGQGTELSVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 50 - protein, 311 aa, synthetic construct
  REGION 1..311: note = Description of sequence: antibody component
MGTRLLCWAALCLLGADHTGAGVSQTPSNKVTEKGKYVELRCDPISGHTALYWYRQSLGQGPEFLIYFQGTGAADDSGLPNDRFFAVRPEGSVSTLKIQRTERGDSAVYLCASSSKTGVVGEQFFGPGTRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 51 - protein, 6 aa, synthetic construct
  REGION 1..6: note = Description of sequence: antibody component
DSSSTY

SEQ ID NO: 52 - protein, 7 aa, synthetic construct
  REGION 1..7: note = Description of sequence: antibody component
IFSNMDM

SEQ ID NO: 53 - protein, 15 aa, synthetic construct
  REGION 1..15: note = Description of sequence: antibody component
CAEISANQAGTALIF

SEQ ID NO: 54 - protein, 5 aa, synthetic construct
  REGION 1..5: note = Description of sequence: antibody component
SEHNR

SEQ ID NO: 55 - protein, 6 aa, synthetic construct
  REGION 1..6: note = Description of sequence: antibody component
FQNEAQ

SEQ ID NO: 56 - protein, 15 aa, synthetic construct
  REGION 1..15: note = Description of sequence: antibody component
CASSPLDSYQETQYF

SEQ ID NO: 57 - protein, 134 aa, synthetic construct
  REGION 1..134: note = Description of sequence: antibody component
MKTFAGFSFLFLWLQLDCMSRGEDVEQSLFLSVREGDSSVINCTYTDSSSTYLYWYKQEPGAGLQLLTYIFSNMDMKQDQRLTVLLNKKDKHLSLRIADTQTGDSAIYFCAEISANQAGTALIFGKGTTLSVSS

SEQ ID NO: 58 - protein, 134 aa, synthetic construct
  REGION 1..134: note = Description of sequence: antibody component
MGTSLLCWMALCLLGADHADTGVSQNPRHKITKRGQNVTFRCDPISEHNRLYWYRQTLGQGPEFLTYFQNEAQLEKSRLLSDRFSAERPKGSFSTLEIQRTEQGDSAMYLCASSPLDSYQETQYFGPGTRLLVL

SEQ ID NO: 59 - protein, 275 aa, synthetic construct
  REGION 1..275: note = Description of sequence: antibody component
MKTFAGFSFLFLWLQLDCMSRGEDVEQSLFLSVREGDSSVINCTYTDSSSTYLYWYKQEPGAGLQLLTYIFSNMDMKQDQRLTVLLNKKDKHLSLRIADTQTGDSAIYFCAEISANQAGTALIFGKGTTLSVSSNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 60 - protein, 311 aa, synthetic construct
  REGION 1..311: note = Description of sequence: antibody component
MGTSLLCWMALCLLGADHADTGVSQNPRHKITKRGQNVTFRCDPISEHNRLYWYRQTLGQGPEFLTYFQNEAQLEKSRLLSDRFSAERPKGSFSTLEIQRTEQGDSAMYLCASSPLDSYQETQYFGPGTRLLVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 61 - protein, 7 aa, synthetic construct
  REGION 1..7: note = Description of sequence: antibody component
TSDQSYG

SEQ ID NO: 62 - protein, 8 aa, synthetic construct
  REGION 1..8: note = Description of sequence: antibody component
QGSYDEQN

SEQ ID NO: 63 - protein, 14 aa, synthetic construct
  REGION 1..14: note = Description of sequence: antibody component
CAMREGRGRRALTF

SEQ ID NO: 64 - protein, 5 aa, synthetic construct
  REGION 1..5: note = Description of sequence: antibody component
LGHNA

SEQ ID NO: 65 - protein, 6 aa, synthetic construct
  REGION 1..6: note = Description of sequence: antibody component
YSLEER

SEQ ID NO: 66 - protein, 20 aa, synthetic construct
  REGION 1..20: note = Description of sequence: antibody component
CASSQDTAWDSGGETDTQYF

SEQ ID NO: 67 - protein, 135 aa, synthetic construct
  REGION 1..135: note = Description of sequence: antibody component
MSLSSLLKVVTASLWLGPGIAQKITQTQPGMFVQEKEAVTLDCTYDTSDQSYGLFWYKQPSSGEMIFLIYQGSYDEQNATEGRYSLNFQKARKSANLVISASQLGDSAMYFCAMREGRGRRALTFGSGTRLQVQP

SEQ ID NO: 68 - protein, 138 aa, synthetic construct
  REGION 1..138: note = Description of sequence: antibody component
MGCRLLCCAVLCLLGAVPMETGVTQTPRHLVMGMTNKKSLKCEQHLGHNAMYWYKQSAKKPLELMFVYSLEERVENNSVPSRFSPECPNSSHLFLHLHTLQPEDSALYLCASSQDTAWDSGGETDTQYFGPGTRLTVL

SEQ ID NO: 69 - protein, 276 aa, synthetic construct
  REGION 1..276: note = Description of sequence: antibody component
MSLSSLLKVVTASLWLGPGIAQKITQTQPGMFVQEKEAVTLDCTYDTSDQSYGLFWYKQPSSGEMIFLIYQGSYDEQNATEGRYSLNFQKARKSANLVISASQLGDSAMYFCAMREGRGRRALTFGSGTRLQVQPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 70 - protein, 315 aa, synthetic construct
  REGION 1..315: note = Description of sequence: antibody component
MGCRLLCCAVLCLLGAVPMETGVTQTPRHLVMGMTNKKSLKCEQHLGHNAMYWYKQSAKKPLELMFVYSLEERVENNSVPSRFSPECPNSSHLFLHLHTLQPEDSALYLCASSQDTAWDSGGETDTQYFGPGTRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 71 - protein, 7 aa, synthetic construct
  REGION 1..7: note = Description of sequence: antibody component
TSESNYY

SEQ ID NO: 72 - protein, 8 aa, synthetic construct
  REGION 1..8: note = Description of sequence: antibody component
QEAYKQQN

SEQ ID NO: 73 - protein, 15 aa, synthetic construct
  REGION 1..15: note = Description of sequence: antibody component
CAFMKWQGGSEKLVF

SEQ ID NO: 74 - protein, 5 aa, synthetic construct
  REGION 1..5: note = Description of sequence: antibody component
LGHDT

SEQ ID NO: 75 - protein, 6 aa, synthetic construct
  REGION 1..6: note = Description of sequence: antibody component
YNNKEL

SEQ ID NO: 76 - protein, 16 aa, synthetic construct
  REGION 1..16: note = Description of sequence: antibody component
CASSQALAGEVDTQYF

SEQ ID NO: 77 - protein, 136 aa, synthetic construct
  REGION 1..136: note = Description of sequence: antibody component
MTRVSLLWAVVVSTCLESGMAQTVTQSQPEMSVQEAETVTLSCTYDTSESNYYLFWYKQPPSRQMILVIRQEAYKQQNATENRFSVNFQKAAKSFSLKISDSQLGDTAMYFCAFMKWQGGSEKLVFGKGTKLTVNP

SEQ ID NO: 78 - protein, 134 aa, synthetic construct
  REGION 1..134: note = Description of sequence: antibody component
MGCRLLCCVVFCLLQAGPLDTAVSQTPKYLVTQMGNDKSIKCEQNLGHDTMYWYKQDSKKFLKIMFSYNNKELIINETVPNRFSPKSPDKAHLNLHINSLELGDSAVYFCASSQALAGEVDTQYFGPGTRLTVL

SEQ ID NO: 79 - protein, 277 aa, synthetic construct
  REGION 1..277: note = Description of sequence: antibody component
MTRVSLLWAVVVSTCLESGMAQTVTQSQPEMSVQEAETVTLSCTYDTSESNYYLFWYKQPPSRQMILVIRQEAYKQQNATENRFSVNFQKAAKSFSLKISDSQLGDTAMYFCAFMKWQGGSEKLVFGKGTKLTVNPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 80 - protein, 311 aa, synthetic construct
  REGION 1..311: note = Description of sequence: antibody component
MGCRLLCCVVFCLLQAGPLDTAVSQTPKYLVTQMGNDKSIKCEQNLGHDTMYWYKQDSKKFLKIMFSYNNKELIINETVPNRFSPKSPDKAHLNLHINSLELGDSAVYFCASSQALAGEVDTQYFGPGTRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 81 - protein, 7 aa, synthetic construct
  REGION 1..7: note = Description of sequence: antibody component
TRDTTYY

SEQ ID NO: 82 - protein, 8 aa, synthetic construct
  REGION 1..8: note = Description of sequence: antibody component
RNSFDEQN

SEQ ID NO: 83 - protein, 13 aa, synthetic construct
  REGION 1..13: note = Description of sequence: antibody component
CALRNSGNTPLVF

SEQ ID NO: 84 - protein, 5 aa, synthetic construct
  REGION 1..5: note = Description of sequence: antibody component
SGDLS

SEQ ID NO: 85 - protein, 6 aa, synthetic construct
  REGION 1..6: note = Description of sequence: antibody component
YYNGEE

SEQ ID NO: 86 - protein, 14 aa, synthetic construct
  REGION 1..14: note = Description of sequence: antibody component
CASSVDWVQETQYF

SEQ ID NO: 87 - protein, 134 aa, synthetic construct
  REGION 1..134: note = Description of sequence: antibody component
MLTASLLRAVIASICVVSSMAQKVTQAQTEISVVEKEDVTLDCVYETRDTTYYLFWYKQPPSGELVFLIRRNSFDEQNEISGRYSWNFQKSTSSFNFTITASQVVDSAVYFCALRNSGNTPLVFGKGTRLSVIA

SEQ ID NO: 88 - protein, 132 aa, synthetic construct
  REGION 1..132: note = Description of sequence: antibody component
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSVDWVQETQYFGPGTRLLVL

SEQ ID NO: 89 - protein, 275 aa, synthetic construct
  REGION 1..275: note = Description of sequence: antibody component
MLTASLLRAVIASICVVSSMAQKVTQAQTEISVVEKEDVTLDCVYETRDTTYYLFWYKQPPSGELVFLIRRNSFDEQNEISGRYSWNFQKSTSSFNFTITASQVVDSAVYFCALRNSGNTPLVFGKGTRLSVIANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 90 - protein, 309 aa, synthetic construct
  REGION 1..309: note = Description of sequence: antibody component
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSVDWVQETQYFGPGTRLLVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 91 - protein, 7 aa, synthetic construct
  REGION 1..7: note = Description of sequence: antibody component
TISGNEY

SEQ ID NO: 92 - protein, 5 aa, synthetic construct
  REGION 1..5: note = Description of sequence: antibody component
GLKNN

SEQ ID NO: 93 - protein, 15 aa, synthetic construct
  REGION 1..15: note = Description of sequence: antibody component
CIVPRGFSNTGKLIF

SEQ ID NO: 94 - protein, 6 aa, synthetic construct
  REGION 1..6: note = Description of sequence: antibody component
DFQATT

SEQ ID NO: 95 - protein, 7 aa, synthetic construct
  REGION 1..7: note = Description of sequence: antibody component
SNEGSKA

SEQ ID NO: 96 - protein, 15 aa, synthetic construct
  REGION 1..15: note = Description of sequence: antibody component
CSASESLALASEQYF

SEQ ID NO: 97 - protein, 129 aa, synthetic construct
  REGION 1..129: note = Description of sequence: antibody component
MRLVARVTVFLTFGTIIDAKTTQPTSMDCAEGRAANLPCNHSTISGNEYVYWYRQIHSQGPQYIIHGLKNNETNEMASLIITEDRKSSTLILPHATLRDTAVYYCIVPRGFSNTGKLIFGQGTTLQVKP

SEQ ID NO: 98 - protein, 131 aa, synthetic construct
  REGION 1..131: note = Description of sequence: antibody component
MLLLLLLLGPGSGLGAVVSQHPSRVICKSGTSVKIECRSLDFQATTMFWYRQFPKQSLMLMATSNEGSKATYEQGVEKDKFLINHASLTLSTLTVTSAHPEDSSFYICSASESLALASEQYFGPGTRLTVT

SEQ ID NO: 99 - protein, 270 aa, synthetic construct
  REGION 1..270: note = Description of sequence: antibody component
MRLVARVTVFLTFGTIIDAKTTQPTSMDCAEGRAANLPCNHSTISGNEYVYWYRQIHSQGPQYIIHGLKNNETNEMASLIITEDRKSSTLILPHATLRDTAVYYCIVPRGFSNTGKLIFGQGTTLQVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 100 - protein, 308 aa, synthetic construct
  REGION 1..308: note = Description of sequence: antibody component
MLLLLLLLGPGSGLGAVVSQHPSRVICKSGTSVKIECRSLDFQATTMFWYRQFPKQSLMLMATSNEGSKATYEQGVEKDKFLINHASLTLSTLTVTSAHPEDSSFYICSASESLALASEQYFGPGTRLTVTEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 101 - protein, 6 aa, synthetic construct
  REGION 1..6: note = Description of sequence: antibody component
NSAFQY

SEQ ID NO: 102 - protein, 6 aa, synthetic construct
  REGION 1..6: note = Description of sequence: antibody component
TYSSGN

SEQ ID NO: 103 - protein, 13 aa, synthetic construct
  REGION 1..13: note = Description of sequence: antibody component
CASSFSGYNKLIF

SEQ ID NO: 104 - protein, 5 aa, synthetic construct
  REGION 1..5: note = Description of sequence: antibody component
SGDLS

SEQ ID NO: 105 - protein, 6 aa, synthetic construct
  REGION 1..6: note = Description of sequence: antibody component
YYNGEE

SEQ ID NO: 106 - protein, 17 aa, synthetic construct
  REGION 1..17: note = Description of sequence: antibody component
CASSERLAGAIADTQYF

SEQ ID NO: 107 - protein, 133 aa, synthetic construct
  REGION 1..133: note = Description of sequence: antibody component
MMKSLRVLLVILWLQLSWVWSQQKEVEQDPGPLSVPEGAIVSLNCTYSNSAFQYFMWYRQYSRKGPELLMYTYSSGNKEDGRFTAQVDKSSKYISLFIRDSQPSDSATYLCASSFSGYNKLIFGAGTRLAVHP

SEQ ID NO: 108 - protein, 135 aa, synthetic construct
  REGION 1..135: note = Description of sequence: antibody component
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSERLAGAIADTQYFGPGTRLTVL

SEQ ID NO: 109 - protein, 274 aa, synthetic construct
  REGION 1..274: note = Description of sequence: antibody component
MMKSLRVLLVILWLQLSWVWSQQKEVEQDPGPLSVPEGAIVSLNCTYSNSAFQYFMWYRQYSRKGPELLMYTYSSGNKEDGRFTAQVDKSSKYISLFIRDSQPSDSATYLCASSFSGYNKLIFGAGTRLAVHPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 110 - protein, 311 aa, synthetic construct
  REGION 1..311: note = Description of sequence: antibody component
MGFRLLCCVAFCLLGAGPVDSGVTQTPKHITATGQRVTLRCSPRSGDLSVYWYQQSLDQGLQFLIQYYNGEERAKGNILERFSAQQFPDLHSELNLSSLELGDSALYFCASSERLAGAIADTQYFGPGTRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 111 - protein, 6 aa, synthetic construct
  REGION 1..6: note = Description of sequence: antibody component
VSGNPY

SEQ ID NO: 112 - protein, 8 aa, synthetic construct
  REGION 1..8: note = Description of sequence: antibody component
YITGDNLV

SEQ ID NO: 113 - protein, 13 aa, synthetic construct
  REGION 1..13: note = Description of sequence: antibody component
CAVRAPGNTPLVF

SEQ ID NO: 114 - protein, 5 aa, synthetic construct
  REGION 1..5: note = Description of sequence: antibody component
LGHDT

SEQ ID NO: 115 - protein, 6 aa, synthetic construct
  REGION 1..6: note = Description of sequence: antibody component
YNNKEL

SEQ ID NO: 116 - protein, 15 aa, synthetic construct
  REGION 1..15: note = Description of sequence: antibody component
CASSQDLGFDEKLFF

SEQ ID NO: 117 - protein, 132 aa, synthetic construct
  REGION 1..132: note = Description of sequence: antibody component
MASAPISMLAMLFTLSGLRAQSVAQPEDQVNVAEGNPLTVKCTYSVSGNPYLFWYVQYPNRGLQFLLKYITGDNLVKGSYGFEAEFNKSQTSFHLKKPSALVSDSALYFCAVRAPGNTPLVFGKGTRLSVIA

SEQ ID NO: 118 - protein, 133 aa, synthetic construct
  REGION 1..133: note = Description of sequence: antibody component
MGCRLLCCVVFCLLQAGPLDTAVSQTPKYLVTQMGNDKSIKCEQNLGHDTMYWYKQDSKKFLKIMFSYNNKELIINETVPNRFSPKSPDKAHLNLHINSLELGDSAVYFCASSQDLGFDEKLFFGSGTQLSVL

SEQ ID NO: 119 - protein, 273 aa, synthetic construct
  REGION 1..273: note = Description of sequence: antibody component
MASAPISMLAMLFTLSGLRAQSVAQPEDQVNVAEGNPLTVKCTYSVSGNPYLFWYVQYPNRGLQFLLKYITGDNLVKGSYGFEAEFNKSQTSFHLKKPSALVSDSALYFCAVRAPGNTPLVFGKGTRLSVIANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 120 - protein, 310 aa, synthetic construct
  REGION 1..310: note = Description of sequence: antibody component
MGCRLLCCVVFCLLQAGPLDTAVSQTPKYLVTQMGNDKSIKCEQNLGHDTMYWYKQDSKKFLKIMFSYNNKELIINETVPNRFSPKSPDKAHLNLHINSLELGDSAVYFCASSQDLGFDEKLFFGSGTQLSVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 121 - protein, 393 aa, synthetic construct [sequence omitted]

SEQ ID NO: 122 - protein, 393 aa, synthetic construct [sequence omitted]

SEQ ID NO: 123 - protein, 141 aa, Homo sapiens
  REGION 1..141: note = Description of sequence: Human alpha chain constant region
NIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 124 - protein, 137 aa, Homo sapiens
  REGION 1..137: note = Description of sequence: Mouse alpha chain constant region
NIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 125 - protein, 134 aa, Mus musculus
  REGION 1..134: note = Description of sequence: Mouse alpha chain constant region (native)
DIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRL

SEQ ID NO: 126 - protein, 177 aa, Homo sapiens
  REGION 1..177: note = Description of sequence: Human Beta chain constant region:
EDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 127 - protein, 173 aa, Mus musculus
  REGION 1..173: note = Description of sequence: Mouse Beta chain constant region:
EDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 128 - skipped

SEQ ID NO: 129 - protein, 179 aa, Homo sapiens
  REGION 1..179: note = Description of sequence: Human Beta chain constant region
EDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 130 - protein, 235 aa, synthetic construct
  REGION 1..235: note = Description of sequence: Beta chain
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 131 - protein, 210 aa, synthetic construct
  REGION 1..210: note = Description of sequence: Beta chain
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQFYK

SEQ ID NO: 132 - protein, 458 aa, synthetic construct
  REGION 1..458: note = Description of sequence: CD4 co-receptor
MNRGVPFRHLLLVLQLALLPAATQGKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 133 - protein, 27 aa, synthetic construct
  REGION 1..27: note = Description of sequence: Furin 2A protein
RAKRSGSGATNFSLLKQAGDVEENPGP

SEQ ID NO: 134 - protein, 605 aa, synthetic construct
  REGION 1..605: note = Description of sequence: T-CELL RECEPTOR 1A
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSLDYGSSYEQYFGPGTRLTVTEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMETLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATGSNYGGSQGNLIFGKGTKLSVKPDIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 135 - protein, 612 aa, synthetic construct
  REGION 1..612: note = Description of sequence: T-CELL RECEPTOR 1B
MGPGLLCWVLLCLLGAGPVDAGVTQSPTHLIKTRGQQVTLRCSPISGHKSVSWYQQVLGQGPQFIFQYYEKEERGRGNFPDRFSARQFPNYSSELNVNALLLGDSALYLCASSDDTGTTKSSGANVLTFGAGSRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMSLSSLLKVVTASLWLGPGIAQKITQTQPGMFVQEKEAVTLDCTYDTSDPSYGLFWYKQPSSGEMIFLIYQGSYDQQNATEGRYSLNFQKARKSANLVISASQLGDSAMYFCAMREGFSGGYNKLIFGAGTRLAVHPDIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 136 - protein, 611 aa, synthetic construct
  REGION 1..611: note = Description of sequence: T-CELL RECEPTOR 1C
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSTPLSGSSTDTQYFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAATVGLAGNMLTFGGGTRLMVKPDIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 137 - protein, 601 aa, synthetic construct
  REGION 1..601: note = Description of sequence: T-CELL RECEPTOR 1D
MGFRLLCCVAFCLLGAGPVDSGVTQTPKHLITATGQRVTLRCSPRSGDLSVYWYQQSLDQGLQFLIHYYNGEERAKGNILERFSAQQFPDLHSELNLSSLELGDSALYFCASSTPLRTRGTDTQYFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMEKMLECAFIVLWLQLGWLSGEDQVTQSPEALRLQEGESSSLNCSYTVSGLRGLFWYRQDPGKGPEFLFTLYSAGEEKEKERLKATLTKKESFLHITAPKPEDSATYLCAVHSNYQLIWGAGTKLIIKPDIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 138 - protein, 601 aa, synthetic construct
  REGION 1..601: note = Description of sequence: T-CELL RECEPTOR 3A
MGTRLLCWAALCLLGADHTGAGVSQTPSNKVTEKGKYVELRCDPISGHTALYWYRQSLGQGPEFLIYFQGTGAADDSGLPNDRFFAVRPEGSVSTLKIQRTERGDSAVYLCASSSKTGVVGEQFFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMKTFAGFSFLFLWLQLDCMSRGEDVEQSLFLSVREGDSSVINCTYTDSSSTYLYWYKQEPGAGLQLLTYIFSNMDMKQDQRLTVLLNKKDKHLSLRIADTQTGDSAIYFCAEGYGGKLIFGQGTELSVKPDIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 139 - protein, 605 aa, synthetic construct
  REGION 1..605: note = Description of sequence: T-CELL RECEPTOR 3B
MGTSLLCWMALCLLGADHADTGVSQNPRHKITKRGQNVTFRCDPISEHNRLYWYRQTLGQGPEFLTYFQNEAQLEKSRLLSDRFSAERPKGSFSTLEIQRTEQGDSAMYLCASSPLDSYQETQYFGPGTRLLVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMKTFAGFSFLFLWLQLDCMSRGEDVEQSLFLSVREGDSSVINCTYTDSSSTYLYWYKQEPGAGLQLLTYIFSNMDMKQDQRLTVLLNKKDKHLSLRIADTQTGDSAIYFCAEISANQAGTALIFGKGTTLSVSSDIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 140 - protein, 610 aa, synthetic construct
  REGION 1..610: note = Description of sequence: T-CELL RECEPTOR 3C
MGCRLLCCAVLCLLGAVPMETGVTQTPRHLVMGMTNKKSLKCEQHLGHNAMYWYKQSAKKPLELMFVYSLEERVENNSVPSRFSPECPNSSHLFLHLHTLQPEDSALYLCASSQDTAWDSGGETDTQYFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMSLSSLLKVVTASLWLGPGIAQKITQTQPGMFVQEKEAVTLDCTYDTSDQSYGLFWYKQPSSGEMIFLIYQGSYDEQNATEGRYSLNFQKARKSANLVISASQLGDSAMYFCAMREGRGRRALTFGSGTRLQVQPDIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 141 - protein, 607 aa, synthetic construct
  REGION 1..607: note = Description of sequence: T-CELL RECEPTOR 4A
MGCRLLCCVVFCLLQAGPLDTAVSQTPKYLVTQMGNDKSIKCEQNLGHDTMYWYKQDSKKFLKIMFSYNNKELIINETVPNRFSPKSPDKAHLNLHINSLELGDSAVYFCASSQALAGEVDTQYFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMTRVSLLWAVVVSTCLESGMAQTVTQSQPEMSVQEAETVTLSCTYDTSESNYYLFWYKQPPSRQMILVIRQEAYKQQNATENRFSVNFQKAAKSFSLKISDSQLGDTAMYFCAFMKWQGGSEKLVFGKGTKLTVNPDIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 142 - protein, 603 aa, synthetic construct
  REGION 1..603: note = Description of sequence: T-CELL RECEPTOR 7A
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSVDWVQETQYFGPGTRLLVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMLTASLLRAVIASICVVSSMAQKVTQAQTEISVVEKEDVTLDCVYETRDTTYYLFWYKQPPSGELVFLIRRNSFDEQNEISGRYSWNFQKSTSSFNFTITASQVVDSAVYFCALRNSGNTPLVFGKGTRLSVIADIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 143 - protein, 597 aa, synthetic construct
  REGION 1..597: note = Description of sequence: T-CELL RECEPTOR 7B
MLLLLLLLGPGSGLGAVVSQHPSRVICKSGTSVKIECRSLDFQATTMFWYRQFPKQSLMLMATSNEGSKATYEQGVEKDKFLINHASLTLSTLTVTSAHPEDSSFYICSASESLALASEQYFGPGTRLTVTEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMRLVARVTVFLTFGTIIDAKTTQPTSMDCAEGRAANLPCNHSTISGNEYVYWYRQIHSQGPQYIIHGLKNNETNEMASLIITEDRKSSTLILPHATLRDTAVYYCIVPRGFSNTGKLIFGQGTTLQVKPDIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 144 - protein, 605 aa, synthetic construct
  REGION 1..605: note = Description of sequence: T-CELL RECEPTOR 7D
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSERLAGAIADTQYFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMMKSLRVLLVILWLQLSWVWSQQKEVEQDPGPLSVPEGAIVSLNCTYSNSAFQYFMWYRQYSRKGPELLMYTYSSGNKEDGRFTAQVDKSSKYISLFIRDSQPSDSATYLCASSFSGYNKLIFGAGTRLAVHPDIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 145 - protein, 602 aa, synthetic construct
  REGION 1..602: note = Description of sequence: T-CELL RECEPTOR 8A
MGCRLLCCVVFCLLQAGPLDTAVSQTPKYLVTQMGNDKSIKCEQNLGHDTMYWYKQDSKKFLKIMFSYNNKELIINETVPNRFSPKSPDKAHLNLHINSLELGDSAVYFCASSQDLGFDEKLFFGSGTQLSVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMASAPISMLAMLFTLSGLRAQSVAQPEDQVNVAEGNPLTVKCTYSVSGNPYLFWYVQYPNRGLQFLLKYITGDNLVKGSYGFEAEFNKSQTSFHLKKPSALVSDSALYFCAVRAPGNTPLVFGKGTRLSVIADIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 146 - protein, 9 aa, synthetic construct
  REGION 1..9: note = Description of sequence: mutant TP53 peptide
HMTEVVRHC

SEQ ID NO: 147 - protein, 9 aa, synthetic construct
  REGION 1..9: note = Description of sequence: mutant TP53 peptide
HMTEVVRRC

SEQ ID NO: 148 - protein, 137 aa, synthetic construct
  REGION 1..137: note = Description of sequence: antibody component
DIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 149 - skipped

SEQ ID NO: 150 - protein, 15 aa, synthetic construct
  REGION 1..15: note = Description of sequence: Antibody component: 1A b_cdr3
CASSLDYGSSYEQYF

SEQ ID NO: 151 - protein, 16 aa, synthetic construct
  REGION 1..16: note = Description of sequence: Antibody component: 1A a_cdr3
CATGSNYGGSQGNLIF

SEQ ID NO: 152 - protein, 20 aa, synthetic construct
  REGION 1..20: note = Description of sequence: Antibody component: IB b_cdr3
CASSDDTGTTKSSGANVLTF

SEQ ID NO: 153 - protein, 16 aa, synthetic construct
  REGION 1..16: note = Description of sequence: Antibody component: IB a_cdr3
CAMREGFSGGYNKLIF

SEQ ID NO: 154 - protein, 17 aa, synthetic construct
  REGION 1..17: note = Description of sequence: Antibody component: 1C b_cdr3
CASSTPLSGSSTDTQYF

SEQ ID NO: 155 - protein, 14 aa, synthetic construct
  REGION 1..14: note = Description of sequence: Antibody component: 1C a_cdr3
CAATVGLAGNMLTF

SEQ ID NO: 156 - protein, 17 aa, synthetic construct
  REGION 1..17: note = Description of sequence: Antibody component: ID b_cdr3
CASSTPLRTRGTDTQYF

SEQ ID NO: 157 - protein, 11 aa, synthetic construct
  REGION 1..11: note = Description of sequence: Antibody component: ID a_cdr3
CAVHSNYQLIW

SEQ ID NO: 158 - protein, 14 aa, synthetic construct
  REGION 1..14: note = Description of sequence: Antibody component: 2A b_cdr3
CASSQATGDQPQHF

SEQ ID NO: 159 - protein, 14 aa, synthetic construct
  REGION 1..14: note = Description of sequence: Antibody component: 2A a_cdr3
CLVGAGGSARQLTF

SEQ ID NO: 160 - protein, 17 aa, synthetic construct
  REGION 1..17: note = Description of sequence: Antibody component: 2B b_cdr3
CASRGLAGGIGTDTQYF

SEQ ID NO: 161 - protein, 15 aa, synthetic construct
  REGION 1..15: note = Description of sequence: Antibody component: 2B a_cdr3
CAVRPVYQAGTALIF

SEQ ID NO: 162 - protein, 15 aa, synthetic construct
  REGION 1..15: note = Description of sequence: Antibody component: 3A b_cdr3
CASSSKTGVVGEQFF

SEQ ID NO: 163 - protein, 11 aa, synthetic construct
  REGION 1..11: note = Description of sequence: Antibody component: 3A a_cdr3
CAEGYGGKLIF

SEQ ID NO: 164 - protein, 15 aa, synthetic construct
  REGION 1..15: note = Description of sequence: Antibody component: 3B b_cdr3
CASSPLDSYQETQYF

SEQ ID NO: 165 - protein, 15 aa, synthetic construct
  REGION 1..15: note = Description of sequence: Antibody component: 3B a_cdr3
CAEISANQAGTALIF

SEQ ID NO: 166 - protein, 20 aa, synthetic construct
  REGION 1..20: note = Description of sequence: Antibody component: 3C b_cdr3
CASSQDTAWDSGGETDTQYF

SEQ ID NO: 167 - protein, 14 aa, synthetic construct
  REGION 1..14: note = Description of sequence: Antibody component: 3C a_cdr3
CAMREGRGRRALTF

SEQ ID NO: 168 - protein, 16 aa, synthetic construct
  REGION 1..16: note = Description of sequence: Antibody component: 4A b_cdr3
CASSQALAGEVDTQYF

SEQ ID NO: 169 - protein, 15 aa, synthetic construct
  REGION 1..15: note = Description of sequence: Antibody component: 4A a_cdr3
CAFMKWQGGSEKLVF

SEQ ID NO: 170 - protein, 11 aa, synthetic construct
  REGION 1..11: note = Description of sequence: Antibody component: 5A b_cdr3
CSAPTPYGYTF

SEQ ID NO: 171 - protein, 16 aa, synthetic construct
  REGION 1..16: note = Description of sequence: Antibody component: 5A a_cdr3
CAVSDSGPGTASKLTF

SEQ ID NO: 172 - protein, 15 aa, synthetic construct
  REGION 1..15: note = Description of sequence: Antibody component: 5B b_cdr3
CASSPSGGGGDTQYF

SEQ ID NO: 173 - protein, 12 aa, synthetic construct
  REGION 1..12: note = Description of sequence: Antibody component: 5B a_cdr3
CAVNTNAGKSTF

SEQ ID NO: 174 - protein, 15 aa, synthetic construct
  REGION 1..15: note = Description of sequence: Antibody component: 5C b_cdr3
CASSARQGPTGELFF

SEQ ID NO: 175 - protein, 16 aa, synthetic construct
  REGION 1..16: note = Description of sequence: Antibody component: 5C a_cdr3
CAFLGAGGTSYGKLTF

SEQ ID NO: 176 - protein, 15 aa, synthetic construct
  REGION 1..15: note = Description of sequence: Antibody component: 5D b_cdr3
CASSARQGPTGELFF

SEQ ID NO: 177 - protein, 13 aa, synthetic construct
  REGION 1..13: note = Description of sequence: Antibody component: 5D a_cdr3
CAGHLYNNNDMRF

SEQ ID NO: 178 - protein, 13 aa, synthetic construct
  REGION 1..13: note = Description of sequence: Antibody component: 6A b_cdr3
CASSEQTSGFKYF

SEQ ID NO: 179 - protein, 13 aa, synthetic construct
  REGION 1..13: note = Description of sequence: Antibody component: 6A a_cdr3
CAVVTNRDDKIIF

SEQ ID NO: 180 - protein, 14 aa, synthetic construct
  REGION 1..14: note = Description of sequence: Antibody component: 6B b_cdr3
CASAPRGHRETQYF

SEQ ID NO: 181 - protein, 14 aa, synthetic construct
  REGION 1..14: note = Description of sequence: Antibody component: 6B a_cdr3
CATDDPSGTYKYIF

SEQ ID NO: 182 - protein, 14 aa, synthetic construct
  REGION 1..14: note = Description of sequence: Antibody component: 7A b_cdr3
CASSVDWVQETQYF

SEQ ID NO: 183 - protein, 13 aa, synthetic construct
  REGION 1..13: note = Description of sequence: Antibody component: 7A a_cdr3
CALRNSGNTPLVF

SEQ ID NO: 184 - protein, 15 aa, synthetic construct
  REGION 1..15: note = Description of sequence: Antibody component: 7B b_cdr3
CSASESLALASEQYF

SEQ ID NO: 185 - protein, 15 aa, synthetic construct
  REGION 1..15: note = Description of sequence: Antibody component: 7B a_cdr3
CIVPRGFSNTGKLIF

SEQ ID NO: 186 - protein, 15 aa, synthetic construct
  REGION 1..15: note = Description of sequence: Antibody component: 7C b_cdr3
CATSRDGLSYNEQFF

SEQ ID NO: 187 - protein, 15 aa, synthetic construct
  REGION 1..15: note = Description of sequence: Antibody component: 7C a_cdr3
CAMRRHTGANSKLTF

SEQ ID NO: 188 - protein, 17 aa, synthetic construct
  REGION 1..17: note = Description of sequence: Antibody component: 7D b_cdr3
CASSERLAGAIADTQYF

SEQ ID NO: 189 - protein, 13 aa, synthetic construct
  REGION 1..13: note = Description of sequence: Antibody component: 7D a_cdr3
CASSFSGYNKLIF

SEQ ID NO: 190 - protein, 17 aa, synthetic construct
  REGION 1..17: note = Description of sequence: Antibody component: 7E b_cdr3
CASSERLAGAIADTQYF

SEQ ID NO: 191 - protein, 15 aa, synthetic construct
  REGION 1..15: note = Description of sequence: Antibody component: 7E a_cdr3
CAVNAHNQAGTALIF

SEQ ID NO: 192 - protein, 15 aa, synthetic construct
  REGION 1..15: note = Description of sequence: Antibody component: 8A b_cdr3
CASSQDLGFDEKLFF

SEQ ID NO: 193 - protein, 13 aa, synthetic construct
  REGION 1..13: note = Description of sequence: Antibody component: 8A a_cdr3
CAVRAPGNTPLVF

SEQ ID NO: 194 - protein, 49 aa, synthetic construct
  REGION 1..49: note = Description of sequence: T-Cell receptor: 1B, >20% of index signal cutoff
AGILMVFWYCNQSTHKRGILMCQTEFWCVILMVFYTMRPSHAGIMVCST

SEQ ID NO: 195 - protein, 34 aa, synthetic construct
  REGION 1..34: note = Description of sequence: T-Cell receptor: 1B, >50% of index signal cutoff
AGILMVFWYCNQSTHKRLMTEWVILMVFRHAIVC

SEQ ID NO: 196 - protein, 83 aa, synthetic construct
  REGION 1..83: note = Description of sequence: T-Cell receptor: 1C, >10% of index signal cutoff
AGILMVFWYCNQSTHKRILMCNQSTAYCNSTWYCNQSTEAVYCNQSTAIMVFWYCNQSTYCNQSRYCNSTHAGILMVYCNQST

SEQ ID NO: 197 - protein, 72 aa, synthetic construct
  REGION 1..72: note = Description of sequence: T-Cell receptor: 1C, >20% of index signal cutoff
AGILMVFWYCNQSTHKRLMNQTYCNSTWYCNQSEAVYCNQSTIMVFWYCNQSTYCQSRYCNHAIMVYCNQST

SEQ ID NO: 198 - protein, 32 aa, synthetic construct
  REGION 1..32: note = Description of sequence: T-Cell receptor: 1C, >50% of index signal cutoff
AGILMFWYNQHKRMTEWYVSVYNSTRYHAIVC

SEQ ID NO: 199 - protein, 53 aa, synthetic construct
  REGION 1..53: note = Description of sequence: T-Cell receptor: 3A, >10% of index signal cutoff
AGILMVFWYCNQSTHKRILMVQTASTEILMVQILMVFQSTQRPFHAILMVCST

SEQ ID NO: 200 - protein, 39 aa, synthetic construct
  REGION 1..39: note = Description of sequence: T-Cell receptor: 3A, >20% of index signal cutoff
AGILMVFWYCNQSTHKRILMSTEIVILMVSTRFHCAILV

SEQ ID NO: 201 - protein, 29 aa, synthetic construct
  REGION 1..29: note = Description of sequence: T-Cell receptor: 3A, >50% of index signal cutoff
AGILMVFWYQSTHLMTEIVIMVRHCAILV

SEQ ID NO: 202 - protein, 133 aa, synthetic construct
  REGION 1..133: note = Description of sequence: T-Cell receptor: 3C, >10% of index signal cutoff
AGILMVFWYCNQSTDEHKRAILMPVWCNQSTDHKRAGILMPVFWYCNQSTDHKAGILMPVFWYCNQSTDEHAILMPVFWYCNQSTDAILMPVFYCNQSTDEHGLWYCNEHKRALMCNSHAGILMPVFWCNQST

SEQ ID NO: 203 - protein, 112 aa, synthetic construct
  REGION 1..112: note = Description of sequence: T-Cell receptor: 3C, >20% of index signal cutoff
AGILMVFWYCNQSTDHKRILMVWCNQSTDHRAGILMPVFWYCNSTDHALMVWYCNQSTDEHAILMVFWYCNQSTDAILMPVCNQSTDHGCNEHKRAMNSHAGILMPVFWCST

SEQ ID NO: 204 - protein, 69 aa, synthetic construct
  REGION 1..69: note = Description of sequence: T-Cell receptor: 4A, >10% of index signal cutoff
AGILMPVFWYCNQSTHKRLMVAFNQSTAVFWYDEAGIMVFWNQSILMVFNQDFDRLFWSHAGIVFCSTD

SEQ ID NO: 205 - protein, 43 aa, synthetic construct
  REGION 1..43: note = Description of sequence: T-Cell receptor: 4A, >20% of index signal cutoff
AGILMVFWYCNQSTHKRLMTASVFEAIVFNILMVFRFWHAIVC

SEQ ID NO: 206 - protein, 30 aa, synthetic construct
  REGION 1..30: note = Description of sequence: T-Cell receptor: 4A, >50% of index signal cutoff
AGILMVFWYSTHKRLMTEAVFIVFRHFCAV

SEQ ID NO: 207 - protein, 48 aa, synthetic construct
  REGION 1..48: note = Description of sequence: T-Cell receptor: 7A, >10% of index signal cutoff
AGILMPVFWYCNQSTHKRILMPVSTEILVMVFWYSTDRHAGILVFCST

SEQ ID NO: 208 - protein, 38 aa, synthetic construct
  REGION 1..38: note = Description of sequence: T-Cell receptor: 7A, >20% of index signal cutoff
AGILMVFWYCNQSTHKRLMVSTELVVFSTRHAILVCST

SEQ ID NO: 209 - protein, 29 aa, synthetic construct
  REGION 1..29: note = Description of sequence: T-Cell receptor: 7A, >50% of index signal cutoff
AGILMVFWYNQSTHKRLMTEVVTRHCAVT

SEQ ID NO: 210 - protein, 64 aa, synthetic construct
  REGION 1..64: note = Description of sequence: T-Cell receptor: 7B, >10% of index signal cutoff
AGILMPVFWYCNQSTDHILMPVCQTASTDEAGVFCSTILMVFTCRILMVFYCNSTHAGIVCNST

SEQ ID NO: 211 - protein, 52 aa, synthetic construct
  REGION 1..52: note = Description of sequence: T-Cell receptor: 7B, >20% of index signal cutoff
AILMPVFWYCNQSTHILMVCQTASTDEAGVCTILVRILMVFYCQSTHAGICS

SEQ ID NO: 212 - protein, 31 aa, synthetic construct
  REGION 1..31: note = Description of sequence: T-Cell receptor: 7B, >50% of index signal cutoff
HAILMVFYCNQSTLMSTEDAVILVRHFQTAC

SEQ ID NO: 213 - protein, 52 aa, synthetic construct
  REGION 1..52: note = Description of sequence: T-Cell receptor: 7D, >10% of index signal cutoff
AGILMVFWYCNQSTHKRILMPVCQTSTFWYNEIVIMVCSTRANSHAGIVCST

SEQ ID NO: 214 - protein, 44 aa, synthetic construct
  REGION 1..44: note = Description of sequence: T-Cell receptor: 7D, >20% of index signal cutoff
AGILMVFWYCNQSTHILMVCQTSTFWYEIVIVCTRASHAGIVCT

SEQ ID NO: 215 - protein, 32 aa, synthetic construct
  REGION 1..32: note = Description of sequence: T-Cell receptor: 7D, >50% of index signal cutoff
HAGILMVFWYCNQSTLMTEFWYVIVTRHAIVC

SEQ ID NO: 216 - protein, 94 aa, synthetic construct
  REGION 1..94: note = Description of sequence: T-Cell receptor: 8A, >10% of index signal cutoff
EAGILMPVFWYCNQSTHKRAILMPVCQTAGILMPVFCNSTDAGILMPVFWYCNQSTDHAILMVFCTAILMVFYCSTGERAVQHAGILMPVWCST

SEQ ID NO: 217 - protein, 78 aa, synthetic construct
  REGION 1..78: note = Description of sequence: T-Cell receptor: 8A, >20% of index signal cutoff
AGILMVFWYCNQSTHKRILMPVCQTAGLPVCSTDAGILMPVFWYCNQSTDEHILMVCTILMVFCSTRQHAGILMVCST

SEQ ID NO: 218 - protein, 65 aa, synthetic construct
  REGION 1..65: note = Description of sequence: T-Cell receptor: 8A, >50% of index signal cutoff
HAGILMVFWYCNQSTILMVQALPVCSTAGILMPVFWYCNQSTDEILMVCTILMVFTRHAILMVCT

SEQ ID NO: 219 - protein, 86 aa, synthetic construct
  REGION 1..86: note = Description of sequence: T-Cell receptor: TCR1 (NCI AV12/BV6, pt 4196), >10% of index signal cutoff
VAGILMVFWYCNQSTDHKRAILMVCQTAGCSTDAGILMPVFWYCNQSTDEHAGILMCSTAILMVFYCNQSTHKRMHAGILMVCNST

SEQ ID NO: 220 - protein, 69 aa, synthetic construct
  REGION 1..69: note = Description of sequence: T-Cell receptor: TCR1 (NCI AV12/BV6, pt 4196), >20% of index signal cutoff
AGILMVFWYCNQSTHRILMVCQTASTAGILMPVFWYCNQSTDEAIMVCTILMVCNQSTHKRMHAIVCST

SEQ ID NO: 221 - protein, 60 aa, synthetic construct
  REGION 1..60: note = Description of sequence: T-Cell receptor: TCR1 (NCI AV12/BV6, pt 4196), >50% of index signal cutoff
AGILMVFWYCNQSTHILMQASTAGILMVFWYCNQSTDEAIVTILMVCNQSTRHMAIVCST

SEQ ID NO: 222 - protein, 133 aa, synthetic construct
  REGION 1..133: note = Description of sequence: T-Cell receptor: TCR2 (NCI AV6/BV11, pt 4196), >10% of index signal cutoff
AGILMVFWYCNQSTDEHKRGILMVWCNQSTDHRAGLMPVWCNQSTDKAILMVFWYCNQSTDEHKRAILVFYCNQSTDEAGILMPVFYCNQSTDHYCNQEKRAILMPVFWYCNQSTEHKAGILMPVFWYCNQST

SEQ ID NO: 223 - protein, 120 aa, synthetic construct
  REGION 1..120: note = Description of sequence: T-Cell receptor: TCR2 (NCI AV6/BV11, pt 4196), >20% of index signal cutoff
AGILMVFWYCNQSTDEHKRILMVWCNQSTDHRAGLMPVWCNSTDAILMFWYCNQSTDEHKAIVYCNQSTDAGILMPVFYCNQSTDHYCNEKRAILMPVFWYCNQSTHKAGILMVFWCQST

SEQ ID NO: 224 - protein, 91 aa, synthetic construct
  REGION 1..91: note = Description of sequence: T-Cell receptor: TCR2 (NCI AV6/BV11, pt 4196), >50% of index signal cutoff
AGILMVFWYCNQSTDHKRILMWCNQTDHAMWCNSTIFWYCNQSTDEHKIVCSTDAILMVFYCNQSTHCNERILMPFWYCNQTHAILMVCST

SEQ ID NO: 225 - protein, 106 aa, synthetic construct
  REGION 1..106: note = Description of sequence: T-Cell receptor: TCR5 (NCI AV38/BV2, pt 4141), >10% of index signal cutoff
TAGILMVFWYCNQSTDHKRAILMPVCQSTDAGCQSTDAGYCNQSTDEAGIVYCNQSAGILMPVFWYCNQSTCQSTERAGILMPFWYCNQSTHAGILMPVFWCNQST

SEQ ID NO: 226 - protein, 75 aa, synthetic construct
  REGION 1..75: note = Description of sequence: T-Cell receptor: TCR5 (NCI AV38/BV2, pt 4141), >20% of index signal cutoff
AGILMVFWYCNQSTHILMVCQTACSTDCQSTDEAGVCQSTAGILMVFYCQTCRAMPFWCNSTHAGILMVFWCNST

SEQ ID NO: 227 - protein, 48 aa, synthetic construct
  REGION 1..48: note = Description of sequence: T-Cell receptor: TCR5 (NCI AV38/BV2, pt 4141), >50% of index signal cutoff
AILMVFYCNQSTHILMVCQTASTSEAVCGILMVYTRMFWHAILMVCST

(19) United States
(12) Patent Application Publication
(10) Pub. No.: US 2026/0232801 A1
(43) Pub. Date: Aug. 13, 2026

(54) T CELL RECEPTORS TARGETING TP53 HOTSPOT MUTATIONS AND USES THEREOF

(71) Applicants: MEMORIAL SLOAN-KETTERING CANCER CENTER, New York, NY (US); SLOAN-KETTERING INSTITUTE FOR CANCER RESEARCH, New York, NY (US); MEMORIAL HOSPITAL FOR CANCER AND ALLIED DISEASES, New York, NY (US)

(72) Inventors: Michael GORMALLY, New Haven, CT (US); Smita S. CHANDRAN, Long Island City, NY (US); Christopher A. KLEBANOFF, New York, NY (US)

(73) Assignees: MEMORIAL SLOAN-KETTERING CANCER CENTER, New York, NY (US); SLOAN-KETTERING INSTITUTE FOR CANCER RESEARCH, New York, NY (US); MEMORIAL HOSPITAL FOR CANCER AND ALLIED DISEASES, New York, NY (US)

(21) Appl. No.: 19/456,345

(22) Filed: Jan. 22, 2026

Related U.S. Application Data
(63) Continuation of application No. PCT/US2024/039524, filed on Jul. 25, 2024.
(60) Provisional application No. 63/515,497, filed on Jul. 25, 2023.

(51) Int. Cl. A61K 40/42; A61K 40/11; A61K 40/15; A61K 40/32; A61P 35/00; C07K 14/705; C07K 16/30; C12N 5/0783; C12N 15/86

(52) U.S. Cl. CPC A61K 40/4242; A61K 40/11; A61K 40/15; A61K 40/32; A61P 35/00; C07K 14/7051; C07K 14/70539; C07K 16/30; C12N 5/0636; C12N 15/86; C12N 2510/00; C12N 2740/13043

(57) ABSTRACT

Provided and featured are novel recombinant T cell receptors (TCRs) that target a mutated tumor suppressor TP53. Also provided are cells comprising such TCRs, and methods of using the recombinant TCRs and cells expressing the recombinant TCRs for treating cancers associated with mutated TP53.

### CROSS-REFERENCE TO RELATED APPLICATIONS

This application is a continuation application of International Patent Application No. PCT/US2024/039524, filed Jul. 25, 2025, which claims priority to U.S. Provisional Application No. 63/515,497, filed Jul. 25, 2023, the contents of each of which are incorporated by reference in their entireties, and to each of which priority is claimed.

### SEQUENCE LISTING

A Sequence Listing conforming to the rules of WIPO Standard ST.26 is hereby incorporated by reference. Said Sequence Listing has been filed as an electronic document via Patent Center encoded as XML in UTF-8 text. The electronic document, created on Jan. 21, 2026, is entitled “0727341891_ST26.xml”, and is 311,196 bytes in size.

### 1. INTRODUCTION

Provided and featured are novel recombinant T cell receptors (TCRs) that target a mutated tumor suppressor TP53. Also provided are cells comprising such TCRs, and methods of using the recombinant TCRs and cells expressing the recombinant TCRs for treating cancers associated with mutated TP53.

### 2. BACKGROUND OF THE INVENTION

Cell-based immunotherapy is a therapy with curative potential for the treatment of cancers. T cells and other immunoresponsive cells (e.g., NK cells) can be modified to target tumor antigens through the introduction of genetic material coding for T cell receptors (TCRs) that confer specificity to selected antigens. Targeted T cell therapy using specific TCRs have shown clinical success in treating multiple solid and hematologic malignancies.

Tumor protein P53 or cellular tumor antigen p53 (encoded by the gene TP53) is a tumor suppressor that regulates cell division. TP53 is the most frequently mutated gene across all subsets of human cancer accounting for more than 40% of cases. Additionally, 8 hotspot mutations in TP53 account for approximately 20% of all cases. R175H is among the most frequently observed TP53 mutations.

Accordingly, the development of TCRs that bind to epitopes derived from TP53 mutations can provide a therapeutic approach, while addressing the need for novel therapeutic strategies capable of inducing potent cancer eradication with minimal risk of normal tissue toxicity.

### 3. SUMMARY OF THE INVENTION

In certain embodiments, the presently disclosed subject matter relates to a recombinant T cell receptor (TCR) that binds to a TP53 peptide. In certain embodiments, the TCR comprises at least one amino acid alteration relative to the sequence of a naturally occurring TCR. In certain embodiments, the amino acid alteration is a cysteine modification. In certain embodiments, the TP53 peptide includes a R175 mutation. In certain embodiments, the TP53 peptide includes a R175H mutation. In certain embodiments, the TP53 peptide includes or consists of the amino acid sequence set forth in SEQ ID NO: 146. In certain embodiments, the TP53 peptide includes or consists of the amino acid sequence set forth in SEQ ID NO: 147.

In certain embodiments, the TP53 peptide is associated with an HLA class I complex. In certain embodiments, the HLA class I complex is selected from an HLA-A, an HLA-B, and an HLA-C. In certain embodiments, the HLA class I complex is an HLA-A. In certain embodiments, the HLA-A is an HLA-A*02 superfamily member. In certain embodiments, the HLA-A*02 superfamily member is selected from the group including HLA-A*02:02, HLA-A*02:03, HLA-A*02:04, HLA-A*02:05, HLA-A*02:06, HLA-A*02:07, HLA-A*02:09, HLA-A*02:11, HLA-A*02:12, HLA-A*02:13, HLA-A*02:0215HLA-A*02:14, HLA-A*02:17, HLA-A*02:71, HLA-A*02:77, HLA-A*68:02, and HLA-A*69:01. In certain embodiments, the HLA-A*02 superfamily member is HLA-A*02:01.

In certain embodiments, the TCR includes an extracellular domain that binds to the TP53 peptide, wherein the extracellular domain includes an α chain and a β chain, wherein the α chain includes an α chain variable region and α chain constant region, and the β chain includes a β chain variable region and a β chain constant region. In certain embodiments, the presently disclosed subject matter relates to a TCR, wherein: a) the α chain variable region includes a CDR3 including the amino acid sequence set forth in SEQ ID NO: 3 or a conservative modification thereof, and the B chain variable region includes a CDR3 including the amino acid sequence set forth in SEQ ID NO: 6 or a conservative modification thereof; b) the α chain variable region includes a CDR3 including the amino acid sequence set forth in SEQ ID NO: 13 or a conservative modification thereof, and the β chain variable region includes a CDR3 including the amino acid sequence set forth in SEQ ID NO: 16 or a conservative modification thereof; c) the α chain variable region includes a CDR3 including the amino acid sequence set forth in SEQ ID NO: 23 or a conservative modification thereof, and the β chain variable region includes a CDR3 including the amino acid sequence set forth in SEQ ID NO: 26 or a conservative modification thereof; d) the α chain variable region includes a CDR3 including the amino acid sequence set forth in SEQ ID NO: 33 or a conservative modification thereof, and the B chain variable region includes a CDR3 including the amino acid sequence set forth in SEQ ID NO: 36 or a conservative modification thereof; e) the a chain variable region includes a CDR3 including the amino acid sequence set forth in SEQ ID NO: 43 or a conservative modification thereof, and the β chain variable region includes a CDR3 including the amino acid sequence set forth in SEQ ID NO: 46 or a conservative modification thereof; f) the α chain variable region includes a CDR3 including the amino acid sequence set forth in SEQ ID NO: 53 or a conservative modification thereof, and the β chain variable region includes a CDR3 including the amino acid sequence set forth in SEQ ID NO: 56 or a conservative modification thereof; g) the α chain variable region includes a CDR3 including the amino acid sequence set forth in SEQ ID NO: 63 or a conservative modification thereof, and the β chain variable region includes a CDR3 including the amino acid sequence set forth in SEQ ID NO: 66 or a conservative modification thereof; h) the α chain variable region includes a CDR3 including the amino acid sequence set forth in SEQ ID NO: 73 or a conservative modification thereof, and the B chain variable region includes a CDR3 including the amino acid sequence set forth in SEQ ID NO: 76 or a conservative modification thereof; i) the α chain variable region includes a CDR3 including the amino acid sequence set forth in SEQ ID NO: 83 or a conservative modification thereof, and the β chain variable region includes a CDR3 including the amino acid sequence set forth in SEQ ID NO: 86 or a conservative modification thereof; j) the α chain variable region includes a CDR3 including the amino acid sequence set forth in SEQ ID NO: 93 or a conservative modification thereof, and the β chain variable region includes a CDR3 including the amino acid sequence set forth in SEQ ID NO: 96 or a conservative modification thereof; k) the α chain variable region includes a CDR3 including the amino acid sequence set forth in SEQ ID NO: 103 or a conservative modification thereof, and the β chain variable region includes a CDR3 including the amino acid sequence set forth in SEQ ID NO: 106 or a conservative modification thereof; or 1) the α chain variable region includes a CDR3 including the amino acid sequence set forth in SEQ ID NO: 113 or a conservative modification thereof, and the β chain variable region includes a CDR3 including the amino acid sequence set forth in SEQ ID NO: 116 or a conservative modification thereof.

In certain embodiments, the presently disclosed subject matter relates to a TCR, wherein: a) the α chain variable region includes a CDR2 including the amino acid sequence set forth in SEQ ID NO: 2 or a conservative modification thereof, and the β chain variable region includes a CDR2 including the amino acid sequence set forth in SEQ ID NO: 5 or a conservative modification thereof; b) the α chain variable region includes a CDR2 including the amino acid sequence set forth in SEQ ID NO: 12 or a conservative modification thereof, and the β chain variable region includes a CDR2 including the amino acid sequence set forth in SEQ ID NO: 15 or a conservative modification thereof; c) the α chain variable region includes a CDR2 including the amino acid sequence set forth in SEQ ID NO: 22 or a conservative modification thereof, and the β chain variable region includes a CDR2 including the amino acid sequence set forth in SEQ ID NO: 25 or a conservative modification thereof; d) the α chain variable region includes a CDR2 including the amino acid sequence set forth in SEQ ID NO: 32 or a conservative modification thereof, and the B chain variable region includes a CDR2 including the amino acid sequence set forth in SEQ ID NO: 35 or a conservative modification thereof; e) the α chain variable region includes a CDR2 including the amino acid sequence set forth in SEQ ID NO: 42 or a conservative modification thereof, and the β chain variable region includes a CDR2 including the amino acid sequence set forth in SEQ ID NO: 45 or a conservative modification thereof; f) the α chain variable region includes a CDR2 including the amino acid sequence set forth in SEQ ID NO: 52 or a conservative modification thereof, and the β chain variable region includes a CDR2 including the amino acid sequence set forth in SEQ ID NO: 55 or a conservative modification thereof; g) the α chain variable region includes a CDR2 including the amino acid sequence set forth in SEQ ID NO: 62 or a conservative modification thereof, and the B chain variable region includes a CDR2 including the amino acid sequence set forth in SEQ ID NO: 65 or a conservative modification thereof; h) the a chain variable region includes a CDR2 including the amino acid sequence set forth in SEQ ID NO: 72 or a conservative modification thereof, and the β chain variable region includes a CDR2 including the amino acid sequence set forth in SEQ ID NO: 75 or a conservative modification thereof; i) the α chain variable region includes a CDR2 including the amino acid sequence set forth in SEQ ID NO: 82 or a conservative modification thereof, and the β chain variable region includes a CDR2 including the amino acid sequence set forth in SEQ ID NO: 85 or a conservative modification thereof; j) the α chain variable region includes a CDR2 including the amino acid sequence set forth in SEQ ID NO: 92 or a conservative modification thereof, and the β chain variable region includes a CDR2 including the amino acid sequence set forth in SEQ ID NO: 95 or a conservative modification thereof, k) the α chain variable region includes a CDR2 including the amino acid sequence set forth in SEQ ID NO: 102 or a conservative modification thereof, and the B chain variable region includes a CDR2 including the amino acid sequence set forth in SEQ ID NO: 105 or a conservative modification thereof, or 1) the α chain variable region includes a CDR2 including the amino acid sequence set forth in SEQ ID NO: 112 or a conservative modification thereof, and the β chain variable region includes a CDR2 including the amino acid sequence set forth in SEQ ID NO: 115 or a conservative modification thereof.

In certain embodiments, the presently disclosed subject matter relates to a TCR, wherein: a) the α chain variable region includes a CDR1 including the amino acid sequence set forth in SEQ ID NO: 1 or a conservative modification thereof, and the β chain variable region includes a CDR1 including the amino acid sequence set forth in SEQ ID NO: 4 or a conservative modification thereof; b) the α chain variable region includes a CDR1 including the amino acid sequence set forth in SEQ ID NO: 11 or a conservative modification thereof, and the β chain variable region includes a CDR1 including the amino acid sequence set forth in SEQ ID NO: 14 or a conservative modification thereof; c) the α chain variable region includes a CDR1 including the amino acid sequence set forth in SEQ ID NO: 21 or a conservative modification thereof, and the β chain variable region includes a CDR1 including the amino acid sequence set forth in SEQ ID NO: 24 or a conservative modification thereof; d) the α chain variable region includes a CDR1 including the amino acid sequence set forth in SEQ ID NO: 31 or a conservative modification thereof, and the β chain variable region includes a CDR1 including the amino acid sequence set forth in SEQ ID NO: 34 or a conservative modification thereof; e) the α chain variable region includes a CDR1 including the amino acid sequence set forth in SEQ ID NO: 41 or a conservative modification thereof, and the β chain variable region includes a CDR1 including the amino acid sequence set forth in SEQ ID NO: 44 or a conservative modification thereof; f) the α chain variable region includes a CDR1 including the amino acid sequence set forth in SEQ ID NO: 51 or a conservative modification thereof, and the β chain variable region includes a CDR1 including the amino acid sequence set forth in SEQ ID NO: 54 or a conservative modification thereof; g) the α chain variable region includes a CDR1 including the amino acid sequence set forth in SEQ ID NO: 61 or a conservative modification thereof, and the β chain variable region includes a CDR1 including the amino acid sequence set forth in SEQ ID NO: 64 or a conservative modification thereof; h) the a chain variable region includes a CDR1 including the amino acid sequence set forth in SEQ ID NO: 71 or a conservative modification thereof, and the β chain variable region includes a CDR1 including the amino acid sequence set forth in SEQ ID NO: 74 or a conservative modification thereof; i) the α chain variable region includes a CDR1 including the amino acid sequence set forth in SEQ ID NO: 81 or a conservative modification thereof, and the β chain variable region includes a CDR1 including the amino acid sequence set forth in SEQ ID NO: 84 or a conservative modification thereof; j) the α chain variable region includes a CDR1 including the amino acid sequence set forth in SEQ ID NO: 91 or a conservative modification thereof, and the β chain variable region includes a CDR1 including the amino acid sequence set forth in SEQ ID NO: 94 or a conservative modification thereof; k) the α chain variable region includes a CDR1 including the amino acid sequence set forth in SEQ ID NO: 101 or a conservative modification thereof, and the B chain variable region includes a CDR1 including the amino acid sequence set forth in SEQ ID NO: 104 or a conservative modification thereof, or 1) the α chain variable region includes a CDR1 including the amino acid sequence set forth in SEQ ID NO: 111 or a conservative modification thereof, and the β chain variable region includes a CDR1 including the amino acid sequence set forth in SEQ ID NO: 114 or a conservative modification thereof.

In certain embodiments, the presently disclosed subject matter relates to a TCR, wherein: a) the α chain variable region includes a CDR1 including the amino acid sequence set forth in SEQ ID NO: 1 or a conservative modification thereof, a CDR2 including the amino acid sequence set forth in SEQ ID NO: 2 or a conservative modification thereof, and a CDR3 including the amino acid sequence set forth in SEQ ID NO: 3 or a conservative modification thereof; and the β chain variable region includes a CDR1 including the amino acid sequence set forth in SEQ ID NO: 4 or a conservative modification thereof, a CDR2 including the amino acid sequence set forth in SEQ ID NO: 5 or a conservative modification thereof, and a CDR3 including the amino acid sequence set forth in SEQ ID NO: 6 or a conservative modification thereof; b) the α chain variable region includes a CDR1 including the amino acid sequence set forth in SEQ ID NO: 11 or a conservative modification thereof, a CDR2 including the amino acid sequence set forth in SEQ ID NO: 12 or a conservative modification thereof, and a CDR3 including the amino acid sequence set forth in SEQ ID NO: 13 or a conservative modification thereof; and the β chain variable region includes a CDR1 including the amino acid sequence set forth in SEQ ID NO: 14 or a conservative modification thereof, a CDR2 including the amino acid sequence set forth in SEQ ID NO: 15 or a conservative modification thereof, and a CDR3 including the amino acid sequence set forth in SEQ ID NO: 16 or a conservative modification thereof; c) the α chain variable region includes a CDR1 including the amino acid sequence set forth in SEQ ID NO: 21 or a conservative modification thereof, a CDR2 including the amino acid sequence set forth in SEQ ID NO: 22 or a conservative modification thereof, and a CDR3 including the amino acid sequence set forth in SEQ ID NO: 23 or a conservative modification thereof; and the β chain variable region includes a CDR1 including the amino acid sequence set forth in SEQ ID NO: 24 or a conservative modification thereof, a CDR2 including the amino acid sequence set forth in SEQ ID NO: 25 or a conservative modification thereof, and a CDR3 including the amino acid sequence set forth in SEQ ID NO: 26 or a conservative modification thereof; d) the α chain variable region includes a CDR1 including the amino acid sequence set forth in SEQ ID NO: 31 or a conservative modification thereof, a CDR2 including the amino acid sequence set forth in SEQ ID NO: 32 or a conservative modification thereof, and a CDR3 including the amino acid sequence set forth in SEQ ID NO: 33 or a conservative modification thereof; and the β chain variable region includes a CDR1 including the amino acid sequence set forth in SEQ ID NO: 34 or a conservative modification thereof, a CDR2 including the amino acid sequence set forth in SEQ ID NO: 35 or a conservative modification thereof, and a CDR3 including the amino acid sequence set forth in SEQ ID NO: 36 or a conservative modification thereof; e) the α chain variable region includes a CDR1 including the amino acid sequence set forth in SEQ ID NO: 41 or a conservative modification thereof, a CDR2 including the amino acid sequence set forth in SEQ ID NO: 42 or a conservative modification thereof, and a CDR3 including the amino acid sequence set forth in SEQ ID NO: 43 or a conservative modification thereof; and the β chain variable region includes a CDR1 including the amino acid sequence set forth in SEQ ID NO: 44 or a conservative modification thereof, a CDR2 including the amino acid sequence set forth in SEQ ID NO: 45 or a conservative modification thereof, and a CDR3 including the amino acid sequence set forth in SEQ ID NO: 46 or a conservative modification thereof; f) the α chain variable region includes a CDR1 including the amino acid sequence set forth in SEQ ID NO: 51 or a conservative modification thereof, a CDR2 including the amino acid sequence set forth in SEQ ID NO: 52 or a conservative modification thereof, and a CDR3 including the amino acid sequence set forth in SEQ ID NO: 53 or a conservative modification thereof; and the β chain variable region includes a CDR1 including the amino acid sequence set forth in SEQ ID NO: 54 or a conservative modification thereof, a CDR2 including the amino acid sequence set forth in SEQ ID NO: 55 or a conservative modification thereof, and a CDR3 including the amino acid sequence set forth in SEQ ID NO: 56 or a conservative modification thereof; g) the α chain variable region includes a CDR1 including the amino acid sequence set forth in SEQ ID NO: 61 or a conservative modification thereof, a CDR2 including the amino acid sequence set forth in SEQ ID NO: 62 or a conservative modification thereof, and a CDR3 including the amino acid sequence set forth in SEQ ID NO: 63 or a conservative modification thereof; and the β chain variable region includes a CDR1 including the amino acid sequence set forth in SEQ ID NO: 64 or a conservative modification thereof, a CDR2 including the amino acid sequence set forth in SEQ ID NO: 65 or a conservative modification thereof, and a CDR3 including the amino acid sequence set forth in SEQ ID NO: 66 or a conservative modification thereof; h) the α chain variable region includes a CDR1 including the amino acid sequence set forth in SEQ ID NO: 71 or a conservative modification thereof, a CDR2 including the amino acid sequence set forth in SEQ ID NO: 72 or a conservative modification thereof, and a CDR3 including the amino acid sequence set forth in SEQ ID NO: 73 or a conservative modification thereof; and the β chain variable region includes a CDR1 including the amino acid sequence set forth in SEQ ID NO: 74 or a conservative modification thereof, a CDR2 including the amino acid sequence set forth in SEQ ID NO: 75 or a conservative modification thereof, and a CDR3 including the amino acid sequence set forth in SEQ ID NO: 76 or a conservative modification thereof; i) the α chain variable region includes a CDR1 including the amino acid sequence set forth in SEQ ID NO: 81 or a conservative modification thereof, a CDR2 including the amino acid sequence set forth in SEQ ID NO: 82 or a conservative modification thereof, and a CDR3 including the amino acid sequence set forth in SEQ ID NO: 83 or a conservative modification thereof; and the β chain variable region includes a CDR1 including the amino acid sequence set forth in SEQ ID NO: 84 or a conservative modification thereof, a CDR2 including the amino acid sequence set forth in SEQ ID NO: 85 or a conservative modification thereof, and a CDR3 including the amino acid sequence set forth in SEQ ID NO: 86 or a conservative modification thereof; j) the α chain variable region includes a CDR1 including the amino acid sequence set forth in SEQ ID NO: 91 or a conservative modification thereof, a CDR2 including the amino acid sequence set forth in SEQ ID NO: 92 or a conservative modification thereof, and a CDR3 including the amino acid sequence set forth in SEQ ID NO: 93 or a conservative modification thereof; and the β chain variable region includes a CDR1 including the amino acid sequence set forth in SEQ ID NO: 94 or a conservative modification thereof, a CDR2 including the amino acid sequence set forth in SEQ ID NO: 95 or a conservative modification thereof, and a CDR3 including the amino acid sequence set forth in SEQ ID NO: 96 or a conservative modification thereof; k) the α chain variable region includes a CDR1 including the amino acid sequence set forth in SEQ ID NO: 101 or a conservative modification thereof, a CDR2 including the amino acid sequence set forth in SEQ ID NO: 102 or a conservative modification thereof, and a CDR3 including the amino acid sequence set forth in SEQ ID NO: 103 or a conservative modification thereof; and the β chain variable region includes a CDR1 including the amino acid sequence set forth in SEQ ID NO: 104 or a conservative modification thereof, a CDR2 including the amino acid sequence set forth in SEQ ID NO: 105 or a conservative modification thereof, and a CDR3 including the amino acid sequence set forth in SEQ ID NO: 106 or a conservative modification thereof; or 1) the α chain variable region includes a CDR 1 including the amino acid sequence set forth in SEQ ID NO: 111 or a conservative modification thereof, a CDR2 including the amino acid sequence set forth in SEQ ID NO: 112 or a conservative modification thereof, and a CDR3 including the amino acid sequence set forth in SEQ ID NO: 113 or a conservative modification thereof; and the β chain variable region includes a CDR1 including the amino acid sequence set forth in SEQ ID NO: 114 or a conservative modification thereof, a CDR2 including the amino acid sequence set forth in SEQ ID NO: 115 or a conservative modification thereof, and a CDR3 including the amino acid sequence set forth in SEQ ID NO: 116 or a conservative modification thereof.

In certain embodiments, the presently disclosed subject matter relates to a TCR, wherein: a) the α chain variable region includes a CDR1 including the amino acid sequence set forth in SEQ ID NO: 1, a CDR2 including the amino acid sequence set forth in SEQ ID NO: 2, and a CDR3 including the amino acid sequence set forth in SEQ ID NO: 3; and the β chain variable region includes a CDR1 including the amino acid sequence set forth in SEQ ID NO: 4, a CDR2 including the amino acid sequence set forth in SEQ ID NO: 5, and a CDR3 including the amino acid sequence set forth in SEQ ID NO: 6; b) the α chain variable region includes a CDR1 including the amino acid sequence set forth in SEQ ID NO: 11, a CDR2 including the amino acid sequence set forth in SEQ ID NO: 12, and a CDR3 including the amino acid sequence set forth in SEQ ID NO: 13; and the β chain variable region includes a CDR1 including the amino acid sequence set forth in SEQ ID NO: 14, a CDR2 including the amino acid sequence set forth in SEQ ID NO: 15, and a CDR3 including the amino acid sequence set forth in SEQ ID NO: 16; c) the α chain variable region includes a CDR1 including the amino acid sequence set forth in SEQ ID NO: 21, a CDR2 including the amino acid sequence set forth in SEQ ID NO: 22, and a CDR3 including the amino acid sequence set forth in SEQ ID NO: 23; and the β chain variable region includes a CDR1 including the amino acid sequence set forth in SEQ ID NO: 24, a CDR2 including the amino acid sequence set forth in SEQ ID NO: 25, and a CDR3 including the amino acid sequence set forth in SEQ ID NO: 26; d) the α chain variable region includes a CDR1 including the amino acid sequence set forth in SEQ ID NO: 31, a CDR2 including the amino acid sequence set forth in SEQ ID NO: 32, and a CDR3 including the amino acid sequence set forth in SEQ ID NO: 33; and the β chain variable region includes a CDR1 including the amino acid sequence set forth in SEQ ID NO: 34, a CDR2 including the amino acid sequence set forth in SEQ ID NO: 35, and a CDR3 including the amino acid sequence set forth in SEQ ID NO: 36; e) the α chain variable region includes a CDR1 including the amino acid sequence set forth in SEQ ID NO: 41, a CDR2 including the amino acid sequence set forth in SEQ ID NO: 42, and a CDR3 including the amino acid sequence set forth in SEQ ID NO: 43; and the β chain variable region includes a CDR1 including the amino acid sequence set forth in SEQ ID NO: 44, a CDR2 including the amino acid sequence set forth in SEQ ID NO: 45, and a CDR3 including the amino acid sequence set forth in SEQ ID NO: 46; f) the α chain variable region includes a CDR1 including the amino acid sequence set forth in SEQ ID NO: 51, a CDR2 including the amino acid sequence set forth in SEQ ID NO: 52, and a CDR3 including the amino acid sequence set forth in SEQ ID NO: 53; and the β chain variable region includes a CDR1 including the amino acid sequence set forth in SEQ ID NO: 54, a CDR2 including the amino acid sequence set forth in SEQ ID NO: 55, and a CDR3 including the amino acid sequence set forth in SEQ ID NO: 56; g) the α chain variable region includes a CDR1 including the amino acid sequence set forth in SEQ ID NO: 61, a CDR2 including the amino acid sequence set forth in SEQ ID NO: 62, and a CDR3 including the amino acid sequence set forth in SEQ ID NO: 63; and the β chain variable region includes a CDR1 including the amino acid sequence set forth in SEQ ID NO: 64, a CDR2 including the amino acid sequence set forth in SEQ ID NO: 65, and a CDR3 including the amino acid sequence set forth in SEQ ID NO: 66; h) the α chain variable region includes a CDR1 including the amino acid sequence set forth in SEQ ID NO: 71, a CDR2 including the amino acid sequence set forth in SEQ ID NO: 72, and a CDR3 including the amino acid sequence set forth in SEQ ID NO: 73; and the β chain variable region includes a CDR1 including the amino acid sequence set forth in SEQ ID NO: 74, a CDR2 including the amino acid sequence set forth in SEQ ID NO: 75, and a CDR3 including the amino acid sequence set forth in SEQ ID NO: 76; i) the α chain variable region includes a CDR1 including the amino acid sequence set forth in SEQ ID NO: 81, a CDR2 including the amino acid sequence set forth in SEQ ID NO: 82, and a CDR3 including the amino acid sequence set forth in SEQ ID NO: 83; and the β chain variable region includes a CDR1 including the amino acid sequence set forth in SEQ ID NO: 84, a CDR2 including the amino acid sequence set forth in SEQ ID NO: 85, and a CDR3 including the amino acid sequence set forth in SEQ ID NO: 86; j) the α chain variable region includes a CDR 1 including the amino acid sequence set forth in SEQ ID NO: 91, a CDR2 including the amino acid sequence set forth in SEQ ID NO: 92, and a CDR3 including the amino acid sequence set forth in SEQ ID NO: 93; and the β chain variable region includes a CDR1 including the amino acid sequence set forth in SEQ ID NO: 94, a CDR2 including the amino acid sequence set forth in SEQ ID NO: 95, and a CDR3 including the amino acid sequence set forth in SEQ ID NO: 96; k) the α chain variable region includes a CDR1 including the amino acid sequence set forth in SEQ ID NO: 101, a CDR2 including the amino acid sequence set forth in SEQ ID NO: 102, and a CDR3 including the amino acid sequence set forth in SEQ ID NO: 103; and the β chain variable region includes a CDR1 including the amino acid sequence set forth in SEQ ID NO: 104, a CDR2 including the amino acid sequence set forth in SEQ ID NO: 105, and a CDR3 including the amino acid sequence set forth in SEQ ID NO: 106; or 1) the α chain variable region includes a CDR1 including the amino acid sequence set forth in SEQ ID NO: 111, a CDR2 including the amino acid sequence set forth in SEQ ID NO: 112, and a CDR3 including the amino acid sequence set forth in SEQ ID NO: 113; and the β chain variable region includes a CDR1 including the amino acid sequence set forth in SEQ ID NO: 114, a CDR2 including the amino acid sequence set forth in SEQ ID NO: 115, and a CDR3 including the amino acid sequence set forth in SEQ ID NO: 116. In certain embodiments, the α chain variable region comprises a CDR1 comprising the amino acid sequence set forth in SEQ ID NO: 81, a CDR2 comprising the amino acid sequence set forth in SEQ ID NO: 82, and a CDR3 comprising the amino acid sequence set forth in SEQ ID NO: 83; and the β chain variable region comprises a CDR1 comprising the amino acid sequence set forth in SEQ ID NO: 84, a CDR2 comprising the amino acid sequence set forth in SEQ ID NO: 85, and a CDR3 comprising the amino acid sequence set forth in SEQ ID NO: 86.

In certain embodiments, the α chain variable region includes an amino acid sequence that is at least about 80% homologous or identical to the amino acid sequence set forth in SEQ ID NO: 7, SEQ ID NO: 17, SEQ ID NO: 27, SEQ ID NO: 37, SEQ ID NO: 47, SEQ ID NO: 57, SEQ ID NO: 67, SEQ ID NO: 77, SEQ ID NO: 87, SEQ ID NO: 97, SEQ ID NO: 107, or SEQ ID NO: 117. In certain embodiments, the α chain variable region includes the amino acid sequence set forth in SEQ ID NO: 7, SEQ ID NO: 17, SEQ ID NO: 27, SEQ ID NO: 37, SEQ ID NO: 47, SEQ ID NO: 57, SEQ ID NO: 67, SEQ ID NO: 77, SEQ ID NO: 87, SEQ ID NO: 97, SEQ ID NO: 107, or SEQ ID NO: 117. In certain embodiments, the β chain variable region includes an amino acid sequence that is at least about 80% homologous or identical to the amino acid sequence set forth in SEQ ID NO: 8, SEQ ID NO: 18, SEQ ID NO: 28, SEQ ID NO: 38, SEQ ID NO: 48, SEQ ID NO: 58, SEQ ID NO: 68, SEQ ID NO: 78, SEQ ID NO: 88, SEQ ID NO: 98, SEQ ID NO: 108, or SEQ ID NO: 118. In certain embodiments, the β chain variable region includes the amino acid sequence set forth in SEQ ID NO: 8, SEQ ID NO: 18, SEQ ID NO: 28, SEQ ID NO: 38, SEQ ID NO: 48, SEQ ID NO: 58, SEQ ID NO: 68, SEQ ID NO: 78, SEQ ID NO: 88, SEQ ID NO: 98, SEQ ID NO: 108, or SEQ ID NO: 118.

In certain embodiments, the presently disclosed subject matter relates to a TCR, wherein: a) the α chain variable region includes an amino acid sequence that is at least about 80% homologous or identical to the amino acid sequence set forth in SEQ ID NO: 7, and the β chain variable region includes an amino acid sequence that is at least about 80% homologous or identical to the amino acid sequence set forth in SEQ ID NO: 8; b) the α chain variable region includes an amino acid sequence that is at least about 80% homologous or identical to the amino acid sequence set forth in SEQ ID NO: 17, and the β chain variable region includes an amino acid sequence that is at least about 80% homologous or identical to the amino acid sequence set forth in SEQ ID NO: 18; c) the α chain variable region includes an amino acid sequence that is at least about 80% homologous or identical to the amino acid sequence set forth in SEQ ID NO: 27, and the β chain variable region includes an amino acid sequence that is at least about 80% homologous or identical to the amino acid sequence set forth in SEQ ID NO: 28; d) the α chain variable region includes an amino acid sequence that is at least about 80% homologous or identical to the amino acid sequence set forth in SEQ ID NO: 37, and the β chain variable region includes an amino acid sequence that is at least about 80% homologous or identical to the amino acid sequence set forth in SEQ ID NO: 38; e) the α chain variable region includes an amino acid sequence that is at least about 80% homologous or identical to the amino acid sequence set forth in SEQ ID NO: 47, and the β chain variable region includes an amino acid sequence that is at least about 80% homologous or identical to the amino acid sequence set forth in SEQ ID NO: 48; f) the α chain variable region includes an amino acid sequence that is at least about 80% homologous or identical to the amino acid sequence set forth in SEQ ID NO: 57, and the β chain variable region includes an amino acid sequence that is at least about 80% homologous or identical to the amino acid sequence set forth in SEQ ID NO: 58; g) the α chain variable region includes an amino acid sequence that is at least about 80% homologous or identical to the amino acid sequence set forth in SEQ ID NO: 67, and the β chain variable region includes an amino acid sequence that is at least about 80% homologous or identical to the amino acid sequence set forth in SEQ ID NO: 68; h) the α chain variable region includes an amino acid sequence that is at least about 80% homologous or identical to the amino acid sequence set forth in SEQ ID NO: 77, and the β chain variable region includes an amino acid sequence that is at least about 80% homologous or identical to the amino acid sequence set forth in SEQ ID NO: 78; i) the α chain variable region includes an amino acid sequence that is at least about 80% homologous or identical to the amino acid sequence set forth in SEQ ID NO: 87, and the β chain variable region includes an amino acid sequence that is at least about 80% homologous or identical to the amino acid sequence set forth in SEQ ID NO: 88; j) the α chain variable region includes an amino acid sequence that is at least about 80% homologous or identical to the amino acid sequence set forth in SEQ ID NO: 97, and the β chain variable region includes an amino acid sequence that is at least about 80% homologous or identical to the amino acid sequence set forth in SEQ ID NO: 98; k) the α chain variable region includes an amino acid sequence that is at least about 80% homologous or identical to the amino acid sequence set forth in SEQ ID NO: 107, and the β chain variable region includes an amino acid sequence that is at least about 80% homologous or identical to the amino acid sequence set forth in SEQ ID NO: 108; or 1) the α chain variable region includes an amino acid sequence that is at least about 80% homologous or identical to the amino acid sequence set forth in SEQ ID NO: 117, and the β chain variable region includes an amino acid sequence that is at least about 80% homologous or identical to the amino acid sequence set forth in SEQ ID NO: 118.

In certain embodiments, the presently disclosed subject matter relates to a TCR, wherein: a) the α chain variable region includes the amino acid sequence set forth in SEQ ID NO: 7, and the B chain variable region includes the amino acid sequence set forth in SEQ ID NO: 8; b) the a chain variable region includes the amino acid sequence set forth in SEQ ID NO: 17, and the β chain variable region includes the amino acid sequence set forth in SEQ ID NO: 18; c) the α chain variable region includes the amino acid sequence set forth in SEQ ID NO: 27, and the β chain variable region includes the amino acid sequence set forth in SEQ ID NO: 28; d) the α chain variable region includes the amino acid sequence set forth in SEQ ID NO: 37, and the β chain variable region includes the amino acid sequence set forth in SEQ ID NO: 38; e) the α chain variable region includes the amino acid sequence set forth in SEQ ID NO: 47, and the β chain variable region includes the amino acid sequence set forth in SEQ ID NO: 48; f) the α chain variable region includes the amino acid sequence set forth in SEQ ID NO: 57, and the β chain variable region includes the amino acid sequence set forth in SEQ ID NO: 58; g) the α chain variable region includes the amino acid sequence set forth in SEQ ID NO: 67, and the β chain variable region includes the amino acid sequence set forth in SEQ ID NO: 68; h) the α chain variable region includes the amino acid sequence set forth in SEQ ID NO: 77, and the β chain variable region includes the amino acid sequence set forth in SEQ ID NO: 78; i) the α chain variable region includes the amino acid sequence set forth in SEQ ID NO: 87, and the β chain variable region includes the amino acid sequence set forth in SEQ ID NO: 88; j) the α chain variable region includes the amino acid sequence set forth in SEQ ID NO: 97, and the β chain variable region includes the amino acid sequence set forth in SEQ ID NO: 98; k) the α chain variable region includes the amino acid sequence set forth in SEQ ID NO: 107, and the β chain variable region includes the amino acid sequence set forth in SEQ ID NO: 108; or 1) the α chain variable region includes the amino acid sequence set forth in SEQ ID NO: 117, and the β chain variable region includes the amino acid sequence set forth in SEQ ID NO: 118. In certain embodiments, the α chain variable region comprises the amino acid sequence set forth in SEQ ID NO: 87, and the β chain variable region comprises the amino acid sequence set forth in SEQ ID NO: 88.

In certain embodiments, the α chain constant region includes an amino acid sequence that is at least about 80% homologous or identical to the amino acid sequence set forth in SEQ ID NO: 123, SEQ ID NO: 124, or SEQ ID NO: 148. In certain embodiments, the α chain constant region includes the amino acid sequence set forth in SEQ ID NO: 123, SEQ ID NO: 124, or SEQ ID NO: 148. In certain embodiments, the β chain constant region includes an amino acid sequence that is at least about 80% homologous or identical to the amino acid sequence set forth in SEQ ID NO: 125, SEQ ID NO: 126, SEQ ID NO: 127, or SEQ ID NO: 129. In certain embodiments, the β chain constant region includes the amino acid sequence set forth in SEQ ID NO: 125, SEQ ID NO: 126, SEQ ID NO: 127, or SEQ ID NO: 129. In certain embodiments, a) the α chain constant region includes an amino acid sequence that is at least about 80% homologous or identical to the amino acid sequence set forth in SEQ ID NO: 123, SEQ ID NO: 124, or SEQ ID NO: 148; and b) the β chain constant region includes an amino acid sequence that is at least about 80% homologous or identical to the amino acid sequence set forth in SEQ ID NO: 125, SEQ ID NO: 126, SEQ ID NO: 127, or SEQ ID NO: 129. In certain embodiments, a) the α chain constant region includes the amino acid sequence set forth in SEQ ID NO: 123, SEQ ID NO: 124, or SEQ ID NO: 148; and b) the B chain constant region includes the amino acid sequence set forth in SEQ ID NO: 125, SEQ ID NO: 126, SEQ ID NO: 127, or SEQ ID NO: 129. In certain embodiments, a) the α chain includes an amino acid sequence that is at least about 80% identical to the amino acid sequence set forth in SEQ ID NO: 9, and the β chain includes an amino acid sequence that is at least about 80% identical to the amino acid sequence set forth in SEQ ID NO: 10; b) the α chain includes an amino acid sequence that is at least about 80% identical to the amino acid sequence set forth in SEQ ID NO: 19, and the B chain includes an amino acid sequence that is at least about 80% identical to the amino acid sequence set forth in SEQ ID NO: 20; c) the α chain includes an amino acid sequence that is at least about 80% identical to the amino acid sequence set forth in SEQ ID NO: 29, and the β chain includes an amino acid sequence that is at least about 80% identical to the amino acid sequence set forth in SEQ ID NO: 30; d) the α chain includes an amino acid sequence that is at least about 80% identical to the amino acid sequence set forth in SEQ ID NO: 39, and the β chain includes an amino acid sequence that is at least about 80% identical to the amino acid sequence set forth in SEQ ID NO: 40; e) the α chain includes an amino acid sequence that is at least about 80% identical to the amino acid sequence set forth in SEQ ID NO: 49, and the β chain includes an amino acid sequence that is at least about 80% identical to the amino acid sequence set forth in SEQ ID NO: 50; f) the α chain includes an amino acid sequence that is at least about 80% identical to the amino acid sequence set forth in SEQ ID NO: 59, and the β chain includes an amino acid sequence that is at least about 80% identical to the amino acid sequence set forth in SEQ ID NO: 60; g) the α chain includes an amino acid sequence that is at least about 80% identical to the amino acid sequence set forth in SEQ ID NO: 69, and the β chain includes an amino acid sequence that is at least about 80% identical to the amino acid sequence set forth in SEQ ID NO: 70; h) the a chain includes an amino acid sequence that is at least about 80% identical to the amino acid sequence set forth in SEQ ID NO: 79, and the β chain includes an amino acid sequence that is at least about 80% identical to the amino acid sequence set forth in SEQ ID NO: 80; i) the α chain includes an amino acid sequence that is at least about 80% identical to the amino acid sequence set forth in SEQ ID NO: 89, and the β chain includes an amino acid sequence that is at least about 80% identical to the amino acid sequence set forth in SEQ ID NO: 90; j) the α chain includes an amino acid sequence that is at least about 80% identical to the amino acid sequence set forth in SEQ ID NO: 99, and the β chain includes an amino acid sequence that is at least about 80% identical to the amino acid sequence set forth in SEQ ID NO: 100; k) the α chain includes an amino acid sequence that is at least about 80% identical to the amino acid sequence set forth in SEQ ID NO: 109, and the β chain includes an amino acid sequence that is at least about 80% identical to the amino acid sequence set forth in SEQ ID NO: 110; or 1) the α chain includes an amino acid sequence that is at least about 80% identical to the amino acid sequence set forth in SEQ ID NO: 119, and the β chain includes an amino acid sequence that is at least about 80% identical to the amino acid sequence set forth in SEQ ID NO: 120.

In certain embodiments, the presently disclosed subject matter relates to a TCR, wherein: a) the α chain includes the amino acid sequence set forth in SEQ ID NO: 9, and the β chain includes the amino acid sequence set forth in SEQ ID NO: 10; b) the α chain includes the amino acid sequence set forth in SEQ ID NO: 19, and the β chain includes the amino acid sequence set forth in SEQ ID NO: 20; c) the α chain includes the amino acid sequence set forth in SEQ ID NO: 29, and the β chain includes the amino acid sequence set forth in SEQ ID NO: 30; d) the α chain includes the amino acid sequence set forth in SEQ ID NO: 39, and the β chain includes the amino acid sequence set forth in SEQ ID NO: 40; e) the α chain includes the amino acid sequence set forth in SEQ ID NO: 49, and the B chain includes the amino acid sequence set forth in SEQ ID NO: 50; f) the α chain includes the amino acid sequence set forth in SEQ ID NO: 59, and the β chain includes the amino acid sequence set forth in SEQ ID NO: 60; g) the α chain includes the amino acid sequence set forth in SEQ ID NO: 69, and the β chain includes the amino acid sequence set forth in SEQ ID NO: 70; h) the α chain includes the amino acid sequence set forth in SEQ ID NO: 79, and the B chain includes the amino acid sequence set forth in SEQ ID NO: 80; i) the a chain includes the amino acid sequence set forth in SEQ ID NO: 89, and the β chain includes the amino acid sequence set forth in SEQ ID NO: 90; j) the α chain includes the amino acid sequence set forth in SEQ ID NO: 99, and the β chain includes the amino acid sequence set forth in SEQ ID NO: 100; k) the α chain includes the amino acid sequence set forth in SEQ ID NO: 109, and the B chain includes the amino acid sequence set forth in SEQ ID NO: 110; or 1) the α chain includes the amino acid sequence set forth in SEQ ID NO: 119, and the β chain includes the amino acid sequence set forth in SEQ ID NO: 120. In certain embodiments, the α chain comprises the amino acid sequence set forth in SEQ ID NO: 89, and the β chain comprises the amino acid sequence set forth in SEQ ID NO: 90.

In certain embodiments, the TCR is recombinant. In certain embodiments, the TCR is recombinantly expressed and/or expressed from a vector.

In certain embodiments, the presently disclosed subject matter relates to a T cell receptor (TCR) including an extracellular domain that binds to the same TP53 peptide as a reference TCR or a functional fragment thereof, wherein the reference TCR or functional fragment thereof includes an α chain variable region and a β chain variable region disclosed herein.

In certain embodiments, the presently disclosed subject matter relates to a cell including the TCR disclosed herein. In certain embodiments, the cell is transduced with the TCR. In certain embodiments, the TCR is constitutively expressed on the surface of the cell. In certain embodiments, the cell is an immunoresponsive cell. In certain embodiments, the cell is selected from the group consisting of a T cell, a Natural Killer (NK) cell, and a pluripotent stem cell from which a lymphoid cell may be differentiated. In certain embodiments, the cell is a T cell. In certain embodiments, the T cell is selected from the group consisting of a cytotoxic T lymphocyte (CTL), a regulatory T cell, a γδ T cell, a Natural Killer-T cell (NK-T), a stem cell memory T cell, a central memory T cell, and an effector memory T cell. In certain embodiments, the T cell is a γδ T cell. In certain embodiments, the T cell is a NK-T cell. In certain embodiments, the cell is an NK cell.

In certain embodiments, the TCR is integrated at a locus within the genome of the cell. In certain embodiments, the locus is selected from a TRAC locus, a TRBC locus, a TRDC locus, and a TRGC locus. In certain embodiments, the locus is a TRAC locus or a TRBC locus. In certain embodiments, the locus is selected from a PDCD1 locus, a CBLB locus, a CISH locus, and a RASA2 locus. In certain embodiments, the locus is selected from a PDCD1 locus. In certain embodiments, the locus is selected from a CBLB locus. In certain embodiments, the locus is selected from a CISH locus. In certain embodiments, the locus is selected from a RASA2 locus. In certain embodiments, the locus is a genomic safe harbor.

In certain embodiments, the cell further includes at least one recombinant or exogenous coreceptor. In certain embodiments, the co-receptor is a CD8 co-receptor. In certain embodiments, the co-receptor in a CD4 co-receptor.

In certain embodiments, the presently disclosed subject matter relates to a composition including the cells disclosed herein. In certain embodiments, the composition is a pharmaceutical composition further including a pharmaceutically acceptable carrier.

In certain embodiments, the presently disclosed subject matter relates to a nucleic acid encoding the T cell receptor (TCR) disclosed herein. In certain embodiments, the presently disclosed subject matter relates to a nucleic acid encoding a) comprising an amino acid sequence that is at least about 80% identical to the amino acid sequence set forth in SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, or SEQ ID NO: 144; or b) a polypeptide including or consisting of the amino acid sequence set forth in SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, or SEQ ID NO: 144. In certain embodiments, the nucleic acid further encodes a CD8 receptor or a CD4 receptor.

In certain embodiments, the presently disclosed subject matter relates to a vector including the nucleic acid disclosed herein. In certain embodiments, the vector is a γ-retroviral vector. In certain embodiments, the vector is a plasmid comprising a backbone comprising fewer than about 500 nucleotides.

In certain embodiments, the presently disclosed subject matter relates to a lipid nanoparticle including the nucleic acid or the vector disclosed herein. In certain embodiments, the presently disclosed subject matter relates to a cell including a nucleic acid, a vector, or a lipid nanoparticle disclosed herein.

Further, the presently disclosed subject matter relates to compositions comprising the nucleic acid, the vector, the lipid nanoparticle, or the cell disclosed herein. In certain embodiments, the composition is a pharmaceutical composition further comprising a pharmaceutically acceptable carrier.

In certain embodiments, the presently disclosed subject matter relates to a method for producing a cell that binds to a TP53 peptide that includes a R175 mutation, including introducing into the cell the nucleic acid, the vector, the lipid nanoparticle, or the composition disclosed herein.

In certain embodiments, the presently disclosed subject matter relates to methods of treating and/or preventing a tumor or a premalignant condition associated with TP53 in a subject, comprising administering to the subject the cell, the nucleic acid, the vector, the lipid nanoparticle, or the composition disclosed herein.

In certain embodiments, the tumor is selected from the group consisting of brain tumors, soft tissue and bone sarcomas, adrenocortical carcinoma, rhabdomyosarcoma, Wilms tumor, pancreatic cancer, breast cancer, endometrial cancer, cervical cancer, anal cancer, bladder cancer, colorectal cancer, cholangiocarcinoma/bile duct cancer, lung cancer, ovarian cancer, esophageal cancer, gastric cancer, head and neck squamous cell carcinoma, nonmelanoma skin cancer, salivary gland cancer, melanoma, multiple myeloma, prostate cancer, T-lymphoblastic leukemia/lymphoma, acute myeloid leukemia, B-cell leukemia, myeloproliferative neoplasm, and histiocytosis. In certain embodiments, the premalignant condition is clonal hematopoiesis. In certain embodiments, the tumor is colorectal cancer. In certain embodiments, the tumor is breast cancer. In certain embodiments, the tumor is esophageal cancer. In certain embodiments, the subject is a human.

In certain embodiments, the subject includes an HLA-A. In certain embodiments, the HLA-A is an HLA-A*02 superfamily member. In certain embodiments, the HLA-A*02 superfamily member is selected from the group including HLA-A*02, HLA-A*02:01, HLA-A*02:02, HLA-A*02:03, HLA-A*02:04, HLA-A*02:05, HLA-A*02:06, HLA-A*02:07, HLA-A*02:09, HLA-A*02:11, HLA-A*02:12, HLA-A*02:13, HLA-A*02:0215HLA-A*02:14, HLA-A*02:17, HLA-A*02:71, HLA-A*02:77, HLA-A*68:02, and HLA-A*69:01 In certain embodiments, the HLA-A*02 superfamily member is HLA-A*02:01.

In certain embodiments, the presently disclosed subject matter relates to a cell, a nucleic acid, a vector, a lipid nanoparticle, or a composition disclosed herein for use in treating and/or preventing a tumor associated with TP53 in a subject.

### 4. BRIEF DESCRIPTION OF THE FIGURES

The following Detailed Description, given by way of example but not intended to limit the invention to specific embodiments described, may be understood in conjunction with the accompanying drawings.

FIGS. 1A-1E illustrate the initial in vitro stimulation screening pipeline and single-cell sequence clonotype retrieval. FIG. 1A illustrates FACS analysis of cells undergone to three iterative rounds of peptide stimulation each followed by stainign with dual-color mutant peptide/HLA dextramers. FIG. 1B shows single-cell TCR sequencing using mutant and wild-type feature barcodes to facilitate de-orphaning of the hit TCR clonotypes. FIG. 1C depicts a summary of all single-cell sequencing results performed on putative hits from in vitro stimulation screen. FIG. 1D lists the 22 novel TCR sequences retrieved. Illustrated sequences are set forth as SEQ ID NOs: 150-193. FIG. 1E demonstrates the validation of dextramer binding for all retrieved TCRs.

FIGS. 2A-2C illustrate functionality and peptide avidity of CD8⁺ T cells transduced with the presently disclosed TCRs and stimulated with T2 cells pulsed with mutant R175H TP53 peptides or wild-type (WT) controls. FIG. 2A shows the dose response curve of IFNγ production. FIG. 2B shows response to wild-type control peptide. FIG. 2C shows the peptide avidity calculated from the dose response curve.

FIGS. 3A-3C illustrate functionality and peptide avidity of CD4⁺ T cells transduced with the presently disclosed TCRs and stimulated with T2 cells pulsed with mutant R175H TP53 peptides or wild-type (WT) controls. FIG. 3A shows the dose response curve of TNFα production. FIG. 3B shows response to wild-type control peptide. FIG. 3C shows the peptide avidity calculated from the dose response curve.

FIG. 4 demonstrates the functional activation of CD8⁺ T cells transduced with the presently disclosed TCRs in co-culture with TYKNU tumor cells.

FIG. 5 demonstrates the functional activation of CD4⁺ T cells transduced with the presently disclosed TCRs in co-culture with KMS26 tumor cells.

FIG. 6 illustrates tumor killing effect of CD8⁺ T cells transduced with the presently disclosed TCRs.

FIG. 7 illustrates tumor killing effect of CD4⁺ T cells transduced with the presently disclosed TCRs.

FIG. 8 depicts the structural analysis of hit TCR receptor sequences to identify an elite class of super long CDR3B loops.

FIG. 9 depicts amino acid mutagenesis (X-scan) results for the presently disclosed TP53 R175H TCRs versus NCI benchmarks.

FIG. 10 depicts absolute IFNγ release for the presently disclosed TP53 R175H TCRs versus NCI benchmarks.

FIG. 11 depicts predicted consensus motifs based on X-scan for the presently disclosed TP53 R175H TCRs versus NCI benchmarks. Illustrated sequences are set forth as SEQ ID Nos: 194-227.

FIG. 12 depicts number of predicted off-targets for the presently disclosed TP53 R175H TCRs versus NCI benchmarks by ScanProsite+/−netMHC filtering. *** Indicates too many off targets to query on ScanProsite or netMHC filter. Refer to next most stringent cutoff.

FIG. 13 depicts in vivo efficacy of the presently disclosed TP53 R175H TCRs versus existing benchmarks.

### 5. DETAILED DESCRIPTION OF THE INVENTION

The presently disclosed subject matter provides TCRs (e.g., recombinant TCRs) targeting TP53 comprising a mutation, e.g., a R175H mutation. Furthermore, the presently disclosed subject matter provides cells (e.g., T cells) comprising a presently disclosed TCR or a heterologous polynucleotide encoding a TCR that bind a TP53 polypeptide (e.g., a TP53 polypeptide comprising a R175H mutation), and methods of using such cells for treating tumors associated with TP53 mutation(s).

For purposes of clarity of disclosure and not by way of limitation, the detailed description is divided into the following subsections:
5.1. Definitions;
5.2 TP53;
5.3. TCRs;
5.4. Cells
5.5. Nucleic Acids and Genetic Modifications of Cell;
5.6. Formulations and Administration;
5.7. Methods of Treatments; and
5.8 Exemplary Embodiments.

### 5.1. Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991).

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

As used herein, the term “cell population” refers to a group of at least two cells expressing similar or different phenotypes. In non-limiting examples, a cell population can include at least about 10, at least about 100, at least about 200, at least about 300, at least about 400, at least about 500, at least about 600, at least about 700, at least about 800, at least about 900, at least about 10⁰⁰ cells expressing similar or different phenotypes.

As used herein, the term “vector” refers to any genetic element, such as a plasmid (e.g., a plasmid having a small backbone of less than about 500 base pairs, such as a Nanoplasmid™ vector system), phage, transposon, cosmid, chromosome, virus, virion, etc., which is capable of replication when associated with the proper control elements and which can transfer gene sequences into cells. Thus, the term includes cloning and expression vehicles, as well as viral vectors and plasmid vectors.

As used herein, the term “expression vector” refers to a recombinant nucleic acid sequence, e.g., a recombinant DNA molecule, containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

As used herein, “CDRs” are defined as the complementarity determining region amino acid sequences of a TCR, which are the hypervariable regions of TCR α-chain and β-chain. Generally, a TCR comprises three CDRs in the α-chain variable region and three CDRs in the β-chain variable region. CDRs provide the majority of contact residues for the binding of the TCR to the antigen or epitope. CDRs regions can be delineated using the Kabat system (Kabat, E. A., et al. (1991) Sequences of Proteins of Immunological Interest, Fifth Edition, U.S. Department of Health and Human Services, NIH Publication No. 91-3242), the Chothia numbering system (Chothia et al., J Mol Biol. (1987) 196:901-17), the AbM numbering system (Abhinandan et al., Mol. Immunol. 2008, 45, 3832-3839), or the IMGT numbering system (accessible at http://www.imgt.org/IMGTScientificChart/Numbering/IMGTIGVLsuperfamily.html #table1, http://www.imgt.org/IMGTScientificChart/Nomenclature/IMGT-FRCDRdefinition.html). In certain embodiments, the CDRs regions are delineated using the IMGT numbering system.

The terms “substantially homologous” or “substantially identical” mean a polypeptide or nucleic acid molecule that exhibits at least 50% homology or identity to a reference amino acid sequence (for example, any one of the amino acid sequences described herein) or nucleic acid sequence (for example, any one of the nucleic acid sequences described herein). For example, such a sequence is at least about 60%, about 65%, about 70%, about 75%, about 80%, about 85%, about 90%, about 95% or even about 99% homologous or identical at the amino acid level or nucleic acid to the sequence used for comparison.

Sequence homology or sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. In an exemplary approach to determining the degree of identity, a BLAST program may be used, with a probability score between e⁻³ and e⁻¹⁰⁰ indicating a closely related sequence.

As used herein, the percent homology between two amino acid sequences is equivalent to the percent identity between the two sequences. The percent identity between the two sequences is a function of the number of identical positions shared by the sequences (i.e., % homology= # of identical positions/total #of positions×100), taking into account the number of gaps, and the length of each gap, which need to be introduced for optimal alignment of the two sequences. The comparison of sequences and determination of percent identity between two sequences can be accomplished using a mathematical algorithm.

The percent homology between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent homology between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6. Additionally or alternatively, the amino acids sequences of the presently disclosed subject matter can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the specified sequences disclosed herein. To obtain gapped alignments for comparison purposes, Gapped BLAST can be utilized as described in Altschul et al., (1997) Nucleic Acids Res. 25 (17): 3389-3402. When utilizing BLAST and Gapped BLAST programs, the default parameters of the respective programs (e.g., XBLAST and NBLAST) can be used.

As used herein, the term “a conservative sequence modification” refers to an amino acid modification that does not significantly affect or alter the binding characteristics of the presently disclosed TCR comprising the amino acid sequence. Conservative modifications can include amino acid substitutions, additions and deletions. Amino acids can be classified into groups according to their physicochemical properties such as charge and polarity. Conservative amino acid substitutions are ones in which the amino acid residue is replaced with an amino acid within the same group. For example, amino acids can be classified by charge: positively-charged amino acids include lysine, arginine, histidine, negatively-charged amino acids include aspartic acid, glutamic acid, neutral charge amino acids include alanine, asparagine, cysteine, glutamine, glycine, isoleucine, leucine, methionine, phenylalanine, proline, serine, threonine, tryptophan, tyrosine, and valine. In addition, amino acids can be classified by polarity: polar amino acids include arginine (basic polar), asparagine, aspartic acid (acidic polar), glutamic acid (acidic polar), glutamine, histidine (basic polar), lysine (basic polar), serine, threonine, and tyrosine; non-polar amino acids include alanine, cysteine, glycine, isoleucine, leucine, methionine, phenylalanine, proline, tryptophan, and valine. Thus, one or more amino acid residues within a CDR region can be replaced with other amino acid residues from the same group and the altered TCR can be tested for retained function (i.e., the functions set forth in (c) through (1) above) using the functional assays described herein. In certain embodiments, no more than one, no more than two, no more than three, no more than four, no more than five residues within a specified sequence or a CDR region are altered.

As used herein, the term “disease” refers to any condition or disorder that damages or interferes with the normal function of a cell, tissue, or organ. Examples of diseases include neoplasm or pathogen infection of a cell.

An “effective amount” (or “therapeutically effective amount”) is an amount sufficient to affect a beneficial or desired clinical result upon treatment. An effective amount can be administered to a subject in one or more doses. In terms of treatment, an effective amount is an amount that is sufficient to palliate, ameliorate, stabilize, reverse or slow the progression of the disease (e.g., a tumor), prevent or delay the recurrence of a tumor, or otherwise reduce the pathological consequences of the disease (e.g., a tumor). The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the immunoresponsive cells administered.

As used herein, the term “tumor” refers to an abnormal mass of tissue that forms when cells grow and divide more than they should or do not die when they should. Tumors include benign tumors and malignant tumors (known as “cancers”). Benign tumors may grow large but do not spread into, or invade, nearby tissues or other parts of the body. Malignant tumors can spread into, or invade, nearby tissues. They can also spread to other parts of the body through the blood and lymph systems. Tumor is also called neoplasm. In certain embodiments, the tumor is cancer.

As used herein, the term “immunoresponsive cell” refers to a cell that functions in an immune response or a progenitor, or progeny thereof.

As used herein, the term “modulate” refers to positively or negatively alter. Exemplary modulations include an about 1%, about 2%, about 5%, about 10%, about 25%, about 50%, about 75%, or about 10⁰% change.

As used herein, the term “increase” refers to alter positively by at least about 5%, including, but not limited to, alter positively by about 5%, by about 10%, by about 25%, by about 30%, by about 50%, by about 75%, or by about 10⁰%.

As used herein, the term “reduce” refers to alter negatively by at least about 5% including, but not limited to, alter negatively by about 5%, by about 10%, by about 25%, by about 30%, by about 50%, by about 75%, or by about 10⁰%.

As used herein, the term “isolated,” “purified,” or “biologically pure” refers to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or polypeptide of the presently disclosed subject matter is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

As used herein, the term “isolated cell” refers to a cell that is separated from the molecular and/or cellular components that naturally accompany the cell.

As used herein, the term “treating” or “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

An “individual” or “subject” herein is a vertebrate, such as a human or non-human animal, for example, a mammal. Mammals include, but are not limited to, humans, primates, farm animals, sport animals, rodents and pets. Non-limiting examples of non-human animal subjects include rodents such as mice, rats, hamsters, guinea pigs, rabbits, dogs, cats, sheep, pigs, goats, cattle, horses; and non-human primates such as apes and monkeys.

As used herein, the terms “recombinant T cell receptor” or “recombinant TCR” refer to a T cell receptor that is produced by genetic engineering (i.e., recombinant DNA technology). In certain embodiments, the term “recombinant TCR” includes TCRs that are expressed in a prokaryotic or eukaryotic cell that contains a cloning vector or expression vector comprising a polynucleotide encoding the TCR. In certain embodiments, the TCR includes at least one amino acid residue not found in a naturally occurring TCR. For example, but without any limitation, a recombinant TCR can have a variable chain or a constant chain that includes an amino acid residue that is not found in the corresponding naturally occurring TCR. In an exemplary embodiment, a recombinant TCR can have an a variable chain or a β variable chain including an amino acid residue that is not found in a naturally occurring TCR. In another exemplary embodiment, a recombinant TCR can have an a constant chain or a β constant chain including an amino acid residue that is not found in a naturally occurring TCR. In certain embodiments, a TCR comprises an alpha or beta constant chain comprising a cysteine modification. Additionally, a recombinant T cell receptor refers to a T cell receptor that is prepared, expressed, generated, and/or isolated using recombinant technologies. Methods and procedures to prepare, express, generate, and/or isolate recombinant TCR are known in the art.

As used herein, the term “neoantigens” or “NeoAgs” refers to peptides derived from the protein products of mutations found in a patient and presented by a patient's complement of human leukocyte antigen (HLA) molecules. As used herein, the term “public neoantigen” refers to neoantigen derived from a common hotspot mutation seen in multiple patients as opposed to a “private neoantigen” which is exclusive to an individual patient.

### 5.2. TP53

TP53 encodes Tumor Protein P53 or cellular tumor antigen p53 (TP53) having a UniProt Reference No: P04637. In certain embodiments, the wild-type form of TP53 comprises the polypeptide sequence set forth in SEQ ID NO: 121. TP53 is a transcription factor that regulates the expression of target genes involved in cell cycle arrest, apoptosis, senescence, DNA repair, and metabolism. TP53 comprises a transactivation domain (TAD), a proline-rich domain (PRD), a DNA-binding domain (DBD), a nuclear localization signal (NLS), a oligomerization domain (OD), and a C-terminal domain (CTD). See Monti et al., Frontiers in oncology 10 (2020): 593383. In certain embodiments, the transactivation domain (TAD) comprises from amino acid 1 to amino acid 61 of SEQ ID NO: 121. In certain embodiments, the proline-rich domain (PRD) comprises from amino acid 62 to amino 93 of SEQ ID NO: 121. In certain embodiments, the DNA-binding domain (DBD) comprises from amino acid 94 to amino acid 290 of SEQ ID NO: 121. In certain embodiments, the nuclear localization signal (NLS) comprises from amino acid 291 to amino acid 324 of SEQ ID NO: 121. In certain embodiments, the oligomerization domain (OD) comprises from amino acid 325 to amino acid 356 of SEQ ID NO: 121. In certain embodiments, the C-terminal domain (CTD) comprises from amino acid 357 to amino acid 393 of SEQ ID NO: 121. TP53 functions as a tumor suppressor protein and plays a role in the formation and growth of tumors and is the most frequently mutated gene in human cancer accounting for over 40% of all subsets of cancer. The majority of TP53 mutations localize in the DNA binding domain and 8 hotspot mutations account for approximately 27% of all cases. In certain embodiments, the TP53 mutation is selected from R175H, G245S, R248Q, R248W, R249S, R273H, R273S, R282W, or a combination thereof. In certain embodiments, the TP53 mutation is R175H. R175H is a commonly mutated amino acid positions. An exemplary polypeptide including the R175H mutation is set forth in SEQ ID NO: 122. SEQ ID Nos: 121 and 122 are provided below.

[SEQ ID NO: 121]
MEEPQSDPSVEPPLSQETFSDLWKLLPENNVLSPLPSQAMDDLMLSPDDIEQWFTEDPGPDEAPRMPEAAPPVAPAP
AAPTPAAPAPAPSWPLSSSVPSQKTYQGSYGFRLGFLHSGTAKSVTCTYSPALNKMFCQLAKTCPVQLWVDSTPPPG
TRVRAMAIYKQSQHMTEVVRRCPHHERCSDSDGLAPPQHLIRVEGNLRVEYLDDRNTFRHSVVVPYEPPEVGSDCTT
IHYNYMCNSSCMGGMNRRPILTIITLEDSSGNLLGRNSFEVRVCACPGRDRRTEEENLRKKGEPHHELPPGSTKRAL
PNNTSSSPQPKKKPLDGEYFTLQIRGRERFEMFRELNEALELKDAQAGKEPGGSRAHSSHLKSKKGQSTSRHKKLMF
KTEGPDSD
[SEQ ID NO: 122]
MEEPQSDPSVEPPLSQETFSDLWKLLPENNVLSPLPSQAMDDLMLSPDDIEQWFTEDPGPDEAPRMPEAAPPVAPAP
AAPTPAAPAPAPSWPLSSSVPSQKTYQGSYGFRLGFLHSGTAKSVTCTYSPALNKMFCQLAKTCPVQLWVDSTPPPG
TRVRAMAIYKQSQHMTEVVRHCPHHERCSDSDGLAPPQHLIRVEGNLRVEYLDDRNTFRHSVVVPYEPPEVGSDCTT
IHYNYMCNSSCMGGMNRRPILTIITLEDSSGNLLGRNSFEVRVCACPGRDRRTEEENLRKKGEPHHELPPGSTKRAL
PNNTSSSPQPKKKPLDGEYFTLQIRGRERFEMFRELNEALELKDAQAGKEPGGSRAHSSHLKSKKGQSTSRHKKLMF
KTEGPDSD

### 5.3. T-Cell Receptor (TCR)

A TCR is a disulfide-linked heterodimeric protein consisting of two variable chains expressed as part of a non-covalent complex with the invariant CD3 chain molecules (CD3δ, CD3ε, CD3γ, CD3ζ). A TCR is found on the surface of T cells, and is responsible for recognizing antigens bound to major histocompatibility complex (MHC) molecules. In certain embodiments, a TCR comprises an α chain and a β chain (encoded by TRA and TRB, respectively). In certain embodiments, a TCR comprises a γ chain and a δ chain (encoded by TRG and TRD, respectively).

Each chain of a TCR comprises two extracellular domains: a variable region and a constant region. The constant region is proximal to the cell membrane, followed by a transmembrane domain and a short cytoplasmic tail (i.e., an intracellular domain). The variable region binds to the peptide/MHC complex. The variable region of both chains each has three complementarity determining regions (CDRs).

In certain embodiments, a TCR can form a receptor complex with three dimeric signaling modules CD3δ/ε, CD3γ/ε and CD247ζ/ζ or δ/η. When a TCR complex engages with its cognate peptide antigen/MHC (peptide/MHC), the T cell expressing the TCR complex is activated.

The presently disclosed subject matter provides recombinant TCRs. In certain embodiments, the recombinant TCR differs from any naturally occurring TCR by at least one amino acid residue. In certain embodiments, the recombinant TCR differs from any naturally occurring TCR by at least 2, 3, 4, 5, 6, 7, 8, 9, 10, 11, 12, 13, 14, 15, 20, 25, 30, 40, 50, 60, 70, 80, 90, 100 or more amino acid residues. In certain embodiments, the recombinant TCR is modified from a naturally occurring TCR by at least one amino acid residue. In certain embodiments, the recombinant TCR is modified from a naturally occurring TCR by at least 2, 3, 4, 5, 6, 7, 8, 9, 10, 11, 12, 13, 14, 15, 20, 25, 30, 40, 50, 60, 70, 80, 90, 100 or more amino acid residues.

In certain embodiments, the presently disclosed TCR targets or binds to a TP53 peptide that comprises a mutation (“a mutant TP53 peptide”). In certain embodiments, the mutation is a point mutation. In certain embodiments, the mutation is a R175 mutation. In certain embodiments, the mutation is a R175H mutation. In certain embodiments, the mutant TP53 peptide comprises or consists of the amino acid sequence set forth in SEQ ID NO: 146. SEQ ID NO: 146 is provided below:

[SEQ ID NO: 146]
HMTEVVRHC

In certain embodiments, the presently disclosed TCR does not bind to a wildtype TP53. In certain embodiments, the presently disclosed TCR does not bind to a wildtype TP53 peptide comprising or consisting of the amino acid sequence set forth in SEQ ID NO: 147. In certain embodiments, the presently disclosed TCR binds to a wildtype TP53. In certain embodiments, the presently disclosed TCR binds to a wildtype TP53 peptide comprising or consisting of the amino acid sequence set forth in SEQ ID NO: 147. SEQ ID NO: 147 is provided below:

[SEQ ID NO: 147]
HMTEVVRRC

In certain embodiments, the presently disclosed TCR targets or binds to a TP53 peptide (e.g., a mutant TP53 peptide) associated with an HLA class I complex, e.g., HLA-A, HLA-B and HLA-C.

In certain embodiments, the presently disclosed TCR targets or binds to a TP53 peptide associated with an HLA-A*02 superfamily (e.g., in an HLA-A*02 superfamily dependent manner). In certain embodiments, the HLA*A02 superfamily members include, but not limited to, alleles and sub-alleles in the HLA-A*02, HLA-A*02:01, HLA-A*02:02, HLA-A*02:03, HLA-A*02:04, HLA-A*02:05, HLA-A*02:06, HLA-A*02:07, HLA-A*02:09, HLA-A*02:11, HLA-A*02:12, HLA-A*02:13, HLA-A*02:0215HLA-A*02:14, HLA-A*02:17, HLA-A*02:71, HLA-A*02:77, HLA-A*68:02, and HLA-A*69:01. In certain embodiments, the presently disclosed TCR targets or binds to a TP53 peptide associated with an HLA-A*02:01 molecule.

### 5.3.1. TCRs

### 5.3.1.1. Variable Regions

In certain embodiments, the extracellular domain of the TCR comprises an α chain variable region comprising a CDR1 comprising the amino acid sequence set forth in SEQ ID NO: 1 or a conservative modification thereof, a CDR2 comprising the amino acid sequence set forth in SEQ ID NO: 2 or a conservative modification thereof, and a CDR3 comprising the amino acid sequence set forth in SEQ ID NO: 3 or a conservative modification thereof. SEQ ID NOS: 1-3 are disclosed in Table 1. In certain embodiments, the α chain variable region comprises a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3.

In certain embodiments, the extracellular domain of the TCR comprises a β chain variable region comprising a CDR1 comprising the amino acid sequence set forth in SEQ ID NO: 4 or a conservative modification thereof, a CDR2 comprising the amino acid sequence set forth in SEQ ID NO: 5 or a conservative modification thereof, and a CDR3 comprising the amino acid sequence set forth in SEQ ID NO: 6 or a conservative modification thereof. SEQ ID NOS: 4-6 are disclosed in Table 1. In certain embodiments, the β chain variable region comprises a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6.

In certain embodiments, the α chain variable region comprises a CDR1 comprising the amino acid sequence set forth in SEQ ID NO: 1 or a conservative modification thereof, a CDR2 comprising the amino acid sequence set forth in SEQ ID NO: 2 or a conservative modification thereof, and a CDR3 comprising the amino acid sequence set forth in SEQ ID NO: 3 or a conservative modification thereof; and the β chain variable region comprises a CDR1 comprising the amino acid sequence set forth in SEQ ID NO: 4 or a conservative modification thereof, a CDR2 comprising the amino acid sequence set forth in SEQ ID NO: 5 or a conservative modification thereof, and a CDR3 comprising the amino acid sequence set forth in SEQ ID NO: 6 or a conservative modification thereof. In certain embodiments, the α chain variable region comprises a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3; and the β chain variable region comprises a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 7. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 7. In certain embodiments, the α chain variable region comprises the amino acid sequence set forth in SEQ ID NO: 7. SEQ ID NO: 7 is provided in Table 1.

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 8. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 8. In certain embodiments, the β chain variable region comprises the amino acid sequence set forth in SEQ ID NO: 8. SEQ ID NO: 8 is provided in Table 1.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 7; and the β chain variable region comprises an amino acid sequence that is at least about 80% (e.g., at least about 85%, at least about 90%, or at least about 95%) homologous or identical to the amino acid sequence set forth in SEQ ID NO: 8. In certain embodiments, the α chain variable region comprises the amino acid sequence set forth in SEQ ID NO: 7; and the β chain variable region comprises the amino acid sequence set forth in SEQ ID NO: 8.

In certain embodiments, the extracellular domain of the TCR comprises an α chain that comprises an α chain variable region and an α chain constant region. In certain embodiments, the α chain comprises an amino acid sequence that is at least about 80% (e.g., at least about 85%, at least about 90%, or at least about 95%) homologous or identical to the amino acid sequence set forth in SEQ ID NO: 9. For example, the α chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 9. In certain embodiments, the α chain comprises the amino acid sequence set forth in SEQ ID NO: 9.

In certain embodiments, the extracellular domain of the TCR comprises a β chain that comprises a β chain variable region and a β chain constant region. In certain embodiments, the β chain comprises an amino acid sequence that is at least about 80% (e.g., at least about 85%, at least about 90%, or at least about 95%) homologous or identical to the amino acid sequence set forth in SEQ ID NO: 10. For example, the β chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 10. In certain embodiments, the β chain comprises the amino acid sequence set forth in SEQ ID NO: 10.

In certain embodiments, the α chain comprises an amino acid sequence that is at least about 80% (e.g., at least about 85%, at least about 90%, or at least about 95%) homologous or identical to the amino acid sequence set forth in SEQ ID NO: 9; and the β chain comprises an amino acid sequence that is at least about 80% (e.g., at least about 85%, at least about 90%, or at least about 95%) homologous or identical to the amino acid sequence set forth in SEQ ID NO: 10. In certain embodiments, the α chain comprises the amino acid sequence set forth in SEQ ID NO: 9; and the β chain comprises the amino acid sequence set forth in SEQ ID NO: 10. In certain embodiments, the TCR is designated as “TCR 1A”. In certain embodiments, the TCR 1A binds to a TP53 peptide comprising a R175H mutation.

In certain embodiments, the CDRs sequences described above including Table 1 are delineated using the IMGT numbering system.

TABLE 1
(TCR 1A)

| CDRs | 1 | 2 | 3 |
|---|---|---|---|
| α-chain | TSINN | IRSNERE | CATGSNYGGSQGNLIF |
| | [SEQ ID NO: 1] | [SEQ ID NO: 2] | [SEQ ID NO: 3] |
| β-chain | SGHAT | FQNNGV | CASSLDYGSSYEQYF |
| | [SEQ ID NO: 4] | [SEQ ID NO: 5] | [SEQ ID NO: 6] |

| | |
|---|---|
| α-chain | METLLGVSLVILWLQLARVNSQQGEEDPQALSIQEGENATMNCSYKTSINNLQWYR |
| variable | QNSGRGLVHLILIRSNEREKHSGRLRVTLDTSKKSSSLLITASRAADTASYFCATG |
| | SNYGGSQGNLIFGKGTKLSVKP [SEQ ID NO: 7] |
| β-chain | MGTRLLCWAALCLLGAELTEAGVAQSPRYKIIEKRQSVAFWCNPISGHATLYWYQQ |
| variable | ILGQGPKLLIQFQNNGVVDDSQLPKDRESAERLKGVDSTLKIQPAKLEDSAVYLCA |
| | SSLDYGSSYEQYFGPGTRLTVT |
| | [SEQ ID NO: 8] |
| Full α- | METLLGVSLVILWLQLARVNSQQGEEDPQALSIQEGENATMNCSYKTSINNLQWYR |
| chain | QNSGRGLVHLILIRSNEREKHSGRLRVTLDTSKKSSSLLITASRAADTASYFCATG |
| | SNYGGSQGNLIFGKGTKLSVKPNIQNPDPAVYQLRDSKSSDKSVCLFTDFDSQTNVSQSK |
| | DSDVYITDKTVLDMRSMDFKSNSAVAWSNKSDFACANAFNNSIIPEDTFFPSPESSCDVKLV |
| | EKSFETDTNLNFQNLSVIGFRILLLKVAGENLLMTLRLWSS |
| | [SEQ ID NO: 9] |
| Full β- | MGTRLLCWAALCLLGAELTEAGVAQSPRYKIIEKRQSVAFWCNPISGHATLYWYQQ |
| chain | ILGQGPKLLIQFQNNGVVDDSQLPKDRFSAERLKGVDSTLKIQPAKLEDSAVYLCA |
| | SSLDYGSSYEQYFGPGTRLTVTEDLNKVFPPEVAVFEPSEAEISHTQKATLVCLATGFFP |
| | DHVELSWWVNGKEVHSGVSTDPQPLKEQPALNDSRYCLSSRLRVSATFWQNPRNHERCQVQF |
| | YGLSENDEWTQDRAKPVTQIVSAEAWGRADCGFTSVSYQQGVLSATILYEILLGKATLYAVL |
| | VSALVLMAMVKRKDF [SEQ ID NO: 10] |

In certain embodiments, the extracellular domain of the TCR comprises an α chain variable region comprising a CDR1 comprising the amino acid sequence set forth in SEQ ID NO: 11 or a conservative modification thereof, a CDR2 comprising the amino acid sequence set forth in SEQ ID NO: 12 or a conservative modification thereof, and a CDR3 comprising the amino acid sequence set forth in SEQ ID NO: 13 or a conservative modification thereof. SEQ ID NOS: 11-13 are disclosed in Table 2. In certain embodiments, the α chain variable region comprises a CDR1 comprising the amino acid sequence set forth in SEQ ID NO: 11, a CDR2 comprising the amino acid sequence set forth in SEQ ID NO: 12, and a CDR3 comprising the amino acid sequence set forth in SEQ ID NO: 13.

In certain embodiments, the extracellular domain of the TCR comprises a β chain variable region comprising a CDR1 comprising the amino acid sequence set forth in SEQ ID NO: 14 or a conservative modification thereof, a CDR2 comprising the amino acid sequence set forth in SEQ ID NO: 15 or a conservative modification thereof, and a CDR3 comprising the amino acid sequence set forth in SEQ ID NO: 16 or a conservative modification thereof. SEQ ID NOS: 14-16 are disclosed in Table 2. In certain embodiments, the β chain variable region comprises a CDR1 comprising the amino acid sequence set forth in SEQ ID NO: 14, a CDR2 comprising the amino acid sequence set forth in SEQ ID NO: 15, and a CDR3 comprising the amino acid sequence set forth in SEQ ID NO: 16.

In certain embodiments, the α chain variable region comprises a CDR1 comprising the amino acid sequence set forth in SEQ ID NO: 11 or a conservative modification thereof, a CDR2 comprising the amino acid sequence set forth in SEQ ID NO: 12 or a conservative modification thereof, and a CDR3 comprising the amino acid sequence set forth in SEQ ID NO: 13 or a conservative modification thereof; and the β chain variable region comprises a CDR1 comprising the amino acid sequence set forth in SEQ ID NO: 14 or a conservative modification thereof, a CDR2 comprising the amino acid sequence set forth in SEQ ID NO: 15 or a conservative modification thereof, and a CDR3 comprising the amino acid sequence set forth in SEQ ID NO: 16 or a conservative modification thereof. In certain embodiments, the α chain variable region comprises a CDR1 comprising the amino acid sequence set forth in SEQ ID NO: 11, a CDR2 comprising the amino acid sequence set forth in SEQ ID NO: 12, and a CDR3 comprising the amino acid sequence set forth in SEQ ID NO: 13; and the β chain variable region comprises a CDR1 comprising the amino acid sequence set forth in SEQ ID NO: 14, a CDR2 comprising the amino acid sequence set forth in SEQ ID NO: 15, and a CDR3 comprising the amino acid sequence set forth in SEQ ID NO: 16.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 17. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 17. In certain embodiments, the α chain variable region comprises the amino acid sequence set forth in SEQ ID NO: 17. SEQ ID NO: 17 is provided in Table 2.

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 18. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 18. In certain embodiments, the β chain variable region comprises the amino acid sequence set forth in SEQ ID NO: 18. SEQ ID NO: 18 is provided in Table 2.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 17; and the β chain variable region comprises an amino acid sequence that is at least about 80% (e.g., at least about 85%, at least about 90%, or at least about 95%) homologous or identical to the amino acid sequence set forth in SEQ ID NO: 18. In certain embodiments, the α chain variable region comprises the amino acid sequence set forth in SEQ ID NO: 17; and the β chain variable region comprises the amino acid sequence set forth in SEQ ID NO: 18.

In certain embodiments, the extracellular domain of the TCR comprises an α chain that comprises an α chain variable region and an α chain constant region. In certain embodiments, the α chain comprises an amino acid sequence that is at least about 80% (e.g., at least about 85%, at least about 90%, or at least about 95%) homologous or identical to the amino acid sequence set forth in SEQ ID NO: 19. For example, the α chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 19. In certain embodiments, the α chain comprises the amino acid sequence set forth in SEQ ID NO: 19.

In certain embodiments, the extracellular domain of the TCR comprises a β chain that comprises a β chain variable region and a β chain constant region. In certain embodiments, the β chain comprises an amino acid sequence that is at least about 80% (e.g., at least about 85%, at least about 90%, or at least about 95%) homologous or identical to the amino acid sequence set forth in SEQ ID NO: 20. For example, the β chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 20. In certain embodiments, the β chain comprises the amino acid sequence set forth in SEQ ID NO: 20.

In certain embodiments, the α chain comprises an amino acid sequence that is at least about 80% (e.g., at least about 85%, at least about 90%, or at least about 95%) homologous or identical to the amino acid sequence set forth in SEQ ID NO: 19; and the β chain comprises an amino acid sequence that is at least about 80% (e.g., at least about 85%, at least about 90%, or at least about 95%) homologous or identical to the amino acid sequence set forth in SEQ ID NO: 20. In certain embodiments, the α chain comprises the amino acid sequence set forth in SEQ ID NO: 19; and the β chain comprises the amino acid sequence set forth in SEQ ID NO: 20. In certain embodiments, the TCR is designated as “TCR 1B”. In certain embodiments, the TCR 1B binds to a TP53 peptide comprising a R175H mutation.

In certain embodiments, the CDRs sequences described above including Table 2 are delineated using the IMGT numbering system.

TABLE 2
(TCR 1B)

| CDRs | 1 | 2 | 3 |
|---|---|---|---|
| α-chain | TSDPSYG | QGSYDQQN | CAMREGFSGGYNKLIF |
| | [SEQ ID NO: 11] | [SEQ ID NO: 12] | [SEQ ID NO: 13] |
| β-chain | SGHKS | YYEKEE | CASSDDTGTTKSSGANVLTF |
| | [SEQ ID NO: 14] | [SEQ ID NO: 15] | [SEQ ID NO: 16] |

| | |
|---|---|
| α-chain | MSLSSLLKVVTASLWLGPGIAQKITQTQPGMFVQEKEAVTLDCTYDTSDPSYGLEW |
| variable | YKQPSSGEMIFLIYQGSYDQQNATEGRYSLNFQKARKSANLVISASQLGDSAMYFC |
| | AMREGFSGGYNKLIFGAGTRLAVHP [SEQ ID NO: 17] |
| β-chain | MGPGLLCWVLLCLLGAGPVDAGVTQSPTHLIKTRGQQVTLRCSPISGHKSVSWYQQ |
| variable | VLGQGPQFIFQYYEKEERGRGNFPDRESARQFPNYSSELNVNALLLGDSALYLCAS |
| | SDDTGTTKSSGANVLTFGAGSRLTVL [SEQ ID NO: 18] |
| Full α- | MSLSSLLKVVTASLWLGPGIAQKITQTQPGMFVQEKEAVTLDCTYDTSDPSYGLFW |
| chain | YKQPSSGEMIFLIYQGSYDQQNATEGRYSLNFQKARKSANLVISASQLGDSAMYFC |
| | AMREGFSGGYNKLIFGAGTRLAVHPNIQNPDPAVYQLRDSKSSDKSVCLFTDFDSQTNV |
| | SQSKDSDVYITDKTVLDMRSMDFKSNSAVAWSNKSDFACANAFNNSIIPEDTFFPSPESSCD |
| | VKLVEKSFETDTNLNFQNLSVIGFRILLLKVAGENLLMTLRLWSS [SEQ ID NO: 19] |
| Full β- | MGPGLLCWVLLCLLGAGPVDAGVTQSPTHLIKTRGQQVTLRCSPISGHKSVSWYQQ |
| chain | VLGQGPQFIFQYYEKEERGRGNFPDRESARQFPNYSSELNVNALLLGDSALYLCAS |
| | SDDTGTTKSSGANVLTFGAGSRLTVLEDLNKVEPPEVAVFEPSEAEISHTQKATLVCLA |
| | TGFFPDHVELSWWVNGKEVHSGVSTDPQPLKEQPALNDSRYCLSSRLRVSATFWQNPRNHER |
| | CQVQFYGLSENDEWTQDRAKPVTQIVSAEAWGRADCGFTSVSYQQGVLSATILYEILLGKAT |
| | LYAVLVSALVLMAMVKRKDF |
| | [SEQ ID NO: 20] |

In certain embodiments, the extracellular domain of the TCR comprises an α chain variable region comprising a CDR1 comprising the amino acid sequence set forth in SEQ ID NO: 21 or a conservative modification thereof, a CDR2 comprising the amino acid sequence set forth in SEQ ID NO: 22 or a conservative modification thereof, and a CDR3 comprising the amino acid sequence set forth in SEQ ID NO: 23 or a conservative modification thereof. SEQ ID NOS: 21-23 are disclosed in Table 3. In certain embodiments, the α chain variable region comprises a CDR1 comprising the amino acid sequence set forth in SEQ ID NO: 21, a CDR2 comprising the amino acid sequence set forth in SEQ ID NO: 22, and a CDR3 comprising the amino acid sequence set forth in SEQ ID NO: 23.

In certain embodiments, the extracellular domain of the TCR comprises a β chain variable region comprising a CDR1 comprising the amino acid sequence set forth in SEQ ID NO: 24 or a conservative modification thereof, a CDR2 comprising the amino acid sequence set forth in SEQ ID NO: 25 or a conservative modification thereof, and a CDR3 comprising the amino acid sequence set forth in SEQ ID NO: 26 or a conservative modification thereof. SEQ ID NOS: 24-26 are disclosed in Table 3. In certain embodiments, the β chain variable region comprises a CDR1 comprising the amino acid sequence set forth in SEQ ID NO: 24, a CDR2 comprising the amino acid sequence set forth in SEQ ID NO: 25, and a CDR3 comprising the amino acid sequence set forth in SEQ ID NO: 26.

In certain embodiments, the α chain variable region comprises a CDR1 comprising the amino acid sequence set forth in SEQ ID NO: 21 or a conservative modification thereof, a CDR2 comprising the amino acid sequence set forth in SEQ ID NO: 22 or a conservative modification thereof, and a CDR3 comprising the amino acid sequence set forth in SEQ ID NO: 23 or a conservative modification thereof; and the β chain variable region comprises a CDR1 comprising the amino acid sequence set forth in SEQ ID NO: 24 or a conservative modification thereof, a CDR2 comprising the amino acid sequence set forth in SEQ ID NO: 25 or a conservative modification thereof, and a CDR3 comprising the amino acid sequence set forth in SEQ ID NO: 26 or a conservative modification thereof. In certain embodiments, the α chain variable region comprises a CDR1 comprising the amino acid sequence set forth in SEQ ID NO: 21, a CDR2 comprising the amino acid sequence set forth in SEQ ID NO: 22, and a CDR3 comprising the amino acid sequence set forth in SEQ ID NO: 23; and the β chain variable region comprises a CDR1 comprising the amino acid sequence set forth in SEQ ID NO: 24, a CDR2 comprising the amino acid sequence set forth in SEQ ID NO: 25, and a CDR3 comprising the amino acid sequence set forth in SEQ ID NO: 26.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 27. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 27. In certain embodiments, the α chain variable region comprises the amino acid sequence set forth in SEQ ID NO: 27. SEQ ID NO: 27 is provided in Table 3.

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 28. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 28. In certain embodiments, the β chain variable region comprises the amino acid sequence set forth in SEQ ID NO: 28. SEQ ID NO: 28 is provided in Table 3.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 27; and the β chain variable region comprises an amino acid sequence that is at least about 80% (e.g., at least about 85%, at least about 90%, or at least about 95%) homologous or identical to the amino acid sequence set forth in SEQ ID NO: 28. In certain embodiments, the α chain variable region comprises the amino acid sequence set forth in SEQ ID NO: 27; and the β chain variable region comprises the amino acid sequence set forth in SEQ ID NO: 28.

In certain embodiments, the extracellular domain of the TCR comprises an α chain that comprises an α chain variable region and an α chain constant region. In certain embodiments, the α chain comprises an amino acid sequence that is at least about 80% (e.g., at least about 85%, at least about 90%, or at least about 95%) homologous or identical to the amino acid sequence set forth in SEQ ID NO: 29. For example, the α chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 29. In certain embodiments, the α chain comprises the amino acid sequence set forth in SEQ ID NO: 29.

In certain embodiments, the extracellular domain of the TCR comprises a β chain that comprises a β chain variable region and a β chain constant region. In certain embodiments, the β chain comprises an amino acid sequence that is at least about 80% (e.g., at least about 85%, at least about 90%, or at least about 95%) homologous or identical to the amino acid sequence set forth in SEQ ID NO: 30. For example, the β chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 30. In certain embodiments, the β chain comprises the amino acid sequence set forth in SEQ ID NO: 30.

In certain embodiments, the α chain comprises an amino acid sequence that is at least about 80% (e.g., at least about 85%, at least about 90%, or at least about 95%) homologous or identical to the amino acid sequence set forth in SEQ ID NO: 29; and the β chain comprises an amino acid sequence that is at least about 80% (e.g., at least about 85%, at least about 90%, or at least about 95%) homologous or identical to the amino acid sequence set forth in SEQ ID NO: 30. In certain embodiments, the α chain comprises the amino acid sequence set forth in SEQ ID NO: 29; and the β chain comprises the amino acid sequence set forth in SEQ ID NO: 30. In certain embodiments, the TCR is designated as “TCR 1C”. In certain embodiments, the TCR 1C binds to a TP53 peptide comprising a R175H mutation.

In certain embodiments, the CDRs sequences described above including Table 3 are delineated using the IMGT numbering system.

TABLE 3
(TCR 1C)

| CDRs | 1 | 2 | 3 |
|---|---|---|---|
| α-chain | NSMFDY | ISSIKDK | CAATVGLAGNMLTE |
| | [SEQ ID NO: 21] | [SEQ ID NO: 22] | [SEQ ID NO: 23] |
| β-chain | SGDLS | YYNGEE | CASSTPLSGSSTDTQYF |
| | [SEQ ID NO: 24] | [SEQ ID NO: 25] | [SEQ ID NO: 26] |

| | |
|---|---|
| α-chain | MAMLLGASVLILWLQPDWVNSQQKNDDQQVKQNSPSLSVQEGRISILNCDYTNSME |
| variable | DYFLWYKKYPAEGPTFLISISSIKDKNEDGRFTVFLNKSAKHLSLHIVPSQPGDSA |
| | VYFCAATVGLAGNMLTFGGGTRLMVKP [SEQ ID NO: 27] |
| β-chain | MGFRLLCCVAFCLLGAGPVDSGVTQTPKHLITATGQRVTLRCSPRSGDLSVYWYQQ |
| variable | SLDQGLQFLIQYYNGEERAKGNILERFSAQQFPDLHSELNLSSLELGDSALYFCAS |
| | STPLSGSSTDTQYFGPGTRLTVL [SEQ ID NO: 28] |
| Full α- | MAMLLGASVLILWLQPDWVNSQQKNDDQQVKQNSPSLSVQEGRISILNCDYTNSMF |
| chain | DYFLWYKKYPAEGPTFLISISSIKDKNEDGRFTVELNKSAKHLSLHIVPSQPGDSA |
| | VYFCAATVGLAGNMLTFGGGTRLMVKPNIQNPDPAVYQLRDSKSSDKSVCLFTDFDSQT |
| | NVSQSKDSDVYITDKTVLDMRSMDFKSNSAVAWSNKSDFACANAFNNSIIPEDTFFPSPESS |
| | CDVKLVEKSFETDTNLNFQNLSVIGFRILLLKVAGENLLMTLRLWSS [SEQ ID NO: 29] |
| Full β- | MGFRLLCCVAFCLLGAGPVDSGVTQTPKHLITATGQRVTLRCSPRSGDLSVYWYQQ |
| chain | SLDQGLQFLIQYYNGEERAKGNILERFSAQQFPDLHSELNLSSLELGDSALYFCAS |
| | STPLSGSSTDTQYFGPGTRLTVLEDLNKVFPPEVAVFEPSEAEISHTQKATLVCLATGFF |
| | PDHVELSWWVNGKEVHSGVSTDPQPLKEQPALNDSRYCLSSRLRVSATFWQNPRNHERCQVQ |
| | FYGLSENDEWTQDRAKPVTQIVSAEAWGRADCGFTSVSYQQGVLSATILYEILLGKATLYAV |
| | LVSALVLMAMVKRKDF [SEQ ID NO: 30] |

In certain embodiments, the extracellular domain of the TCR comprises an α chain variable region comprising a CDR1 comprising the amino acid sequence set forth in SEQ ID NO: 31 or a conservative modification thereof, a CDR2 comprising the amino acid sequence set forth in SEQ ID NO: 32 or a conservative modification thereof, and a CDR3 comprising the amino acid sequence set forth in SEQ ID NO: 33 or a conservative modification thereof. SEQ ID NOS: 31-33 are disclosed in Table 4. In certain embodiments, the α chain variable region comprises a CDR1 comprising the amino acid sequence set forth in SEQ ID NO: 31, a CDR2 comprising the amino acid sequence set forth in SEQ ID NO: 32, and a CDR3 comprising the amino acid sequence set forth in SEQ ID NO: 33.

In certain embodiments, the extracellular domain of the TCR comprises a β chain variable region comprising a CDR1 comprising the amino acid sequence set forth in SEQ ID NO: 34 or a conservative modification thereof, a CDR2 comprising the amino acid sequence set forth in SEQ ID NO: 35 or a conservative modification thereof, and a CDR3 comprising the amino acid sequence set forth in SEQ ID NO: 36 or a conservative modification thereof. SEQ ID NOS: 34-36 are disclosed in Table 4. In certain embodiments, the β chain variable region comprises a CDR1 comprising the amino acid sequence set forth in SEQ ID NO: 34, a CDR2 comprising the amino acid sequence set forth in SEQ ID NO: 35, and a CDR3 comprising the amino acid sequence set forth in SEQ ID NO: 36.

In certain embodiments, the α chain variable region comprises a CDR1 comprising the amino acid sequence set forth in SEQ ID NO: 31 or a conservative modification thereof, a CDR2 comprising the amino acid sequence set forth in SEQ ID NO: 32 or a conservative modification thereof, and a CDR3 comprising the amino acid sequence set forth in SEQ ID NO: 33 or a conservative modification thereof; and the β chain variable region comprises a CDR1 comprising the amino acid sequence set forth in SEQ ID NO: 34 or a conservative modification thereof, a CDR2 comprising the amino acid sequence set forth in SEQ ID NO: 35 or a conservative modification thereof, and a CDR3 comprising the amino acid sequence set forth in SEQ ID NO: 36 or a conservative modification thereof. In certain embodiments, the α chain variable region comprises a CDR1 comprising the amino acid sequence set forth in SEQ ID NO: 31, a CDR2 comprising the amino acid sequence set forth in SEQ ID NO: 32, and a CDR3 comprising the amino acid sequence set forth in SEQ ID NO: 33; and the β chain variable region comprises a CDR1 comprising the amino acid sequence set forth in SEQ ID NO: 34, a CDR2 comprising the amino acid sequence set forth in SEQ ID NO: 35, and a CDR3 comprising the amino acid sequence set forth in SEQ ID NO: 36.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 37. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 37. In certain embodiments, the α chain variable region comprises the amino acid sequence set forth in SEQ ID NO: 37. SEQ ID NO: 37 is provided in Table 4.

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 38. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 38. In certain embodiments, the β chain variable region comprises the amino acid sequence set forth in SEQ ID NO: 38. SEQ ID NO: 38 is provided in Table 4.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 37; and the β chain variable region comprises an amino acid sequence that is at least about 80% (e.g., at least about 85%, at least about 90%, or at least about 95%) homologous or identical to the amino acid sequence set forth in SEQ ID NO: 38. In certain embodiments, the α chain variable region comprises the amino acid sequence set forth in SEQ ID NO: 37; and the β chain variable region comprises the amino acid sequence set forth in SEQ ID NO: 38.

In certain embodiments, the extracellular domain of the TCR comprises an α chain that comprises an α chain variable region and an α chain constant region. In certain embodiments, the α chain comprises an amino acid sequence that is at least about 80% (e.g., at least about 85%, at least about 90%, or at least about 95%) homologous or identical to the amino acid sequence set forth in SEQ ID NO: 39. For example, the α chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 39. In certain embodiments, the α chain comprises the amino acid sequence set forth in SEQ ID NO: 39.

In certain embodiments, the extracellular domain of the TCR comprises a β chain that comprises a β chain variable region and a β chain constant region. In certain embodiments, the β chain comprises an amino acid sequence that is at least about 80% (e.g., at least about 85%, at least about 90%, or at least about 95%) homologous or identical to the amino acid sequence set forth in SEQ ID NO: 40. For example, the β chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 40. In certain embodiments, the β chain comprises the amino acid sequence set forth in SEQ ID NO: 40.

In certain embodiments, the α chain comprises an amino acid sequence that is at least about 80% (e.g., at least about 85%, at least about 90%, or at least about 95%) homologous or identical to the amino acid sequence set forth in SEQ ID NO: 39; and the β chain comprises an amino acid sequence that is at least about 80% (e.g., at least about 85%, at least about 90%, or at least about 95%) homologous or identical to the amino acid sequence set forth in SEQ ID NO: 40. In certain embodiments, the α chain comprises the amino acid sequence set forth in SEQ ID NO: 39; and the β chain comprises the amino acid sequence set forth in SEQ ID NO: 40. In certain embodiments, the TCR is designated as “TCR 1D”. In certain embodiments, the TCR 1D binds to a TP53 peptide comprising a R175H mutation.

In certain embodiments, the CDRs sequences described above including Table 4 are delineated using the IMGT numbering system.

TABLE 4
(TCR 1D)

| CDRs | 1 | 2 | 3 |
|---|---|---|---|
| α-chain | VSGLRG | LYSAGEE | CAVHSNYQLIW |
| | [SEQ ID NO: 31] | [SEQ ID NO: 32] | [SEQ ID NO: 33] |
| β-chain | SGDLS | YYNGEE | CASSTPLRTRGTDTQYF |
| | [SEQ ID NO: 34] | [SEQ ID NO: 35] | [SEQ ID NO: 36] |

| | |
|---|---|
| α-chain | MEKMLECAFIVLWLQLGWLSGEDQVTQSPEALRLQEGESSSLNCSYTVSGLRGLEW |
| variable | YRQDPGKGPEFLFTLYSAGEEKEKERLKATLTKKESFLHITAPKPEDSATYLCAVH |
| | SNYQLIWGAGTKLIIKP [SEQ ID NO: 37] |
| β-chain | MGFRLLCCVAFCLLGAGPVDSGVTQTPKHLITATGQRVTLRCSPRSGDLSVYWYQQ |
| variable | SLDQGLQFLIHYYNGEERAKGNILERFSAQQFPDLHSELNLSSLELGDSALYFCAS |
| | STPLRTRGTDTQYFGPGTRLTVL |
| | [SEQ ID NO: 38] |
| Full α- | MEKMLECAFIVLWLQLGWLSGEDQVTQSPEALRLQEGESSSLNCSYTVSGLRGLFW |
| chain | YRQDPGKGPEFLFTLYSAGEEKEKERLKATLTKKESFLHITAPKPEDSATYLCAVH |
| | SNYQLIWGAGTKLIIKPNIQNPDPAVYQLRDSKSSDKSVCLFTDFDSQTNVSQSKDSDVY |
| | ITDKTVLDMRSMDFKSNSAVAWSNKSDFACANAFNNSIIPEDTFFPSPESSCDVKLVEKSFE |
| | TDTNLNFQNLSVIGFRILLLKVAGENLLMTLRLWSS [SEQ ID NO: 39] |
| Full β- | MGFRLLCCVAFCLLGAGPVDSGVTQTPKHLITATGQRVTLRCSPRSGDLSVYWYQQ |
| chain | SLDQGLQFLIHYYNGEERAKGNILERFSAQQFPDLHSELNLSSLELGDSALYFCAS |
| | STPLRTRGTDTQYFGPGTRLTVLEDLNKVFPPEVAVFEPSEAEISHTQKATLVCLATGFF |
| | PDHVELSWWVNGKEVHSGVSTDPQPLKEQPALNDSRYCLSSRLRVSATFWQNPRNHERCQVQ |
| | FYGLSENDEWTQDRAKPVTQIVSAEAWGRADCGFTSVSYQQGVLSATILYEILLGKATLYAV |
| | LVSALVLMAMVKRKDF [SEQ ID NO: 40] |

In certain embodiments, the extracellular domain of the TCR comprises an α chain variable region comprising a CDR1 comprising the amino acid sequence set forth in SEQ ID NO: 41 or a conservative modification thereof, a CDR2 comprising the amino acid sequence set forth in SEQ ID NO: 42 or a conservative modification thereof, and a CDR3 comprising the amino acid sequence set forth in SEQ ID NO: 43 or a conservative modification thereof. SEQ ID NOS: 41-43 are disclosed in Table 5. In certain embodiments, the α chain variable region comprises a CDR1 comprising the amino acid sequence set forth in SEQ ID NO: 41, a CDR2 comprising the amino acid sequence set forth in SEQ ID NO: 42, and a CDR3 comprising the amino acid sequence set forth in SEQ ID NO: 43.

In certain embodiments, the extracellular domain of the TCR comprises a β chain variable region comprising a CDR1 comprising the amino acid sequence set forth in SEQ ID NO: 44 or a conservative modification thereof, a CDR2 comprising the amino acid sequence set forth in SEQ ID NO: 45 or a conservative modification thereof, and a CDR3 comprising the amino acid sequence set forth in SEQ ID NO: 46 or a conservative modification thereof. SEQ ID NOS: 44-46 are disclosed in Table 5. In certain embodiments, the β chain variable region comprises a CDR1 comprising the amino acid sequence set forth in SEQ ID NO: 44, a CDR2 comprising the amino acid sequence set forth in SEQ ID NO: 45, and a CDR3 comprising the amino acid sequence set forth in SEQ ID NO: 46.

In certain embodiments, the α chain variable region comprises a CDR1 comprising the amino acid sequence set forth in SEQ ID NO: 41 or a conservative modification thereof, a CDR2 comprising the amino acid sequence set forth in SEQ ID NO: 42 or a conservative modification thereof, and a CDR3 comprising the amino acid sequence set forth in SEQ ID NO: 43 or a conservative modification thereof; and the β chain variable region comprises a CDR1 comprising the amino acid sequence set forth in SEQ ID NO: 44 or a conservative modification thereof, a CDR2 comprising the amino acid sequence set forth in SEQ ID NO: 45 or a conservative modification thereof, and a CDR3 comprising the amino acid sequence set forth in SEQ ID NO: 46 or a conservative modification thereof. In certain embodiments, the α chain variable region comprises a CDR1 comprising the amino acid sequence set forth in SEQ ID NO: 41, a CDR2 comprising the amino acid sequence set forth in SEQ ID NO: 42, and a CDR3 comprising the amino acid sequence set forth in SEQ ID NO: 43; and the β chain variable region comprises a CDR1 comprising the amino acid sequence set forth in SEQ ID NO: 44, a CDR2 comprising the amino acid sequence set forth in SEQ ID NO: 45, and a CDR3 comprising the amino acid sequence set forth in SEQ ID NO: 46.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 47. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 47. In certain embodiments, the α chain variable region comprises the amino acid sequence set forth in SEQ ID NO: 47. SEQ ID NO: 47 is provided in Table 5.

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 48. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 48. In certain embodiments, the β chain variable region comprises the amino acid sequence set forth in SEQ ID NO: 48. SEQ ID NO: 48 is provided in Table 5.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 47; and the β chain variable region comprises an amino acid sequence that is at least about 80% (e.g., at least about 85%, at least about 90%, or at least about 95%) homologous or identical to the amino acid sequence set forth in SEQ ID NO: 48. In certain embodiments, the α chain variable region comprises the amino acid sequence set forth in SEQ ID NO: 47; and the β chain variable region comprises the amino acid sequence set forth in SEQ ID NO: 48.

In certain embodiments, the extracellular domain of the TCR comprises an α chain that comprises an α chain variable region and an α chain constant region. In certain embodiments, the α chain comprises an amino acid sequence that is at least about 80% (e.g., at least about 85%, at least about 90%, or at least about 95%) homologous or identical to the amino acid sequence set forth in SEQ ID NO: 49. For example, the α chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 49. In certain embodiments, the α chain comprises the amino acid sequence set forth in SEQ ID NO: 49.

In certain embodiments, the extracellular domain of the TCR comprises a β chain that comprises a β chain variable region and a β chain constant region. In certain embodiments, the β chain comprises an amino acid sequence that is at least about 80% (e.g., at least about 85%, at least about 90%, or at least about 95%) homologous or identical to the amino acid sequence set forth in SEQ ID NO: 50. For example, the β chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 50. In certain embodiments, the β chain comprises the amino acid sequence set forth in SEQ ID NO: 50.

In certain embodiments, the α chain comprises an amino acid sequence that is at least about 80% (e.g., at least about 85%, at least about 90%, or at least about 95%) homologous or identical to the amino acid sequence set forth in SEQ ID NO: 49; and the β chain comprises an amino acid sequence that is at least about 80% (e.g., at least about 85%, at least about 90%, or at least about 95%) homologous or identical to the amino acid sequence set forth in SEQ ID NO: 50. In certain embodiments, the α chain comprises the amino acid sequence set forth in SEQ ID NO: 49; and the β chain comprises the amino acid sequence set forth in SEQ ID NO: 50. In certain embodiments, the TCR is designated as “TCR 3C”. In certain embodiments, the TCR 3C binds to a TP53 peptide comprising a R175H mutation.

In certain embodiments, the CDRs sequences described above including Table 5 are delineated using the IMGT numbering system.

TABLE 5
(TCR 3C)

| CDRs | 1 | 2 | 3 |
|---|---|---|---|
| α-chain | DSSSTY | IFSNMDM | CAEGYGGKLIF |
| | [SEQ ID NO: 41] | [SEQ ID NO: 42] | [SEQ ID NO: 43] |
| β-chain | SGHTA | FQGTGA | CASSSKTGVVGEQFF |
| | [SEQ ID NO: 44] | [SEQ ID NO: 45] | [SEQ ID NO: 46] |

| | |
|---|---|
| α-chain | MKTFAGFSFLFLWLQLDCMSRGEDVEQSLFLSVREGDSSVINCTYTDSSSTYLYWY |
| variable | KQEPGAGLQLLTYIFSNMDMKQDQRLTVLLNKKDKHLSLRIADTQTGDSAIYFCAE |
| | GYGGKLIFGQGTELSVKP [SEQ ID NO: 47] |
| β-chain | MGTRLLCWAALCLLGADHTGAGVSQTPSNKVTEKGKYVELRCDPISGHTALYWYRQ |
| variable | SLGQGPEFLIYFQGTGAADDSGLPNDREFAVRPEGSVSTLKIQRTERGDSAVYLCA |
| | SSSKTGVVGEQFFGPGTRLTVL [SEQ ID NO: 48] |
| Full α- | MKTFAGESFLFLWLQLDCMSRGEDVEQSLFLSVREGDSSVINCTYTDSSSTYLYWY |
| chain | KQEPGAGLQLLTYIFSNMDMKQDQRLTVLLNKKDKHLSLRIADTQTGDSAIYFCAE |
| | GYGGKLIFGQGTELSVKPNIQNPDPAVYQLRDSKSSDKSVCLFTDFDSQTNVSQSKDSDV |
| | YITDKTVLDMRSMDFKSNSAVAWSNKSDFACANAFNNSIIPEDTFFPSPESSCDVKLVEKSF |
| | ETDTNLNFQNLSVIGFRILLLKVAGENLLMTLRLWSS [SEQ ID NO: 49] |
| Full β- | MGTRLLCWAALCLLGADHTGAGVSQTPSNKVTEKGKYVELRCDPISGHTALYWYRQ |
| chain | SLGQGPEFLIYFQGTGAADDSGLPNDRFFAVRPEGSVSTLKIQRTERGDSAVYLCA |
| | SSSKTGVVGEQFFGPGTRLTVLEDLNKVFPPEVAVFEPSEAEISHTQKATLVCLATGFFP |
| | DHVELSWWVNGKEVHSGVSTDPQPLKEQPALNDSRYCLSSRLRVSATFWQNPRNHFRCQVQF |
| | YGLSENDEWTQDRAKPVTQIVSAEAWGRADCGFTSVSYQQGVLSATILYEILLGKATLYAVL |
| | VSALVLMAMVKRKDF [SEQ ID NO: 50] |

In certain embodiments, the extracellular domain of the TCR comprises an α chain variable region comprising a CDR1 comprising the amino acid sequence set forth in SEQ ID NO: 51 or a conservative modification thereof, a CDR2 comprising the amino acid sequence set forth in SEQ ID NO: 52 or a conservative modification thereof, and a CDR3 comprising the amino acid sequence set forth in SEQ ID NO: 53 or a conservative modification thereof. SEQ ID NOS: 51-53 are disclosed in Table 6. In certain embodiments, the α chain variable region comprises a CDR1 comprising the amino acid sequence set forth in SEQ ID NO: 51, a CDR2 comprising the amino acid sequence set forth in SEQ ID NO: 52, and a CDR3 comprising the amino acid sequence set forth in SEQ ID NO: 53.

In certain embodiments, the extracellular domain of the TCR comprises a β chain variable region comprising a CDR1 comprising the amino acid sequence set forth in SEQ ID NO: 54 or a conservative modification thereof, a CDR2 comprising the amino acid sequence set forth in SEQ ID NO: 55 or a conservative modification thereof, and a CDR3 comprising the amino acid sequence set forth in SEQ ID NO: 56 or a conservative modification thereof. SEQ ID NOS: 54-56 are disclosed in Table 6. In certain embodiments, the β chain variable region comprises a CDR1 comprising the amino acid sequence set forth in SEQ ID NO: 54, a CDR2 comprising the amino acid sequence set forth in SEQ ID NO: 55, and a CDR3 comprising the amino acid sequence set forth in SEQ ID NO: 56.

In certain embodiments, the α chain variable region comprises a CDR1 comprising the amino acid sequence set forth in SEQ ID NO: 51 or a conservative modification thereof, a CDR2 comprising the amino acid sequence set forth in SEQ ID NO: 52 or a conservative modification thereof, and a CDR3 comprising the amino acid sequence set forth in SEQ ID NO: 53 or a conservative modification thereof, and the β chain variable region comprises a CDR1 comprising the amino acid sequence set forth in SEQ ID NO: 54 or a conservative modification thereof, a CDR2 comprising the amino acid sequence set forth in SEQ ID NO: 55 or a conservative modification thereof, and a CDR3 comprising the amino acid sequence set forth in SEQ ID NO: 56 or a conservative modification thereof. In certain embodiments, the α chain variable region comprises a CDR1 comprising the amino acid sequence set forth in SEQ ID NO: 51, a CDR2 comprising the amino acid sequence set forth in SEQ ID NO: 52, and a CDR3 comprising the amino acid sequence set forth in SEQ ID NO: 53; and the β chain variable region comprises a CDR1 comprising the amino acid sequence set forth in SEQ ID NO: 54, a CDR2 comprising the amino acid sequence set forth in SEQ ID NO: 55, and a CDR3 comprising the amino acid sequence set forth in SEQ ID NO: 56.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 57. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 57. In certain embodiments, the α chain variable region comprises the amino acid sequence set forth in SEQ ID NO: 57. SEQ ID NO: 57 is provided in Table 6.

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 58. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 58. In certain embodiments, the β chain variable region comprises the amino acid sequence set forth in SEQ ID NO: 58. SEQ ID NO: 58 is provided in Table 6.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 57; and the β chain variable region comprises an amino acid sequence that is at least about 80% (e.g., at least about 85%, at least about 90%, or at least about 95%) homologous or identical to the amino acid sequence set forth in SEQ ID NO: 58. In certain embodiments, the α chain variable region comprises the amino acid sequence set forth in SEQ ID NO: 57; and the β chain variable region comprises the amino acid sequence set forth in SEQ ID NO: 58.

In certain embodiments, the extracellular domain of the TCR comprises an α chain that comprises an α chain variable region and an α chain constant region. In certain embodiments, the α chain comprises an amino acid sequence that is at least about 80% (e.g., at least about 85%, at least about 90%, or at least about 95%) homologous or identical to the amino acid sequence set forth in SEQ ID NO: 59. For example, the α chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 59. In certain embodiments, the α chain comprises the amino acid sequence set forth in SEQ ID NO: 59.

In certain embodiments, the extracellular domain of the TCR comprises a β chain that comprises a β chain variable region and a β chain constant region. In certain embodiments, the β chain comprises an amino acid sequence that is at least about 80% (e.g., at least about 85%, at least about 90%, or at least about 95%) homologous or identical to the amino acid sequence set forth in SEQ ID NO: 60. For example, the β chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 60. In certain embodiments, the β chain comprises the amino acid sequence set forth in SEQ ID NO: 60.

In certain embodiments, the α chain comprises an amino acid sequence that is at least about 80% (e.g., at least about 85%, at least about 90%, or at least about 95%) homologous or identical to the amino acid sequence set forth in SEQ ID NO: 59; and the β chain comprises an amino acid sequence that is at least about 80% (e.g., at least about 85%, at least about 90%, or at least about 95%) homologous or identical to the amino acid sequence set forth in SEQ ID NO: 60. In certain embodiments, the α chain comprises the amino acid sequence set forth in SEQ ID NO: 59; and the β chain comprises the amino acid sequence set forth in SEQ ID NO: 60. In certain embodiments, the TCR is designated as “TCR 3A”. In certain embodiments, the TCR 3A binds to a TP53 peptide comprising a R175H mutation.

In certain embodiments, the CDRs sequences described above including Table 6 are delineated using the IMGT numbering system.

TABLE 6
(TCR 3A)

| CDRs | 1 | 2 | 3 |
|---|---|---|---|
| α-chain | DSSSTY | IFSNMDM | CAEISANQAGTALIF |
| | [SEQ ID NO: 51] | [SEQ ID NO: 52] | [SEQ ID NO: 53] |
| β-chain | SEHNR | FQNEAQ | CASSPLDSYQETQYF |
| | [SEQ ID NO: 54] | [SEQ ID NO: 55] | [SEQ ID NO: 56] |

| | |
|---|---|
| α-chain | MKTFAGFSFLFLWLQLDCMSRGEDVEQSLFLSVREGDSSVINCTYTDSSSTYLYWYKQEPGA |
| variable | GLQLLTYIFSNMDMKQDQRLTVLLNKKDKHLSLRIADTQTGDSAIYFCAEISANQAGTALIF |
| | GKGTTLSVSS [SEQ ID NO: 57] |
| β-chain | MGTSLLCWMALCLLGADHADTGVSQNPRHKITKRGQNVTFRCDPISEHNRLYWYRQTLGQGP |
| variable | EFLTYFQNEAQLEKSRLLSDRFSAERPKGSFSTLEIQRTEQGDSAMYLCASSPLDSYQETQY |
| | FGPGTRLLVL [SEQ ID NO: 58] |
| Full α- | MKTFAGFSFLFLWLQLDCMSRGEDVEQSLFLSVREGDSSVINCTYTDSSSTYLYWYKQEPGA |
| chain | GLQLLTYIFSNMDMKQDQRLTVLLNKKDKHLSLRIADTQTGDSAIYFCAEISANQAGTALIF |
| | GKGTTLSVSSNIQNPDPAVYQLRDSKSSDKSVCLFTDFDSQTNVSQSKDSDVYITDKTVLDM |
| | RSMDFKSNSAVAWSNKSDFACANAFNNSIIPEDTFFPSPESSCDVKLVEKSFETDTNLNFQN |
| | LSVIGFRILLLKVAGENLLMTLRLWSS [SEQ ID NO: 59] |
| Full β- | MGTSLLCWMALCLLGADHADTGVSQNPRHKITKRGQNVTFRCDPISEHNRLYWYRQTLGQGP |
| chain | EFLTYFQNEAQLEKSRLLSDRESAERPKGSFSTLEIQRTEQGDSAMYLCASSPLDSYQETQY |
| | FGPGTRLLVLEDLNKVFPPEVAVFEPSEAEISHTQKATLVCLATGFFPDHVELSWWVNGKEV |
| | HSGVSTDPQPLKEQPALNDSRYCLSSRLRVSATFWQNPRNHFRCQVQFYGLSENDEWTQDRA |
| | KPVTQIVSAEAWGRADCGFTSVSYQQGVLSATILYEILLGKATLYAVLVSALVLMAMVKRKD |
| | F [SEQ ID NO: 60] |

In certain embodiments, the extracellular domain of the TCR comprises an α chain variable region comprising a CDR1 comprising the amino acid sequence set forth in SEQ ID NO: 61 or a conservative modification thereof, a CDR2 comprising the amino acid sequence set forth in SEQ ID NO: 62 or a conservative modification thereof, and a CDR3 comprising the amino acid sequence set forth in SEQ ID NO: 63 or a conservative modification thereof. SEQ ID NOS: 61-63 are disclosed in Table 7. In certain embodiments, the α chain variable region comprises a CDR1 comprising the amino acid sequence set forth in SEQ ID NO: 61, a CDR2 comprising the amino acid sequence set forth in SEQ ID NO: 62, and a CDR3 comprising the amino acid sequence set forth in SEQ ID NO: 63.

In certain embodiments, the extracellular domain of the TCR comprises a β chain variable region comprising a CDR1 comprising the amino acid sequence set forth in SEQ ID NO: 64 or a conservative modification thereof, a CDR2 comprising the amino acid sequence set forth in SEQ ID NO: 65 or a conservative modification thereof, and a CDR3 comprising the amino acid sequence set forth in SEQ ID NO: 66 or a conservative modification thereof. SEQ ID NOS: 64-66 are disclosed in Table 7. In certain embodiments, the β chain variable region comprises a CDR1 comprising the amino acid sequence set forth in SEQ ID NO: 64, a CDR2 comprising the amino acid sequence set forth in SEQ ID NO: 65, and a CDR3 comprising the amino acid sequence set forth in SEQ ID NO: 66.

In certain embodiments, the α chain variable region comprises a CDR1 comprising the amino acid sequence set forth in SEQ ID NO: 61 or a conservative modification thereof, a CDR2 comprising the amino acid sequence set forth in SEQ ID NO: 62 or a conservative modification thereof, and a CDR3 comprising the amino acid sequence set forth in SEQ ID NO: 63 or a conservative modification thereof; and the β chain variable region comprises a CDR1 comprising the amino acid sequence set forth in SEQ ID NO: 64 or a conservative modification thereof, a CDR2 comprising the amino acid sequence set forth in SEQ ID NO: 65 or a conservative modification thereof, and a CDR3 comprising the amino acid sequence set forth in SEQ ID NO: 66 or a conservative modification thereof. In certain embodiments, the α chain variable region comprises a CDR1 comprising the amino acid sequence set forth in SEQ ID NO: 61, a CDR2 comprising the amino acid sequence set forth in SEQ ID NO: 62, and a CDR3 comprising the amino acid sequence set forth in SEQ ID NO: 63; and the β chain variable region comprises a CDR1 comprising the amino acid sequence set forth in SEQ ID NO: 64, a CDR2 comprising the amino acid sequence set forth in SEQ ID NO: 65, and a CDR3 comprising the amino acid sequence set forth in SEQ ID NO: 66.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 67. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 67. In certain embodiments, the α chain variable region comprises the amino acid sequence set forth in SEQ ID NO: 67. SEQ ID NO: 67 is provided in Table 7.

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 68. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 68. In certain embodiments, the β chain variable region comprises the amino acid sequence set forth in SEQ ID NO: 68. SEQ ID NO: 68 is provided in Table 7.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 67; and the β chain variable region comprises an amino acid sequence that is at least about 80% (e.g., at least about 85%, at least about 90%, or at least about 95%) homologous or identical to the amino acid sequence set forth in SEQ ID NO: 68. In certain embodiments, the α chain variable region comprises the amino acid sequence set forth in SEQ ID NO: 67; and the β chain variable region comprises the amino acid sequence set forth in SEQ ID NO: 68.

In certain embodiments, the extracellular domain of the TCR comprises an α chain that comprises an α chain variable region and an α chain constant region. In certain embodiments, the α chain comprises an amino acid sequence that is at least about 80% (e.g., at least about 85%, at least about 90%, or at least about 95%) homologous or identical to the amino acid sequence set forth in SEQ ID NO: 69. For example, the α chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 69. In certain embodiments, the α chain comprises the amino acid sequence set forth in SEQ ID NO: 69.

In certain embodiments, the extracellular domain of the TCR comprises a β chain that comprises a β chain variable region and a β chain constant region. In certain embodiments, the β chain comprises an amino acid sequence that is at least about 80% (e.g., at least about 85%, at least about 90%, or at least about 95%) homologous or identical to the amino acid sequence set forth in SEQ ID NO: 70. For example, the β chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 70. In certain embodiments, the β chain comprises the amino acid sequence set forth in SEQ ID NO: 70.

In certain embodiments, the α chain comprises an amino acid sequence that is at least about 80% (e.g., at least about 85%, at least about 90%, or at least about 95%) homologous or identical to the amino acid sequence set forth in SEQ ID NO: 69; and the β chain comprises an amino acid sequence that is at least about 80% (e.g., at least about 85%, at least about 90%, or at least about 95%) homologous or identical to the amino acid sequence set forth in SEQ ID NO: 70. In certain embodiments, the α chain comprises the amino acid sequence set forth in SEQ ID NO: 69; and the β chain comprises the amino acid sequence set forth in SEQ ID NO: 70. In certain embodiments, the TCR is designated as “TCR 3B”. In certain embodiments, the TCR 3B binds to a TP53 peptide comprising a R175H mutation.

In certain embodiments, the CDRs sequences described above including Table 7 are delineated using the IMGT numbering system.

TABLE 7
(TCR 3B)

| CDRs | 1 | 2 | 3 |
|---|---|---|---|
| α-chain | TSDQSYG | QGSYDEQN | CAMREGRGRRALTF |
| | [SEQ ID NO: 61] | [SEQ ID NO: 62] | [SEQ ID NO: 63] |
| β-chain | LGHNA | YSLEER | CASSQDTAWDSGGETDTQYE |
| | [SEQ ID NO: 64] | [SEQ ID NO: 65] | [SEQ ID NO: 66] |

| | |
|---|---|
| α-chain | MSLSSLLKVVTASLWLGPGIAQKITQTQPGMFVQEKEAVTLDCTYDTSDQSYGLFWYKQPSS |
| variable | GEMIFLIYQGSYDEQNATEGRYSLNFQKARKSANLVISASQLGDSAMYFCAMREGRGRRALT |
| | FGSGTRLQVQP [SEQ ID NO: 67] |
| β-chain | MGCRLLCCAVLCLLGAVPMETGVTQTPRHLVMGMTNKKSLKCEQHLGHNAMYWYKQSAKKPL |
| variable | ELMFVYSLEERVENNSVPSRFSPECPNSSHLFLHLHTLQPEDSALYLCASSQDTAWDSGGET |
| | DTQYFGPGTRLTVL [SEQ ID NO: 68] |
| Full α- | MSLSSLLKVVTASLWLGPGIAQKITQTQPGMFVQEKEAVTLDCTYDTSDQSYGLFWYKQPSS |
| chain | GEMIFLIYQGSYDEQNATEGRYSLNFQKARKSANLVISASQLGDSAMYFCAMREGRGRRALT |
| | FGSGTRLQVQPNIQNPDPAVYQLRDSKSSDKSVCLFTDFDSQTNVSQSKDSDVYITDKTVLD |
| | MRSMDFKSNSAVAWSNKSDFACANAFNNSIIPEDTFFPSPESSCDVKLVEKSFETDTNLNFQ |
| | NLSVIGFRILLLKVAGENLLMTLRLWSS [SEQ ID NO: 69] |
| Full β- | MGCRLLCCAVLCLLGAVPMETGVTQTPRHLVMGMTNKKSLKCEQHLGHNAMYWYKQSAKKPL |
| chain | ELMFVYSLEERVENNSVPSRFSPECPNSSHLFLHLHTLQPEDSALYLCASSQDTAWDSGGET |
| | DTQYFGPGTRLTVLEDLNKVFPPEVAVFEPSEAEISHTQKATLVCLATGFFPDHVELSWWVN |
| | GKEVHSGVSTDPQPLKEQPALNDSRYCLSSRLRVSATFWQNPRNHERCQVQFYGLSENDEWT |
| | QDRAKPVTQIVSAEAWGRADCGFTSVSYQQGVLSATILYEILLGKATLYAVLVSALVLMAMV |
| | KRKDF [SEQ ID NO: 70] |

In certain embodiments, the extracellular domain of the TCR comprises an α chain variable region comprising a CDR1 comprising the amino acid sequence set forth in SEQ ID NO: 71 or a conservative modification thereof, a CDR2 comprising the amino acid sequence set forth in SEQ ID NO: 72 or a conservative modification thereof, and a CDR3 comprising the amino acid sequence set forth in SEQ ID NO: 73 or a conservative modification thereof. SEQ ID NOS: 71-73 are disclosed in Table 8. In certain embodiments, the α chain variable region comprises a CDR1 comprising the amino acid sequence set forth in SEQ ID NO: 71, a CDR2 comprising the amino acid sequence set forth in SEQ ID NO: 72, and a CDR3 comprising the amino acid sequence set forth in SEQ ID NO: 73.

In certain embodiments, the extracellular domain of the TCR comprises a β chain variable region comprising a CDR1 comprising the amino acid sequence set forth in SEQ ID NO: 74 or a conservative modification thereof, a CDR2 comprising the amino acid sequence set forth in SEQ ID NO: 75 or a conservative modification thereof, and a CDR3 comprising the amino acid sequence set forth in SEQ ID NO: 76 or a conservative modification thereof. SEQ ID NOS: 74-76 are disclosed in Table 8. In certain embodiments, the β chain variable region comprises a CDR1 comprising the amino acid sequence set forth in SEQ ID NO: 74, a CDR2 comprising the amino acid sequence set forth in SEQ ID NO: 75, and a CDR3 comprising the amino acid sequence set forth in SEQ ID NO: 76.

In certain embodiments, the α chain variable region comprises a CDR1 comprising the amino acid sequence set forth in SEQ ID NO: 71 or a conservative modification thereof, a CDR2 comprising the amino acid sequence set forth in SEQ ID NO: 72 or a conservative modification thereof, and a CDR3 comprising the amino acid sequence set forth in SEQ ID NO: 73 or a conservative modification thereof; and the β chain variable region comprises a CDR1 comprising the amino acid sequence set forth in SEQ ID NO: 74 or a conservative modification thereof, a CDR2 comprising the amino acid sequence set forth in SEQ ID NO: 75 or a conservative modification thereof, and a CDR3 comprising the amino acid sequence set forth in SEQ ID NO: 76 or a conservative modification thereof. In certain embodiments, the α chain variable region comprises a CDR1 comprising the amino acid sequence set forth in SEQ ID NO: 71, a CDR2 comprising the amino acid sequence set forth in SEQ ID NO: 72, and a CDR3 comprising the amino acid sequence set forth in SEQ ID NO: 73; and the β chain variable region comprises a CDR1 comprising the amino acid sequence set forth in SEQ ID NO: 74, a CDR2 comprising the amino acid sequence set forth in SEQ ID NO: 75, and a CDR3 comprising the amino acid sequence set forth in SEQ ID NO: 76.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 77. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 77. In certain embodiments, the α chain variable region comprises the amino acid sequence set forth in SEQ ID NO: 77. SEQ ID NO: 77 is provided in Table 8.

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 78. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 78. In certain embodiments, the β chain variable region comprises the amino acid sequence set forth in SEQ ID NO: 78. SEQ ID NO: 78 is provided in Table 8.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 77; and the β chain variable region comprises an amino acid sequence that is at least about 80% (e.g., at least about 85%, at least about 90%, or at least about 95%) homologous or identical to the amino acid sequence set forth in SEQ ID NO: 78. In certain embodiments, the α chain variable region comprises the amino acid sequence set forth in SEQ ID NO: 77; and the β chain variable region comprises the amino acid sequence set forth in SEQ ID NO: 78.

In certain embodiments, the extracellular domain of the TCR comprises an α chain that comprises an α chain variable region and an α chain constant region. In certain embodiments, the α chain comprises an amino acid sequence that is at least about 80% (e.g., at least about 85%, at least about 90%, or at least about 95%) homologous or identical to the amino acid sequence set forth in SEQ ID NO: 79. For example, the α chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 79. In certain embodiments, the α chain comprises the amino acid sequence set forth in SEQ ID NO: 79.

In certain embodiments, the extracellular domain of the TCR comprises a β chain that comprises a β chain variable region and a β chain constant region. In certain embodiments, the β chain comprises an amino acid sequence that is at least about 80% (e.g., at least about 85%, at least about 90%, or at least about 95%) homologous or identical to the amino acid sequence set forth in SEQ ID NO: 80. For example, the β chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 80. In certain embodiments, the β chain comprises the amino acid sequence set forth in SEQ ID NO: 80.

In certain embodiments, the α chain comprises an amino acid sequence that is at least about 80% (e.g., at least about 85%, at least about 90%, or at least about 95%) homologous or identical to the amino acid sequence set forth in SEQ ID NO: 79; and the β chain comprises an amino acid sequence that is at least about 80% (e.g., at least about 85%, at least about 90%, or at least about 95%) homologous or identical to the amino acid sequence set forth in SEQ ID NO: 80. In certain embodiments, the α chain comprises the amino acid sequence set forth in SEQ ID NO: 79; and the β chain comprises the amino acid sequence set forth in SEQ ID NO: 80. In certain embodiments, the TCR is designated as “TCR 4A”. In certain embodiments, the TCR 4A binds to a TP53 peptide comprising a R175H mutation.

In certain embodiments, the CDRs sequences described above including Table 8 are delineated using the IMGT numbering system.

TABLE 8
(TCR 4A)

| CDRs | 1 | 2 | 3 |
|---|---|---|---|
| α-chain | TSESNYY | QEAYKQQN | CAFMKWQGGSEKLVF |
| | [SEQ ID NO: 71] | [ SEQ ID NO: 72] | [SEQ ID NO: 73] |
| β-chain | LGHDT | YNNKEL | CASSQALAGEVDTQYF |
| | [SEQ ID NO: 74] | [SEQ ID NO: 75] | [SEQ ID NO: 76] |

| | |
|---|---|
| α-chain | MTRVSLLWAVVVSTCLESGMAQTVTQSQPEMSVQEAETVTLSCTYDTSESNYYLFWYKQPPS |
| variable | RQMILVIRQEAYKQQNATENRFSVNFQKAAKSFSLKISDSQLGDTAMYFCAFMKWQGGSEKL |
| | VFGKGTKLTVNP [SEQ ID NO: 77] |
| β-chain | MGCRLLCCVVFCLLQAGPLDTAVSQTPKYLVTQMGNDKSIKCEQNLGHDTMYWYKQDSKKEL |
| variable | KIMFSYNNKELIINETVPNRFSPKSPDKAHLNLHINSLELGDSAVYFCASSQALAGEVDTQY |
| | FGPGTRLTVL [SEQ ID NO: 78] |
| Full α- | MTRVSLLWAVVVSTCLESGMAQTVTQSQPEMSVQEAETVTLSCTYDTSESNYYLFWYKQPPS |
| chain | RQMILVIRQEAYKQQNATENRFSVNFQKAAKSFSLKISDSQLGDTAMYFCAFMKWQGGSEKL |
| | VFGKGTKLTVNPNIQNPDPAVYQLRDSKSSDKSVCLFTDFDSQTNVSQSKDSDVYITDKTVL |
| | DMRSMDFKSNSAVAWSNKSDFACANAFNNSIIPEDTFFPSPESSCDVKLVEKSFETDTNLNF |
| | QNLSVIGFRILLLKVAGENLLMTLRLWSS |
| | [SEQ ID NO: 79] |
| Full β- | MGCRLLCCVVFCLLQAGPLDTAVSQTPKYLVTQMGNDKSIKCEQNLGHDTMYWYKQDSKKEL |
| chain | KIMFSYNNKELIINETVPNRFSPKSPDKAHLNLHINSLELGDSAVYFCASSQALAGEVDTQY |
| | FGPGTRLTVLEDLNKVFPPEVAVFEPSEAEISHTQKATLVCLATGFFPDHVELSWWVNGKEV |
| | HSGVSTDPQPLKEQPALNDSRYCLSSRLRVSATFWQNPRNHERCQVQFYGLSENDEWTQDRA |
| | KPVTQIVSAEAWGRADCGFTSVSYQQGVLSATILYEILLGKATLYAVLVSALVLMAMVKRKD |
| | F [SEQ ID NO: 80] |

In certain embodiments, the extracellular domain of the TCR comprises an α chain variable region comprising a CDR1 comprising the amino acid sequence set forth in SEQ ID NO: 81 or a conservative modification thereof, a CDR2 comprising the amino acid sequence set forth in SEQ ID NO: 82 or a conservative modification thereof, and a CDR3 comprising the amino acid sequence set forth in SEQ ID NO: 83 or a conservative modification thereof. SEQ ID NOS: 81-83 are disclosed in Table 9. In certain embodiments, the α chain variable region comprises a CDR1 comprising the amino acid sequence set forth in SEQ ID NO: 81, a CDR2 comprising the amino acid sequence set forth in SEQ ID NO: 82, and a CDR3 comprising the amino acid sequence set forth in SEQ ID NO: 83.

In certain embodiments, the extracellular domain of the TCR comprises a β chain variable region comprising a CDR1 comprising the amino acid sequence set forth in SEQ ID NO: 84 or a conservative modification thereof, a CDR2 comprising the amino acid sequence set forth in SEQ ID NO: 85 or a conservative modification thereof, and a CDR3 comprising the amino acid sequence set forth in SEQ ID NO: 86 or a conservative modification thereof. SEQ ID NOS: 84-86 are disclosed in Table 9. In certain embodiments, the β chain variable region comprises a CDR1 comprising the amino acid sequence set forth in SEQ ID NO: 84, a CDR2 comprising the amino acid sequence set forth in SEQ ID NO: 85, and a CDR3 comprising the amino acid sequence set forth in SEQ ID NO: 86.

In certain embodiments, the α chain variable region comprises a CDR1 comprising the amino acid sequence set forth in SEQ ID NO: 81 or a conservative modification thereof, a CDR2 comprising the amino acid sequence set forth in SEQ ID NO: 82 or a conservative modification thereof, and a CDR3 comprising the amino acid sequence set forth in SEQ ID NO: 83 or a conservative modification thereof; and the β chain variable region comprises a CDR1 comprising the amino acid sequence set forth in SEQ ID NO: 84 or a conservative modification thereof, a CDR2 comprising the amino acid sequence set forth in SEQ ID NO: 85 or a conservative modification thereof, and a CDR3 comprising the amino acid sequence set forth in SEQ ID NO: 86 or a conservative modification thereof. In certain embodiments, the α chain variable region comprises a CDR1 comprising the amino acid sequence set forth in SEQ ID NO: 81, a CDR2 comprising the amino acid sequence set forth in SEQ ID NO: 82, and a CDR3 comprising the amino acid sequence set forth in SEQ ID NO: 83; and the β chain variable region comprises a CDR1 comprising the amino acid sequence set forth in SEQ ID NO: 84, a CDR2 comprising the amino acid sequence set forth in SEQ ID NO: 85, and a CDR3 comprising the amino acid sequence set forth in SEQ ID NO: 86.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 87. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 87. In certain embodiments, the α chain variable region comprises the amino acid sequence set forth in SEQ ID NO: 87. SEQ ID NO: 87 is provided in Table 9.

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 88. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 88. In certain embodiments, the β chain variable region comprises the amino acid sequence set forth in SEQ ID NO: 88. SEQ ID NO: 88 is provided in Table 9.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 87; and the β chain variable region comprises an amino acid sequence that is at least about 80% (e.g., at least about 85%, at least about 90%, or at least about 95%) homologous or identical to the amino acid sequence set forth in SEQ ID NO: 88. In certain embodiments, the α chain variable region comprises the amino acid sequence set forth in SEQ ID NO: 87; and the β chain variable region comprises the amino acid sequence set forth in SEQ ID NO: 88.

In certain embodiments, the extracellular domain of the TCR comprises an α chain that comprises an α chain variable region and an α chain constant region. In certain embodiments, the α chain comprises an amino acid sequence that is at least about 80% (e.g., at least about 85%, at least about 90%, or at least about 95%) homologous or identical to the amino acid sequence set forth in SEQ ID NO: 89. For example, the α chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 89. In certain embodiments, the α chain comprises the amino acid sequence set forth in SEQ ID NO: 89.

In certain embodiments, the extracellular domain of the TCR comprises a β chain that comprises a β chain variable region and a β chain constant region. In certain embodiments, the β chain comprises an amino acid sequence that is at least about 80% (e.g., at least about 85%, at least about 90%, or at least about 95%) homologous or identical to the amino acid sequence set forth in SEQ ID NO: 90. For example, the β chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 90. In certain embodiments, the β chain comprises the amino acid sequence set forth in SEQ ID NO: 90.

In certain embodiments, the α chain comprises an amino acid sequence that is at least about 80% (e.g., at least about 85%, at least about 90%, or at least about 95%) homologous or identical to the amino acid sequence set forth in SEQ ID NO: 89; and the β chain comprises an amino acid sequence that is at least about 80% (e.g., at least about 85%, at least about 90%, or at least about 95%) homologous or identical to the amino acid sequence set forth in SEQ ID NO: 90. In certain embodiments, the α chain comprises the amino acid sequence set forth in SEQ ID NO: 89; and the β chain comprises the amino acid sequence set forth in SEQ ID NO: 90. In certain embodiments, the TCR is designated as “TCR 7A”. In certain embodiments, the TCR 7A binds to a TP53 peptide comprising a R175H mutation.

In certain embodiments, the CDRs sequences described above including Table 9 are delineated using the IMGT numbering system.

TABLE 9
(TCR 7A)

| CDRs | 1 | 2 | 3 |
|---|---|---|---|
| α-chain | TRDTTYY | RNSFDEQN | CALRNSGNTPLVE |
| | [SEQ ID NO: 81] | [SEQ ID NO: 82] | [SEQ ID NO: 83] |
| β-chain | SGDLS | YYNGEE | CASSVDWVQETQYF |
| | [SEQ ID NO: 84] | [SEQ ID NO: 85] | [SEQ ID NO: 86] |

| | |
|---|---|
| α-chain | MLTASLLRAVIASICVVSSMAQKVTQAQTEISVVEKEDVTLDCVYETRDTTYYLFW |
| variable | YKQPPSGELVFLIRRNSFDEQNEISGRYSWNFQKSTSSENFTITASQVVDSAVYFC |
| | ALRNSGNTPLVFGKGTRLSVIA [SEQ ID NO: 87] |
| β-chain | MGFRLLCCVAFCLLGAGPVDSGVTQTPKHLITATGQRVTLRCSPRSGDLSVYWYQQ |
| variable | SLDQGLQFLIQYYNGEERAKGNILERFSAQQFPDLHSELNLSSLELGDSALYFCAS |
| | SVDWVQETQYFGPGTRLLVL [SEQ ID NO: 88] |
| Full α- | MLTASLLRAVIASICVVSSMAQKVTQAQTEISVVEKEDVTLDCVYETRDTTYYLEW |
| chain | YKQPPSGELVFLIRRNSFDEQNEISGRYSWNFQKSTSSENFTITASQVVDSAVYFC |
| | ALRNSGNTPLVFGKGTRLSVIANIQNPDPAVYQLRDSKSSDKSVCLFTDFDSQTNVSQSK |
| | DSDVYITDKTVLDMRSMDFKSNSAVAWSNKSDFACANAFNNSIIPEDTFFPSPESSCDVKLV |
| | EKSFETDTNLNFQNLSVIGFRILLLKVAGENLLMTLRLWSS [SEQ ID NO: 89] |
| Full β- | MGFRLLCCVAFCLLGAGPVDSGVTQTPKHLITATGQRVTLRCSPRSGDLSVYWYQQ |
| chain | SLDQGLQFLIQYYNGEERAKGNILERFSAQQFPDLHSELNLSSLELGDSALYFCAS |
| | SVDWVQETQYFGPGTRLLVLEDLNKVFPPEVAVFEPSEAEISHTQKATLVCLATGFFPDH |
| | VELSWWVNGKEVHSGVSTDPQPLKEQPALNDSRYCLSSRLRVSATFWQNPRNHFRCQVQFYG |
| | LSENDEWTQDRAKPVTQIVSAEAWGRADCGFTSVSYQQGVLSATILYEILLGKATLYAVLVS |
| | ALVLMAMVKRKDF [SEQ ID NO: 90] |

In certain embodiments, the extracellular domain of the TCR comprises an α chain variable region comprising a CDR1 comprising the amino acid sequence set forth in SEQ ID NO: 91 or a conservative modification thereof, a CDR2 comprising the amino acid sequence set forth in SEQ ID NO: 92 or a conservative modification thereof, and a CDR3 comprising the amino acid sequence set forth in SEQ ID NO: 93 or a conservative modification thereof. SEQ ID NOS: 91-93 are disclosed in Table 10. In certain embodiments, the α chain variable region comprises a CDR1 comprising the amino acid sequence set forth in SEQ ID NO: 91, a CDR2 comprising the amino acid sequence set forth in SEQ ID NO: 92, and a CDR3 comprising the amino acid sequence set forth in SEQ ID NO: 93.

In certain embodiments, the extracellular domain of the TCR comprises a β chain variable region comprising a CDR1 comprising the amino acid sequence set forth in SEQ ID NO: 94 or a conservative modification thereof, a CDR2 comprising the amino acid sequence set forth in SEQ ID NO: 95 or a conservative modification thereof, and a CDR3 comprising the amino acid sequence set forth in SEQ ID NO: 96 or a conservative modification thereof. SEQ ID NOS: 94-96 are disclosed in Table 10. In certain embodiments, the β chain variable region comprises a CDR1 comprising the amino acid sequence set forth in SEQ ID NO: 94, a CDR2 comprising the amino acid sequence set forth in SEQ ID NO: 95, and a CDR3 comprising the amino acid sequence set forth in SEQ ID NO: 96.

In certain embodiments, the α chain variable region comprises a CDR1 comprising the amino acid sequence set forth in SEQ ID NO: 91 or a conservative modification thereof, a CDR2 comprising the amino acid sequence set forth in SEQ ID NO: 92 or a conservative modification thereof, and a CDR3 comprising the amino acid sequence set forth in SEQ ID NO: 93 or a conservative modification thereof; and the β chain variable region comprises a CDR1 comprising the amino acid sequence set forth in SEQ ID NO: 94 or a conservative modification thereof, a CDR2 comprising the amino acid sequence set forth in SEQ ID NO: 95 or a conservative modification thereof, and a CDR3 comprising the amino acid sequence set forth in SEQ ID NO: 96 or a conservative modification thereof. In certain embodiments, the α chain variable region comprises a CDR1 comprising the amino acid sequence set forth in SEQ ID NO: 91, a CDR2 comprising the amino acid sequence set forth in SEQ ID NO: 92, and a CDR3 comprising the amino acid sequence set forth in SEQ ID NO: 93; and the β chain variable region comprises a CDR1 comprising the amino acid sequence set forth in SEQ ID NO: 94, a CDR2 comprising the amino acid sequence set forth in SEQ ID NO: 95, and a CDR3 comprising the amino acid sequence set forth in SEQ ID NO: 96.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 97. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 97. In certain embodiments, the α chain variable region comprises the amino acid sequence set forth in SEQ ID NO: 97. SEQ ID NO: 97 is provided in Table 10.

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 98. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 98. In certain embodiments, the β chain variable region comprises the amino acid sequence set forth in SEQ ID NO: 98. SEQ ID NO: 98 is provided in Table 10.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 97; and the β chain variable region comprises an amino acid sequence that is at least about 80% (e.g., at least about 85%, at least about 90%, or at least about 95%) homologous or identical to the amino acid sequence set forth in SEQ ID NO: 98. In certain embodiments, the α chain variable region comprises the amino acid sequence set forth in SEQ ID NO: 97; and the β chain variable region comprises the amino acid sequence set forth in SEQ ID NO: 98.

In certain embodiments, the extracellular domain of the TCR comprises an α chain that comprises an α chain variable region and an α chain constant region. In certain embodiments, the α chain comprises an amino acid sequence that is at least about 80% (e.g., at least about 85%, at least about 90%, or at least about 95%) homologous or identical to the amino acid sequence set forth in SEQ ID NO: 99. For example, the α chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 99. In certain embodiments, the α chain comprises the amino acid sequence set forth in SEQ ID NO: 99.

In certain embodiments, the extracellular domain of the TCR comprises a β chain that comprises a β chain variable region and a β chain constant region. In certain embodiments, the β chain comprises an amino acid sequence that is at least about 80% (e.g., at least about 85%, at least about 90%, or at least about 95%) homologous or identical to the amino acid sequence set forth in SEQ ID NO: 100. For example, the β chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 100. In certain embodiments, the β chain comprises the amino acid sequence set forth in SEQ ID NO: 100.

In certain embodiments, the α chain comprises an amino acid sequence that is at least about 80% (e.g., at least about 85%, at least about 90%, or at least about 95%) homologous or identical to the amino acid sequence set forth in SEQ ID NO: 99; and the β chain comprises an amino acid sequence that is at least about 80% (e.g., at least about 85%, at least about 90%, or at least about 95%) homologous or identical to the amino acid sequence set forth in SEQ ID NO: 100. In certain embodiments, the α chain comprises the amino acid sequence set forth in SEQ ID NO: 99; and the β chain comprises the amino acid sequence set forth in SEQ ID NO: 100. In certain embodiments, the TCR is designated as “TCR 7B”. In certain embodiments, the TCR 7B binds to a TP53 peptide comprising a R175H mutation.

In certain embodiments, the CDRs sequences described above including Table 10 are delineated using the IMGT numbering system.

TABLE 10
(TCR 7B)

| CDRs | 1 | 2 | 3 |
|---|---|---|---|
| α-chain | TISGNEY | GLKNN | CIVPRGESNTGKLIF |
| | [SEQ ID NO: 91] | [SEQ ID NO: 92] | [SEQ ID NO: 93] |
| β-chain | DFQATT | SNEGSKA | CSASESLALASEQYF |
| | [SEQ ID NO: 94] | [SEQ ID NO: 95] | [SEQ ID NO: 96] |

| | |
|---|---|
| α-chain | MRLVARVTVFLTFGTIIDAKTTQPTSMDCAEGRAANLPCNHSTISGNEYVYWYRQI |
| variable | HSQGPQYIIHGLKNNETNEMASLIITEDRKSSTLILPHATLRDTAVYYCIVPRGFS |
| | NTGKLIFGQGTTLQVKP [SEQ ID NO: 97] |
| β-chain | MLLLLLLLGPGSGLGAVVSQHPSRVICKSGTSVKIECRSLDFQATTMFWYRQFPKQ |
| variable | SLMLMATSNEGSKATYEQGVEKDKFLINHASLTLSTLTVTSAHPEDSSFYICSASE |
| | SLALASEQYFGPGTRLTVT [SEQ ID NO: 98] |
| Full α- | MRLVARVTVELTFGTIIDAKTTQPTSMDCAEGRAANLPCNHSTISGNEYVYWYRQI |
| chain | HSQGPQYIIHGLKNNETNEMASLIITEDRKSSTLILPHATLRDTAVYYCIVPRGFS |
| | NTGKLIFGQGTTLQVKPNIQNPDPAVYQLRDSKSSDKSVCLFTDFDSQTNVSQSKDSDVY |
| | ITDKTVLDMRSMDFKSNSAVAWSNKSDFACANAFNNSIIPEDTFFPSPESSCDVKLVEKSFE |
| | TDTNLNFQNLSVIGFRILLLKVAGENLLMTLRLWSS [SEQ ID NO: 99] |
| Full β- | MLLLLLLLGPGSGLGAVVSQHPSRVICKSGTSVKIECRSLDFQATTMFWYRQFPKQ |
| chain | SLMLMATSNEGSKATYEQGVEKDKFLINHASLTLSTLTVTSAHPEDSSFYICSASE |
| | SLALASEQYFGPGTRLTVTEDLNKVFPPEVAVFEPSEAEISHTQKATLVCLATGFFPDHV |
| | ELSWWVNGKEVHSGVSTDPQPLKEQPALNDSRYCLSSRLRVSATFWQNPRNHERCQVQFYGL |
| | SENDEWTQDRAKPVTQIVSAEAWGRADCGFTSVSYQQGVLSATILYEILLGKATLYAVLVSA |
| | LVLMAMVKRKDF [SEQ ID NO: 100] |

In certain embodiments, the extracellular domain of the TCR comprises an α chain variable region comprising a CDR1 comprising the amino acid sequence set forth in SEQ ID NO: 101 or a conservative modification thereof, a CDR2 comprising the amino acid sequence set forth in SEQ ID NO: 102 or a conservative modification thereof, and a CDR3 comprising the amino acid sequence set forth in SEQ ID NO: 103 or a conservative modification thereof. SEQ ID NOS: 101-103 are disclosed in Table 11. In certain embodiments, the α chain variable region comprises a CDR1 comprising the amino acid sequence set forth in SEQ ID NO: 101, a CDR2 comprising the amino acid sequence set forth in SEQ ID NO: 102, and a CDR3 comprising the amino acid sequence set forth in SEQ ID NO: 103.

In certain embodiments, the extracellular domain of the TCR comprises a β chain variable region comprising a CDR1 comprising the amino acid sequence set forth in SEQ ID NO: 104 or a conservative modification thereof, a CDR2 comprising the amino acid sequence set forth in SEQ ID NO: 105 or a conservative modification thereof, and a CDR3 comprising the amino acid sequence set forth in SEQ ID NO: 106 or a conservative modification thereof. SEQ ID NOS: 104-106 are disclosed in Table 11. In certain embodiments, the β chain variable region comprises a CDR1 comprising the amino acid sequence set forth in SEQ ID NO: 104, a CDR2 comprising the amino acid sequence set forth in SEQ ID NO: 105, and a CDR3 comprising the amino acid sequence set forth in SEQ ID NO: 106.

In certain embodiments, the α chain variable region comprises a CDR1 comprising the amino acid sequence set forth in SEQ ID NO: 101 or a conservative modification thereof, a CDR2 comprising the amino acid sequence set forth in SEQ ID NO: 102 or a conservative modification thereof, and a CDR3 comprising the amino acid sequence set forth in SEQ ID NO: 103 or a conservative modification thereof; and the β chain variable region comprises a CDR1 comprising the amino acid sequence set forth in SEQ ID NO: 104 or a conservative modification thereof, a CDR2 comprising the amino acid sequence set forth in SEQ ID NO: 105 or a conservative modification thereof, and a CDR3 comprising the amino acid sequence set forth in SEQ ID NO: 106 or a conservative modification thereof. In certain embodiments, the α chain variable region comprises a CDR1 comprising the amino acid sequence set forth in SEQ ID NO: 101, a CDR2 comprising the amino acid sequence set forth in SEQ ID NO: 102, and a CDR3 comprising the amino acid sequence set forth in SEQ ID NO: 103; and the β chain variable region comprises a CDR1 comprising the amino acid sequence set forth in SEQ ID NO: 104, a CDR2 comprising the amino acid sequence set forth in SEQ ID NO: 105, and a CDR3 comprising the amino acid sequence set forth in SEQ ID NO: 106.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 107. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 107. In certain embodiments, the α chain variable region comprises the amino acid sequence set forth in SEQ ID NO: 107. SEQ ID NO: 107 is provided in Table 11.

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 108. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 108. In certain embodiments, the β chain variable region comprises the amino acid sequence set forth in SEQ ID NO: 108. SEQ ID NO: 108 is provided in Table 11.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 107; and the β chain variable region comprises an amino acid sequence that is at least about 80% (e.g., at least about 85%, at least about 90%, or at least about 95%) homologous or identical to the amino acid sequence set forth in SEQ ID NO: 108. In certain embodiments, the α chain variable region comprises the amino acid sequence set forth in SEQ ID NO: 107; and the β chain variable region comprises the amino acid sequence set forth in SEQ ID NO: 108.

In certain embodiments, the extracellular domain of the TCR comprises an α chain that comprises an α chain variable region and an α chain constant region. In certain embodiments, the α chain comprises an amino acid sequence that is at least about 80% (e.g., at least about 85%, at least about 90%, or at least about 95%) homologous or identical to the amino acid sequence set forth in SEQ ID NO: 109. For example, the α chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 109. In certain embodiments, the α chain comprises the amino acid sequence set forth in SEQ ID NO: 109.

In certain embodiments, the extracellular domain of the TCR comprises a β chain that comprises a β chain variable region and a β chain constant region. In certain embodiments, the β chain comprises an amino acid sequence that is at least about 80% (e.g., at least about 85%, at least about 90%, or at least about 95%) homologous or identical to the amino acid sequence set forth in SEQ ID NO: 110. For example, the β chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 110. In certain embodiments, the β chain comprises the amino acid sequence set forth in SEQ ID NO: 110.

In certain embodiments, the α chain comprises an amino acid sequence that is at least about 80% (e.g., at least about 85%, at least about 90%, or at least about 95%) homologous or identical to the amino acid sequence set forth in SEQ ID NO: 109; and the β chain comprises an amino acid sequence that is at least about 80% (e.g., at least about 85%, at least about 90%, or at least about 95%) homologous or identical to the amino acid sequence set forth in SEQ ID NO: 110. In certain embodiments, the α chain comprises the amino acid sequence set forth in SEQ ID NO: 109; and the β chain comprises the amino acid sequence set forth in SEQ ID NO: 110. In certain embodiments, the TCR is designated as “TCR 7D”. In certain embodiments, the TCR 7D binds to a TP53 peptide comprising a R175H mutation.

In certain embodiments, the CDRs sequences described above including Table 11 are delineated using the IMGT numbering system.

TABLE 11
(TCR 7D)

| CDRs | 1 | 2 | 3 |
|---|---|---|---|
| α-chain | NSAFQY | TYSSGN | CASSESGYNKLIF |
| | [SEQ ID NO: | [SEQ ID NO: 102] | [SEQ ID NO: 103] |
| | 101] | | |
| β-chain | SGDLS | YYNGEE | CASSERLAGAIADTQYE |
| | [SEQ ID NO: | [SEQ ID NO: 105] | [SEQ ID NO: 106] |
| | 104] | | |

| | |
|---|---|
| α-chain | MMKSLRVLLVILWLQLSWVWSQQKEVEQDPGPLSVPEGAIVSLNCTYSNSAFQYFM |
| variable | WYRQYSRKGPELLMYTYSSGNKEDGRETAQVDKSSKYISLFIRDSQPSDSATYLCA |
| | SSFSGYNKLIFGAGTRLAVHP [SEQ ID NO: 107] |
| β-chain | MGFRLLCCVAFCLLGAGPVDSGVTQTPKHLITATGQRVTLRCSPRSGDLSVYWYQQ |
| variable | SLDQGLQFLIQYYNGEERAKGNILERFSAQQFPDLHSELNLSSLELGDSALYFCAS |
| | SERLAGAIADTQYFGPGTRLTVL [SEQ ID NO: 108] |
| Full α- | MMKSLRVLLVILWLQLSWVWSQQKEVEQDPGPLSVPEGAIVSLNCTYSNSAFQYFM |
| chain | WYRQYSRKGPELLMYTYSSGNKEDGRFTAQVDKSSKYISLFIRDSQPSDSATYLCA |
| | SSESGYNKLIFGAGTRLAVHPNIQNPDPAVYQLRDSKSSDKSVCLFTDFDSQTNVSQSKD |
| | SDVYITDKTVLDMRSMDFKSNSAVAWSNKSDFACANAFNNSIIPEDTFFPSPESSCDVKLVE |
| | KSFETDTNLNFQNLSVIGFRILLLKVAGENLLMTLRLWSS [SEQ ID NO: 109] |
| Full β- | MGFRLLCCVAFCLLGAGPVDSGVTQTPKHITATGQRVTLRCSPRSGDLSVYWYQQS |
| chain | LDQGLQFLIQYYNGEERAKGNILERFSAQQFPDLHSELNLSSLELGDSALYFCASS |
| | ERLAGAIADTQYFGPGTRLTVLEDLNKVFPPEVAVFEPSEAEISHTQKATLVCLATGFFP |
| | DHVELSWWVNGKEVHSGVSTDPQPLKEQPALNDSRYCLSSRLRVSATFWQNPRNHERCQVQF |
| | YGLSENDEWTQDRAKPVTQIVSAEAWGRADCGFTSVSYQQGVLSATILYEILLGKATLYAVL |
| | VSALVLMAMVKRKDF [SEQ ID NO: 110] |

In certain embodiments, the extracellular domain of the TCR comprises an α chain variable region comprising a CDR1 comprising the amino acid sequence set forth in SEQ ID NO: 111 or a conservative modification thereof, a CDR2 comprising the amino acid sequence set forth in SEQ ID NO: 112 or a conservative modification thereof, and a CDR3 comprising the amino acid sequence set forth in SEQ ID NO: 113 or a conservative modification thereof. SEQ ID NOS: 111-113 are disclosed in Table 12. In certain embodiments, the α chain variable region comprises a CDR1 comprising the amino acid sequence set forth in SEQ ID NO: 111, a CDR2 comprising the amino acid sequence set forth in SEQ ID NO: 112, and a CDR3 comprising the amino acid sequence set forth in SEQ ID NO: 113.

In certain embodiments, the extracellular domain of the TCR comprises a β chain variable region comprising a CDR1 comprising the amino acid sequence set forth in SEQ ID NO: 114 or a conservative modification thereof, a CDR2 comprising the amino acid sequence set forth in SEQ ID NO: 115 or a conservative modification thereof, and a CDR3 comprising the amino acid sequence set forth in SEQ ID NO: 116 or a conservative modification thereof. SEQ ID NOS: 114-116 are disclosed in Table 12. In certain embodiments, the β chain variable region comprises a CDR1 comprising the amino acid sequence set forth in SEQ ID NO: 114, a CDR2 comprising the amino acid sequence set forth in SEQ ID NO: 115, and a CDR3 comprising the amino acid sequence set forth in SEQ ID NO: 116.

In certain embodiments, the α chain variable region comprises a CDR1 comprising the amino acid sequence set forth in SEQ ID NO: 111 or a conservative modification thereof, a CDR2 comprising the amino acid sequence set forth in SEQ ID NO: 112 or a conservative modification thereof, and a CDR3 comprising the amino acid sequence set forth in SEQ ID NO: 113 or a conservative modification thereof; and the β chain variable region comprises a CDR1 comprising the amino acid sequence set forth in SEQ ID NO: 114 or a conservative modification thereof, a CDR2 comprising the amino acid sequence set forth in SEQ ID NO: 115 or a conservative modification thereof, and a CDR3 comprising the amino acid sequence set forth in SEQ ID NO: 116 or a conservative modification thereof. In certain embodiments, the α chain variable region comprises a CDR1 comprising the amino acid sequence set forth in SEQ ID NO: 111, a CDR2 comprising the amino acid sequence set forth in SEQ ID NO: 112, and a CDR3 comprising the amino acid sequence set forth in SEQ ID NO: 113; and the β chain variable region comprises a CDR1 comprising the amino acid sequence set forth in SEQ ID NO: 114, a CDR2 comprising the amino acid sequence set forth in SEQ ID NO: 115, and a CDR3 comprising the amino acid sequence set forth in SEQ ID NO: 116.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 117. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 117. In certain embodiments, the α chain variable region comprises the amino acid sequence set forth in SEQ ID NO: 117. SEQ ID NO: 117 is provided in Table 12.

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 118. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 118. In certain embodiments, the β chain variable region comprises the amino acid sequence set forth in SEQ ID NO: 118. SEQ ID NO: 118 is provided in Table 12.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 117; and the β chain variable region comprises an amino acid sequence that is at least about 80% (e.g., at least about 85%, at least about 90%, or at least about 95%) homologous or identical to the amino acid sequence set forth in SEQ ID NO: 118. In certain embodiments, the α chain variable region comprises the amino acid sequence set forth in SEQ ID NO: 117; and the β chain variable region comprises the amino acid sequence set forth in SEQ ID NO: 118.

In certain embodiments, the extracellular domain of the TCR comprises an α chain that comprises an α chain variable region and an α chain constant region. In certain embodiments, the α chain comprises an amino acid sequence that is at least about 80% (e.g., at least about 85%, at least about 90%, or at least about 95%) homologous or identical to the amino acid sequence set forth in SEQ ID NO: 119. For example, the α chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 119. In certain embodiments, the α chain comprises the amino acid sequence set forth in SEQ ID NO: 119.

In certain embodiments, the extracellular domain of the TCR comprises a β chain that comprises a β chain variable region and a β chain constant region. In certain embodiments, the β chain comprises an amino acid sequence that is at least about 80% (e.g., at least about 85%, at least about 90%, or at least about 95%) homologous or identical to the amino acid sequence set forth in SEQ ID NO: 120. For example, the β chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 120. In certain embodiments, the β chain comprises the amino acid sequence set forth in SEQ ID NO: 120.

In certain embodiments, the α chain comprises an amino acid sequence that is at least about 80% (e.g., at least about 85%, at least about 90%, or at least about 95%) homologous or identical to the amino acid sequence set forth in SEQ ID NO: 119; and the β chain comprises an amino acid sequence that is at least about 80% (e.g., at least about 85%, at least about 90%, or at least about 95%) homologous or identical to the amino acid sequence set forth in SEQ ID NO: 120. In certain embodiments, the α chain comprises the amino acid sequence set forth in SEQ ID NO: 119; and the β chain comprises the amino acid sequence set forth in SEQ ID NO: 120. In certain embodiments, the TCR is designated as “TCR 8A”. In certain embodiments, the TCR 8A binds to a TP53 peptide comprising a R175H mutation.

In certain embodiments, the CDRs sequences described above including Table 12 are delineated using the IMGT numbering system.

TABLE 12
(TCR 8A)

| CDRs | 1 | 2 | 3 |
|---|---|---|---|
| α-chain | VSGNPY | YITGDNLV | CAVRAPGNTPLVF [SEQ ID NO: |
| | [SEQ ID NO: | [SEQ ID NO: 112] | 113] |
| | 111] | | |
| β-chain | LGHDT | YNNKEL | CASSQDLGFDEKLFF [SEQ ID |
| | [SEQ ID NO: | [SEQ ID NO: 115] | NO: 116] |
| | 114] | | |

| | |
|---|---|
| α-chain | MASAPISMLAML FTLSGLRAQSVAQPEDQVNVAEGNPLTVKCTYSVSGNPYLFWYV |
| variable | QYPNRGLQFLLKYITGDNLVKGSYGFEAEENKSQTSFHLKKPSALVSDSALYFCAV |
| | RAPGNTPLVFGKGIRLSVIA [SEQ ID NO: 117] |
| β-chain | MGCRLLCCVVFCLLQAGPLDTAVSQTPKYLVTQMGNDKSIKCEQNLGHDTMYWYKQ |
| variable | DSKKFLKIMESYNNKELIINETVPNRESPKSPDKAHLNLHINSLELGDSAVYFCAS |
| | SQDLGFDEKLFFGSGTQLSVL [SEQ ID NO: 118] |
| Full α- | MASAPISMLAML FTLSGLRAQSVAQPEDQVNVAEGNPLTVKCTYSVSGNPYLFWYV |
| chain | QYPNRGLQFLLKYITGDNLVKGSYGFEAEENKSQTSFHLKKPSALVSDSALYFCAV |
| | RAPGNTPLVFGKGTRLSVIANIQNPDPAVYQLRDSKSSDKSVCLFTDFDSQTNVSQSKDS |
| | DVYITDKTVLDMRSMDFKSNSAVAWSNKSDFACANAFNNSIIPEDTFFPSPESSCDVKLVEK |
| | SFETDTNLNFQNLSVIGFRILLLKVAGENLLMTLRLWSS [SEQ ID NO: 119] |
| Full β- | MGCRLLCCVVFCLLQAGPLDTAVSQTPKYLVTQMGNDKSIKCEQNLGHDTMYWYKQ |
| chain | DSKKFLKIMFSYNNKELIINETVPNRESPKSPDKAHLNLHINSLELGDSAVYFCAS |
| | SQDLGFDEKLFFGSGTQLSVLEDLNKVEPPEVAVFEPSEAEISHTQKATLVCLATGFFPD |
| | HVELSWWVNGKEVHSGVSTDPQPLKEQPALNDSRYCLSSRLRVSATFWQNPRNHFRCQVQFY |
| | GLSENDEWTQDRAKPVTQIVSAEAWGRADCGFTSVSYQQGVLSATILYEILLGKATLYAVLV |
| | SALVLMAMVKRKDF [SEQ ID NO: 120] |

In certain embodiments, the α chain variable region and/or the β chain variable region amino acid sequences have at least about 80%, at least about 85%, at least about 90%, or at least about 95% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) homology or identity to the specified sequences (e.g., SEQ ID NO: 7, SEQ ID NO: 8, SEQ ID NO: 17, SEQ ID NO: 18, SEQ ID NO: 27, SEQ ID NO: 28, SEQ ID NO: 37, SEQ ID NO: 38, SEQ ID NO: 47, SEQ ID NO: 48, SEQ ID NO: 57, SEQ ID NO: 58, SEQ ID NO: 67, SEQ ID NO: 68, SEQ ID NO: 77, SEQ ID NO: 78, SEQ ID NO: 87, SEQ ID NO: 88, SEQ ID NO: 97, SEQ ID NO: 98, SEQ ID NO: 107, SEQ ID NO: 108, SEQ ID NO: 117, and SEQ ID NO: 118) comprise modifications, including, but not limited to, substitutions (e.g., conservative substitutions), insertions, or deletions relative to the specified sequence(s), but retain the ability to bind to a mutant TP53 peptide (e.g., a R175H mutant TP53 peptide). In certain embodiments, such modifications are not within the CDR domains of the variable regions.

In certain embodiments, a total of 1 to 10 amino acids are substituted, inserted and/or deleted in SEQ ID NO: 7, SEQ ID NO: 8, SEQ ID NO: 17, SEQ ID NO: 18, SEQ ID NO: 27, SEQ ID NO: 28, SEQ ID NO: 37, SEQ ID NO: 38, SEQ ID NO: 47, SEQ ID NO: 48, SEQ ID NO: 57, SEQ ID NO: 58, SEQ ID NO: 67, SEQ ID NO: 68, SEQ ID NO: 77, SEQ ID NO: 78, SEQ ID NO: 87, SEQ ID NO: 88, SEQ ID NO: 97, SEQ ID NO: 98, SEQ ID NO: 107, SEQ ID NO: 108, SEQ ID NO: 117, or SEQ ID NO: 118. In certain embodiments, substitutions, insertions, or deletions occur in regions outside the CDRs of the extracellular domain. In certain embodiments, the extracellular domain comprises an α chain variable region and/or a β chain variable region sequence selected from the group consisting of SEQ ID NO: 7, SEQ ID NO: 8, SEQ ID NO: 17, SEQ ID NO: 18, SEQ ID NO: 27, SEQ ID NO: 28, SEQ ID NO: 37, SEQ ID NO: 38, SEQ ID NO: 47, SEQ ID NO: 48, SEQ ID NO: 57, SEQ ID NO: 58, SEQ ID NO: 67, SEQ ID NO: 68, SEQ ID NO: 77, SEQ ID NO: 78, SEQ ID NO: 87, SEQ ID NO: 88, SEQ ID NO: 97, SEQ ID NO: 98, SEQ ID NO: 107, SEQ ID NO: 108, SEQ ID NO: 117, or SEQ ID NO: 118 including post-translational modifications of that sequence (SEQ ID NO: 7, SEQ ID NO: 8, SEQ ID NO: 17, SEQ ID NO: 18, SEQ ID NO: 27, SEQ ID NO: 28, SEQ ID NO: 37, SEQ ID NO: 38, SEQ ID NO: 47, SEQ ID NO: 48, SEQ ID NO: 57, SEQ ID NO: 58, SEQ ID NO: 67, SEQ ID NO: 68, SEQ ID NO: 77, SEQ ID NO: 78, SEQ ID NO: 87, SEQ ID NO: 88, SEQ ID NO: 97, SEQ ID NO: 98, SEQ ID NO: 107, SEQ ID NO: 108, SEQ ID NO: 117, or SEQ ID NO: 118).

### 5.3.1.2. Constant Regions

In certain embodiments, the presently disclosed TCR comprises an α chain constant region that comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 123, SEQ ID NO: 124, SEQ ID NO: 125, or SEQ ID NO: 148. In certain embodiments, the α chain constant region comprises the amino acid sequence set forth in SEQ ID NO: 123. In certain embodiments, the a chain constant region comprises the amino acid sequence set forth in SEQ ID NO: 124. In certain embodiments, the α chain constant region comprises the amino acid sequence set forth in SEQ ID NO: 125. In certain embodiments, the α chain constant region comprises the amino acid sequence set forth in SEQ ID NO: 148.

In certain embodiments, the presently disclosed TCR comprises a β chain constant region that comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 126, SEQ ID NO:
127, or SEQ ID NO: 129. In certain embodiments, the β chain constant region comprises the amino acid sequence set forth in SEQ ID NO: 126. In certain embodiments, the β chain constant region comprises the amino acid sequence set forth in SEQ ID NO: 127. In certain embodiments, the β chain constant region comprises the amino acid sequence set forth in SEQ ID NO: 129. SEQ ID NO: 123, SEQ ID NO: 124, SEQ ID NO: 125, SEQ ID NO: 126, SEQ ID NO: 127, SEQ ID NO: 129, and SEQ ID NO: 148 are provided below:

Human α chain constant region:

[SEQ ID NO: 123]
NIQNPDPAVYQLRDSKSSDKSVCLFTDFDSQTNVSQSKDSDVYITDKTVL
DMRSMDFKSNSAVAWSNKSDFACANAFNNSIIPEDTFFPSPESSCDVKLV
EKSFETDTNLNFQNLSVIGFRILLLKVAGENLLMTLRLWSS

Mouse α chain constant region (cysteine-modification and LVL modification in transmembrane domain underlined):

[SEQ ID NO: 124]
NIQNPEPAVYQLKDPRSQDSTLCLFTDFDSQINVPKTMESGTFITDKCVL
DMKAMDSKSNGAIAWSNQTSFTCQDIFKETNATYPSSDVPCDATLTEKSF
ETDMNLNFQNLLVIVLRILLLKVAGENLLMTLRLWSS

Mouse α chain constant region (native):

[SEQ ID NO: 125]
DIQNPEPAVYQLKDPRSQDSTLCLFTDFDSQINVPKTMESGTFITDKTVL
DMKAMDSKSNGAIAWSNQTSFTCQDIFKETNATYPSSDVPCDATLTEKSF
ETDMNLNFQNLSVMGLRILLLKVAGENLLMTLRL

Human β chain constant region:

[SEQ ID NO: 126]
EDLNKVFPPEVAVFEPSEAEISHTQKATLVCLATGFFPDHVELSWWVNGK
EVHSGVSTDPQPLKEQPALNDSRYCLSSRLRVSATFWQNPRNHFRCQVQF
YGLSENDEWTQDRAKPVTQIVSAEAWGRADCGFTSVSYQQGVLSATILYE
ILLGKATLYAVLVSALVLMAMVKRKDF

Mouse β chain constant region (cysteine-modification underlined):

[SEQ ID NO: 127]
EDLRNVTPPKVSLFEPSKAEIANKQKATLVCLARGFFPDHVELSWWVNGK
EVHSGVCTDPQAYKESNYSYCLSSRLRVSATFWHNPRNHFRCQVQFHGLS
EEDKWPEGSPKPVTQNISAEAWGRADCGITSASYQQGVLSATILYEILLG
KATLYAVLVSTLVVMAMVKRKNS

Human β chain constant region:

[SEQ ID NO: 129]
EDLKNVFPPEVAVFEPSEAEISHTQKATLVCLATGFYPDHVELSWWVNGK
EVHSGVSTDPQPLKEQPALNDSRYCLSSRLRVSATFWQNPRNHFRCQVQF
YGLSENDEWTQDRAKPVTQIVSAEAWGRADCGFTSESYQQGVLSATILYE
ILLGKATLYAVLVSALVLMAMVKRKDSRG

Mouse α chain constant region (cysteine-modification and LVL modification in transmembrane domain underlined):

[SEQ ID NO: 148]
DIQNPEPAVYQLKDPRSQDSTLCLFTDFDSQINVPKTMESGTFITDKCVL
DMKAMDSKSNGAIAWSNQTSFTCQDIFKETNATYPSSDVPCDATLTEKSF
ETDMNLNFQNLLVIVLRILLLKVAGENLLMTLRLWSS

5.3.2. TCRs that Bind to the Same TP53 Peptide as TCR Clonotypes

The presently disclosed subject matter further provides TCRs that bind to the same TP53 peptide (e.g., a R175H mutant TP53 peptide) as a TCR disclosed herein (e.g., a TCR disclosed in Section 5.3.1). In certain embodiments, the TCR binds to the same TP53 peptide (e.g., a R175H mutant TP53 peptide) as a reference TCR or a functional fragment thereof comprising the α chain variable region comprising a CDR1, a CDR2, and a CDR3 and the β chain variable region comprising a CDR1, a CDR2, and a CDR3 of, for example, the TCRs disclosed herein (e.g., disclosed in Section 5.3.1). In certain embodiments, the TCR binds to the same TP53 peptide (e.g., a R175H mutant TP53 peptide) as a reference TCR or a functional fragment thereof comprising the α chain variable region and the β chain variable region sequences of, for example, the presently disclosed TCR (e.g., disclosed in Section 5.3.1).

5.3.3. TCRs with Modifications within CDRs

In certain embodiments, a presently disclosed TCR (or a functional fragment thereof) comprises an α chain variable region comprising a CDR1, a CDR2 and a CDR3 sequences and a β chain variable region comprising a CDR1, a CDR2 and a CDR3 sequences, wherein one or more of these CDR sequences comprise specified amino acid sequences based on the TCR (or a functional fragments thereof) described herein, or modifications thereof, and wherein the TCR (or a functional fragments thereof) retains the desired functional properties of the mutant TP53 peptide-specific TCR (or a functional fragments thereof) of the presently disclosed subject matter.

In certain embodiments, a presently disclosed TCR (or a functional fragment thereof) comprises an α chain constant region and a β chain constant region, wherein at least one of the constant regions comprises specified amino acid sequences based on the TCR (or a functional fragments thereof) described herein, or modifications thereof, and wherein the TCR (or a functional fragment thereof) retains the desired functional properties of the mutant TP53 peptide-specific TCRs (or a functional fragments thereof) of the presently disclosed subject matter.

In certain embodiments, such modifications do not significantly affect or alter the binding characteristics of the TCR comprising the amino acid sequence. Non-limiting examples of such modifications include amino acid substitutions, additions and deletions. Modifications can be introduced into the presently disclosed TCR or a functional fragment thereof by standard techniques known in the art, such as site-directed mutagenesis and PCR-mediated mutagenesis.

The modifications can be conservative modifications, non-conservative modifications, or mixtures of conservative and non-conservative modifications. As discussed above, conservative amino acid substitutions are ones in which the amino acid residue is replaced with an amino acid residue having a similar side chain. Families of amino acid residues having similar side chains have been defined in the art. Exemplary conservative amino acid substitutions are shown in Table 13. In certain embodiments, amino acid substitutions may be introduced into a TCR of interest and the products screened for a desired activity, e.g., retained/improved antigen binding, decreased immunogenicity, or improved ADCC or CDC.

TABLE 13

| Original Residue | Exemplary conservative amino acid Substitutions |
|---|---|
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe |
| Leu (L) | Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala |

Amino acids may be classified according to common side-chain properties:
hydrophobic: Norleucine, Met, Ala, Val, Leu, Ile;
neutral hydrophilic: Cys, Ser, Thr, Asn, Gln;
acidic: Asp, Glu;
basic: His, Lys, Arg;
residues that influence chain orientation: Gly, Pro;
aromatic: Trp, Tyr, Phe.

In certain embodiments, one or more amino acid residues within a CDR region can be replaced with other amino acid residues from the same group and the altered TCR can be tested for retained function using the functional assays described herein.

Non-conservative substitutions entail exchanging a member of one of these classes for another class.

In certain embodiments, no more than one, no more than two, no more than three, no more than four, no more than five residues within a specified sequence or a CDR region are altered.

In certain embodiments, one or more amino acid residues within a constant region of a TCR can be modified to enhance stability and/or cell surface expression of the TCR. In certain embodiments, no more than one, no more than two, no more than three, no more than four, no more than five residues within a specified sequence or a constant region are altered. In certain embodiments, the modification includes but is not limited to, murinization, cysteine modification and transmembrane modification (see Cohen et al. Enhanced antitumor activity of murine-human hybrid T-cell receptor (TCR) in human lymphocytes is associated with improved pairing and TCR/CD3 stability, Cancer Res. 2006; 66 (17): 8878-8886; Cohen et al. Enhanced antitumor activity of T cells engineered to express T-cell receptors with a second disulfide bond, Cancer Res. 2007; 67 (8): 3898-3903; Kuball et al. Facilitating matched pairing and expression of TCR chains introduced into human T cells, Blood 2007; 109 (6): 2331-2338; Haga-Friedman et al. Incorporation of transmembrane hydrophobic mutations in the TCR enhance its surface expression and T cell functional avidity, Journal of immunology 2012; 188 (11): 5538-5546, the contents of each of which are incorporated by reference in their entireties).

### 5.3.4. Bispecific Molecules

The presently disclosed subject matter provides bispecific molecules comprising a presently disclosed TCR (or a functional fragment thereof). A presently disclosed TCR or a functional fragment thereof can be derivatized or linked to another functional molecule, e.g., another peptide or protein (e.g., another antibody or ligand for a receptor) to generate a bispecific molecule that binds to at least two different binding sites or target molecules. The presently disclosed TCR or a functional fragment thereof can in fact be derivatized or linked to more than one other functional molecule to generate multi-specific molecules that bind to more than two different binding sites and/or target molecules; such multi-specific molecules are also intended to be encompassed by the term “bispecific molecule” as used herein. To create a bispecific molecule, a presently disclosed TCR or a functional fragment thereof can be functionally linked (e.g., by chemical coupling, genetic fusion, noncovalent association or otherwise) to one or more other binding molecules, such as another antibody, antibody fragment, peptide or binding mimetic.

The presently disclosed subject matter provides bispecific molecules comprising at least a first binding specificity for a mutant TP53 peptide and a second binding specificity for a second target peptide region. The second target epitope region can be a second TP53 peptide, or a non-TP53 peptide, e.g., a different antigen. In certain embodiments, the bispecific molecule is multi-specific, e.g., the molecule can further include a third binding specificity. Where a first portion of a bispecific molecule, e.g., antibody, binds to an antigen on a tumor cell for example and a second portion of a bispecific molecule recognizes an antigen on the surface of a human immune effector cell, the bispecific molecule is capable of recruiting the activity of that effector cell by specifically binding to the effector antigen on the human immune effector cell. In certain embodiments, bispecific molecules are able to form a link between effector cells, for example, T cells and tumor cells, thereby enhancing effector function. In certain embodiments, a presently disclosed bispecific molecule comprises at least a first binding to a mutant TP53 peptide and at least a second binding to an immune cell or a molecule associated with an immune cell.

The bispecific molecules of the presently disclosed subject matter can be prepared by conjugating the constituent binding specificities using methods known in the art. For example, each binding specificity of the bispecific molecule can be generated separately and then conjugated to one another. When the binding specificities are proteins or peptides, a variety of coupling or cross-linking agents can be used for covalent conjugation. Non-limiting examples of cross-linking agents include protein A, carbodiimide, N-succinimidyl-S-acetyl-thioacetate (SATA), 5,5′-dithiobis(2-nitrobenzoic acid) (DTNB), o-phenylenedimaleimide (oPDM), N-succinimidyl-3-(2-pyridyldithio) propionate (SPDP), and sulfosuccinimidyl 4-(N-maleimidomethyl) cyclohaxane-1-carboxylate (sulfo-SMCC) (see e.g., Karpovsky et al. (1984) J. Exp. Med. 160:1686; Liu, M A et al. (1985) Proc. Natl. Acad. Sci. USA 82:8648). Other methods include those described in Paulus (1985) Behring Ins. Mitt. No. 78, 118-132; Brennan et al. (1985) Science 229:81-83), and Glennie et al. (1987) J. Immunol. 139:2367-2375). Conjugating agents can be SATA and sulfo-SMCC, both available from Pierce Chemical Co. (Rockford, IL).

When the binding specificities are antibodies, they can be conjugated via sulfhydryl bonding of the C-terminus hinge regions of the two heavy chains. In certain embodiments, the hinge region is modified to contain an odd number of sulfhydryl residues, preferably one, prior to conjugation.

Alternatively, both binding specificities can be encoded in the same vector and expressed and assembled in the same host cell. This method is particularly useful where the bispecific molecule is a mAb and a mAb, a mAb and a Fab, a Fab and a F(ab′) 2, or a ligand and a Fab fusion protein.

Binding of the bispecific molecules to their specific targets can be confirmed by, for example, enzyme-linked immunosorbent assay (ELISA), radioimmunoassay (RIA), FACS analysis, bioassay (e.g., growth inhibition), or Western Blot assay. Each of these assays generally detects the presence of protein-antibody complexes of particular interest by employing a labeled reagent (e.g., an antibody) specific for the complex of interest. Alternatively, the complexes can be detected using any of a variety of other immunoassays. For example, the antibody can be radioactively labeled and used in a radioimmunoassay (RIA) (see, for example, Weintraub, B., Principles of Radioimmunoassays, Seventh Training Course on Radioligand Assay Techniques, The Endocrine Society, March 1986, which is incorporated by reference herein). The radioactive isotope can be detected by such means as the use of a γ counter or a scintillation counter or by autoradiography.

### 5.4. Cells

The presently disclosed subject matter provides cells comprising a presently disclosed TCR (e.g., one disclosed in Section 5.3). In certain embodiments, the cell is selected from the group consisting of cells of lymphoid lineage, cells of myeloid lineage, stem cells from which cells of lymphoid lineage can be derived, and stem cells from which cells of myeloid lineage can be derived. In certain embodiments, the cell is an immunoresponsive cell. In certain embodiments, the immunoresponsive cell is a cell of lymphoid lineage.

In certain embodiments, the cell is a cell of the lymphoid lineage. Cells of the lymphoid lineage can provide production of antibodies, regulation of cellular immune system, detection of foreign agents in the blood, detection of cells foreign to the host, and the like. Non-limiting examples of cells of the lymphoid lineage include T cells and/or stem cells from which lymphoid cells may be differentiated. In certain embodiments, the stem cell is a pluripotent stem cell (e.g., embryonic stem cell).

In certain embodiments, the cell is a T cell. T cells can be lymphocytes that mature in the thymus and are chiefly responsible for cell-mediated immunity. T cells are involved in the adaptive immune system. The T cells of the presently disclosed subject matter can be any type of T cells, including, but not limited to, helper T cells, cytotoxic T cells, memory T cells (including central memory T cells, stem-cell-like memory T cells (or stem-like memory T cells), and two types of effector memory T cells: e.g., TEM cells and TEMRA cells, Regulatory T cells (also known as suppressor T cells), tumor-infiltrating lymphocyte (TIL), Natural killer T cells, Mucosal associated invariant T cells, and γδ T cells. Cytotoxic T cells (CTL or killer T cells) are a subset of T lymphocytes capable of inducing the death of infected somatic or tumor cells. A patient's own T cells may be genetically modified to target specific antigens through the introduction of an antigen-recognizing receptor, e.g., a CAR. In certain embodiments, the immunoresponsive cell is a T cell. The T cell can be a CD4⁺ T cell or a CD8⁺ T cell. In certain embodiments, the T cell is a CD4⁺ T cell. In certain embodiments, the T cell is a CD8⁺ T cell. In certain embodiments, the TCR-expressing T cells express Foxp3 to achieve and maintain a T regulatory phenotype.

In certain embodiments, the T cell is a NK-T cell. Natural killer (NK) T cells can be lymphocytes that are part of cell-mediated immunity and act during the innate immune response. NK-T cells do not require prior activation in order to perform their cytotoxic effect on target cells.

In certain embodiments, the cell is a Natural Killer (NK) cell. NK cells constitute the predominant innate lymphocyte subset that physiologically mediates the anti-viral and anti-tumor immune responses. NK cells use an array of innate receptors to sense their environment and to respond to infections, cellular stress, and transformation. The resulting NK cell activation, including cytotoxicity and cytokine production, is a fundamental component of the early immune response. Types of human lymphocytes of the presently disclosed subject matter include, without limitation, peripheral donor lymphocytes. e.g., those disclosed in Sadelain et al., Nat Rev Cancer (2003); 3:35-45 (disclosing peripheral donor lymphocytes genetically modified to express CARs), in Morgan, R. A., et al. 2006 Science 314:126-129 (disclosing peripheral donor lymphocytes genetically modified to express a full-length tumor antigen-recognizing T cell receptor complex comprising the α and β heterodimer), in Panelli et al., J Immunol (2000); 164:495-504; Panelli et al., J Immunol (2000); 164:4382-4392 (disclosing lymphocyte cultures derived from tumor infiltrating lymphocytes (TILs) in tumor biopsies), and in Dupont et al., Cancer Res (2005); 65:5417-5427; Papanicolaou et al., Blood (2003); 102:2498-2505 (disclosing selectively in vitro-expanded antigen-specific peripheral blood leukocytes employing artificial antigen-presenting cells (AAPCs) or pulsed dendritic cells).

The cells (e.g., T cells) can be autologous, non-autologous (e.g., allogeneic), or derived in vitro from engineered progenitor or stem cells.

The cells of the presently disclosed subject matter can be cells of the myeloid lineage. Non-limiting examples of cells of the myeloid lineage include monocytes, macrophages, neutrophils, dendritic cells, basophils, neutrophils, eosinophils, megakaryocytes, mast cell, erythrocyte, thrombocytes, and stem cells from which myeloid cells may be differentiated. In certain embodiments, the stem cell is a pluripotent stem cell (e.g., an embryonic stem cell or an induced pluripotent stem cell).

In certain embodiments, the cell further comprises at least one recombinant or exogenous co-receptor. For example, a presently disclosed cell can be further transduced with at least one co-receptor, such that the cell co-expresses or is induced to co-express the presently disclosed TCR and the at least one co-receptor. The interaction between the presently disclosed TCR and at least one co-receptor with the MHC complex of the target cell (e.g., tumor cells expressing TP53) improves the antigen-specific signal required for full activation of an immunoresponsive cell (e.g., T cell).

In certain embodiments, the co-receptor is a CD8 co-receptor. In certain embodiments, the CD8 co-receptor comprises an α chain and a β chain. In certain embodiments, the α chain of the CD8 co-receptor comprises or consists of an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence having a UniProt Reference No: P01732, or fragments thereof, and/or may optionally comprise up to one or up to two or up to three conservative amino acid substitutions. In certain embodiments, the a chain of the CD8 co-receptor comprises or consists of an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 130. In certain embodiments, the α chain of the CD8 co-receptor comprises or consists of the amino acid sequence set forth in SEQ ID NO: 130. In certain embodiments, the β chain of the CD8 co-receptor comprises or consists of an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence having a UniProt Reference No: P10966, or fragments thereof, and/or may optionally comprise up to one or up to two or up to three conservative amino acid substitutions. In certain embodiments, the β chain of the CD8 co-receptor comprises or consists of an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 131. In certain embodiments, the β chain of the CD8 co-receptor comprises or consists of the amino acid sequence set forth in SEQ ID NO: 131.

In certain embodiments, the CD8 co-receptor comprises an α chain comprising or consists of an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 130; and a β chain comprising or consists of an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 131. In certain embodiments, the CD8 co-receptor comprises an α chain comprising or consists of the amino acid sequence set forth in SEQ ID NO: 130; and a β chain comprising or consists of the amino acid sequence set forth in SEQ ID NO: 131. SEQ ID NO: 130 and SEQ ID NO: 131 are provided below.

[SEQ ID NO: 130]
MALPVTALLLPLALLLHAARPSQFRVSPLDRTWNLGETVELKCQVLLSNP
TSGCSWLFQPRGAAASPTELLYLSQNKPKAAEGLDTQRFSGKRLGDTFVL
TLSDFRRENEGYYFCSALSNSIMYFSHFVPVFLPAKPTTTPAPRPPTPAP
TIASQPLSLRPEACRPAAGGAVHTRGLDFACDIYIWAPLAGTCGVLLLSL
VITLYCNHRNRRRVCKCPRPVVKSGDKPSLSARYV
[SEQ ID NO: 131]
MRPRLWLLLAAQLTVLHGNSVLQQTPAYIKVQTNKMVMLSCEAKISLSNM
RIYWLRQRQAPSSDSHHEFLALWDSAKGTIHGEEVEQEKIAVFRDASRFI
LNLTSVKPEDSGIYFCMIVGSPELTFGKGTQLSVVDFLPTTAQPTKKSTL
KKRVCRLPRPETQKGPLCSPITLGLLVAGVLVLLVSLGVAIHLCCRRRRA
RLREMKQFYK

In certain embodiments, the co-receptor is a CD4 co-receptor. In certain embodiments, the CD4 co-receptor comprises a polypeptide comprising or consisting of an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence having a UniProt Reference No: P01730, or fragments thereof, and/or may optionally comprise up to one or up to two or up to three conservative amino acid substitutions. In certain embodiments, the CD4 co-receptor comprises a polypeptide comprising or consisting of an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 132. In certain embodiments, the CD4 co-receptor comprises a polypeptide comprising or consisting of the amino acid sequence set forth in SEQ ID NO: 132. SEQ ID NO: 132 is provided below:

[SEQ ID NO: 132]
MNRGVPFRHLLLVLQLALLPAATQGKKVVLGKKGDTVELTCTASQKKSIQ
FHWKNSNQIKILGNQGSFLTKGPSKLNDRADSRRSLWDQGNFPLIIKNLK
IEDSDTYICEVEDQKEEVQLLVFGLTANSDTHLLQGQSLTLTLESPPGSS
PSVQCRSPRGKNIQGGKTLSVSQLELQDSGTWTCTVLQNQKKVEFKIDIV
VLAFQKASSIVYKKEGEQVEFSFPLAFTVEKLTGSGELWWQAERASSSKS
WITFDLKNKEVSVKRVTQDPKLQMGKKLPLHLTLPQALPQYAGSGNLTLA
LEAKTGKLHQEVNLVVMRATQLQKNLTCEVWGPTSPKLMLSLKLENKEAK
VSKREKAVWVLNPEAGMWQCLLSDSGQVLLESNIKVLPTWSTPVQPMALI
VLGGVAGLLLFIGLGIFFCVRCRHRRRQAERMSQIKRLLSEKKTCQCPHR
FQKTCSPI

In certain embodiments, cell further comprises at least one recombinant or exogenous co-stimulatory ligand. For example, a presently disclosed cell can be further transduced with at least one co-stimulatory ligand, such that the cell co-expresses or is induced to co-express the presently disclosed TCR and the at least one co-stimulatory ligand. The interaction between the presently disclosed TCR and at least one co-stimulatory ligand provides a non-antigen-specific signal important for full activation of an immunoresponsive cell (e.g., T cell). Co-stimulatory ligands include, but are not limited to, members of the tumor necrosis factor (TNF) superfamily, and immunoglobulin (Ig) superfamily ligands. TNF is a cytokine involved in systemic inflammation and stimulates the acute phase reaction. Its primary role is in the regulation of immune cells. Members of TNF superfamily share a number of common features. The majority of TNF superfamily members are synthesized as type II transmembrane proteins (extracellular C-terminus) containing a short cytoplasmic segment and a relatively long extracellular region. TNF superfamily members include, without limitation, nerve growth factor (NGF), CD40L (CD40L)/CD154, CD137L/4-1BBL, TNF-α, CD134L/OX40L/CD252, CD27L/CD70, Fas ligand (FasL), CD30L/CD153, tumor necrosis factor beta (TNF-β)/lymphotoxin-alpha (LTa), lymphotoxin-beta (LTB), CD257/B cell-activating factor (BAFF)/Blys/THANK/Tall-1, glucocorticoid-induced TNF Receptor ligand (GITRL), and TNF-related apoptosis-inducing ligand (TRAIL), LIGHT (TNFSF14). The immunoglobulin (Ig) superfamily is a large group of cell surface and soluble proteins that are involved in the recognition, binding, or adhesion processes of cells. These proteins share structural features with immunoglobulins-they possess an immunoglobulin domain (fold). Immunoglobulin superfamily ligands include, but are not limited to, CD80 and CD86, both ligands for CD28, PD-L1/(B7-H1) that ligands for PD-1. In certain embodiments, the at least one co-stimulatory ligand is selected from the group consisting of 4-1BBL, CD80, CD86, CD70, OX40L, CD48, TNFRSF14, PD-L1, and combinations thereof. In certain embodiments, the cell comprises one recombinant co-stimulatory ligand that is 4-1BBL. In certain embodiments, the cell comprises two recombinant co-stimulatory ligands that are 4-1BBL and CD80.

In certain embodiments, a presently disclosed cell further comprises at least one exogenous cytokine. For example, a presently disclosed cell can be further transduced with at least one cytokine, such that the cell secretes the at least one cytokine as well as expresses the presently disclosed TCR. In certain embodiments, the at least one cytokine is selected from the group consisting of IL-2, IL-3, IL-6, IL-7, IL-11, IL-12, IL-15, IL-17, IL-18, and IL-21. In certain embodiments, the cytokine is IL-12.

### 5.5. Nucleic Acids and Genetic Modifications of Cells

The present discloses subject matter provides a nucleic acid encoding a presently disclosed TCR (e.g., one disclosed in Section 5.3). Further provided are cells comprising a vector comprising such nucleic acids. In certain embodiments, a promoter is operably linked to a polynucleotide encoding a presently disclosed TCR.

In certain embodiments, the promoter is endogenous or exogenous. In certain embodiments, the exogenous promoter is selected from the group consisting of a long terminal repeat (LTR) promoter, an elongation factor (EF)-1 promoter, a cytomegalovirus immediate-early promoter (CMV) promoter, a simian virus 40 early promoter (SV40) promoter, a phosphoglycerate kinase (PGK) promoter, and a metallothionein promoter. In certain embodiment, the exogenous promoter is a LTR promoter. In certain embodiments, the promoter is an inducible promoter. In certain embodiment, the inducible promoter is selected from the group consisting of a NFAT transcriptional response element (TRE) promoter, a CD69 promoter, a CD25 promoter, and an IL-2 promoter.

In certain embodiments, the nucleic acid encodes both the α chain and the β chain of a presently disclosed TCR. In certain embodiments, the α chain and the β chain are separated by a self-cleavage peptide, e.g., a 2A-peptide. In certain embodiments, the α chain and the β chain are separated by a furin-2A-peptide. In certain embodiments, the peptide comprises the amino acid sequence set forth in SEQ ID NO: 133. SEQ ID NO: 133 is provided below:

[SEQ ID NO: 133]
RAKRSGSGATNFSLLKQAGDVEENPGP

In certain embodiments, the nucleic acid encodes a polypeptide comprising an α chain and a β chain of one of the presently disclosed TCRs. In certain embodiments, the polypeptide further comprises a self-cleavable peptide (e.g., a self-cleavable peptide set forth in SEQ ID NO: 133). In certain embodiments, the nucleic acid encodes a polypeptide comprising an amino acid sequence that is at least about 80%, about 85%, about 90%, and about 95% identical to the amino acid sequence set forth in SEQ ID Nos. 134-145. In certain embodiments, the nucleic acid encodes a polypeptide comprising or consisting of the amino acid sequence set forth in SEQ ID Nos. 134-145. SEQ ID Nos. 134-145 are provided in Table 14.

TABLE 14

| TCR | Sequence |
|---|---|
| 1A | MGTRLLCWAALCLLGAELTEAGVAQSPRYKIIEKRQSVAFWCNPISGHATLYWYQQILGQGPKLLIQFQNN |
| | GVVDDSQLPKDRFSAERLKGVDSTLKIQPAKLEDSAVYLCASSLDYGSSYEQYFGPGTRLTVTEDLRNVTP |
| | PKVSLFEPSKAEIANKQKATLVCLARGFFPDHVELSWWVNGKEVHSGVCTDPQAYKESNYSYCLSSRLRVS |
| | ATFWHNPRNHFRCQVQFHGLSEEDKWPEGSPKPVTQNISAEAWGRADCGITSASYQQGVLSATILYEILLG |
| | KATLYAVLVSTLVVMAMVKRKNSRAKRSGSGATNFSLLKQAGDVEENPGPMETLLGVSLVILWLQLARVNS |
| | QQGEEDPQALSIQEGENATMNCSYKTSINNLQWYRQNSGRGLVHLILIRSNEREKHSGRLRVTLDTSKKSS |
| | SLLITASRAADTASYFCATGSNYGGSQGNLIFGKGTKLSVKPDIQNPEPAVYQLKDPRSQDSTLCLFTDED |
| | SQINVPKTMESGTFITDKCVLDMKAMDSKSNGAIAWSNQTSFTCQDIFKETNATYPSSDVPCDATLTEKSF |
| | ETDMNLNFQNLLVIVLRILLLKVAGENLLMTLRLWSS [SEQ ID NO: 134] |
| 1B | MGPGLLCWVLLCLLGAGPVDAGVTQSPTHLIKTRGQQVTLRCSPISGHKSVSWYQQVLGQGPQFIFQYYEK |
| | EERGRGNFPDRFSARQFPNYSSELNVNALLLGDSALYLCASSDDTGTTKSSGANVLTFGAGSRLTVLEDLR |
| | NVTPPKVSLFEPSKAEIANKQKATLVCLARGFFPDHVELSWWVNGKEVHSGVCTDPQAYKESNYSYCLSSR |
| | LRVSATFWHNPRNHFRCQVQFHGLSEEDKWPEGSPKPVTQNISAEAWGRADCGITSASYQQGVLSATILYE |
| | ILLGKATLYAVLVSTLVVMAMVKRKNSRAKRSGSGATNFSLLKQAGDVEENPGPMSLSSLLKVVTASLWLG |
| | PGIAQKITQTQPGMFVQEKEAVTLDCTYDTSDPSYGLFWYKQPSSGEMIFLIYQGSYDQQNATEGRYSLNF |
| | QKARKSANLVISASQLGDSAMYFCAMREGFSGGYNKLIFGAGTRLAVHPDIQNPEPAVYQLKDPRSQDSTL |
| | CLFTDFDSQINVPKTMESGTFITDKCVLDMKAMDSKSNGAIAWSNQTSFTCQDIFKETNATYPSSDVPCDA |
| | TLTEKSFETDMNLNFQNLLVIVLRILLLKVAGENLLMTLRLWSS [SEQ ID NO: 135] |
| 1C | MGFRLLCCVAFCLLGAGPVDSGVTQTPKHLITATGQRVTLRCSPRSGDLSVYWYQQSLDQGLQFLIQYYNG |
| | EERAKGNILERFSAQQFPDLHSELNLSSLELGDSALYFCASSTPLSGSSTDTQYFGPGTRLTVLEDLRNVT |
| | PPKVSLFEPSKAEIANKQKATLVCLARGFFPDHVELSWWVNGKEVHSGVCTDPQAYKESNYSYCLSSRLRV |
| | SATFWHNPRNHFRCQVQFHGLSEEDKWPEGSPKPVTQNISAEAWGRADCGITSASYQQGVLSATILYEILL |
| | GKATLYAVLVSTLVVMAMVKRKNSRAKRSGSGATNFSLLKQAGDVEENPGPMAMLLGASVLILWLQPDWVN |
| | SQQKNDDQQVKQNSPSLSVQEGRISILNCDYTNSMEDYFLWYKKYPAEGPTFLISISSIKDKNEDGRFTVF |
| | LNKSAKHLSLHIVPSQPGDSAVYFCAATVGLAGNMLTFGGGTRLMVKPDIQNPEPAVYQLKDPRSQDSTLC |
| | LFTDFDSQINVPKTMESGTFITDKCVLDMKAMDSKSNGAIAWSNQTSFTCQDIFKETNATYPSSDVPCDAT |
| | LTEKSFETDMNLNFQNLLVIVLRILLLKVAGENLLMTLRLWSS [SEQ ID NO: 136] |
| 1D | MGFRLLCCVAFCLLGAGPVDSGVTQTPKHLITATGQRVTLRCSPRSGDLSVYWYQQSLDQGLQFLIHYYNG |
| | EERAKGNILERFSAQQFPDLHSELNLSSLELGDSALYFCASSTPLRTRGTDTQYFGPGTRLTVLEDLRNVT |
| | PPKVSLFEPSKAEIANKQKATLVCLARGFFPDHVELSWWVNGKEVHSGVCTDPQAYKESNYSYCLSSRLRV |
| | SATFWHNPRNHFRCQVQFHGLSEEDKWPEGSPKPVTQNISAEAWGRADCGITSASYQQGVLSATILYEILL |
| | GKATLYAVLVSTLVVMAMVKRKNSRAKRSGSGATNFSLLKQAGDVEENPGPMEKMLECAFIVLWLQLGWLS |
| | GEDQVTQSPEALRLQEGESSSLNCSYTVSGLRGLFWYRQDPGKGPEFLFTLYSAGEEKEKERLKATLTKKE |
| | SFLHITAPKPEDSATYLCAVHSNYQLIWGAGTKLIIKPDIQNPEPAVYQLKDPRSQDSTLCLFTDFDSQIN |
| | VPKTMESGTFITDKCVLDMKAMDSKSNGAIAWSNQTSFTCQDIFKETNATYPSSDVPCDATLTEKSFETDM |
| | NLNFQNLLVIVLRILLLKVAGENLLMTLRLWSS [SEQ ID NO: 137] |
| 3C | MGTRLLCWAALCLLGADHTGAGVSQTPSNKVTEKGKYVELRCDPISGHTALYWYRQSLGQGPEFLIYFQGT |
| | GAADDSGLPNDRFFAVRPEGSVSTLKIQRTERGDSAVYLCASSSKTGVVGEQFFGPGTRLTVLEDLRNVTP |
| | PKVSLFEPSKAEIANKQKATLVCLARGFFPDHVELSWWVNGKEVHSGVCTDPQAYKESNYSYCLSSRLRVS |
| | ATFWHNPRNHFRCQVQFHGLSEEDKWPEGSPKPVTQNISAEAWGRADCGITSASYQQGVLSATILYEILLG |
| | KATLYAVLVSTLVVMAMVKRKNSRAKRSGSGATNFSLLKQAGDVEENPGPMKTFAGFSFLFLWLQLDCMSR |
| | GEDVEQSLFLSVREGDSSVINCTYTDSSSTYLYWYKQEPGAGLQLLTYIFSNMDMKQDQRLTVLLNKKDKH |
| | LSLRIADTQTGDSAIYFCAEGYGGKLIFGQGTELSVKPDIQNPEPAVYQLKDPRSQDSTLCLFTDFDSQIN |
| | VPKTMESGTFITDKCVLDMKAMDSKSNGAIAWSNQTSFTCQDIFKETNATYPSSDVPCDATLTEKSFETDM |
| | NLNFQNLLVIVLRILLLKVAGENLLMTLRLWSS [SEQ ID NO: 138] |
| 3A | MGTSLLCWMALCLLGADHADTGVSQNPRHKITKRGQNVTFRCDPISEHNRLYWYRQTLGQGPEFLTYFQNE |
| | AQLEKSRLLSDRESAERPKGSFSTLEIQRTEQGDSAMYLCASSPLDSYQETQYFGPGTRLLVLEDLRNVTP |
| | PKVSLEEPSKAEIANKQKATLVCLARGFFPDHVELSWWVNGKEVHSGVCTDPQAYKESNYSYCLSSRLRVS |
| | ATFWHNPRNHFRCQVQFHGLSEEDKWPEGSPKPVTQNISAEAWGRADCGITSASYQQGVLSATILYEILLG |
| | KATLYAVLVSTLVVMAMVKRKNSRAKRSGSGATNFSLLKQAGDVEENPGPMKTFAGFSFLFLWLQLDCMSR |
| | GEDVEQSLFLSVREGDSSVINCTYTDSSSTYLYWYKQEPGAGLQLLTYIFSNMDMKQDQRLTVLLNKKDKH |
| | LSLRIADTQTGDSAIYFCAEISANQAGTALIFGKGTTLSVSSDIQNPEPAVYQLKDPRSQDSTLCLFTDED |
| | SQINVPKTMESGTFITDKCVLDMKAMDSKSNGAIAWSNQTSFTCQDIFKETNATYPSSDVPCDATLTEKSF |
| | ETDMNLNFQNLLVIVLRILLLKVAGENLLMTLRLWSS [SEQ ID NO: 139] |
| 3B | MGCRLLCCAVLCLLGAVPMETGVTQTPRHLVMGMTNKKSLKCEQHLGHNAMYWYKQSAKKPLELMFVYSLE |
| | ERVENNSVPSRFSPECPNSSHLFLHLHTLQPEDSALYLCASSQDTAWDSGGETDTQYFGPGTRLTVLEDLR |
| | NVTPPKVSLFEPSKAEIANKQKATLVCLARGFFPDHVELSWWVNGKEVHSGVCTDPQAYKESNYSYCLSSR |
| | LRVSATFWHNPRNHFRCQVQFHGLSEEDKWPEGSPKPVTQNISAEAWGRADCGITSASYQQGVLSATILYE |
| | ILLGKATLYAVLVSTLVVMAMVKRKNSRAKRSGSGATNFSLLKQAGDVEENPGPMSLSSLLKVVTASLWLG |
| | PGIAQKITQTQPGMFVQEKEAVTLDCTYDTSDQSYGLFWYKQPSSGEMIFLIYQGSYDEQNATEGRYSLNF |
| | QKARKSANLVISASQLGDSAMYFCAMREGRGRRALTFGSGTRLQVQPDIQNPEPAVYQLKDPRSQDSTLCL |
| | FTDFDSQINVPKTMESGTFITDKCVLDMKAMDSKSNGAIAWSNQTSFTCQDIFKETNATYPSSDVPCDATL |
| | TEKSFETDMNLNFQNLLVIVLRILLLKVAGENLLMTLRLWSS [SEQ ID NO: 140] |
| 4A | MGCRLLCCVVFCLLQAGPLDTAVSQTPKYLVTQMGNDKSIKCEQNLGHDTMYWYKQDSKKFLKIMFSYNNK |
| | ELIINETVPNRFSPKSPDKAHLNLHINSLELGDSAVYFCASSQALAGEVDTQYFGPGTRLTVLEDLRNVTP |
| | PKVSLFEPSKAEIANKQKATLVCLARGFFPDHVELSWWVNGKEVHSGVCTDPQAYKESNYSYCLSSRLRVS |
| | ATFWHNPRNHFRCQVQFHGLSEEDKWPEGSPKPVTQNISAEAWGRADCGITSASYQQGVLSATILYEILLG |
| | KATLYAVLVSTLVVMAMVKRKNSRAKRSGSGATNFSLLKQAGDVEENPGPMTRVSLLWAVVVSTCLESGMA |
| | QTVTQSQPEMSVQEAETVTLSCTYDTSESNYYLFWYKQPPSRQMILVIRQEAYKQQNATENRFSVNFQKAA |
| | KSFSLKISDSQLGDTAMYFCAFMKWQGGSEKLVFGKGTKLTVNPDIQNPEPAVYQLKDPRSQDSTLCLFTD |
| | FDSQINVPKTMESGTFITDKCVLDMKAMDSKSNGAIAWSNQTSFTCQDIFKETNATYPSSDVPCDATLTEK |
| | SFETDMNLNFQNLLVIVLRILLLKVAGENLLMTLRLWSS [SEQ ID NO: 141] |
| 7A | MGFRLLCCVAFCLLGAGPVDSGVTQTPKHLITATGQRVTLRCSPRSGDLSVYWYQQSLDQGLQFLIQYYNG |
| | EERAKGNILERFSAQQFPDLHSELNLSSLELGDSALYFCASSVDWVQETQYFGPGTRLLVLEDLRNVTPPK |
| | VSLFEPSKAEIANKQKATLVCLARGFFPDHVELSWWVNGKEVHSGVCTDPQAYKESNYSYCLSSRLRVSAT |
| | FWHNPRNHFRCQVQFHGLSEEDKWPEGSPKPVTQNISAEAWGRADCGITSASYQQGVLSATILYEILLGKA |
| | TLYAVLVSTLVVMAMVKRKNSRAKRSGSGATNFSLLKQAGDVEENPGPMLTASLLRAVIASICVVSSMAQK |
| | VTQAQTEISVVEKEDVTLDCVYETRDTTYYLFWYKQPPSGELVFLIRRNSFDEQNEISGRYSWNFQKSTSS |
| | FNFTITASQVVDSAVYFCALRNSGNTPLVFGKGTRLSVIADIQNPEPAVYQLKDPRSQDSTLCLFTDEDSQ |
| | INVPKTMESGTFITDKCVLDMKAMDSKSNGAIAWSNQTSFTCQDIFKETNATYPSSDVPCDATLTEKSFET |
| | DMNLNFQNLLVIVLRILLLKVAGENLLMTLRLWSS [SEQ ID NO: 142] |
| 7B | MLLLLLLLGPGSGLGAVVSQHPSRVICKSGTSVKIECRSLDFQATTMFWYRQFPKQSLMLMATSNEGSKAT |
| | YEQGVEKDKFLINHASLTLSTLTVTSAHPEDSSFYICSASESLALASEQYFGPGTRLTVTEDLRNVTPPKV |
| | SLEEPSKAEIANKQKATLVCLARGFFPDHVELSWWVNGKEVHSGVCTDPQAYKESNYSYCLSSRLRVSATF |
| | WHNPRNHFRCQVQFHGLSEEDKWPEGSPKPVTQNISAEAWGRADCGITSASYQQGVLSATILYEILLGKAT |
| | LYAVLVSTLVVMAMVKRKNSRAKRSGSGATNFSLLKQAGDVEENPGPMRLVARVTVFLTFGTIIDAKTTQP |
| | TSMDCAEGRAANLPCNHSTISGNEYVYWYRQIHSQGPQYIIHGLKNNETNEMASLIITEDRKSSTLILPHA |
| | TLRDTAVYYCIVPRGFSNTGKLIFGQGTTLQVKPDIQNPEPAVYQLKDPRSQDSTLCLFTDFDSQINVPKT |
| | MESGTFITDKCVLDMKAMDSKSNGAIAWSNQTSFTCQDIFKETNATYPSSDVPCDATLTEKSFETDMNLNF |
| | QNLLVIVLRILLLKVAGENLLMTLRLWSS [SEQ ID NO: 143] |
| 7D | MGFRLLCCVAFCLLGAGPVDSGVTQTPKHLITATGQRVTLRCSPRSGDLSVYWYQQSLDQGLQFLIQYYNG |
| | EERAKGNILERFSAQQFPDLHSELNLSSLELGDSALYFCASSERLAGAIADTQYFGPGTRLTVLEDLRNVT |
| | PPKVSLFEPSKAEIANKQKATLVCLARGFFPDHVELSWWVNGKEVHSGVCTDPQAYKESNYSYCLSSRLRV |
| | SATFWHNPRNHFRCQVQFHGLSEEDKWPEGSPKPVTQNISAEAWGRADCGITSASYQQGVLSATILYEILL |
| | GKATLYAVLVSTLVVMAMVKRKNSRAKRSGSGATNFSLLKQAGDVEENPGPMMKSLRVLLVILWLQLSWVW |
| | SQQKEVEQDPGPLSVPEGAIVSLNCTYSNSAFQYFMWYRQYSRKGPELLMYTYSSGNKEDGRFTAQVDKSS |
| | KYISLFIRDSQPSDSATYLCASSESGYNKLIFGAGTRLAVHPDIQNPEPAVYQLKDPRSQDSTLCLFTDED |
| | SQINVPKTMESGTFITDKCVLDMKAMDSKSNGAIAWSNQTSFTCQDIFKETNATYPSSDVPCDATLTEKSF |
| | ETDMNLNFQNLLVIVLRILLLKVAGENLLMTLRLWSS [SEQ ID NO: 144] |
| 8A | MGCRLLCCVVFCLLQAGPLDTAVSQTPKYLVTQMGNDKSIKCEQNLGHDTMYWYKQDSKKFLKIMESYNNK |
| | ELIINETVPNRFSPKSPDKAHLNLHINSLELGDSAVYFCASSQDLGFDEKLFFGSGTQLSVLEDLRNVTPP |
| | KVSLFEPSKAEIANKQKATLVCLARGFFPDHVELSWWVNGKEVHSGVCTDPQAYKESNYSYCLSSRLRVSA |
| | TFWHNPRNHFRCQVQFHGLSEEDKWPEGSPKPVTQNISAEAWGRADCGITSASYQQGVLSATILYEILLGK |
| | ATLYAVLVSTLVVMAMVKRKNSRAKRSGSGATNFSLLKQAGDVEENPGPMASAPISMLAMLFTLSGLRAQS |
| | VAQPEDQVNVAEGNPLTVKCTYSVSGNPYLFWYVQYPNRGLQFLLKYITGDNLVKGSYGFEAEFNKSQTSF |
| | HLKKPSALVSDSALYFCAVRAPGNTPLVFGKGTRLSVIADIQNPEPAVYQLKDPRSQDSTLCLFTDFDSQI |
| | NVPKTMESGTFITDKCVLDMKAMDSKSNGAIAWSNQTSFTCQDIFKETNATYPSSDVPCDATLTEKSFETD |
| | MNLNFQNLLVIVLRILLLKVAGENLLMTLRLWSS [SEQ ID NO: 145] |

In certain embodiments, the nucleic acid encodes a functional portion/fragment of a presently disclosed TCR. As used herein, the term “functional portion” or “functional fragment” refers to any portion, part or fragment of a presently disclosed TCR, which portion, part or fragment retains the biological activity of the TCR (the parent TCR). For example, functional portions encompass the portions, parts or fragments of a presently disclosed TCR that retains the ability to recognize the TP53 peptide (e.g., a TP53 peptide comprising a R175H mutation) to a similar, same, or even a higher extent as the parent TCR. In certain embodiments, the nucleic acid encoding a functional portion of a presently disclosed TCR encodes a protein comprising, e.g., about 10%, about 20%, about 25%, about 30%, about 35%, about 40%, about 45%, about 50%, about 55%, about 60%, about 65%, about 70%, about 75%, about 80%, about 85%, about 90%, and about 95%, or more of the parent TCR.

Genetic modification of a cell (e.g., a T cell) can be accomplished by transducing a substantially homogeneous cell composition with a recombinant DNA or RNA construct. In certain embodiments, a retroviral vector (e.g., gamma-retroviral vector or lentiviral vector) is employed for the introduction of the DNA or RNA construct into the cell. For example, a polynucleotide encoding a presently disclosed TCR can be cloned into a retroviral vector and expression can be driven from its endogenous promoter, from the retroviral long terminal repeat, or from an alternative internal promoter, or from a promoter specific for a target cell type of interest. Non-viral vectors or RNA may be used as well. Random chromosomal integration, or targeted integration (e.g., using a nuclease, transcription activator-like effector nucleases (TALENs), Zinc-finger nucleases (ZFNs), and/or clustered regularly interspaced short palindromic repeats (CRISPRs), or transgene expression (e.g., using a natural or chemically modified RNA) can be used. For initial genetic modification of a cell to include a presently disclosed TCR, a retroviral vector can be employed for transduction, however any other suitable viral vector or non-viral delivery system can be used. The TCR can be constructed in a single, multicistronic expression cassette, in multiple expression cassettes of a single vector, or in multiple vectors. Examples of elements that create polycistronic expression cassette include, but is not limited to, various viral and non-viral Internal Ribosome Entry Sites (IRES, e.g., FGF-1 IRES, FGF-2 IRES, VEGF IRES, IGF-II IRES, NF-κB IRES, RUNX1 IRES, p53 IRES, hepatitis A IRES, hepatitis C IRES, pestivirus IRES, aphthovirus IRES, picornavirus IRES, poliovirus IRES and encephalomyocarditis virus IRES) and cleavable linkers (e.g., 2A peptides, e.g., P2A, T2A, E2A and F2A peptides). Combinations of retroviral vector and an appropriate packaging line are also suitable, where the capsid proteins will be functional for infecting human cells. Various amphotropic virus-producing cell lines are known, including, but not limited to, PA12 (Miller et al., (1985) Mol Cell Biol (1985); 5:431-437); PA317 (Miller., et al., Mol Cell Biol (1986); 6:2895-2902); and CRIP (Danos et al., Proc Natl Acad Sci USA (1988); 85:6460-6464). Non-amphotropic particles are suitable too, e.g., particles pseudotyped with VSVG, RD114 or GALV envelope and any other known in the art.

Possible methods of transduction also include direct co-culture of the cells with producer cells (Bregni et al., Blood (1992); 80:1418-1422), or culturing with viral supernatant alone or concentrated vector stocks with or without appropriate growth factors and polycations (Xu et al., Exp Hemat (1994); 22:223-230; and Hughes et al. J Clin Invest (1992); 89:1817).

Other transducing viral vectors can be used to modify a cell. In certain embodiments, the chosen vector exhibits high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). Other viral vectors that can be used include, for example, adenoviral, lentiviral, and adena-associated viral vectors, vaccinia virus, a bovine papilloma virus, or a herpes virus, such as Epstein-Barr Virus (also see, for example, the vectors of Miller, Human Gene Thera (1990); 15-14; Friedman, Science 244:1275-1281, 1989; Eglitis et al., BioTechniques (1988); 6:608-614; Tolstoshev et al., Cur Opin Biotechnol (1990); 1:55-61; Sharp, The Lancet (1991); 337:1277-78; Cornetta et al., Nucleic Acid Research and Molecular Biology 36:311-22, 1987; Anderson, Science (1984); 226:401-409; Moen, Blood Cells 17:407-16, 1991; Miller et al., Biotechnol (1989); 7:980-90; LeGal La Salle et al., Science (1993); 259:988-90; and Johnson, Chest (1995) 107:77S-83S). Retroviral vectors are particularly well developed and have been used in clinical settings (Rosenberg et al., N Engl J Med (1990); 323:370, 1990; Anderson et al., U.S. Pat. No. 5,399,346).

Non-viral approaches can also be employed for genetic modification of a cell. For example, a nucleic acid molecule can be introduced into a cell by administering the nucleic acid in the presence of lipofection (Feigner et al., Proc Natl Acad Sci U.S.A. (1987); 84:7413; Ono et al., Neurosci Lett (1990); 17:259; Brigham et al., Am J Med Sci (1989); 298:278; Staubinger et al., Methods in Enzymol (1983); 101:512, Wu et al., J Biol Chem (1988); 263:14621; Wu et al., J Biol Chem (1989); 264:16985), or by micro-injection under surgical conditions (Wolff et al., Science (1990); 247:1465). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically. Recombinant receptors can also be derived or obtained using transposases or targeted nucleases (e.g. Zinc finger nucleases, meganucleases, or TALE nucleases, CRISPR). Transient expression may be obtained by RNA electroporation.

In certain embodiments, a presently disclosed TCR can be integrated into a selected locus of the genome of a cell. Any targeted genome editing methods can also be used to deliver a presently disclosed TCR to a cell or a subject. In certain embodiments, a CRISPR system is used to deliver a presently disclosed TCR. In certain embodiments, zinc-finger nucleases are used to deliver presently disclosed TCR. In certain embodiments, a TALEN system is used to deliver a presently disclosed TCR.

In certain embodiments, a presently disclosed TCR can be integrated at a locus encoding a T cell receptor. Non-limiting examples of the loci include a TRAC locus, a TRBC locus, a TRDC locus, and a TRGC locus. In certain embodiments, the locus is a TRAC locus or a TRBC locus. Methods of targeting a TCR to a site within the genome of T cell can be found in WO2017180989 and Eyquem et al., Nature. (2017 Mar. 2); 543 (7643): 113-117, both of which are incorporated by reference in their entireties.

In certain embodiments, a presently disclosed TCR can be integrated at a locus encoding an immune-checkpoint. Non-limiting examples of the loci include a PDCD1 locus, a CBLB locus, a CISH locus, or a RASA2 locus. In certain embodiments, the locus is a PDCD1 locus. In certain embodiments, the locus is a CBLB locus. In certain embodiments, the locus is a CISH locus. In certain embodiments, the locus is a RASA2 locus. Non-limiting examples of methods of integrating a presently disclosed TCR to a locus encoding an immune-checkpoint CRISPR systems, zinc-finger nucleases, and TALEN systems.

In certain embodiments, a presently disclosed TCR can be integrated at a genomic safe harbor locus. As used herein, a “genomic safe harbor” or “GSH” refers to a chromosome location where an integrated transgene (e.g., encoding a presently disclosed TCR) can be predictably expressed without adversely affecting endogenous gene structure or expression. In certain embodiments, integrating a transgene at the GSH does not alter cell behavior and/or promote malignant transformation of the host cell or the organism. In certain embodiments, the GSH permits sufficient transgene expression to yield desirable levels of protein or non-coding RNA encoded by the transgene. Additional information on genomic safe harbor sites can be found in International Patent Publication No. WO 2021/055616, Sadelain et al., Nature Reviews Cancer 12.1 (2012): 51-58, and Aznauryan et al., Cell Reports Methods 2.1 (2022), the contents of each of which are incorporated by reference in their entirety.

In certain embodiments, the expression of the TCR is driven by an endogenous promoter/enhancer within or near the locus. In certain embodiments, the expression of the TCR is driven by an exogenous promoter integrated into the locus. The locus where the TCR is integrated is selected based on the expression level of the genes within the locus, and timing of the gene expression of the genes within the locus. The expression level and timing can vary under different stages of cell differentiation and mitogen/cytokine microenvironment, which are among the factors to be considered when making the selection.

In certain embodiments, the CRISPR system is used to integrate the TCR in selected loci of the genome of a cell. In certain embodiments, the CRISPR system uses a DNA donor-template guided homology directed repair at a defined genetic locus, e.g., a TRAC locus. Clustered regularly-interspaced short palindromic repeats (CRISPR) system is a genome editing tool discovered in prokaryotic cells. When utilized for genome editing, the system includes Cas9 (a protein able to modify DNA utilizing crRNA as its guide), CRISPR RNA (crRNA, contains the RNA used by Cas9 to guide it to the correct section of host DNA along with a region that binds to tracrRNA (generally in a hairpin loop form) forming an active complex with Cas9), trans-activating crRNA (tracrRNA, binds to crRNA and forms an active complex with Cas9), and an optional section of DNA repair template (DNA that guides the cellular repair process allowing insertion of a specific DNA sequence). CRISPR/Cas9 often employs a plasmid to transfect the target cells. In certain embodiments, CRISPR/Cas9 is a recombinant ribonucleoprotein complex that is transfected into target cells. The crRNA needs to be designed for each application as this is the sequence that Cas9 uses to identify and directly bind to the target DNA in a cell. The repair template carrying TCR expression cassette need also be designed for each application, as it must overlap with the sequences on either side of the cut and code for the insertion sequence. Multiple crRNA's and the tracrRNA can be packaged together to form a single-guide RNA (sgRNA). This sgRNA can be joined together with the Cas9 gene and made into a plasmid in order to be transfected into cells. Methods of using the CRISPR system are described, for example, in WO 2014093661 A2, WO 2015123339 A1 and WO 2015089354 A1, which are incorporated by reference in their entireties.

In certain embodiments, zinc-finger nucleases are used to integrate the TCR in selected loci of the genome of a cell. A zinc-finger nuclease (ZFN) is an artificial restriction enzyme, which is generated by combining a zinc finger DNA-binding domain with a DNA-cleavage domain. A zinc finger domain can be engineered to target specific DNA sequences which allows a zinc-finger nuclease to target desired sequences within genomes. The DNA-binding domains of individual ZFNs typically contain a plurality of individual zinc finger repeats and can each recognize a plurality of base pairs. The most common method to generate new zinc-finger domain is to combine smaller zinc-finger “modules” of known specificity. The most common cleavage domain in ZFNs is the non-specific cleavage domain from the type IIs restriction endonuclease FokI. Using the endogenous homologous recombination (HR) machinery and a homologous DNA template carrying TCR expression cassette, ZFNs can be used to insert the TCR expression cassette into genome. When the targeted sequence is cleaved by ZFNs, the HR machinery searches for homology between the damaged chromosome and the homologous DNA template, and then copies the sequence of the template between the two broken ends of the chromosome, whereby the homologous DNA template is integrated into the genome. Methods of using the ZFN system are described, for example, in WO 2009146179 A1, WO 2008060510 A2 and CN 102174576 A, which are incorporated by reference in their entireties.

In certain embodiments, the TALEN system is used to integrate the TCR in selected loci of the genome of an immunoresponsive cell. Transcription activator-like effector nucleases (TALEN) are restriction enzymes that can be engineered to cut specific sequences of DNA. TALEN system operates on almost the same principle as ZFNs. They are generated by combining a transcription activator-like effectors DNA-binding domain with a DNA cleavage domain. Transcription activator-like effectors (TALEs) are composed of 33-34 amino acid repeating motifs with two variable positions that have a strong recognition for specific nucleotides. By assembling arrays of these TALEs, the TALE DNA-binding domain can be engineered to bind desired DNA sequence, and thereby guide the nuclease to cut at specific locations in genome. Methods of using the TALEN system are described, for example, in WO 2014134412 A1, WO 2013163628 A2 and WO 2014040370 A1, which are incorporated by reference in their entireties.

cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element or intron (e.g. the elongation factor 1a enhancer/promoter/intron structure). For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

Methods for delivering the genome editing agents/systems can vary depending on the need. In certain embodiments, the components of a selected genome editing method are delivered as DNA constructs in one or more plasmids. In certain embodiments, the components are delivered via viral vectors. Common delivery methods include but is not limited to, electroporation, microinjection, gene gun, impalefection, hydrostatic pressure, continuous infusion, sonication, magnetofection, adeno-associated viruses, envelope protein pseudotyping of viral vectors, replication-competent vectors cis and trans-acting elements, herpes simplex virus, and chemical vehicles (e.g., oligonucleotides, lipoplexes, polymersomes, polyplexes, dendrimers, inorganic Nanoparticles, and cell-penetrating peptides).

In certain embodiments, the delivery methods include the use of colloids. As used herein, the term “colloid” refers to systems in which there are two or more phases, with one phase (e.g., the dispersed phase) distributed in the other phase (e.g., the continuous phase). Moreover, at least one of the phases has small dimensions (in the range of about 10-9 to about 10-6 m). Non-limiting examples of colloids encompassed by the presently disclosed subject matter include macromolecule complexes, nanocapsules, microspheres, beads, and lipid-based systems (e.g., micelles, liposomes, and lipid nanoparticles).

In certain embodiments, the delivery methods include the use of liposomes. The term “liposome,” as used herein, refers to single- or multi-layered spherical lipid bilayer structures produced from lipids dissolved in organic solvents and then dispersed in aqueous media. Experimentally and therapeutically used for delivering an active pharmaceutical ingredient (e.g., nucleic acid compositions disclosed herein) to cells, liposomes fuse with cell membranes so the contents are transferred into the cytoplasm.

In certain embodiments, the delivery methods include the use of lipid nanoparticles. As used herein, the term “lipid nanoparticle” refers to a particle having at least one dimension in the order of nanometers (e.g., from about 1 nm to about 1,000 nm) and including at least one lipid. In certain embodiments, the lipid nanoparticles can include an active pharmaceutical ingredient (e.g., nucleic acid compositions disclosed herein) for delivering to cells. The morphology of the lipid nanoparticles can be different from liposomes. While liposomes are characterized by a lipid bilayer surrounding a hydrophilic core, lipid nanoparticles have an electron-dense core where cationic lipids and/or ionizable lipids are organized into inverted micelles around an active pharmaceutical ingredient (e.g., nucleic acid compositions disclosed herein). Additional information on the morphology and properties of lipid nanoparticles and liposomes can be found in Wilczewska, et al., Pharmacological reports 64, no. 5 (2012): 1020-1037; Eygeris et al., Accounts of Chemical Research 55, no. 1 (2021): 2-12; Zhang et al., Chemical Reviews 121, no. 20 (2021): 12181-12277; and Fan et al., Journal of pharmaceutical and biomedical analysis 192 (2021): 113642.

In certain embodiments, the lipid nanoparticles have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 10⁰ nm, from about 80 nm to about 10⁰ nm, from about 90 nm to about 10⁰ nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm.

In certain embodiments, the lipid nanoparticles can include a cationic lipid or an ionizable lipid. The term “cationic lipid” refers to lipids including a head group with permanent positive charges. Non-limiting examples of cationic lipids encompassed by the presently disclosed subject matter include 1,2-di-O-octadecenyl-3-trimethylammonium-propane (DOTMA), 1,2-dioleoyl-3-trimethylammonium-propane (DOTAP), 2,3-dioleyloxy-N-[2-(sperminecarboxamido)ethyl]-N,N-dimethyl-1-propanaminium trifluoroacetate (DOSPA), and ethylphosphatidylcholine (ePC).

As used herein, the term “ionizable lipid” refers to lipids that are protonated at low pH and are neutral at physiological pH. The pH-sensitivity of ionizable lipids is particularly beneficial for delivery in vivo (e.g., delivery of nucleic acid compositions disclosed herein), because neutral lipids have less interactions with the anionic membranes of blood cells and, thus, improve the biocompatibility of the lipid nanoparticles. Once trapped in endosomes, ionizable lipids are protonated and promote membrane destabilization to allow the endosomal escape of the nanoparticles. Non-limiting example of ionizable lipids encompassed by the presently disclosed subject matter include tetrakis(8-methylnonyl) 3,3′,3″,3′-(((methylazanediyl) bis(propane-3,1 diyl)) bis(azanetriyl)) tetrapropionate; decyl(2-(dioctylammonio) ethyl) phosphate; ((4-hydroxybutyl)azanediyl)bis(hexane-6,1-diyl) bis(2-hexyldecanoate); bis(2-(dodecyldisulfanyl)ethyl) 3,3 1-((3-methyl-9-oxo-10-oxa-13,14-dithia-3,6-diazahexacosyl)azanediyl) dipropionate; 1,1′-((2-(4-(2-((2-(bis(2-hydroxydodecyl)amino)ethyl) (2-hydroxydodecyl)amino)ethyl) piperazin-1-yl)ethyl)azanediyl)bis(dodecan-2-ol); cKK-E12,3,6-bis(4-(bis(2-hydroxydodecyl)amino)butyl) piperazine-2,5-dione; (6Z,9Z,28Z,31Z)-heptatriaconta-6,9,28,31-tetraen-19-yl 4-(dimethylamino)butanoate; hexa (octan-3-yl) 9,9′,9″,9″,9 ″″,″″-(((benzene-1,3,5-tricarbonyl)yris(azanediyl)tris(propane-3,1-diyl) tris(azanetriyl))hexanonanoate; heptadecan-9-yl 8-((2-hydroxyethyl) (6-oxo-6-(undecyloxy)hexyl)amino) octanoate; and (((3,6-dioxopiperazine-2,5-diyl) bis(butane-4,1-diyl)) bis(azanetriyl)) tetrakis(ethane-2,1-diyl) (9Z,9 ‘Z,9″Z,9″Z,12Z,12’ Z,12″Z,12″Z)-tetrakis(octadeca-9,12-dienoate).

Additionally, in certain embodiments, the lipid nanoparticles can include other lipids. For example, but without any limitation, the lipid nanoparticles of the presently disclosed subject matter can include phospholipids, cholesterol, polyethylene glycol (PEG)-functionalized lipids (PEG-lipids). These lipids can improve certain properties of the lipid nanoparticles (e.g., stability, biodistribution, etc.). For example, cholesterol enhances the stability of the lipid nanoparticles by modulating their integrity and rigidity. Non-limiting examples of other lipids present in lipid nanoparticles include cholesterol, DC-cholesterol, β-sitosterol, BHEM-cholesterol, ALC-0159, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoylphosphatidylethanolamine (DSPE), 16-O-monomethyl PE, 16-O-dimethyl PE, 18-1-trans PE, 1-stearioyl-2-oleoyl-phosphatidyethanol amine (SOPE), and 1,2-dielaidoyl-sn-glycero-3-phophoethanolamine (transDOPE).

In certain embodiments, the lipid nanoparticles can include a targeting moiety that binds to a ligand. The use of the targeting moieties allows selective delivery of an active pharmaceutical ingredient (e.g., nucleic acid compositions disclosed herein) to target cells expressing the ligand (e.g., T cells). In certain embodiments, the targeting moiety can be an antibody or antigen-binding fragment thereof that binds to a cell surface receptor. For example, but without any limitation, the targeting domain is an antibody or antigen-binding fragment thereof that binds to a receptor expressed on the surface of a T cell (e.g., CD3, CD4, CD8, CD16, CD40L, CD95, FasL, CTLA-4, OX40, GITR, LAG3, ICOS, and PD-1).

In certain embodiments, the delivery methods are in vivo delivery methods. In certain embodiments, the delivery methods are ex vivo delivery methods.

Modification can be made anywhere within the selected locus, or anywhere that can influence gene expression of the integrated TCR. In certain embodiments, the modification is introduced upstream of the transcriptional start site of the integrated TCR. In certain embodiments, the modification is introduced between the transcriptional start site and the protein coding region of the integrated TCR). In certain embodiments, the modification is introduced downstream of the protein coding region of the integrated TCR.

### 5.6. Formulations and Administration

The presently disclosed subject matter also provides compositions comprising the presently disclosed cells (e.g., those disclosed in Section 5.4). In certain embodiments, the composition is a pharmaceutical composition that further comprises a pharmaceutically acceptable carrier.

Compositions comprising the presently disclosed cells can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Compositions comprising the presently disclosed cells can be provided systemically or directly to a subject for inducing and/or enhancing an immune response to an antigen and/or treating and/or preventing a tumor. In certain embodiments, the presently disclosed cells or compositions comprising thereof are directly injected into an organ of interest (e.g., an organ affected by a neoplasm). Alternatively, the presently disclosed cells or compositions comprising thereof are provided indirectly to the organ of interest, for example, by administration into the circulatory system (e.g., the tumor vasculature). Expansion and differentiation agents can be provided prior to, during or after administration of the cells or compositions to increase production of cells in vitro or in vivo.

The quantity of cells to be administered can vary for the subject being treated. In certain embodiments, between about 10⁴ and about 10¹¹, between about 10⁴ and about 10⁷, between about 105 and about 10⁷, between about 10⁵ and about 10⁹, or between about 10⁶ and about 10⁸ of the presently disclosed cells are administered to a subject. In certain embodiments, at least about 1×10⁵ cells can be administered, eventually reaching about 1×10¹⁰ or more. In certain embodiments, at least about 1×10⁶ cells can be administered. In certain embodiments, from about 10⁴ to about 1011, from about 10⁵ to about 10⁹, or from about 10⁶ to about 10⁸ the presently disclosed cells are administered to a subject. More effective cells may be administered in even smaller numbers. In certain embodiments, at least about 1×10⁸, about 2×10⁸, about 3×10⁸, about 4×10⁸, and about 5×10⁸ the presently disclosed cells are administered to a subject. The precise determination of what would be considered an effective dose can be based on factors individual to each subject, including their size, age, sex, weight, and condition of the particular subject. Dosages can be readily ascertained by those skilled in the art from this disclosure and the knowledge in the art.

The presently disclosed cells and compositions can be administered by any method known in the art including, but not limited to, intravenous administration, subcutaneous administration, intranodal administration, intratumoral administration, intrathecal administration, intrapleural administration, intraosseous administration, intraperitoneal administration, pleural administration, and direct administration to the subject. The presently disclosed cells can be administered in any physiologically acceptable vehicle, normally intravascularly, although they may also be introduced into bone or other convenient site where the cells may find an appropriate site for regeneration and differentiation (e.g., thymus).

### 5.7. Methods of Treatment

The presently disclosed subject matter provides various methods of using the presently disclosed cells or compositions comprising thereof. The presently disclosed cells and compositions comprising thereof can be used in a therapy or medicament. For example, the presently disclosed subject matter provides methods for inducing and/or increasing an immune response in a subject in need thereof. The presently disclosed cells and compositions comprising thereof can be used for reducing tumor burden in a subject. The presently disclosed cells and compositions comprising thereof can reduce the number of tumor cells, reduce tumor size, and/or eradicate the tumor in the subject. The presently disclosed cells and compositions comprising thereof can be used for treating and/or preventing a tumor in a subject. The presently disclosed cells and compositions comprising thereof can be used for prolonging the survival of a subject suffering from a tumor. In certain embodiments, each of the above-noted methods comprises administering the presently disclosed cells or a composition (e.g., a pharmaceutical composition) comprising thereof to achieve the desired effect, e.g., palliation of an existing condition or prevention of recurrence of tumor. For treatment, the amount administered is an amount effective in producing the desired effect. An effective amount can be provided in one or a series of administrations. An effective amount can be provided in a bolus or by continuous perfusion.

In certain embodiments, the tumor is associated with TP53. In certain embodiments, the tumor is associated with a TP53 mutation or a TP53 mutant. In certain embodiments, the TP53 mutation is a R175 mutation. In certain embodiments, the TP53 mutation is a R175H mutation.

In certain embodiments, the tumor is a cancer. In certain embodiments, the tumor is selected from the group consisting of brain tumors, soft tissue and bone sarcomas, adrenocortical carcinoma, rhabdomyosarcoma, Wilms tumor, pancreatic cancer, breast cancer, endometrial cancer, cervical cancer, anal cancer, bladder cancer, colorectal cancer, cholangiocarcinoma/bile duct cancer, lung cancer, ovarian cancer, esophageal cancer, gastric cancer (also known as “stomach cancer”), head and neck squamous cell carcinoma, nonmelanoma skin cancer, salivary gland cancer, melanoma, multiple myeloma, prostate cancer, T-lymphoblastic leukemia/lymphoma, acute myeloid leukemia, B-cell leukemia, myeloproliferative neoplasm, and histiocytosis. In certain embodiments, the cancer is colorectal cancer. In certain embodiments, the tumor is breast cancer. In certain embodiments, the tumor is esophageal cancer.

Additionally, the presently disclosed cells and compositions comprising thereof can be used for treating and/or preventing a premalignant condition in a subject. The presently disclosed cells and compositions comprising thereof can be used for prolonging the survival of a subject suffering from a premalignant condition. As used herein, the term “premalignant condition” refers to any condition that, if left untreated, can turn into cancer. In certain embodiments, the premalignant condition can be characterized by a precancerous lesion that is a morphologically altered tissue in which cancer is more likely to occur than in its apparently normal counterpart.

In certain embodiments, the premalignant condition is associated with TP53. In certain embodiments, the premalignant condition is associated with a TP53 mutation or a TP53 mutant. In certain embodiments, the TP53 mutation is a R175 mutation. In certain embodiments, the TP53 mutation is a R175H mutation.

In certain embodiments, the premalignant condition is clonal hematopoiesis. Clonal hematopoiesis (CH) occurs when a hematopoietic stem cell, which can develop into different types of blood cells, starts making cells with the same genetic mutation. These blood cells have a different genetic pattern than the rest of the blood cells. CH can lead to blood cancers including, for example and without any limitation, myelodysplastic syndrome and acute myeloid leukemia.

In certain embodiments, the subject is a human subject. The subjects can have an advanced form of disease, in which case the treatment objective can include mitigation or reversal of disease progression, and/or amelioration of side effects. The subjects can have a history of the condition, for which they have already been treated, in which case the therapeutic objective will typically include a decrease or delay in the risk of recurrence.

In certain embodiments, the subject comprises an HLA-A. In certain embodiments, the HLA-A is an HLA-A*02 superfamily member. In certain embodiments, the HLA-A*02 superfamily is selected from the group comprising HLA-A*02, HLA-A*02:01, HLA-A*02:02, HLA-A*02:03, HLA-A*02:04, HLA-A*02:05, HLA-A*02:06, HLA-A*02:07, HLA-A*02:09, HLA-A*02:11, HLA-A*02:12, HLA-A*02:13, HLA-A*02:0215HLA-A*02:14, HLA-A*02:17, HLA-A*02:71, HLA-A*02:77, HLA-A*68:02, and HLA-A*69:01. In certain embodiments, the HLA-A*02 superfamily member is HLA-A*02:01.

### 5.8. Exemplary Embodiments

In certain embodiments, the presently disclosed subject matter relates to a recombinant T cell receptor (TCR) that binds to a TP53 peptide, wherein the TP53 peptide comprises a R175 mutation.

In certain embodiments of the recombinant TCRs disclosed herein, the TCR comprises at least one amino acid alteration relative to the sequence of a naturally occurring TCR.

In certain embodiments of the recombinant TCRs disclosed herein, the amino acid alteration is a cysteine modification.

In certain embodiments of the recombinant TCRs disclosed herein, the TP53 peptide comprises a R175H mutation.

In certain embodiments of the recombinant TCRs disclosed herein, the TP53 peptide comprises or consists of the amino acid sequence set forth in SEQ ID NO: 146.

In certain embodiments of the recombinant TCRs disclosed herein, the TP53 peptide comprises or consists of the amino acid sequence set forth in SEQ ID NO: 147.

In certain embodiments of the recombinant TCRs disclosed herein, the TP53 peptide is associated with an HLA class I complex.

In certain embodiments of the recombinant TCRs disclosed herein, the HLA class I complex is selected from an HLA-A, an HLA-B, and an HLA-C.

In certain embodiments of the recombinant TCRs disclosed herein, the HLA class I complex is an HLA-A.

In certain embodiments of the recombinant TCRs disclosed herein, the HLA-A is an HLA-A*02 superfamily member.

In certain embodiments of the recombinant TCRs disclosed herein, the HLA-A*02 superfamily member is selected from the group comprising HLA-A*02:02, HLA-A*02:03, HLA-A*02:04, HLA-A*02:05, HLA-A*02:06, HLA-A*02:07, HLA-A*02:09, HLA-A*02:11, HLA-A*02:12, HLA-A*02:13, HLA-A*02:0215HLA-A*02:14, HLA-A*02:17, HLA-A*02:71, HLA-A*02:77, HLA-A*68:02, and HLA-A*69:01.

In certain embodiments of the recombinant TCRs disclosed herein, the HLA-A*02 superfamily member is HLA-A*02:01.

In certain embodiments of the recombinant TCRs disclosed herein, the TCR comprises an extracellular domain that binds to the TP53 peptide, wherein the extracellular domain comprises an α chain and a β chain, wherein the α chain comprises an α chain variable region and α chain constant region, and the β chain comprises a β chain variable region and a β chain constant region.

In certain embodiments of the recombinant TCRs disclosed herein, a) the α chain variable region comprises a CDR3 comprising the amino acid sequence set forth in SEQ ID NO: 3 or a conservative modification thereof, and the β chain variable region comprises a CDR3 comprising the amino acid sequence set forth in SEQ ID NO: 6 or a conservative modification thereof; b) the α chain variable region comprises a CDR3 comprising the amino acid sequence set forth in SEQ ID NO: 13 or a conservative modification thereof, and the β chain variable region comprises a CDR3 comprising the amino acid sequence set forth in SEQ ID NO: 16 or a conservative modification thereof; c) the α chain variable region comprises a CDR3 comprising the amino acid sequence set forth in SEQ ID NO: 23 or a conservative modification thereof, and the β chain variable region comprises a CDR3 comprising the amino acid sequence set forth in SEQ ID NO: 26 or a conservative modification thereof; d) the α chain variable region comprises a CDR3 comprising the amino acid sequence set forth in SEQ ID NO: 33 or a conservative modification thereof, and the β chain variable region comprises a CDR3 comprising the amino acid sequence set forth in SEQ ID NO: 36 or a conservative modification thereof; e) the α chain variable region comprises a CDR3 comprising the amino acid sequence set forth in SEQ ID NO: 43 or a conservative modification thereof, and the β chain variable region comprises a CDR3 comprising the amino acid sequence set forth in SEQ ID NO: 46 or a conservative modification thereof; f) the α chain variable region comprises a CDR3 comprising the amino acid sequence set forth in SEQ ID NO: 53 or a conservative modification thereof, and the β chain variable region comprises a CDR3 comprising the amino acid sequence set forth in SEQ ID NO: 56 or a conservative modification thereof; g) the α chain variable region comprises a CDR3 comprising the amino acid sequence set forth in SEQ ID NO: 63 or a conservative modification thereof, and the β chain variable region comprises a CDR3 comprising the amino acid sequence set forth in SEQ ID NO: 66 or a conservative modification thereof; h) the α chain variable region comprises a CDR3 comprising the amino acid sequence set forth in SEQ ID NO: 73 or a conservative modification thereof, and the β chain variable region comprises a CDR3 comprising the amino acid sequence set forth in SEQ ID NO: 76 or a conservative modification thereof; i) the α chain variable region comprises a CDR3 comprising the amino acid sequence set forth in SEQ ID NO: 83 or a conservative modification thereof, and the β chain variable region comprises a CDR3 comprising the amino acid sequence set forth in SEQ ID NO: 86 or a conservative modification thereof; j) the α chain variable region comprises a CDR3 comprising the amino acid sequence set forth in SEQ ID NO: 93 or a conservative modification thereof, and the β chain variable region comprises a CDR3 comprising the amino acid sequence set forth in SEQ ID NO: 96 or a conservative modification thereof; k) the α chain variable region comprises a CDR3 comprising the amino acid sequence set forth in SEQ ID NO: 103 or a conservative modification thereof, and the β chain variable region comprises a CDR3 comprising the amino acid sequence set forth in SEQ ID NO: 106 or a conservative modification thereof; or 1) the α chain variable region comprises a CDR3 comprising the amino acid sequence set forth in SEQ ID NO: 113 or a conservative modification thereof, and the β chain variable region comprises a CDR3 comprising the amino acid sequence set forth in SEQ ID NO: 116 or a conservative modification thereof.

In certain embodiments of the recombinant TCRs disclosed herein, a) the α chain variable region comprises a CDR2 comprising the amino acid sequence set forth in SEQ ID NO: 2 or a conservative modification thereof, and the β chain variable region comprises a CDR2 comprising the amino acid sequence set forth in SEQ ID NO: 5 or a conservative modification thereof; b) the α chain variable region comprises a CDR2 comprising the amino acid sequence set forth in SEQ ID NO: 12 or a conservative modification thereof, and the β chain variable region comprises a CDR2 comprising the amino acid sequence set forth in SEQ ID NO: 15 or a conservative modification thereof; c) the α chain variable region comprises a CDR2 comprising the amino acid sequence set forth in SEQ ID NO: 22 or a conservative modification thereof, and the β chain variable region comprises a CDR2 comprising the amino acid sequence set forth in SEQ ID NO: 25 or a conservative modification thereof; d) the α chain variable region comprises a CDR2 comprising the amino acid sequence set forth in SEQ ID NO: 32 or a conservative modification thereof, and the β chain variable region comprises a CDR2 comprising the amino acid sequence set forth in SEQ ID NO: 35 or a conservative modification thereof; e) the α chain variable region comprises a CDR2 comprising the amino acid sequence set forth in SEQ ID NO: 42 or a conservative modification thereof, and the β chain variable region comprises a CDR2 comprising the amino acid sequence set forth in SEQ ID NO: 45 or a conservative modification thereof; f) the α chain variable region comprises a CDR2 comprising the amino acid sequence set forth in SEQ ID NO: 52 or a conservative modification thereof, and the β chain variable region comprises a CDR2 comprising the amino acid sequence set forth in SEQ ID NO: 55 or a conservative modification thereof; g) the α chain variable region comprises a CDR2 comprising the amino acid sequence set forth in SEQ ID NO: 62 or a conservative modification thereof, and the β chain variable region comprises a CDR2 comprising the amino acid sequence set forth in SEQ ID NO: 65 or a conservative modification thereof; h) the α chain variable region comprises a CDR2 comprising the amino acid sequence set forth in SEQ ID NO: 72 or a conservative modification thereof, and the β chain variable region comprises a CDR2 comprising the amino acid sequence set forth in SEQ ID NO: 75 or a conservative modification thereof; i) the α chain variable region comprises a CDR2 comprising the amino acid sequence set forth in SEQ ID NO: 82 or a conservative modification thereof, and the β chain variable region comprises a CDR2 comprising the amino acid sequence set forth in SEQ ID NO: 85 or a conservative modification thereof; j) the α chain variable region comprises a CDR2 comprising the amino acid sequence set forth in SEQ ID NO: 92 or a conservative modification thereof, and the β chain variable region comprises a CDR2 comprising the amino acid sequence set forth in SEQ ID NO: 95 or a conservative modification thereof; k) the α chain variable region comprises a CDR2 comprising the amino acid sequence set forth in SEQ ID NO: 102 or a conservative modification thereof, and the β chain variable region comprises a CDR2 comprising the amino acid sequence set forth in SEQ ID NO: 105 or a conservative modification thereof; or 1) the α chain variable region comprises a CDR2 comprising the amino acid sequence set forth in SEQ ID NO: 112 or a conservative modification thereof, and the β chain variable region comprises a CDR2 comprising the amino acid sequence set forth in SEQ ID NO: 115 or a conservative modification thereof.

In certain embodiments of the recombinant TCRs disclosed herein, a) the α chain variable region comprises a CDR1 comprising the amino acid sequence set forth in SEQ ID NO: 1 or a conservative modification thereof, and the β chain variable region comprises a CDR1 comprising the amino acid sequence set forth in SEQ ID NO: 4 or a conservative modification thereof; b) the α chain variable region comprises a CDR1 comprising the amino acid sequence set forth in SEQ ID NO: 11 or a conservative modification thereof, and the β chain variable region comprises a CDR1 comprising the amino acid sequence set forth in SEQ ID NO: 14 or a conservative modification thereof; c) the α chain variable region comprises a CDR1 comprising the amino acid sequence set forth in SEQ ID NO: 21 or a conservative modification thereof, and the β chain variable region comprises a CDR1 comprising the amino acid sequence set forth in SEQ ID NO: 24 or a conservative modification thereof; d) the α chain variable region comprises a CDR1 comprising the amino acid sequence set forth in SEQ ID NO: 31 or a conservative modification thereof, and the β chain variable region comprises a CDR1 comprising the amino acid sequence set forth in SEQ ID NO: 34 or a conservative modification thereof; e) the α chain variable region comprises a CDR1 comprising the amino acid sequence set forth in SEQ ID NO: 41 or a conservative modification thereof, and the β chain variable region comprises a CDR1 comprising the amino acid sequence set forth in SEQ ID NO: 44 or a conservative modification thereof; f) the α chain variable region comprises a CDR1 comprising the amino acid sequence set forth in SEQ ID NO: 51 or a conservative modification thereof, and the β chain variable region comprises a CDR1 comprising the amino acid sequence set forth in SEQ ID NO: 54 or a conservative modification thereof; g) the α chain variable region comprises a CDR1 comprising the amino acid sequence set forth in SEQ ID NO: 61 or a conservative modification thereof, and the β chain variable region comprises a CDR1 comprising the amino acid sequence set forth in SEQ ID NO: 64 or a conservative modification thereof; h) the α chain variable region comprises a CDR1 comprising the amino acid sequence set forth in SEQ ID NO: 71 or a conservative modification thereof, and the β chain variable region comprises a CDR1 comprising the amino acid sequence set forth in SEQ ID NO: 74 or a conservative modification thereof; i) the α chain variable region comprises a CDR1 comprising the amino acid sequence set forth in SEQ ID NO: 81 or a conservative modification thereof, and the β chain variable region comprises a CDR1 comprising the amino acid sequence set forth in SEQ ID NO: 84 or a conservative modification thereof; j) the α chain variable region comprises a CDR1 comprising the amino acid sequence set forth in SEQ ID NO: 91 or a conservative modification thereof, and the β chain variable region comprises a CDR1 comprising the amino acid sequence set forth in SEQ ID NO: 94 or a conservative modification thereof; k) the α chain variable region comprises a CDR1 comprising the amino acid sequence set forth in SEQ ID NO: 101 or a conservative modification thereof, and the β chain variable region comprises a CDR1 comprising the amino acid sequence set forth in SEQ ID NO: 104 or a conservative modification thereof; or 1) the α chain variable region comprises a CDR1 comprising the amino acid sequence set forth in SEQ ID NO: 111 or a conservative modification thereof, and the β chain variable region comprises a CDR1 comprising the amino acid sequence set forth in SEQ ID NO: 114 or a conservative modification thereof.

In certain embodiments of the recombinant TCRs disclosed herein, a) the α chain variable region comprises a CDR1 comprising the amino acid sequence set forth in SEQ ID NO: 1 or a conservative modification thereof, a CDR2 comprising the amino acid sequence set forth in SEQ ID NO: 2 or a conservative modification thereof, and a CDR3 comprising the amino acid sequence set forth in SEQ ID NO: 3 or a conservative modification thereof; and the β chain variable region comprises a CDR1 comprising the amino acid sequence set forth in SEQ ID NO: 4 or a conservative modification thereof, a CDR2 comprising the amino acid sequence set forth in SEQ ID NO: 5 or a conservative modification thereof, and a CDR3 comprising the amino acid sequence set forth in SEQ ID NO: 6 or a conservative modification thereof; b) the α chain variable region comprises a CDR1 comprising the amino acid sequence set forth in SEQ ID NO: 11 or a conservative modification thereof, a CDR2 comprising the amino acid sequence set forth in SEQ ID NO: 12 or a conservative modification thereof, and a CDR3 comprising the amino acid sequence set forth in SEQ ID NO: 13 or a conservative modification thereof; and the β chain variable region comprises a CDR1 comprising the amino acid sequence set forth in SEQ ID NO: 14 or a conservative modification thereof, a CDR2 comprising the amino acid sequence set forth in SEQ ID NO: 15 or a conservative modification thereof, and a CDR3 comprising the amino acid sequence set forth in SEQ ID NO: 16 or a conservative modification thereof; c) the α chain variable region comprises a CDR1 comprising the amino acid sequence set forth in SEQ ID NO: 21 or a conservative modification thereof, a CDR2 comprising the amino acid sequence set forth in SEQ ID NO: 22 or a conservative modification thereof, and a CDR3 comprising the amino acid sequence set forth in SEQ ID NO: 23 or a conservative modification thereof; and the β chain variable region comprises a CDR1 comprising the amino acid sequence set forth in SEQ ID NO: 24 or a conservative modification thereof, a CDR2 comprising the amino acid sequence set forth in SEQ ID NO: 25 or a conservative modification thereof, and a CDR3 comprising the amino acid sequence set forth in SEQ ID NO: 26 or a conservative modification thereof; d) the α chain variable region comprises a CDR1 comprising the amino acid sequence set forth in SEQ ID NO: 31 or a conservative modification thereof, a CDR2 comprising the amino acid sequence set forth in SEQ ID NO: 32 or a conservative modification thereof, and a CDR3 comprising the amino acid sequence set forth in SEQ ID NO: 33 or a conservative modification thereof; and the β chain variable region comprises a CDR1 comprising the amino acid sequence set forth in SEQ ID NO: 34 or a conservative modification thereof, a CDR2 comprising the amino acid sequence set forth in SEQ ID NO: 35 or a conservative modification thereof, and a CDR3 comprising the amino acid sequence set forth in SEQ ID NO: 36 or a conservative modification thereof; e) the α chain variable region comprises a CDR1 comprising the amino acid sequence set forth in SEQ ID NO: 41 or a conservative modification thereof, a CDR2 comprising the amino acid sequence set forth in SEQ ID NO: 42 or a conservative modification thereof, and a CDR3 comprising the amino acid sequence set forth in SEQ ID NO: 43 or a conservative modification thereof; and the β chain variable region comprises a CDR1 comprising the amino acid sequence set forth in SEQ ID NO: 44 or a conservative modification thereof, a CDR2 comprising the amino acid sequence set forth in SEQ ID NO: 45 or a conservative modification thereof, and a CDR3 comprising the amino acid sequence set forth in SEQ ID NO: 46 or a conservative modification thereof; f) the α chain variable region comprises a CDR1 comprising the amino acid sequence set forth in SEQ ID NO: 51 or a conservative modification thereof, a CDR2 comprising the amino acid sequence set forth in SEQ ID NO: 52 or a conservative modification thereof, and a CDR3 comprising the amino acid sequence set forth in SEQ ID NO: 53 or a conservative modification thereof; and the β chain variable region comprises a CDR1 comprising the amino acid sequence set forth in SEQ ID NO: 54 or a conservative modification thereof, a CDR2 comprising the amino acid sequence set forth in SEQ ID NO: 55 or a conservative modification thereof, and a CDR3 comprising the amino acid sequence set forth in SEQ ID NO: 56 or a conservative modification thereof; g) the α chain variable region comprises a CDR1 comprising the amino acid sequence set forth in SEQ ID NO: 61 or a conservative modification thereof, a CDR2 comprising the amino acid sequence set forth in SEQ ID NO: 62 or a conservative modification thereof, and a CDR3 comprising the amino acid sequence set forth in SEQ ID NO: 63 or a conservative modification thereof; and the β chain variable region comprises a CDR1 comprising the amino acid sequence set forth in SEQ ID NO: 64 or a conservative modification thereof, a CDR2 comprising the amino acid sequence set forth in SEQ ID NO: 65 or a conservative modification thereof, and a CDR3 comprising the amino acid sequence set forth in SEQ ID NO: 66 or a conservative modification thereof; h) the α chain variable region comprises a CDR1 comprising the amino acid sequence set forth in SEQ ID NO: 71 or a conservative modification thereof, a CDR2 comprising the amino acid sequence set forth in SEQ ID NO: 72 or a conservative modification thereof, and a CDR3 comprising the amino acid sequence set forth in SEQ ID NO: 73 or a conservative modification thereof; and the β chain variable region comprises a CDR1 comprising the amino acid sequence set forth in SEQ ID NO: 74 or a conservative modification thereof, a CDR2 comprising the amino acid sequence set forth in SEQ ID NO: 75 or a conservative modification thereof, and a CDR3 comprising the amino acid sequence set forth in SEQ ID NO: 76 or a conservative modification thereof; i) the α chain variable region comprises a CDR1 comprising the amino acid sequence set forth in SEQ ID NO: 81 or a conservative modification thereof, a CDR2 comprising the amino acid sequence set forth in SEQ ID NO: 82 or a conservative modification thereof, and a CDR3 comprising the amino acid sequence set forth in SEQ ID NO: 83 or a conservative modification thereof; and the β chain variable region comprises a CDR1 comprising the amino acid sequence set forth in SEQ ID NO: 84 or a conservative modification thereof, a CDR2 comprising the amino acid sequence set forth in SEQ ID NO: 85 or a conservative modification thereof, and a CDR3 comprising the amino acid sequence set forth in SEQ ID NO: 86 or a conservative modification thereof; j) the α chain variable region comprises a CDR1 comprising the amino acid sequence set forth in SEQ ID NO: 91 or a conservative modification thereof, a CDR2 comprising the amino acid sequence set forth in SEQ ID NO: 92 or a conservative modification thereof, and a CDR3 comprising the amino acid sequence set forth in SEQ ID NO: 93 or a conservative modification thereof; and the β chain variable region comprises a CDR1 comprising the amino acid sequence set forth in SEQ ID NO: 94 or a conservative modification thereof, a CDR2 comprising the amino acid sequence set forth in SEQ ID NO: 95 or a conservative modification thereof, and a CDR3 comprising the amino acid sequence set forth in SEQ ID NO: 96 or a conservative modification thereof; k) the α chain variable region comprises a CDR1 comprising the amino acid sequence set forth in SEQ ID NO: 101 or a conservative modification thereof, a CDR2 comprising the amino acid sequence set forth in SEQ ID NO: 102 or a conservative modification thereof, and a CDR3 comprising the amino acid sequence set forth in SEQ ID NO: 103 or a conservative modification thereof; and the β chain variable region comprises a CDR1 comprising the amino acid sequence set forth in SEQ ID NO: 104 or a conservative modification thereof, a CDR2 comprising the amino acid sequence set forth in SEQ ID NO: 105 or a conservative modification thereof, and a CDR3 comprising the amino acid sequence set forth in SEQ ID NO: 106 or a conservative modification thereof; or 1) the α chain variable region comprises a CDR1 comprising the amino acid sequence set forth in SEQ ID NO: 111 or a conservative modification thereof, a CDR2 comprising the amino acid sequence set forth in SEQ ID NO: 112 or a conservative modification thereof, and a CDR3 comprising the amino acid sequence set forth in SEQ ID NO: 113 or a conservative modification thereof; and the β chain variable region comprises a CDR1 comprising the amino acid sequence set forth in SEQ ID NO: 114 or a conservative modification thereof, a CDR2 comprising the amino acid sequence set forth in SEQ ID NO: 115 or a conservative modification thereof, and a CDR3 comprising the amino acid sequence set forth in SEQ ID NO: 116 or a conservative modification thereof.

In certain embodiments of the recombinant TCRs disclosed herein, a) the α chain variable region comprises a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3; and the β chain variable region comprises a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6; b) the α chain variable region comprises a CDR1 comprising the amino acid sequence set forth in SEQ ID NO: 11, a CDR2 comprising the amino acid sequence set forth in SEQ ID NO: 12, and a CDR3 comprising the amino acid sequence set forth in SEQ ID NO: 13; and the β chain variable region comprises a CDR1 comprising the amino acid sequence set forth in SEQ ID NO: 14, a CDR2 comprising the amino acid sequence set forth in SEQ ID NO: 15, and a CDR3 comprising the amino acid sequence set forth in SEQ ID NO: 16; c) the α chain variable region comprises a CDR1 comprising the amino acid sequence set forth in SEQ ID NO: 21, a CDR2 comprising the amino acid sequence set forth in SEQ ID NO: 22, and a CDR3 comprising the amino acid sequence set forth in SEQ ID NO: 23; and the β chain variable region comprises a CDR1 comprising the amino acid sequence set forth in SEQ ID NO: 24, a CDR2 comprising the amino acid sequence set forth in SEQ ID NO: 25, and a CDR3 comprising the amino acid sequence set forth in SEQ ID NO: 26; d) the α chain variable region comprises a CDR1 comprising the amino acid sequence set forth in SEQ ID NO: 31, a CDR2 comprising the amino acid sequence set forth in SEQ ID NO: 32, and a CDR3 comprising the amino acid sequence set forth in SEQ ID NO: 33; and the β chain variable region comprises a CDR1 comprising the amino acid sequence set forth in SEQ ID NO: 34, a CDR2 comprising the amino acid sequence set forth in SEQ ID NO: 35, and a CDR3 comprising the amino acid sequence set forth in SEQ ID NO: 36; e) the α chain variable region comprises a CDR1 comprising the amino acid sequence set forth in SEQ ID NO: 41, a CDR2 comprising the amino acid sequence set forth in SEQ ID NO: 42, and a CDR3 comprising the amino acid sequence set forth in SEQ ID NO: 43; and the β chain variable region comprises a CDR1 comprising the amino acid sequence set forth in SEQ ID NO: 44, a CDR2 comprising the amino acid sequence set forth in SEQ ID NO: 45, and a CDR3 comprising the amino acid sequence set forth in SEQ ID NO: 46; f) the α chain variable region comprises a CDR1 comprising the amino acid sequence set forth in SEQ ID NO: 51, a CDR2 comprising the amino acid sequence set forth in SEQ ID NO: 52, and a CDR3 comprising the amino acid sequence set forth in SEQ ID NO: 53; and the β chain variable region comprises a CDR1 comprising the amino acid sequence set forth in SEQ ID NO: 54, a CDR2 comprising the amino acid sequence set forth in SEQ ID NO: 55, and a CDR3 comprising the amino acid sequence set forth in SEQ ID NO: 56; g) the α chain variable region comprises a CDR1 comprising the amino acid sequence set forth in SEQ ID NO: 61, a CDR2 comprising the amino acid sequence set forth in SEQ ID NO: 62, and a CDR3 comprising the amino acid sequence set forth in SEQ ID NO: 63; and the β chain variable region comprises a CDR1 comprising the amino acid sequence set forth in SEQ ID NO: 64, a CDR2 comprising the amino acid sequence set forth in SEQ ID NO: 65, and a CDR3 comprising the amino acid sequence set forth in SEQ ID NO: 66; h) the α chain variable region comprises a CDR1 comprising the amino acid sequence set forth in SEQ ID NO: 71, a CDR2 comprising the amino acid sequence set forth in SEQ ID NO: 72, and a CDR3 comprising the amino acid sequence set forth in SEQ ID NO: 73; and the β chain variable region comprises a CDR1 comprising the amino acid sequence set forth in SEQ ID NO: 74, a CDR2 comprising the amino acid sequence set forth in SEQ ID NO: 75, and a CDR3 comprising the amino acid sequence set forth in SEQ ID NO: 76; i) the α chain variable region comprises a CDR1 comprising the amino acid sequence set forth in SEQ ID NO: 81, a CDR2 comprising the amino acid sequence set forth in SEQ ID NO: 82, and a CDR3 comprising the amino acid sequence set forth in SEQ ID NO: 83; and the β chain variable region comprises a CDR1 comprising the amino acid sequence set forth in SEQ ID NO: 84, a CDR2 comprising the amino acid sequence set forth in SEQ ID NO: 85, and a CDR3 comprising the amino acid sequence set forth in SEQ ID NO: 86; j) the α chain variable region comprises a CDR1 comprising the amino acid sequence set forth in SEQ ID NO: 91, a CDR2 comprising the amino acid sequence set forth in SEQ ID NO: 92, and a CDR3 comprising the amino acid sequence set forth in SEQ ID NO: 93; and the β chain variable region comprises a CDR1 comprising the amino acid sequence set forth in SEQ ID NO: 94, a CDR2 comprising the amino acid sequence set forth in SEQ ID NO: 95, and a CDR3 comprising the amino acid sequence set forth in SEQ ID NO: 96; k) the α chain variable region comprises a CDR1 comprising the amino acid sequence set forth in SEQ ID NO: 101, a CDR2 comprising the amino acid sequence set forth in SEQ ID NO: 102, and a CDR3 comprising the amino acid sequence set forth in SEQ ID NO: 103; and the β chain variable region comprises a CDR1 comprising the amino acid sequence set forth in SEQ ID NO: 104, a CDR2 comprising the amino acid sequence set forth in SEQ ID NO: 105, and a CDR3 comprising the amino acid sequence set forth in SEQ ID NO: 106; or 1) the α chain variable region comprises a CDR1 comprising the amino acid sequence set forth in SEQ ID NO: 111, a CDR2 comprising the amino acid sequence set forth in SEQ ID NO: 112, and a CDR3 comprising the amino acid sequence set forth in SEQ ID NO: 113; and the β chain variable region comprises a CDR1 comprising the amino acid sequence set forth in SEQ ID NO: 114, a CDR2 comprising the amino acid sequence set forth in SEQ ID NO: 115, and a CDR3 comprising the amino acid sequence set forth in SEQ ID NO: 116.

In certain embodiments of the recombinant TCRs disclosed herein, the α chain variable region comprises a CDR1 comprising the amino acid sequence set forth in SEQ ID NO: 81, a CDR2 comprising the amino acid sequence set forth in SEQ ID NO: 82, and a CDR3 comprising the amino acid sequence set forth in SEQ ID NO: 83; and the β chain variable region comprises a CDR1 comprising the amino acid sequence set forth in SEQ ID NO: 84, a CDR2 comprising the amino acid sequence set forth in SEQ ID NO: 85, and a CDR3 comprising the amino acid sequence set forth in SEQ ID NO: 86.

In certain embodiments of the recombinant TCRs disclosed herein, the α chain variable region comprises an amino acid sequence that is at least about 80% homologous or identical to the amino acid sequence set forth in SEQ ID NO: 7, SEQ ID NO: 17, SEQ ID NO: 27, SEQ ID NO: 37, SEQ ID NO: 47, SEQ ID NO: 57, SEQ ID NO: 67, SEQ ID NO: 77, SEQ ID NO: 87, SEQ ID NO: 97, SEQ ID NO: 107, or SEQ ID NO: 117.

In certain embodiments of the recombinant TCRs disclosed herein, the α chain variable region comprises the amino acid sequence set forth in SEQ ID NO: 7, SEQ ID NO: 17, SEQ ID NO: 27, SEQ ID NO: 37, SEQ ID NO: 47, SEQ ID NO: 57, SEQ ID NO: 67, SEQ ID NO: 77, SEQ ID NO: 87, SEQ ID NO: 97, SEQ ID NO: 107, or SEQ ID NO: 117.

In certain embodiments of the recombinant TCRs disclosed herein, the β chain variable region comprises an amino acid sequence that is at least about 80% homologous or identical to the amino acid sequence set forth in SEQ ID NO: 8, SEQ ID NO: 18, SEQ ID NO: 28, SEQ ID NO: 38, SEQ ID NO: 48, SEQ ID NO: 58, SEQ ID NO: 68, SEQ ID NO: 78, SEQ ID NO: 88, SEQ ID NO: 98, SEQ ID NO: 108, or SEQ ID NO: 118.

In certain embodiments of the recombinant TCRs disclosed herein, the β chain variable region comprises the amino acid sequence set forth in SEQ ID NO: 8, SEQ ID NO: 18, SEQ ID NO: 28, SEQ ID NO: 38, SEQ ID NO: 48, SEQ ID NO: 58, SEQ ID NO: 68, SEQ ID NO: 78, SEQ ID NO: 88, SEQ ID NO: 98, SEQ ID NO: 108, or SEQ ID NO: 118.

In certain embodiments of the recombinant TCRs disclosed herein, a) the α chain variable region comprises an amino acid sequence that is at least about 80% homologous or identical to the amino acid sequence set forth in SEQ ID NO: 7, and the β chain variable region comprises an amino acid sequence that is at least about 80% homologous or identical to the amino acid sequence set forth in SEQ ID NO: 8; b) the α chain variable region comprises an amino acid sequence that is at least about 80% homologous or identical to the amino acid sequence set forth in SEQ ID NO: 17, and the β chain variable region comprises an amino acid sequence that is at least about 80% homologous or identical to the amino acid sequence set forth in SEQ ID NO: 18; c) the α chain variable region comprises an amino acid sequence that is at least about 80% homologous or identical to the amino acid sequence set forth in SEQ ID NO: 27, and the β chain variable region comprises an amino acid sequence that is at least about 80% homologous or identical to the amino acid sequence set forth in SEQ ID NO: 28; d) the α chain variable region comprises an amino acid sequence that is at least about 80% homologous or identical to the amino acid sequence set forth in SEQ ID NO: 37, and the β chain variable region comprises an amino acid sequence that is at least about 80% homologous or identical to the amino acid sequence set forth in SEQ ID NO: 38; e) the α chain variable region comprises an amino acid sequence that is at least about 80% homologous or identical to the amino acid sequence set forth in SEQ ID NO: 47, and the β chain variable region comprises an amino acid sequence that is at least about 80% homologous or identical to the amino acid sequence set forth in SEQ ID NO: 48; f) the α chain variable region comprises an amino acid sequence that is at least about 80% homologous or identical to the amino acid sequence set forth in SEQ ID NO: 57, and the β chain variable region comprises an amino acid sequence that is at least about 80% homologous or identical to the amino acid sequence set forth in SEQ ID NO: 58; g) the α chain variable region comprises an amino acid sequence that is at least about 80% homologous or identical to the amino acid sequence set forth in SEQ ID NO: 67, and the β chain variable region comprises an amino acid sequence that is at least about 80% homologous or identical to the amino acid sequence set forth in SEQ ID NO: 68; h) the α chain variable region comprises an amino acid sequence that is at least about 80% homologous or identical to the amino acid sequence set forth in SEQ ID NO: 77, and the β chain variable region comprises an amino acid sequence that is at least about 80% homologous or identical to the amino acid sequence set forth in SEQ ID NO: 78; i) the α chain variable region comprises an amino acid sequence that is at least about 80% homologous or identical to the amino acid sequence set forth in SEQ ID NO: 87, and the β chain variable region comprises an amino acid sequence that is at least about 80% homologous or identical to the amino acid sequence set forth in SEQ ID NO: 88; j) the α chain variable region comprises an amino acid sequence that is at least about 80% homologous or identical to the amino acid sequence set forth in SEQ ID NO: 97, and the β chain variable region comprises an amino acid sequence that is at least about 80% homologous or identical to the amino acid sequence set forth in SEQ ID NO: 98; k) the α chain variable region comprises an amino acid sequence that is at least about 80% homologous or identical to the amino acid sequence set forth in SEQ ID NO: 107, and the β chain variable region comprises an amino acid sequence that is at least about 80% homologous or identical to the amino acid sequence set forth in SEQ ID NO: 108; or 1) the α chain variable region comprises an amino acid sequence that is at least about 80% homologous or identical to the amino acid sequence set forth in SEQ ID NO: 117, and the β chain variable region comprises an amino acid sequence that is at least about 80% homologous or identical to the amino acid sequence set forth in SEQ ID NO: 118.

In certain embodiments of the recombinant TCRs disclosed herein, a) the α chain variable region comprises the amino acid sequence set forth in SEQ ID NO: 7, and the β chain variable region comprises the amino acid sequence set forth in SEQ ID NO: 8; b) the α chain variable region comprises the amino acid sequence set forth in SEQ ID NO: 17, and the β chain variable region comprises the amino acid sequence set forth in SEQ ID NO: 18; c) the α chain variable region comprises the amino acid sequence set forth in SEQ ID NO: 27, and the β chain variable region comprises the amino acid sequence set forth in SEQ ID NO: 28; d) the α chain variable region comprises the amino acid sequence set forth in SEQ ID NO: 37, and the β chain variable region comprises the amino acid sequence set forth in SEQ ID NO: 38; e) the α chain variable region comprises the amino acid sequence set forth in SEQ ID NO: 47, and the β chain variable region comprises the amino acid sequence set forth in SEQ ID NO: 48; f) the α chain variable region comprises the amino acid sequence set forth in SEQ ID NO: 57, and the β chain variable region comprises the amino acid sequence set forth in SEQ ID NO: 58; g) the α chain variable region comprises the amino acid sequence set forth in SEQ ID NO: 67, and the β chain variable region comprises the amino acid sequence set forth in SEQ ID NO: 68; h) the α chain variable region comprises the amino acid sequence set forth in SEQ ID NO: 77, and the β chain variable region comprises the amino acid sequence set forth in SEQ ID NO: 78; i) the α chain variable region comprises the amino acid sequence set forth in SEQ ID NO: 87, and the β chain variable region comprises the amino acid sequence set forth in SEQ ID NO: 88; j) the α chain variable region comprises the amino acid sequence set forth in SEQ ID NO: 97, and the β chain variable region comprises the amino acid sequence set forth in SEQ ID NO: 98; k) the α chain variable region comprises the amino acid sequence set forth in SEQ ID NO: 107, and the β chain variable region comprises the amino acid sequence set forth in SEQ ID NO: 108; or 1) the α chain variable region comprises the amino acid sequence set forth in SEQ ID NO: 117, and the β chain variable region comprises the amino acid sequence set forth in SEQ ID NO: 118.

In certain embodiments of the recombinant TCRs disclosed herein, the α chain variable region comprises the amino acid sequence set forth in SEQ ID NO: 87, and the β chain variable region comprises the amino acid sequence set forth in SEQ ID NO: 88.

In certain embodiments of the recombinant TCRs disclosed herein, the α chain constant region comprises an amino acid sequence that is at least about 80% homologous or identical to the amino acid sequence set forth in SEQ ID NO: 123, SEQ ID NO: 124, or SEQ ID NO: 148.

In certain embodiments of the recombinant TCRs disclosed herein, the α chain constant region comprises the amino acid sequence set forth in SEQ ID NO: 123, SEQ ID NO: 124, or SEQ ID NO: 148.

In certain embodiments of the recombinant TCRs disclosed herein, the β chain constant region comprises an amino acid sequence that is at least about 80% homologous or identical to the amino acid sequence set forth in SEQ ID NO: 125, SEQ ID NO: 126, SEQ ID NO: 127, or SEQ ID NO: 129.

In certain embodiments of the recombinant TCRs disclosed herein, the β chain constant region comprises the amino acid sequence set forth in SEQ ID NO: 125, SEQ ID NO: 126, SEQ ID NO: 127, or SEQ ID NO: 129.

In certain embodiments of the recombinant TCRs disclosed herein, a) the α chain constant region comprises an amino acid sequence that is at least about 80% homologous or identical to the amino acid sequence set forth in SEQ ID NO: 123, SEQ ID NO: 124, or SEQ ID NO: 148; and b) the β chain constant region comprises an amino acid sequence that is at least about 80% homologous or identical to the amino acid sequence set forth in SEQ ID NO: 125, SEQ ID NO: 126, SEQ ID NO: 127, or SEQ ID NO: 129.

In certain embodiments of the recombinant TCRs disclosed herein, a) the α chain constant region comprises the amino acid sequence set forth in SEQ ID NO: 123, SEQ ID NO: 124, or SEQ ID NO: 148; and b) the β chain constant region comprises the amino acid sequence set forth in SEQ ID NO: 125, SEQ ID NO: 126, SEQ ID NO: 127, or SEQ ID NO: 129.

In certain embodiments of the recombinant TCRs disclosed herein, a) the α chain comprises an amino acid sequence that is at least about 80% identical to the amino acid sequence set forth in SEQ ID NO: 9, and the β chain comprises an amino acid sequence that is at least about 80% identical to the amino acid sequence set forth in SEQ ID NO: 10; b) the α chain comprises an amino acid sequence that is at least about 80% identical to the amino acid sequence set forth in SEQ ID NO: 19, and the β chain comprises an amino acid sequence that is at least about 80% identical to the amino acid sequence set forth in SEQ ID NO: 20; c) the α chain comprises an amino acid sequence that is at least about 80% identical to the amino acid sequence set forth in SEQ ID NO: 29, and the β chain comprises an amino acid sequence that is at least about 80% identical to the amino acid sequence set forth in SEQ ID NO: 30; d) the α chain comprises an amino acid sequence that is at least about 80% identical to the amino acid sequence set forth in SEQ ID NO: 39, and the β chain comprises an amino acid sequence that is at least about 80% identical to the amino acid sequence set forth in SEQ ID NO: 40; e) the α chain comprises an amino acid sequence that is at least about 80% identical to the amino acid sequence set forth in SEQ ID NO: 49, and the β chain comprises an amino acid sequence that is at least about 80% identical to the amino acid sequence set forth in SEQ ID NO: 50; f) the α chain comprises an amino acid sequence that is at least about 80% identical to the amino acid sequence set forth in SEQ ID NO: 59, and the β chain comprises an amino acid sequence that is at least about 80% identical to the amino acid sequence set forth in SEQ ID NO: 60; g) the α chain comprises an amino acid sequence that is at least about 80% identical to the amino acid sequence set forth in SEQ ID NO: 69, and the β chain comprises an amino acid sequence that is at least about 80% identical to the amino acid sequence set forth in SEQ ID NO: 70; h) the α chain comprises an amino acid sequence that is at least about 80% identical to the amino acid sequence set forth in SEQ ID NO: 79, and the β chain comprises an amino acid sequence that is at least about 80% identical to the amino acid sequence set forth in SEQ ID NO: 80; i) the α chain comprises an amino acid sequence that is at least about 80% identical to the amino acid sequence set forth in SEQ ID NO: 89, and the β chain comprises an amino acid sequence that is at least about 80% identical to the amino acid sequence set forth in SEQ ID NO: 90; j) the α chain comprises an amino acid sequence that is at least about 80% identical to the amino acid sequence set forth in SEQ ID NO: 99, and the β chain comprises an amino acid sequence that is at least about 80% identical to the amino acid sequence set forth in SEQ ID NO: 100; k) the α chain comprises an amino acid sequence that is at least about 80% identical to the amino acid sequence set forth in SEQ ID NO: 109, and the β chain comprises an amino acid sequence that is at least about 80% identical to the amino acid sequence set forth in SEQ ID NO: 110; or 1) the α chain comprises an amino acid sequence that is at least about 80% identical to the amino acid sequence set forth in SEQ ID NO: 119, and the β chain comprises an amino acid sequence that is at least about 80% identical to the amino acid sequence set forth in SEQ ID NO: 120.

In certain embodiments of the recombinant TCRs disclosed herein, a) the α chain comprises the amino acid sequence set forth in SEQ ID NO: 9, and the β chain comprises the amino acid sequence set forth in SEQ ID NO: 10; b) the α chain comprises the amino acid sequence set forth in SEQ ID NO: 19, and the β chain comprises the amino acid sequence set forth in SEQ ID NO: 20; c) the α chain comprises the amino acid sequence set forth in SEQ ID NO: 29, and the β chain comprises the amino acid sequence set forth in SEQ ID NO: 30; d) the α chain comprises the amino acid sequence set forth in SEQ ID NO: 39, and the β chain comprises the amino acid sequence set forth in SEQ ID NO: 40; e) the α chain comprises the amino acid sequence set forth in SEQ ID NO: 49, and the β chain comprises the amino acid sequence set forth in SEQ ID NO: 50; f) the a chain comprises the amino acid sequence set forth in SEQ ID NO: 59, and the β chain comprises the amino acid sequence set forth in SEQ ID NO: 60; g) the α chain comprises the amino acid sequence set forth in SEQ ID NO: 69, and the β chain comprises the amino acid sequence set forth in SEQ ID NO: 70; h) the α chain comprises the amino acid sequence set forth in SEQ ID NO: 79, and the β chain comprises the amino acid sequence set forth in SEQ ID NO: 80; i) the α chain comprises the amino acid sequence set forth in SEQ ID NO: 89, and the β chain comprises the amino acid sequence set forth in SEQ ID NO: 90; j) the α chain comprises the amino acid sequence set forth in SEQ ID NO: 99, and the β chain comprises the amino acid sequence set forth in SEQ ID NO: 100; k) the α chain comprises the amino acid sequence set forth in SEQ ID NO: 109, and the β chain comprises the amino acid sequence set forth in SEQ ID NO: 110; or 1) the α chain comprises the amino acid sequence set forth in SEQ ID NO: 119, and the β chain comprises the amino acid sequence set forth in SEQ ID NO: 120.

In certain embodiments of the recombinant TCRs disclosed herein, the α chain comprises the amino acid sequence set forth in SEQ ID NO: 89, and the β chain comprises the amino acid sequence set forth in SEQ ID NO: 90.

In certain embodiments of the recombinant TCRs disclosed herein, the TCR is recombinant.

In certain embodiments of the recombinant TCRs disclosed herein, the TCR is recombinantly expressed and/or expressed from a vector.

In certain embodiments, the presently disclosed subject matter relates to a T cell receptor (TCR) comprising an extracellular domain that binds to the same TP53 peptide as a reference TCR or a functional fragment thereof, wherein the reference TCR or functional fragment thereof comprises an α chain variable region and a β chain variable region, wherein: a) the α chain variable region comprises a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3; and the β chain variable region comprises a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6; b) the α chain variable region comprises a CDR1 comprising the amino acid sequence set forth in SEQ ID NO: 11, a CDR2 comprising the amino acid sequence set forth in SEQ ID NO: 12, and a CDR3 comprising the amino acid sequence set forth in SEQ ID NO: 13; and the β chain variable region comprises a CDR1 comprising the amino acid sequence set forth in SEQ ID NO: 14, a CDR2 comprising the amino acid sequence set forth in SEQ ID NO: 15, and a CDR3 comprising the amino acid sequence set forth in SEQ ID NO: 16; c) the α chain variable region comprises a CDR1 comprising the amino acid sequence set forth in SEQ ID NO: 21, a CDR2 comprising the amino acid sequence set forth in SEQ ID NO: 22, and a CDR3 comprising the amino acid sequence set forth in SEQ ID NO: 23; and the β chain variable region comprises a CDR1 comprising the amino acid sequence set forth in SEQ ID NO: 24, a CDR2 comprising the amino acid sequence set forth in SEQ ID NO: 25, and a CDR3 comprising the amino acid sequence set forth in SEQ ID NO: 26; d) the α chain variable region comprises a CDR1 comprising the amino acid sequence set forth in SEQ ID NO: 31, a CDR2 comprising the amino acid sequence set forth in SEQ ID NO: 32, and a CDR3 comprising the amino acid sequence set forth in SEQ ID NO: 33; and the β chain variable region comprises a CDR1 comprising the amino acid sequence set forth in SEQ ID NO: 34, a CDR2 comprising the amino acid sequence set forth in SEQ ID NO: 35, and a CDR3 comprising the amino acid sequence set forth in SEQ ID NO: 36; e) the α chain variable region comprises a CDR1 comprising the amino acid sequence set forth in SEQ ID NO: 41, a CDR2 comprising the amino acid sequence set forth in SEQ ID NO: 42, and a CDR3 comprising the amino acid sequence set forth in SEQ ID NO: 43; and the β chain variable region comprises a CDR1 comprising the amino acid sequence set forth in SEQ ID NO: 44, a CDR2 comprising the amino acid sequence set forth in SEQ ID NO: 45, and a CDR3 comprising the amino acid sequence set forth in SEQ ID NO: 46; f) the α chain variable region comprises a CDR1 comprising the amino acid sequence set forth in SEQ ID NO: 51, a CDR2 comprising the amino acid sequence set forth in SEQ ID NO: 52, and a CDR3 comprising the amino acid sequence set forth in SEQ ID NO: 53; and the β chain variable region comprises a CDR1 comprising the amino acid sequence set forth in SEQ ID NO: 54, a CDR2 comprising the amino acid sequence set forth in SEQ ID NO: 55, and a CDR3 comprising the amino acid sequence set forth in SEQ ID NO: 56; g) the α chain variable region comprises a CDR1 comprising the amino acid sequence set forth in SEQ ID NO: 61, a CDR2 comprising the amino acid sequence set forth in SEQ ID NO: 62, and a CDR3 comprising the amino acid sequence set forth in SEQ ID NO: 63; and the β chain variable region comprises a CDR1 comprising the amino acid sequence set forth in SEQ ID NO: 64, a CDR2 comprising the amino acid sequence set forth in SEQ ID NO: 65, and a CDR3 comprising the amino acid sequence set forth in SEQ ID NO: 66; h) the α chain variable region comprises a CDR1 comprising the amino acid sequence set forth in SEQ ID NO: 71, a CDR2 comprising the amino acid sequence set forth in SEQ ID NO: 72, and a CDR3 comprising the amino acid sequence set forth in SEQ ID NO: 73; and the β chain variable region comprises a CDR1 comprising the amino acid sequence set forth in SEQ ID NO: 74, a CDR2 comprising the amino acid sequence set forth in SEQ ID NO: 75, and a CDR3 comprising the amino acid sequence set forth in SEQ ID NO: 76; i) the α chain variable region comprises a CDR1 comprising the amino acid sequence set forth in SEQ ID NO: 81, a CDR2 comprising the amino acid sequence set forth in SEQ ID NO: 82, and a CDR3 comprising the amino acid sequence set forth in SEQ ID NO: 83; and the β chain variable region comprises a CDR1 comprising the amino acid sequence set forth in SEQ ID NO: 84, a CDR2 comprising the amino acid sequence set forth in SEQ ID NO: 85, and a CDR3 comprising the amino acid sequence set forth in SEQ ID NO: 86; j) the α chain variable region comprises a CDR1 comprising the amino acid sequence set forth in SEQ ID NO: 91, a CDR2 comprising the amino acid sequence set forth in SEQ ID NO: 92, and a CDR3 comprising the amino acid sequence set forth in SEQ ID NO: 93; and the β chain variable region comprises a CDR1 comprising the amino acid sequence set forth in SEQ ID NO: 94, a CDR2 comprising the amino acid sequence set forth in SEQ ID NO: 95, and a CDR3 comprising the amino acid sequence set forth in SEQ ID NO: 96; k) the α chain variable region comprises a CDR1 comprising the amino acid sequence set forth in SEQ ID NO: 101, a CDR2 comprising the amino acid sequence set forth in SEQ ID NO: 102, and a CDR3 comprising the amino acid sequence set forth in SEQ ID NO: 103; and the β chain variable region comprises a CDR1 comprising the amino acid sequence set forth in SEQ ID NO: 104, a CDR2 comprising the amino acid sequence set forth in SEQ ID NO: 105, and a CDR3 comprising the amino acid sequence set forth in SEQ ID NO: 106; or 1) the α chain variable region comprises a CDR1 comprising the amino acid sequence set forth in SEQ ID NO: 111, a CDR2 comprising the amino acid sequence set forth in SEQ ID NO: 112, and a CDR3 comprising the amino acid sequence set forth in SEQ ID NO: 113; and the β chain variable region comprises a CDR1 comprising the amino acid sequence set forth in SEQ ID NO: 114, a CDR2 comprising the amino acid sequence set forth in SEQ ID NO: 115, and a CDR3 comprising the amino acid sequence set forth in SEQ ID NO: 116.

In certain embodiments, the presently disclosed subject matter relates to a cell comprising the TCR of any one of embodiments 1-36.

In certain embodiments of the cells disclosed herein, the cell is transduced with the TCR. In certain embodiments of the cells disclosed herein, the TCR is constitutively expressed on the surface of the cell.

In certain embodiments of the cells disclosed herein, the cell is an immunoresponsive cell. In certain embodiments of the cells disclosed herein, the cell is selected from the group consisting of a T cell, a Natural Killer (NK) cell, and a pluripotent stem cell from which a lymphoid cell may be differentiated.

In certain embodiments of the cells disclosed herein, the cell is a T cell.

In certain embodiments of the cells disclosed herein, the T cell is selected from the group consisting of a cytotoxic T lymphocyte (CTL), a regulatory T cell, a γδ T cell, a Natural Killer-T cell (NK-T), a stem cell memory T cell, a central memory T cell, and an effector memory T cell.

In certain embodiments of the cells disclosed herein, the T cell is a γδ T cell.

In certain embodiments of the cells disclosed herein, the T cell is a NK-T cell.

In certain embodiments of the cells disclosed herein, the cell is an NK cell.

In certain embodiments of the cells disclosed herein, the TCR is integrated at a locus within the genome of the cell.

In certain embodiments of the cells disclosed herein, the locus is selected from a TRAC locus, a TRBC locus, a TRDC locus, and a TRGC locus.

In certain embodiments of the cells disclosed herein, the locus is a TRAC locus or a TRBC locus.

In certain embodiments of the cells disclosed herein, the locus is selected from a PDCD1 locus, a CBLB locus, a CISH locus, and a RASA2 locus.

In certain embodiments of the cells disclosed herein, the locus is selected from a PDCD1 locus.

In certain embodiments of the cells disclosed herein, the locus is selected from a CBLB locus.

In certain embodiments of the cells disclosed herein, the locus is selected from a CISH locus.

In certain embodiments of the cells disclosed herein, the locus is selected from a RASA2 locus.

In certain embodiments of the cells disclosed herein, the locus is a genomic safe harbor.

In certain embodiments of the cells disclosed herein, the cell further comprises at least one recombinant or exogenous coreceptor.

In certain embodiments of the cells disclosed herein, the co-receptor is a CD8 co-receptor.

In certain embodiments of the cells disclosed herein, the co-receptor in a CD4 co-receptor.

In certain embodiments, the presently disclosed subject matter relates to a composition comprising the cell of any one of embodiments 37-58.

In certain embodiments of the compositions disclosed herein, the composition is a pharmaceutical composition further comprising a pharmaceutically acceptable carrier.

In certain embodiments, the presently disclosed subject matter relates to a nucleic acid encoding the T cell receptor (TCR) of any one of embodiments 1-36.

In certain embodiments, the presently disclosed subject matter relates to a nucleic acid encoding a polypeptide comprising an amino acid sequence that is at least about 80% identical to the amino acid sequence set forth in SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, or SEQ ID NO: 144.

In certain embodiments, the presently disclosed subject matter relates to a nucleic acid encoding a polypeptide comprising or consisting of the amino acid sequence set forth in SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, or SEQ ID NO: 144.

In certain embodiments of the nucleic acids disclosed herein, the nucleic acid further encodes a CD8 receptor or a CD4 receptor.

In certain embodiments, the presently disclosed subject matter relates to a vector comprising the nucleic acid of any one of embodiments 61-64.

In certain embodiments of the vectors disclosed herein, the vector is a γ-retroviral vector.

In certain embodiments of the vectors disclosed herein, the vector is a plasmid comprising a backbone comprising fewer than about 500 nucleotides.

In certain embodiments, the presently disclosed subject matter relates to a lipid nanoparticle comprising the nucleic acid or the vector disclosed herein.

In certain embodiments, the presently disclosed subject matter relates to a cell comprising the nucleic acid, the vector, or the lipid nanoparticle disclosed herein.

In certain embodiments, the presently disclosed subject matter relates to a composition comprising the nucleic acid, the vector, the lipid nanoparticle, or the cell disclosed herein.

In certain embodiments of the compositions disclosed herein, the composition is a pharmaceutical composition further comprising a pharmaceutically acceptable carrier.

In certain embodiments, the presently disclosed subject matter relates to a method for producing a cell that binds to a TP53 peptide that comprises a R175 mutation, comprising introducing into the cell the nucleic acid, the vector, the lipid nanoparticle, or the composition of disclosed herein.

In certain embodiments, the presently disclosed subject matter relates to a method of treating and/or preventing a tumor or a premalignant condition associated with TP53 in a subject, comprising administering to the subject the cell, the nucleic acid of claim, the vector, the lipid nanoparticle, or the composition disclosed herein.

In certain embodiments of the methods disclosed herein, the TP53 mutation is a R175H mutation.

In certain embodiments of the methods disclosed herein, the tumor is selected from the group consisting of brain tumors, soft tissue and bone sarcomas, adrenocortical carcinoma, rhabdomyosarcoma, Wilms tumor, pancreatic cancer, breast cancer, endometrial cancer, cervical cancer, anal cancer, bladder cancer, colorectal cancer, cholangiocarcinoma/bile duct cancer, lung cancer, ovarian cancer, esophageal cancer, gastric cancer, head and neck squamous cell carcinoma, nonmelanoma skin cancer, salivary gland cancer, melanoma, multiple myeloma, prostate cancer, T-lymphoblastic leukemia/lymphoma, acute myeloid leukemia, B-cell leukemia, myeloproliferative neoplasm, and histiocytosis.

In certain embodiments of the methods disclosed herein, the tumor is colorectal cancer. In certain embodiments of the methods disclosed herein, the tumor is breast cancer. In certain embodiments of the methods disclosed herein, the tumor is esophageal cancer. In certain embodiments of the methods disclosed herein, the premalignant condition is clonal hematopoiesis.

In certain embodiments of the methods disclosed herein, the subject is a human.

In certain embodiments of the methods disclosed herein, the subject comprises an HLA-A. In certain embodiments of the methods disclosed herein, the HLA-A is an HLA-A*02 superfamily member.

In certain embodiments of the methods disclosed herein, the HLA-A*02 superfamily member is selected from the group comprising HLA-A*02, HLA-A*02:01, HLA-A*02:02, HLA-A*02:03, HLA-A*02:04, HLA-A*02:05, HLA-A*02:06, HLA-A*02:07, HLA-A*02:09, HLA-A*02:11, HLA-A*02:12, HLA-A*02:13, HLA-A*02:0215HLA-A*02:14, HLA-A*02:17, HLA-A*02:71, HLA-A*02:77, HLA-A*68:02, and HLA-A*69:01.

In certain embodiments of the methods disclosed herein, the HLA-A*02 superfamily member is HLA-A*02:01.

In certain embodiments, the presently disclosed subject matter relates to the cells, the nucleic acids, the vectors, the lipid nanoparticles, or the compositions disclosed herein for use in treating and/or preventing a tumor or a premalignant condition associated with TP53 in a subject.

In certain embodiments of the cells, nucleic acids, vectors, lipid nanoparticles, or compositions for use disclosed herein, the TP53 mutation is a R175H mutation.

In certain embodiments of the cells, nucleic acids, vectors, lipid nanoparticles, or compositions for use disclosed herein, the tumor is selected from the group consisting of brain tumors, soft tissue and bone sarcomas, adrenocortical carcinoma, rhabdomyosarcoma, Wilms tumor, pancreatic cancer, breast cancer, endometrial cancer, cervical cancer, anal cancer, bladder cancer, colorectal cancer, cholangiocarcinoma/bile duct cancer, lung cancer, ovarian cancer, esophageal cancer, gastric cancer, head and neck squamous cell carcinoma, nonmelanoma skin cancer, salivary gland cancer, melanoma, multiple myeloma, prostate cancer, T-lymphoblastic leukemia/lymphoma, acute myeloid leukemia, B-cell leukemia, myeloproliferative neoplasm, histiocytosis, and clonal hematopoiesis.

In certain embodiments of the cells, nucleic acids, vectors, lipid nanoparticles, or compositions for use disclosed herein, the tumor is colorectal cancer.

In certain embodiments of the cells, nucleic acids, vectors, lipid nanoparticles, or compositions for use disclosed herein, the tumor is breast cancer.

In certain embodiments of the cells, nucleic acids, vectors, lipid nanoparticles, or compositions for use disclosed herein, the tumor is esophageal cancer.

In certain embodiments of the cells, nucleic acids, vectors, lipid nanoparticles, or compositions for use disclosed herein, the premalignant condition is clonal hematopoiesis.

In certain embodiments of the cells, nucleic acids, vectors, lipid nanoparticles, or compositions for use disclosed herein, the subject is a human.

In certain embodiments of the cells, nucleic acids, vectors, lipid nanoparticles, or compositions for use disclosed herein, the subject comprises an HLA-A.

In certain embodiments of the cells, nucleic acids, vectors, lipid nanoparticles, or compositions for use disclosed herein, the HLA-A is an HLA-A*02 superfamily member.

In certain embodiments of the cells, nucleic acids, vectors, lipid nanoparticles, or compositions for use disclosed herein, the HLA-A*02 superfamily member is selected from the group comprising HLA-A*02, HLA-A*02:01, HLA-A*02:02, HLA-A*02:03, HLA-A*02:04, HLA-A*02:05, HLA-A*02:06, HLA-A*02:07, HLA-A*02:09, HLA-A*02:11, HLA-A*02:12, HLA-A*02:13, HLA-A*02:0215HLA-A*02:14, HLA-A*02:17, HLA-A*02:71, HLA-A*02:77, HLA-A*68:02, and HLA-A*69:01.

In certain embodiments of the cells, nucleic acids, vectors, lipid nanoparticles, or compositions for use disclosed herein, the HLA-A*02 superfamily member is HLA-A*02:01.

### EXAMPLES

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the compositions, and assay, screening, and therapeutic methods of the invention, and are not intended to limit the scope of what the inventors regard as their invention.

### Example 1

Immunotherapy can induce durable remissions in patients with highly mutated tumors. However, most malignancies are modestly mutated and fail to respond to current immunotherapy options. Therefore, innovative approaches that boost primary response rates and overcome immune evasion of tumors are urgently needed to bring the curative potential of immunotherapy to more patients with cancer. Previously, detailed immune monitoring studies of exceptional patient responders were performed to resolve the mechanisms of successful immunotherapy. These revealed that T cells from responders often recognize neoantigens (NeoAgs), peptides derived from the protein products of somatic mutations found in a patient's tumor and presented by a patient's complement of human leukocyte antigen (HLA) molecules. In most cases, NeoAgs are “private” and exclusive to an individual patient as they result from random passenger mutations that do not contribute to cancer cell fitness. Such private NeoAgs present a significant challenge to therapeutically target in a time- and cost-efficient manner because they require bespoke production. But NeoAgs can also derive from common hotspot mutations, a unique and elite subset we have termed “public” NeoAgs. T cell receptors (TCRs) that specifically recognize public NeoAgs are highly desirable as they would be cancer-specific yet effective against many tumors sharing a hotspot mutation, enabling design of off-the-shelf therapeutics.

Empiric discovery of public NeoAg-specific TCR clones is challenging because it requires a large biorepository of mutation- and HLA-matched patient samples to search through. The presently disclosed subject matter relates to the discovery of a panel of 12 novel T cell receptors that specifically recognize the R175H TP53 hotspot mutation restricted by human leukocyte antigen (HLA) A2. The tumor suppressor TP53 is a supremely attractive target for such an approach as it is presently undruggable, while also constituting the most frequently (>40% of cases) mutated gene across all subsets of human cancer. Additionally, 8 hotspots comprise ~20% of all cases. The aforementioned HLA-mutation profile encompasses ~1% of all human cancers. The presently disclosed subject matter stems from a large biorepository of patients with the R175H mutation and requisite HLA-A*02:01 type. This biorepository was screened using specific in vitro stimulation protocol capable of expanding rare mutant-reactive clones from patient samples, first detecting them using a novel dextramer-based FACS assay, and de-orphaning candidate TCR clonotypes using a unique mutant feature-barcoded single cell sequencing approach. Retrieved TCR sequences were then cloned into a retroviral vector for validation and downstream assays to evaluate the functional capacity and cross reactivity in tumor cell lines, resulting in a panel of 12 functionally validated receptors from 5 unique patients (named herein 1A, 1B, 1C, 1D, 3A, 3B, 3C, 4A, 7A, 7B, 7D, 8A, where #denotes unique, de-identified patient of origin) that constitute the present invention. These novel receptors were further evaluated in in vivo models with ultimate plans to develop these receptors as adoptive cellular therapy products for the treatment of human malignancies.

### Results

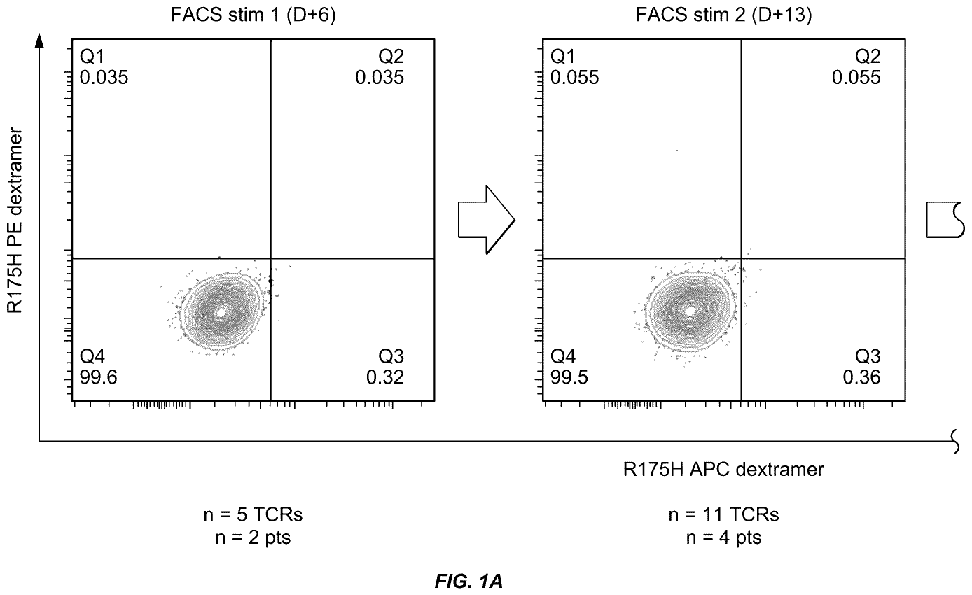

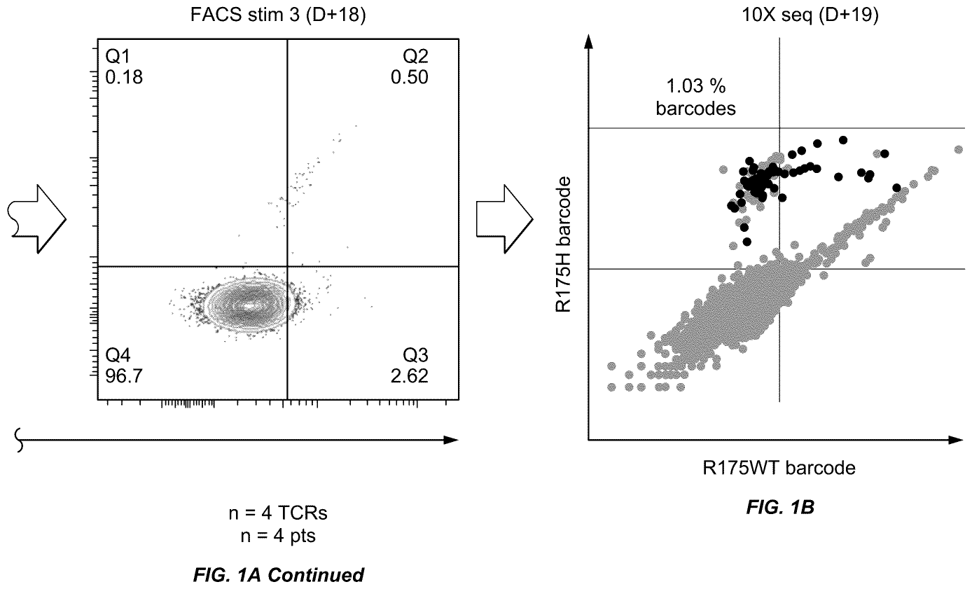

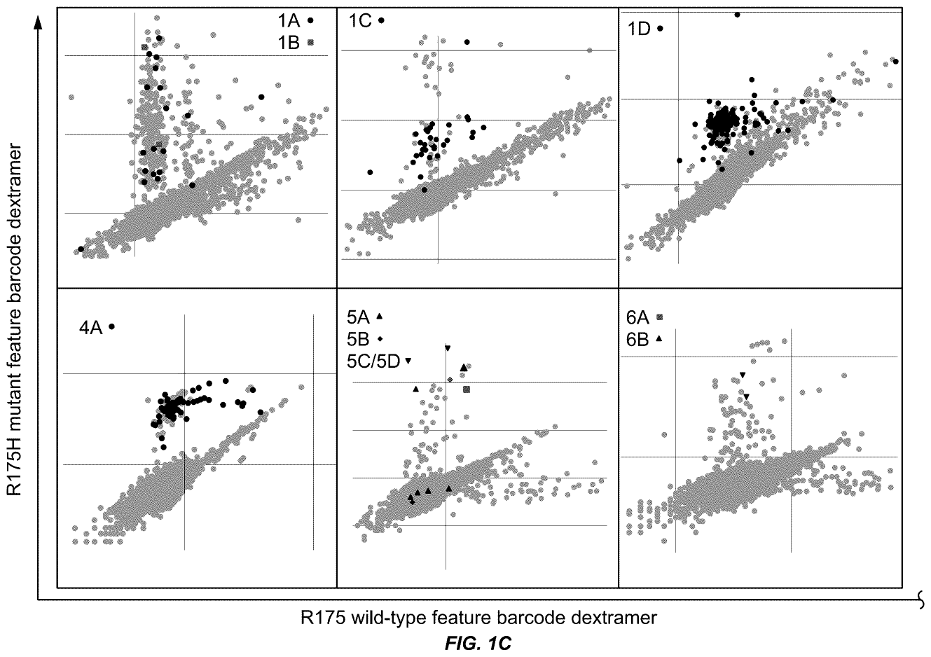

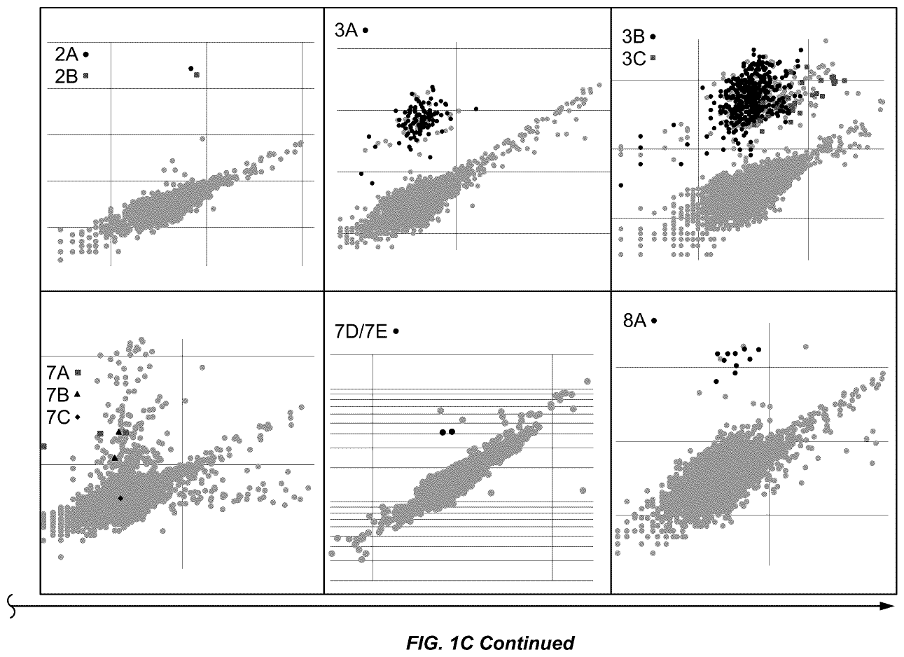

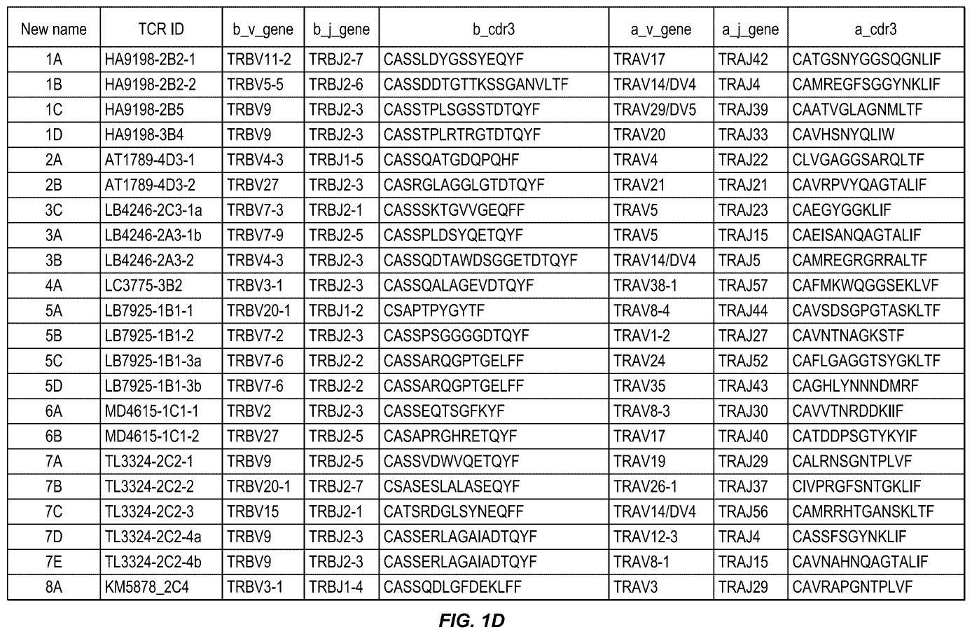

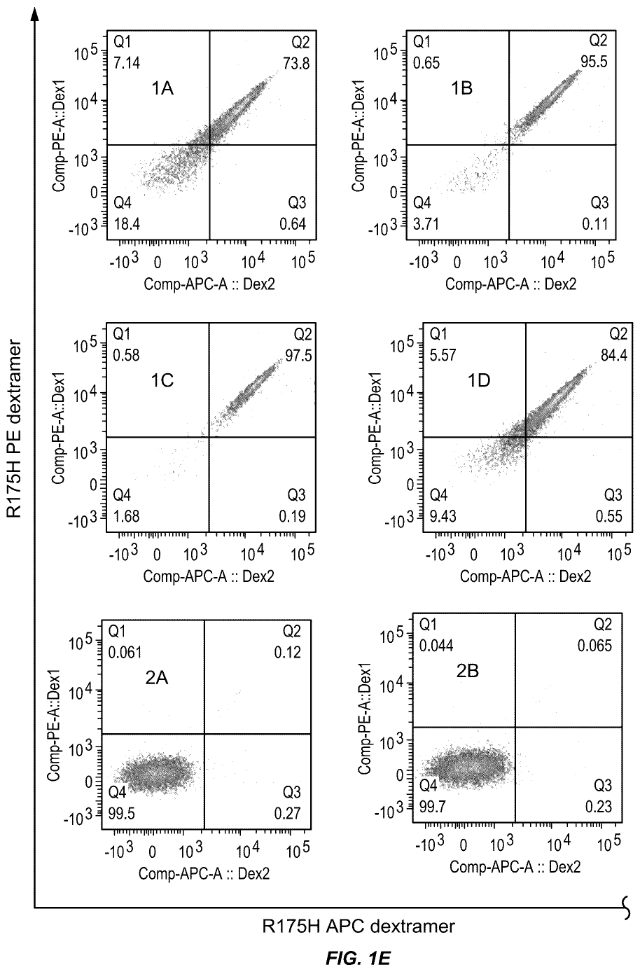

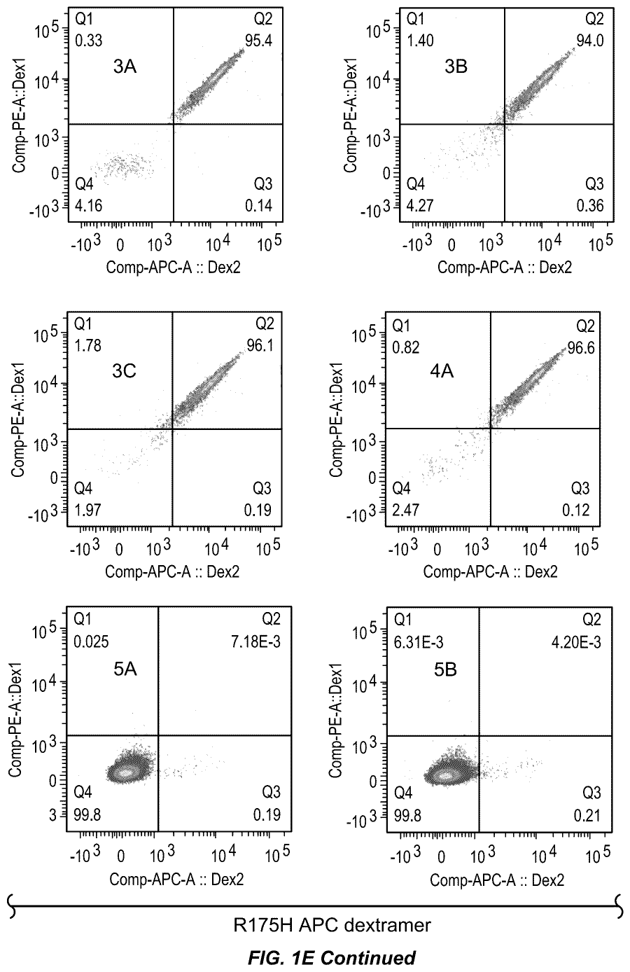

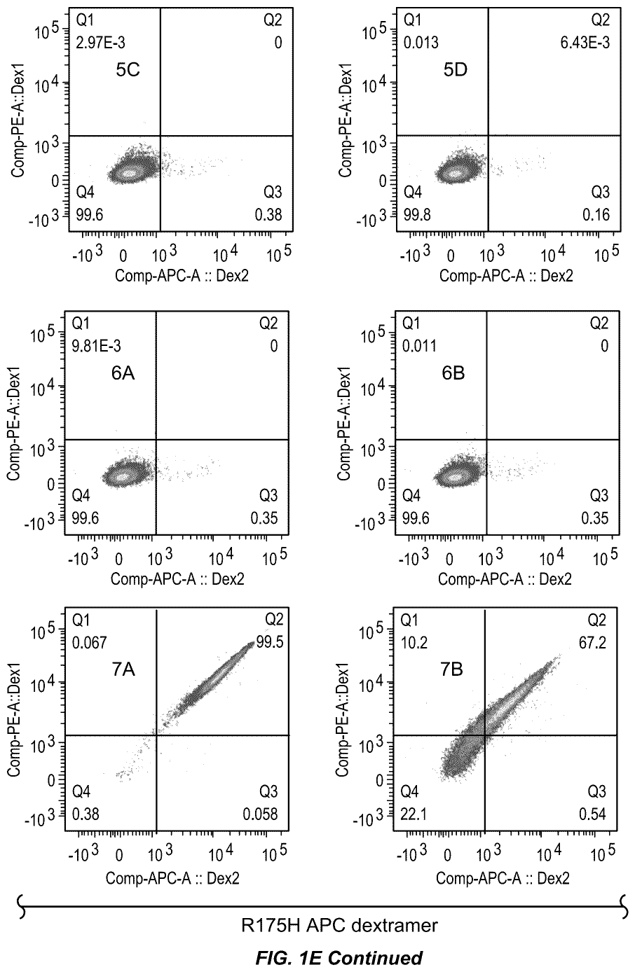

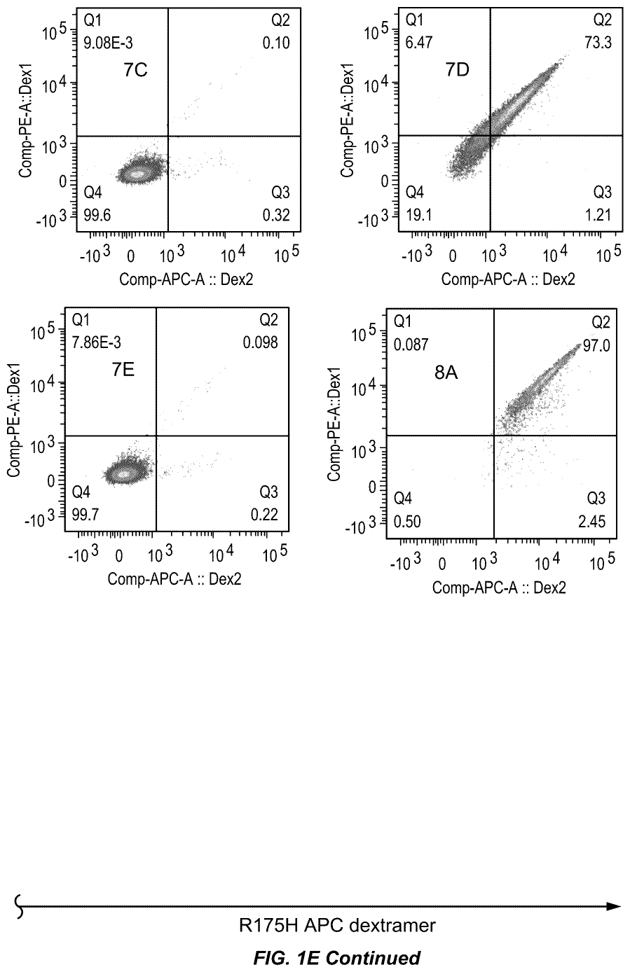

Cells were stimulated through three iterative rounds of peptide stimulation each followed by staining with dual-color mutant peptide/HLA dextramers and analyzed for double-positive populations by flow cytometry (FIG. 1A). Double-positive (hit) populations of cells were isolated by FACS and hit wells were processed by single-cell TCR sequencing using mutant and wild-type feature barcodes to facilitate de-orphaning hit TCR clonotypes (FIG. 1B).

All hit wells from the in vitro stimulation screen were processed by single-cell gene expression, TCR, and feature barcode sequencing (FIG. 1C). As shown in FIG. 1C, the feature barcode unique molecular identifier (UMI) plots report per-cell counts of mutant (y-axis) and wild-type (x-axis) feature barcode reads for each cell sequenced. Hit clonotypes in colors are distinguished from non-specific clonotypes in gray. Unique names that identify each hit hereinafter are provided in the inset with colors corresponding to the respective hit. Each number corresponds to a unique patient of origin; each letter a unique TCR from that given patient. All 22 putative hits were sent for cloning and evaluated in downstream assays.

Next, the 22 retrieved TCR sequences (FIG. 1D) were cloned into a retroviral vector constructed with N-terminal β followed by α chains. The human αβ constant regions were substituted for murine αβ constant regions with a cysteine dimerization domain, and a LVL aliphatic substitution.

The resulting vectors were further validated. CD8 T cells were transduced with each TCR recovered from single cell sequencing. Similar to the FACS assay used for the initial screening, T cells were stained with dual-color mutant peptide/HLA (R175H/A*02:01) dextramers and analyzed for neoantigen specific binding by flow cytometry. Of the 22 hits identified in the initial screen, 12 hits were validated as mutant dextramer binders (FIG. 1E).

Here, the presently disclosed subject matter describes 12 novel T cell receptors (TCRs) isolated from a biorepository of cancer patient cells that recognizes mutated R175H TP53 in the context of HLA-A*02. The 12 TCRs derived from 5 unique patients (named herein 1A, 1B, 1C, 1D, 3A, 3B, 3C, 4A, 7A, 7B, 7D, 8A, where #denotes unique, de-identified patient of origin) were validated as R175H/A*02:01 specific receptors (FIG. 1E).

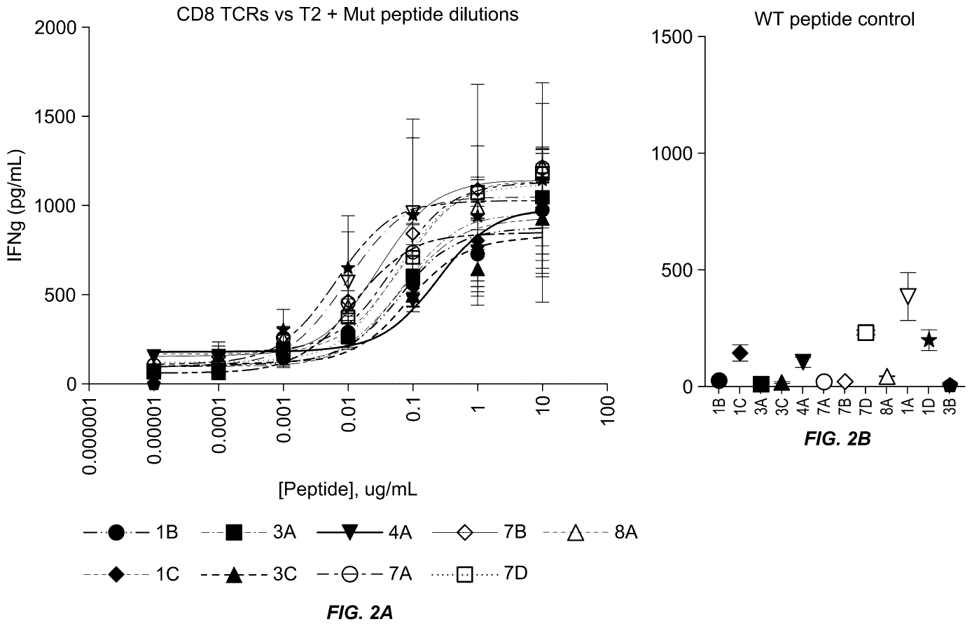

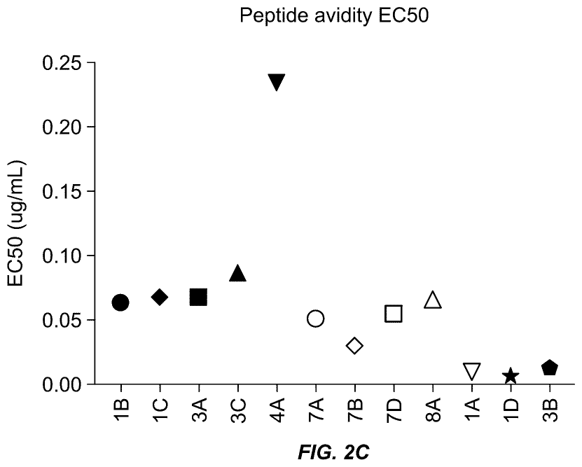

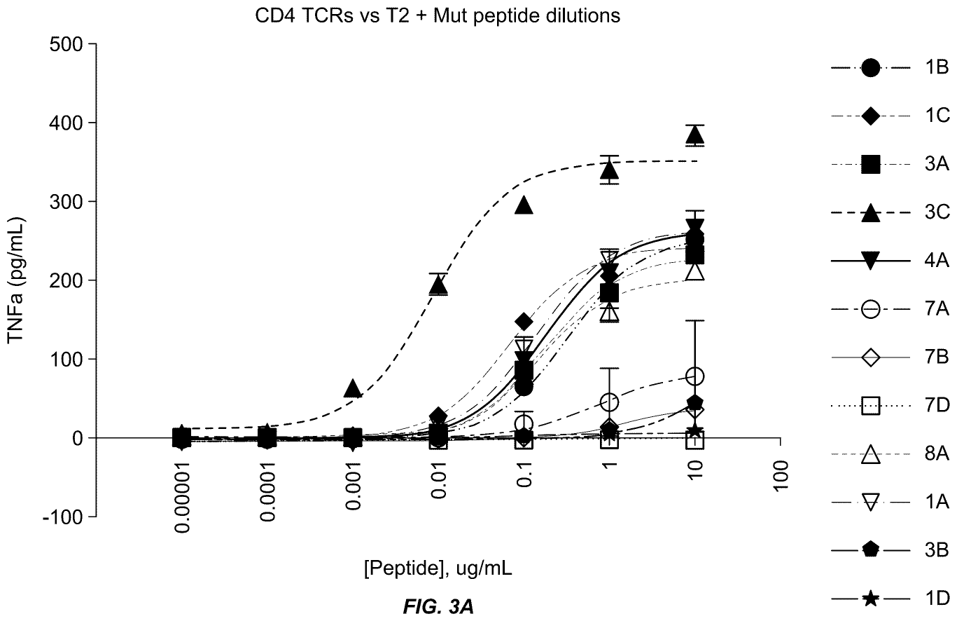

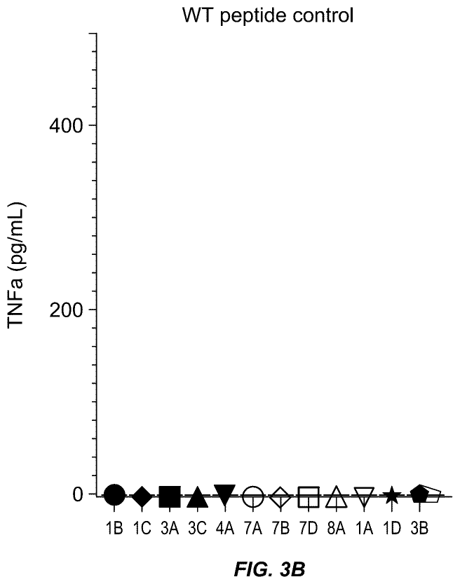

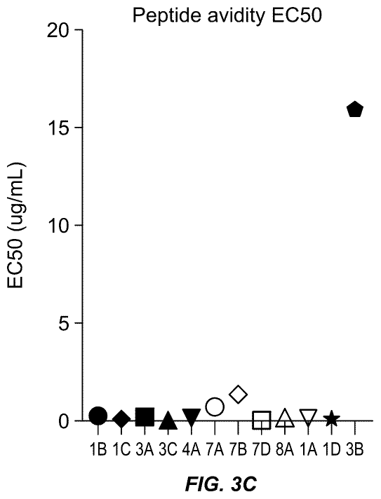

To study the properties of these TCRs, T2 cells, which endogenously express HLA-A*02:01, were pulsed with various concentrations of mutant peptide (e.g., a mutant TP53 peptide disclosed herein), washed and then co-cultured overnight with CD8⁺transduced T cells, and interferon gamma (IFNγ) production was then quantified by ELISA (FIG. 2A). As shown in FIG. 2A, cells expressing the presently disclosed TCRs had a significant secretion of IFNγ upon antigen recognition. Notably, as shown in FIG. 2B, control T cells incubated with T2 cells pulsed with control wild-type peptide at 10 μg/mL did not have increased IFNγ secretion. Thus, these data demonstrate that the activation is mutant-specific. Next, peptide avidity was calculated from the EC50 values for each TCR calculated by fitting data to a 3 parameter dose-response curve (FIG. 2C). Next, similar experiments were conducted with CD4⁺ T cells. CD4⁺ T cells were transduced with the retroviral constructs encoding the TCRs and, as described above, were cocultured with peptide pulsed T2 cells. Tumor necrosis factor alpha (TNFa) production was significantly increased in presence of mutated peptides. (FIGS. 3A and 3B) These data demonstrates that the activation was mutant-specific (FIG. 3B). Further, peptide avidity was calculated from the EC50 values for each TCR calculated by fitting data to a 3 parameter dose-response curve (FIG. 3C).

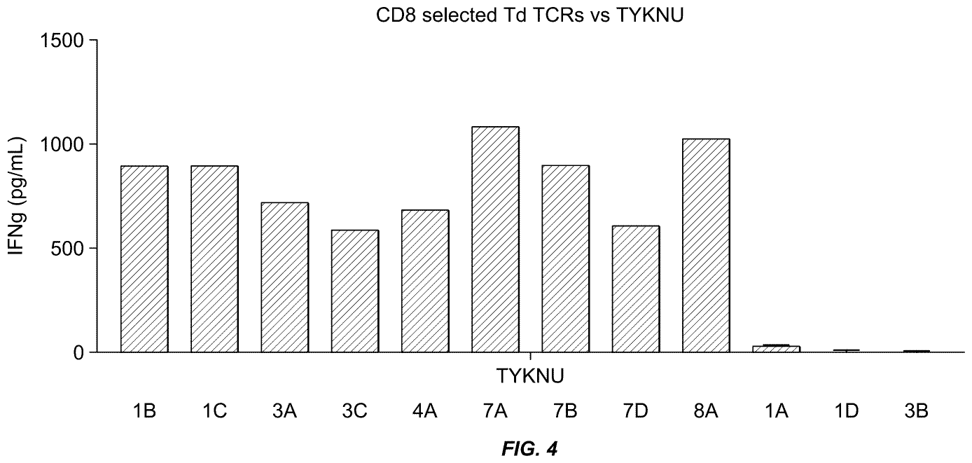

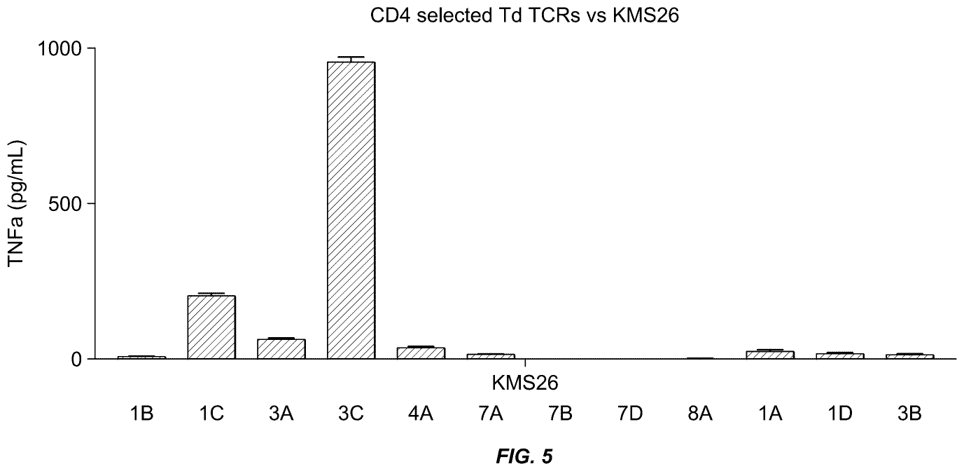

Next, the presently disclosed subject matter studied the functional activation of CD8⁺ or CD4⁺ T cells transduced with each hit TCR of interest and incubated with cancer cells that endogenously express both HLA-A*02:01 and TP53 R175H. CD8⁺ T cells were transduced with each one of the presently disclosed TCRs. T cells were then co-cultured with TYKNU human ovarian cancer cells, which endogenously express both HLA-A*02:01 and TP53 R175H. After overnight incubation, IFNγ production was measured by ELISA (FIG. 4). Similarly, CD4⁺T cells were transduced with each one of the presently disclosed TCRs and cocultured with KMS26 human myeloma cells. The resulting TNFa production was assessed after overnight incubation (FIG. 5). As can be seen in FIGS. 4 and 5, the presently disclosed TCRs were able to elicit a specific response (i.e., increased IFNγ production and TNFa production) in presence of cells expressing the mutant TP53 peptides disclosed herein.

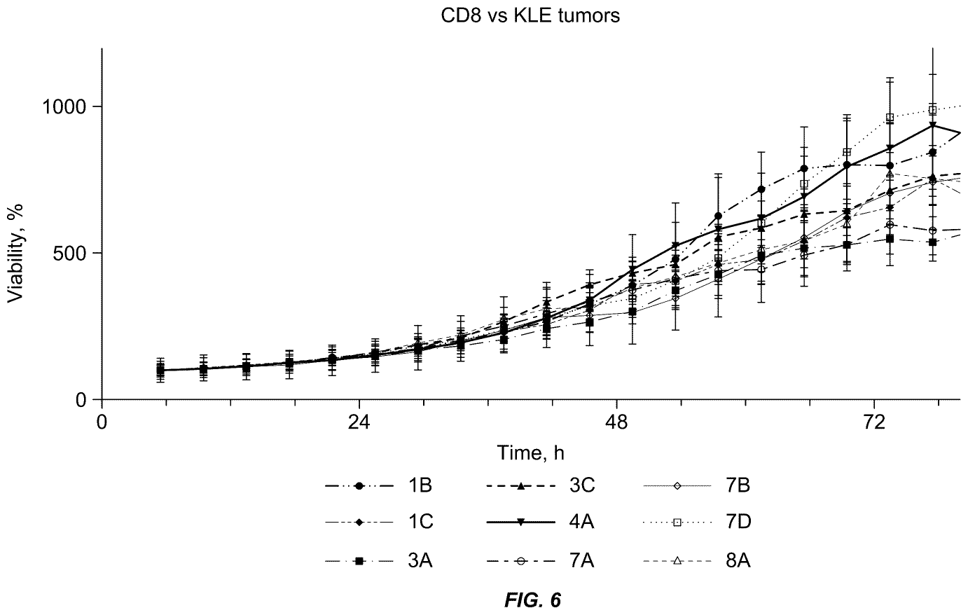

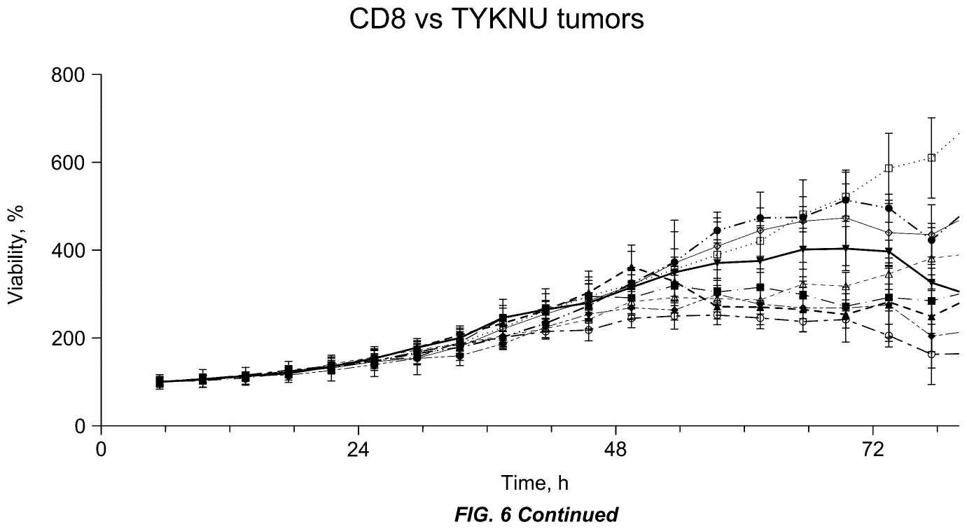

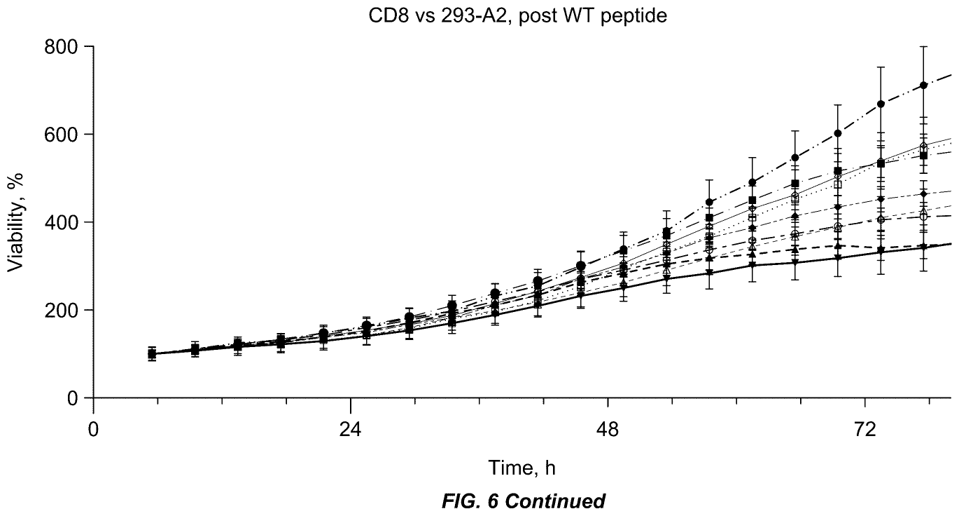

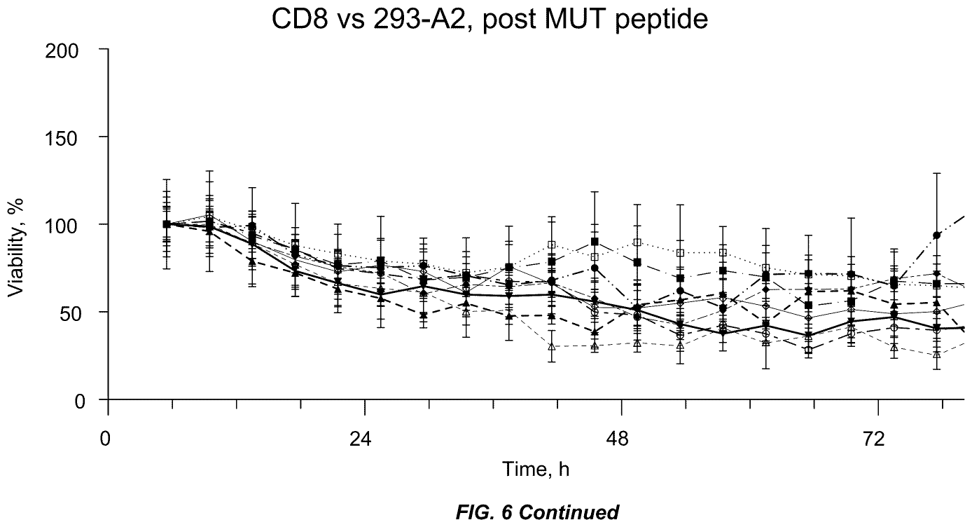

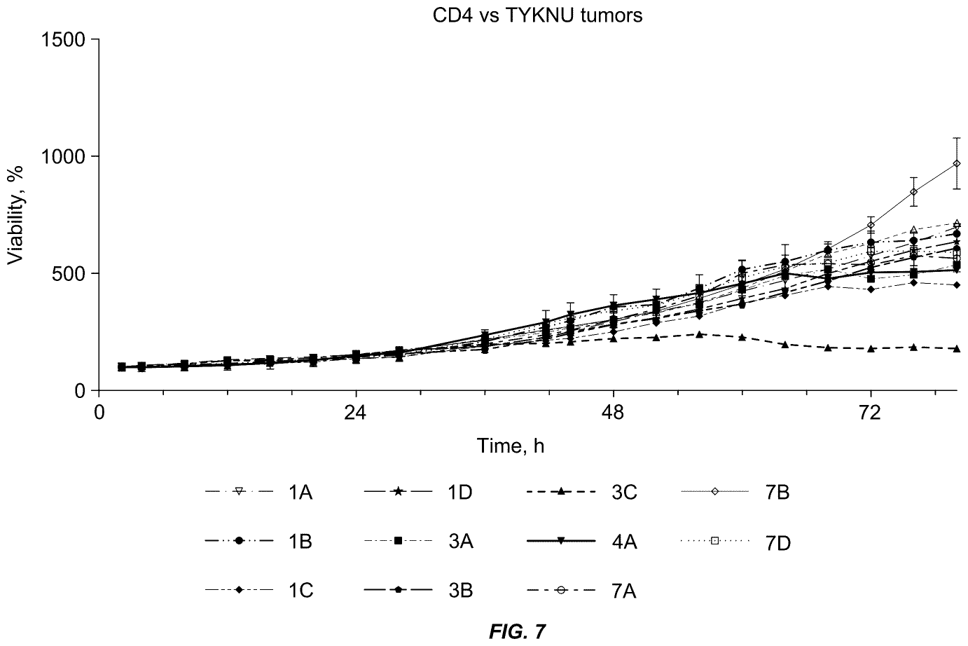

Next, the tumor killing ability of transduced CD8⁺ T cells or CD4⁺ T cells was evaluated for the subset of the presently disclosed TCRs (9 of 12 TCRs). Fluorescently labeled KLE cells (endogenously HLA-A2⁺/TP53_R175H⁺), TYKNU cells (endogenously HLA-A2⁺/TP53_R175H⁺), and 293-A2 cells (transduced with vectors in order to be HLA-A2+) were prepared. 293-A2 tumor cells were pretreated with either wild-type (control) or mutant (experimental) peptide and washed 2 h prior to plating. T cells were then co-cultured with each tumor cell line for over 72 hours and viability was measured in real time. As seen in FIGS. 6 and 7, T cells transduced with the presently disclosed TCRs showed functional ability to reduce viability of tumor cells expressing a mutant TP53 peptide when expressed within a HLA-A2 complex.

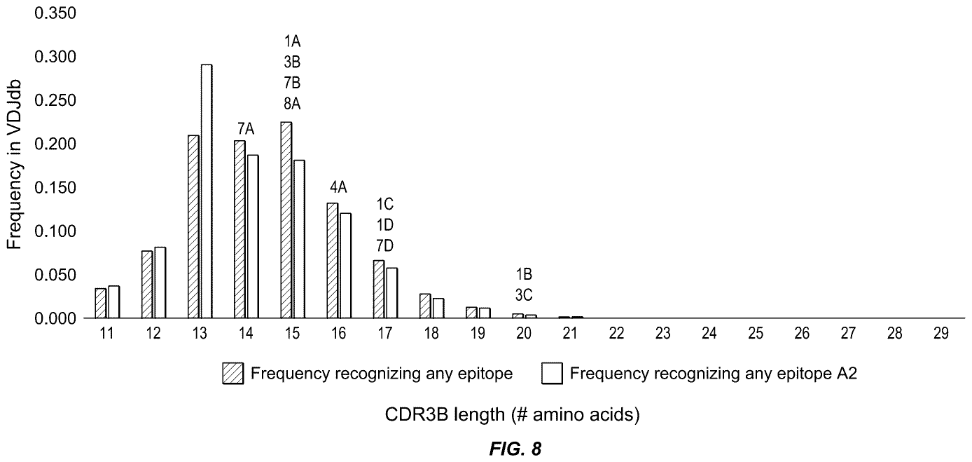

Finally, structural analysis of the TCRs was performed by comparing the receptor sequences to a curated database of T-cell receptor sequences with known antigen specificity (VDJdb). This analysis revealed an elite class of super long CDR3B loops within the novel TCR sequences (FIG. 8). As depicted in FIG. 8, bar plots show the gaussian distribution of CDR3B loop lengths of all reported clonotypes reported on VDJdb recognizing any epitope (orange bars, median=14 amino acids) or recognizing specifically HLA-A*02-restricted epitopes (yellow bars, median=14 amino acids). CDR3B loop lengths for each of the 12 functionally validated TCRs are reported above each respective bar, with 11/12 receptors exhibiting loops >median distribution. Overall, these findings establish the immunogenicity and therapeutic potential of mutant TP53 peptides and the recognition of these peptides by the presently disclosed TCRs.

### Example 2

The present example shows extensive cross-reactivity profiling of the presently disclosed TCRs in order to characterize safety. In addition, the present example shows completed initial in vivo experiments in order to characterize the anti-tumor efficacy of a presently disclosed TCR (7A) versus the most potent TCR known in the art (6-11/2 aka TCR2).

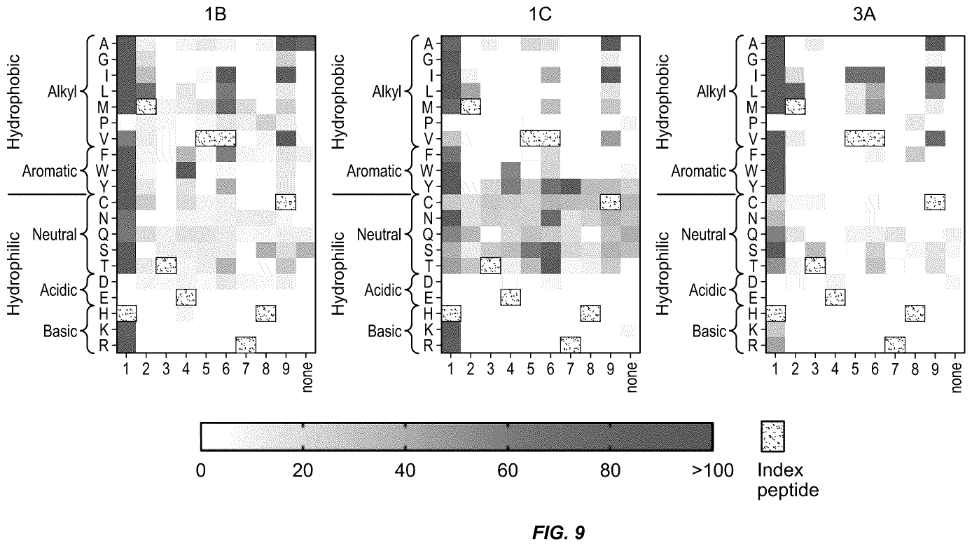

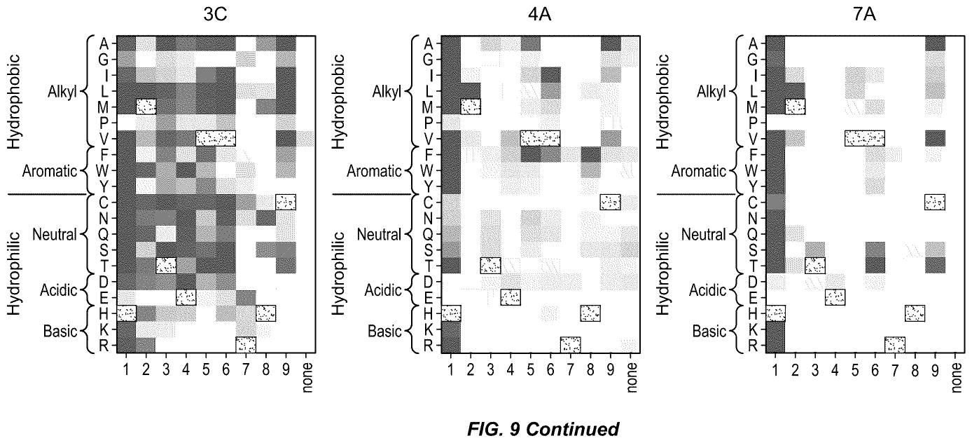

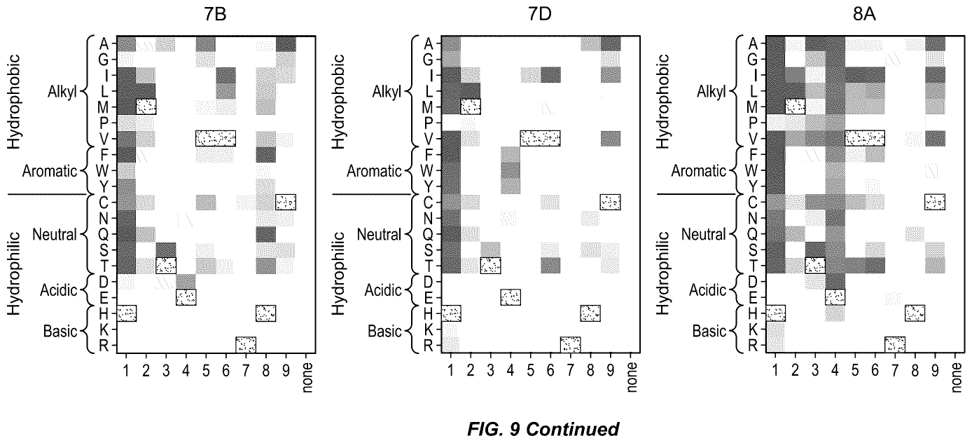

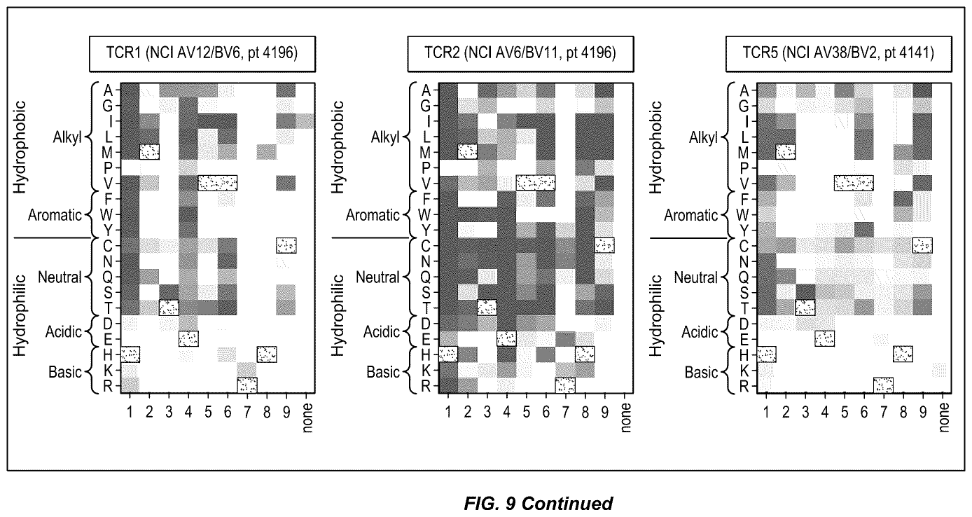

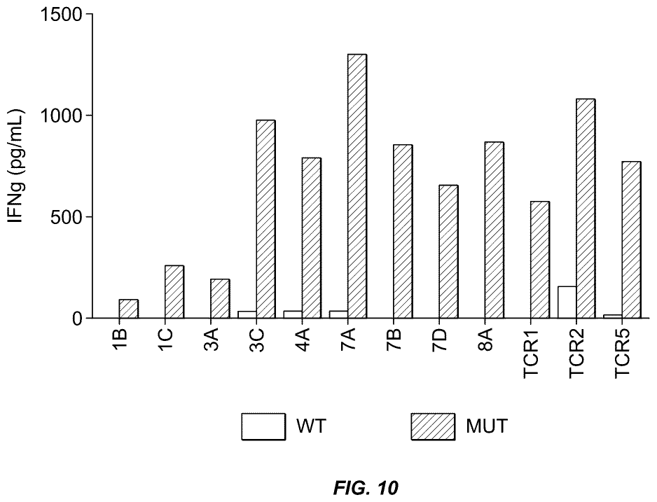

To start, X-scan of the presently disclosed HLA A*02:01/TP53 (R175H)-specific TCRs and known NCI TCRs as benchmarks was performed. As seen in FIG. 9, for each TCR, a 9×20 heatmap reports IFNg production following overnight co-culture with T2 cells previously pulsed with 1 μM of the index peptide (HMTEVVRHC; SEQ ID NO: 146) and all 171 single amino acid variants. Next, the index peptide (HMTEVVRHC; SEQ ID NO: 146) and the wild-type variant (HMTEVVRRC; SEQ ID NO: 147) signals were extracted from the X-scan runs for each TCR to quantify the absolute IFNg release (FIG. 10). Overall, these data show that the presently disclosed TCR can specifically recognize the index peptide.

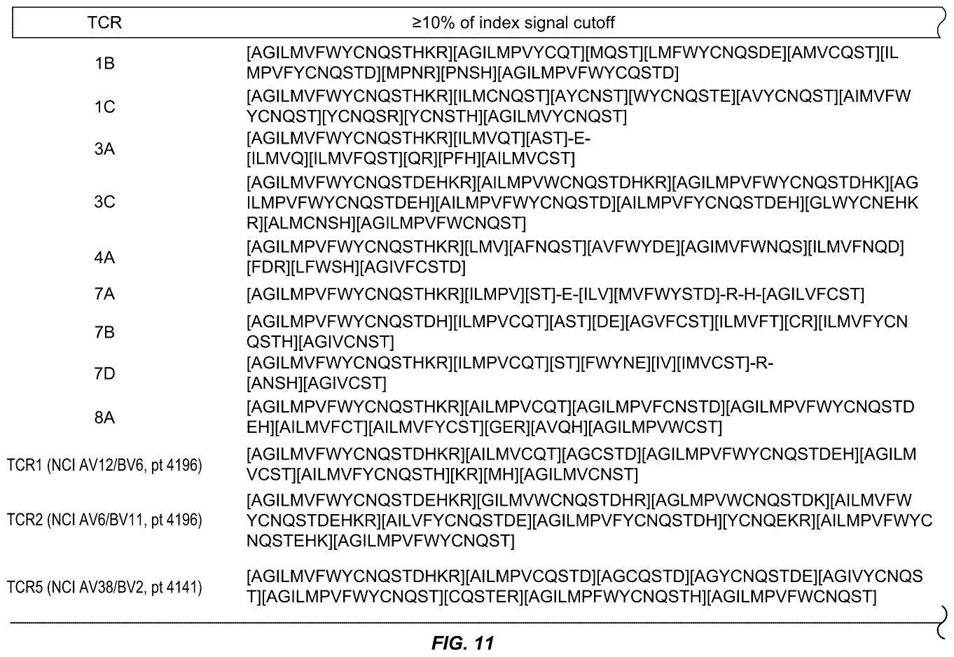

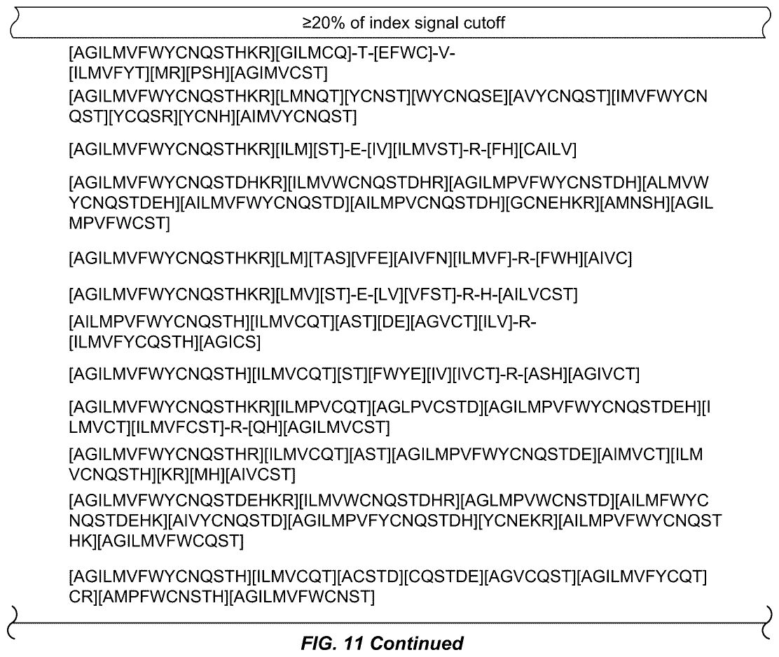

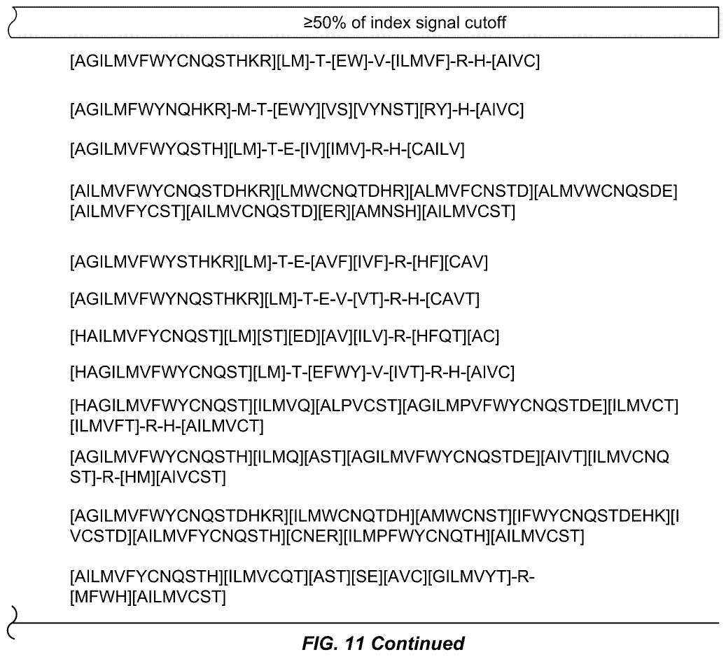

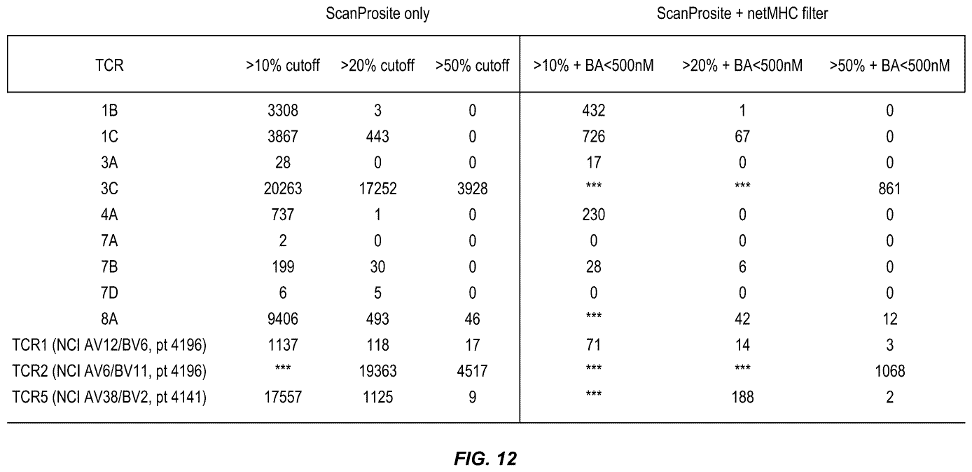

The X-scan data were used to construct putative consensus motifs based on permissive amino acid substitutions at cutoffs of 10%, 20%, and 50% of index peptide signal. If the normalized IFNg signal for a specific variant peptide was >the indicated percentage cutoff, the variant amino acid was classed as permissive and included in the consensus sequence at that position. Otherwise, if the signal was <the indicated percentage cutoff, the variant amino acid was classed as non-permissive and excluded from the consensus sequence at that position. Data on predicted consensus motif are illustrated in FIG. 11. Consensus motifs based on permissive amino acid substitutions at cutoffs of 10%, 20%, and 50% of index peptide signal were queried against the human proteome using ScanProsite to identify putative off targets. For each TCR/cutoff threshold, predicted off targets were additionally filtered through netMHC to identify the subset of predicted off targets that are also predicted to be processed and presented in the context of HLA A*02:01 with a binding affinity of ≤500 nM. Collectively, these data show that the presently disclosed TCRs have reduced off target effects as compared to the NCI benchmarks. Importantly, the presently disclosed TCR 7A and TCR 7D did not show any off-target effects at the most stringent cutoff.

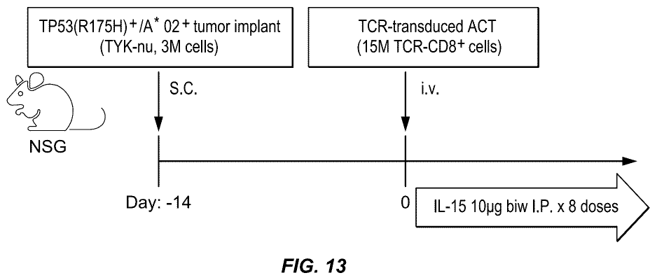

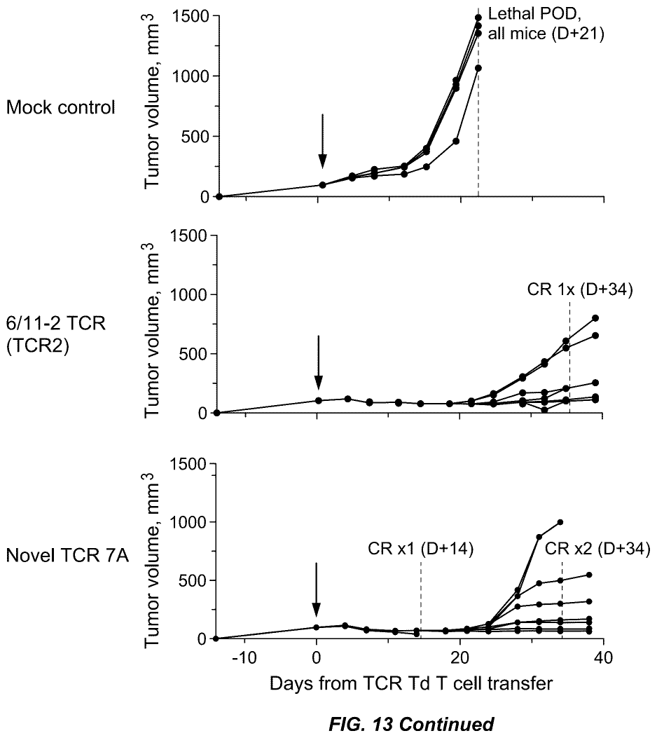

Next, the in vivo efficacy of the presently disclosed TCR was studied. Briefly, TP53 R175H⁺xenograft tumors were subcutaneously implanted in NSG mice and allowed to grow for 2-weeks. CD8⁺ T cells were isolated from a healthy donor and then transduced with the indicated TCR or mock control. When tumor reached >100 mm³, TCR transduced T cells were administered to each group of mice. To mimic the conditions of lymphodepletion, the mice were additionally treated with intraperitoneal IL-15 receptor complex starting on day of T cell transfer and continuing every 3-4 days for 8 doses up to tolerance. Tumor volume was measured by calipers 1-2 times weekly. Moribund mice, or those with tumor growth >1000 mm³ were euthanized according to established animal husbandry protocols. As seen in FIG. 13, T cells transduced with and expressing the TCR 7A elicited a significant reduction of the tumor volume in the mouse model as compared to the NCI benchmark.

### Embodiments of the Presently Disclosed Subject Matter

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or sub-combination) of listed elements. The recitation of an embodiment herein includes that embodiment as any single embodiment or in combination with any other embodiments or portions thereof.

All patents and publications and sequences referred to by accession or reference number mentioned in this specification are herein incorporated by reference to the same extent as if each independent patent and publication and sequence was specifically and individually indicated to be incorporated by reference.

What is claimed is:

1. A recombinant T cell receptor (TCR) comprising an extracellular domain that binds to a TP53 peptide, wherein the TP53 peptide comprises a R175 mutation,
wherein the extracellular domain comprises an α chain comprising an α chain variable region and α chain constant region, and a β chain comprising a β chain variable region and a β chain constant region, wherein
a) the α chain variable region comprises a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3; and the β chain variable region comprises a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6;
b) the α chain variable region comprises a CDR1 comprising the amino acid sequence set forth in SEQ ID NO: 11, a CDR2 comprising the amino acid sequence set forth in SEQ ID NO: 12, and a CDR3 comprising the amino acid sequence set forth in SEQ ID NO: 13; and the β chain variable region comprises a CDR1 comprising the amino acid sequence set forth in SEQ ID NO: 14, a CDR2 comprising the amino acid sequence set forth in SEQ ID NO: 15, and a CDR3 comprising the amino acid sequence set forth in SEQ ID NO: 16;
c) the α chain variable region comprises a CDR1 comprising the amino acid sequence set forth in SEQ ID NO: 21, a CDR2 comprising the amino acid sequence set forth in SEQ ID NO: 22, and a CDR3 comprising the amino acid sequence set forth in SEQ ID NO: 23; and the β chain variable region comprises a CDR1 comprising the amino acid sequence set forth in SEQ ID NO: 24, a CDR2 comprising the amino acid sequence set forth in SEQ ID NO: 25, and a CDR3 comprising the amino acid sequence set forth in SEQ ID NO: 26;
d) the α chain variable region comprises a CDR1 comprising the amino acid sequence set forth in SEQ ID NO: 31, a CDR2 comprising the amino acid sequence set forth in SEQ ID NO: 32, and a CDR3 comprising the amino acid sequence set forth in SEQ ID NO: 33; and the β chain variable region comprises a CDR1 comprising the amino acid sequence set forth in SEQ ID NO: 34, a CDR2 comprising the amino acid sequence set forth in SEQ ID NO: 35, and a CDR3 comprising the amino acid sequence set forth in SEQ ID NO: 36;
e) the α chain variable region comprises a CDR1 comprising the amino acid sequence set forth in SEQ ID NO: 41, a CDR2 comprising the amino acid sequence set forth in SEQ ID NO: 42, and a CDR3 comprising the amino acid sequence set forth in SEQ ID NO: 43; and the β chain variable region comprises a CDR1 comprising the amino acid sequence set forth in SEQ ID NO: 44, a CDR2 comprising the amino acid sequence set forth in SEQ ID NO: 45, and a CDR3 comprising the amino acid sequence set forth in SEQ ID NO: 46;
f) the α chain variable region comprises a CDR1 comprising the amino acid sequence set forth in SEQ ID NO: 51, a CDR2 comprising the amino acid sequence set forth in SEQ ID NO: 52, and a CDR3 comprising the amino acid sequence set forth in SEQ ID NO: 53; and the β chain variable region comprises a CDR1 comprising the amino acid sequence set forth in SEQ ID NO: 54, a CDR2 comprising the amino acid sequence set forth in SEQ ID NO: 55, and a CDR3 comprising the amino acid sequence set forth in SEQ ID NO: 56;
g) the α chain variable region comprises a CDR1 comprising the amino acid sequence set forth in SEQ ID NO: 61, a CDR2 comprising the amino acid sequence set forth in SEQ ID NO: 62, and a CDR3 comprising the amino acid sequence set forth in SEQ ID NO: 63; and the β chain variable region comprises a CDR1 comprising the amino acid sequence set forth in SEQ ID NO: 64, a CDR2 comprising the amino acid sequence set forth in SEQ ID NO: 65, and a CDR3 comprising the amino acid sequence set forth in SEQ ID NO: 66;
h) the α chain variable region comprises a CDR1 comprising the amino acid sequence set forth in SEQ ID NO: 71, a CDR2 comprising the amino acid sequence set forth in SEQ ID NO: 72, and a CDR3 comprising the amino acid sequence set forth in SEQ ID NO: 73; and the β chain variable region comprises a CDR1 comprising the amino acid sequence set forth in SEQ ID NO: 74, a CDR2 comprising the amino acid sequence set forth in SEQ ID NO: 75, and a CDR3 comprising the amino acid sequence set forth in SEQ ID NO: 76;
i) the α chain variable region comprises a CDR1 comprising the amino acid sequence set forth in SEQ ID NO: 81, a CDR2 comprising the amino acid sequence set forth in SEQ ID NO: 82, and a CDR3 comprising the amino acid sequence set forth in SEQ ID NO: 83; and the β chain variable region comprises a CDR1 comprising the amino acid sequence set forth in SEQ ID NO: 84, a CDR2 comprising the amino acid sequence set forth in SEQ ID NO: 85, and a CDR3 comprising the amino acid sequence set forth in SEQ ID NO: 86;
j) the α chain variable region comprises a CDR1 comprising the amino acid sequence set forth in SEQ ID NO: 91, a CDR2 comprising the amino acid sequence set forth in SEQ ID NO: 92, and a CDR3 comprising the amino acid sequence set forth in SEQ ID NO: 93; and the β chain variable region comprises a CDR1 comprising the amino acid sequence set forth in SEQ ID NO: 94, a CDR2 comprising the amino acid sequence set forth in SEQ ID NO: 95, and a CDR3 comprising the amino acid sequence set forth in SEQ ID NO: 96;
k) the α chain variable region comprises a CDR1 comprising the amino acid sequence set forth in SEQ ID NO: 101, a CDR2 comprising the amino acid sequence set forth in SEQ ID NO: 102, and a CDR3 comprising the amino acid sequence set forth in SEQ ID NO: 103; and the β chain variable region comprises a CDR1 comprising the amino acid sequence set forth in SEQ ID NO: 104, a CDR2 comprising the amino acid sequence set forth in SEQ ID NO: 105, and a CDR3 comprising the amino acid sequence set forth in SEQ ID NO: 106; or
l) the α chain variable region comprises a CDR1 comprising the amino acid sequence set forth in SEQ ID NO: 111, a CDR2 comprising the amino acid sequence set forth in SEQ ID NO: 112, and a CDR3 comprising the amino acid sequence set forth in SEQ ID NO: 113; and the β chain variable region comprises a CDR1 comprising the amino acid sequence set forth in SEQ ID NO: 114, a CDR2 comprising the amino acid sequence set forth in SEQ ID NO: 115, and a CDR3 comprising the amino acid sequence set forth in SEQ ID NO: 116.

2. The recombinant TCR of claim 1, wherein the α chain variable region comprises a CDR1 comprising the amino acid sequence set forth in SEQ ID NO: 81, a CDR2 comprising the amino acid sequence set forth in SEQ ID NO: 82, and a CDR3 comprising the amino acid sequence set forth in SEQ ID NO: 83; and the β chain variable region comprises a CDR1 comprising the amino acid sequence set forth in SEQ ID NO: 84, a CDR2 comprising the amino acid sequence set forth in SEQ ID NO: 85, and a CDR3 comprising the amino acid sequence set forth in SEQ ID NO: 86.

3. The recombinant TCR of claim 1, wherein the TP53 peptide comprises a R175H mutation.

4. The recombinant TCR of claim 1, wherein the TP53 peptide comprises or consists of the amino acid sequence set forth in SEQ ID NO: 146 or SEQ ID NO: 147.

5. The recombinant TCR of claim 1, wherein the TP53 peptide is associated with an HLA class I complex.

6. The recombinant TCR of claim 1, wherein the HLA class I complex is an HLA-A.

7. The recombinant TCR of claim 1, wherein the HLA-A is an HLA-A*02 superfamily member selected from the group comprising HLA-A*02:02, HLA-A*02:03, HLA-A*02:04, HLA-A*02:05, HLA-A*02:06, HLA-A*02:07, HLA-A*02:09, HLA-A*02:11, HLA-A*02:12, HLA-A*02:13, HLA-A*02:0215HLA-A*02:14, HLA-A*02:17, HLA-A*02:71, HLA-A*02:77, HLA-A*68:02, and HLA-A*69:01.

8. The recombinant TCR of claim 1, wherein:
a) the α chain variable region comprises an amino acid sequence that is at least about 80% homologous or identical to the amino acid sequence set forth in SEQ ID NO: 7, and the β chain variable region comprises an amino acid sequence that is at least about 80% homologous or identical to the amino acid sequence set forth in SEQ ID NO: 8;
b) the α chain variable region comprises an amino acid sequence that is at least about 80% homologous or identical to the amino acid sequence set forth in SEQ ID NO: 17, and the β chain variable region comprises an amino acid sequence that is at least about 80% homologous or identical to the amino acid sequence set forth in SEQ ID NO: 18;
c) the α chain variable region comprises an amino acid sequence that is at least about 80% homologous or identical to the amino acid sequence set forth in SEQ ID NO: 27, and the β chain variable region comprises an amino acid sequence that is at least about 80% homologous or identical to the amino acid sequence set forth in SEQ ID NO: 28;
d) the α chain variable region comprises an amino acid sequence that is at least about 80% homologous or identical to the amino acid sequence set forth in SEQ ID NO: 37, and the β chain variable region comprises an amino acid sequence that is at least about 80% homologous or identical to the amino acid sequence set forth in SEQ ID NO: 38;
e) the α chain variable region comprises an amino acid sequence that is at least about 80% homologous or identical to the amino acid sequence set forth in SEQ ID NO: 47, and the β chain variable region comprises an amino acid sequence that is at least about 80% homologous or identical to the amino acid sequence set forth in SEQ ID NO: 48;
f) the α chain variable region comprises an amino acid sequence that is at least about 80% homologous or identical to the amino acid sequence set forth in SEQ ID NO: 57, and the β chain variable region comprises an amino acid sequence that is at least about 80% homologous or identical to the amino acid sequence set forth in SEQ ID NO: 58;
g) the α chain variable region comprises an amino acid sequence that is at least about 80% homologous or identical to the amino acid sequence set forth in SEQ ID NO: 67, and the β chain variable region comprises an amino acid sequence that is at least about 80% homologous or identical to the amino acid sequence set forth in SEQ ID NO: 68;
h) the α chain variable region comprises an amino acid sequence that is at least about 80% homologous or identical to the amino acid sequence set forth in SEQ ID NO: 77, and the β chain variable region comprises an amino acid sequence that is at least about 80% homologous or identical to the amino acid sequence set forth in SEQ ID NO: 78;
i) the α chain variable region comprises an amino acid sequence that is at least about 80% homologous or identical to the amino acid sequence set forth in SEQ ID NO: 87, and the β chain variable region comprises an amino acid sequence that is at least about 80% homologous or identical to the amino acid sequence set forth in SEQ ID NO: 88;
j) the α chain variable region comprises an amino acid sequence that is at least about 80% homologous or identical to the amino acid sequence set forth in SEQ ID NO: 97, and the β chain variable region comprises an amino acid sequence that is at least about 80% homologous or identical to the amino acid sequence set forth in SEQ ID NO: 98;
k) the α chain variable region comprises an amino acid sequence that is at least about 80% homologous or identical to the amino acid sequence set forth in SEQ ID NO: 107, and the β chain variable region comprises an amino acid sequence that is at least about 80% homologous or identical to the amino acid sequence set forth in SEQ ID NO: 108; or
l) the α chain variable region comprises an amino acid sequence that is at least about 80% homologous or identical to the amino acid sequence set forth in SEQ ID NO: 117, and the β chain variable region comprises an amino acid sequence that is at least about 80% homologous or identical to the amino acid sequence set forth in SEQ ID NO: 118.

9. The recombinant TCR of claim 1, wherein:
a) the α chain variable region comprises the amino acid sequence set forth in SEQ ID NO: 7, and the β chain variable region comprises the amino acid sequence set forth in SEQ ID NO: 8;
b) the α chain variable region comprises the amino acid sequence set forth in SEQ ID NO: 17, and the β chain variable region comprises the amino acid sequence set forth in SEQ ID NO: 18;
c) the α chain variable region comprises the amino acid sequence set forth in SEQ ID NO: 27, and the β chain variable region comprises the amino acid sequence set forth in SEQ ID NO: 28;
d) the α chain variable region comprises the amino acid sequence set forth in SEQ ID NO: 37, and the β chain variable region comprises the amino acid sequence set forth in SEQ ID NO: 38;
e) the α chain variable region comprises the amino acid sequence set forth in SEQ ID NO: 47, and the β chain variable region comprises the amino acid sequence set forth in SEQ ID NO: 48;
f) the α chain variable region comprises the amino acid sequence set forth in SEQ ID NO: 57, and the β chain variable region comprises the amino acid sequence set forth in SEQ ID NO: 58;
g) the α chain variable region comprises the amino acid sequence set forth in SEQ ID NO: 67, and the β chain variable region comprises the amino acid sequence set forth in SEQ ID NO: 68;
h) the α chain variable region comprises the amino acid sequence set forth in SEQ ID NO: 77, and the β chain variable region comprises the amino acid sequence set forth in SEQ ID NO: 78;
i) the α chain variable region comprises the amino acid sequence set forth in SEQ ID NO: 87, and the β chain variable region comprises the amino acid sequence set forth in SEQ ID NO: 88;
j) the α chain variable region comprises the amino acid sequence set forth in SEQ ID NO: 97, and the β chain variable region comprises the amino acid sequence set forth in SEQ ID NO: 98;
k) the α chain variable region comprises the amino acid sequence set forth in SEQ ID NO: 107, and the β chain variable region comprises the amino acid sequence set forth in SEQ ID NO: 108; or
l) the α chain variable region comprises the amino acid sequence set forth in SEQ ID NO: 117, and the β chain variable region comprises the amino acid sequence set forth in SEQ ID NO: 118.

10. The recombinant TCR of claim 1, wherein:
a) the α chain comprises an amino acid sequence that is at least about 80% identical to the amino acid sequence set forth in SEQ ID NO: 9, and the β chain comprises an amino acid sequence that is at least about 80% identical to the amino acid sequence set forth in SEQ ID NO: 10;
b) the α chain comprises an amino acid sequence that is at least about 80% identical to the amino acid sequence set forth in SEQ ID NO: 19, and the β chain comprises an amino acid sequence that is at least about 80% identical to the amino acid sequence set forth in SEQ ID NO: 20;
c) the α chain comprises an amino acid sequence that is at least about 80% identical to the amino acid sequence set forth in SEQ ID NO: 29, and the β chain comprises an amino acid sequence that is at least about 80% identical to the amino acid sequence set forth in SEQ ID NO: 30;
d) the α chain comprises an amino acid sequence that is at least about 80% identical to the amino acid sequence set forth in SEQ ID NO: 39, and the β chain comprises an amino acid sequence that is at least about 80% identical to the amino acid sequence set forth in SEQ ID NO: 40;
e) the α chain comprises an amino acid sequence that is at least about 80% identical to the amino acid sequence set forth in SEQ ID NO: 49, and the β chain comprises an amino acid sequence that is at least about 80% identical to the amino acid sequence set forth in SEQ ID NO: 50;
f) the α chain comprises an amino acid sequence that is at least about 80% identical to the amino acid sequence set forth in SEQ ID NO: 59, and the β chain comprises an amino acid sequence that is at least about 80% identical to the amino acid sequence set forth in SEQ ID NO: 60;
g) the α chain comprises an amino acid sequence that is at least about 80% identical to the amino acid sequence set forth in SEQ ID NO: 69, and the β chain comprises an amino acid sequence that is at least about 80% identical to the amino acid sequence set forth in SEQ ID NO: 70;
h) the α chain comprises an amino acid sequence that is at least about 80% identical to the amino acid sequence set forth in SEQ ID NO: 79, and the β chain comprises an amino acid sequence that is at least about 80% identical to the amino acid sequence set forth in SEQ ID NO: 80;
i) the α chain comprises an amino acid sequence that is at least about 80% identical to the amino acid sequence set forth in SEQ ID NO: 89, and the β chain comprises an amino acid sequence that is at least about 80% identical to the amino acid sequence set forth in SEQ ID NO: 90;
j) the α chain comprises an amino acid sequence that is at least about 80% identical to the amino acid sequence set forth in SEQ ID NO: 99, and the β chain comprises an amino acid sequence that is at least about 80% identical to the amino acid sequence set forth in SEQ ID NO: 100;
k) the α chain comprises an amino acid sequence that is at least about 80% identical to the amino acid sequence set forth in SEQ ID NO: 109, and the β chain comprises an amino acid sequence that is at least about 80% identical to the amino acid sequence set forth in SEQ ID NO: 110; or
l) the α chain comprises an amino acid sequence that is at least about 80% identical to the amino acid sequence set forth in SEQ ID NO: 119, and the β chain comprises an amino acid sequence that is at least about 80% identical to the amino acid sequence set forth in SEQ ID NO: 120.

11. The recombinant TCR of claim 1, wherein:
a) the α chain comprises the amino acid sequence set forth in SEQ ID NO: 9, and the β chain comprises the amino acid sequence set forth in SEQ ID NO: 10;
b) the α chain comprises the amino acid sequence set forth in SEQ ID NO: 19, and the β chain comprises the amino acid sequence set forth in SEQ ID NO: 20;
c) the α chain comprises the amino acid sequence set forth in SEQ ID NO: 29, and the β chain comprises the amino acid sequence set forth in SEQ ID NO: 30;
d) the α chain comprises the amino acid sequence set forth in SEQ ID NO: 39, and the β chain comprises the amino acid sequence set forth in SEQ ID NO: 40;
e) the α chain comprises the amino acid sequence set forth in SEQ ID NO: 49, and the β chain comprises the amino acid sequence set forth in SEQ ID NO: 50;
f) the α chain comprises the amino acid sequence set forth in SEQ ID NO: 59, and the β chain comprises the amino acid sequence set forth in SEQ ID NO: 60;
g) the α chain comprises the amino acid sequence set forth in SEQ ID NO: 69, and the β chain comprises the amino acid sequence set forth in SEQ ID NO: 70;
h) the α chain comprises the amino acid sequence set forth in SEQ ID NO: 79, and the β chain comprises the amino acid sequence set forth in SEQ ID NO: 80;
i) the α chain comprises the amino acid sequence set forth in SEQ ID NO: 89, and the β chain comprises the amino acid sequence set forth in SEQ ID NO: 90;
j) the α chain comprises the amino acid sequence set forth in SEQ ID NO: 99, and the β chain comprises the amino acid sequence set forth in SEQ ID NO: 100;
k) the α chain comprises the amino acid sequence set forth in SEQ ID NO: 109, and the β chain comprises the amino acid sequence set forth in SEQ ID NO: 110; or
l) the α chain comprises the amino acid sequence set forth in SEQ ID NO: 119, and the β chain comprises the amino acid sequence set forth in SEQ ID NO: 120.

12. An immunoresponsive cell comprising the TCR of claim 1.

13. The cell of claim 12, wherein the cell is selected from the group consisting of a T cell, a Natural Killer (NK) cell, and a pluripotent stem cell from which a lymphoid cell may be differentiated.

14. The cell of claim 13, wherein the cell is a T cell or an NK cell.

15. The cell of claim 14, wherein the T cell is selected from the group consisting of a cytotoxic T lymphocyte (CTL), a regulatory T cell, a γδ T cell, a Natural Killer-T cell (NK-T), a stem cell memory T cell, a central memory T cell, and an effector memory T cell.

16. A composition comprising the cell of claim 12.

17. The composition of claim 16, which is a pharmaceutical composition further comprising a pharmaceutically acceptable carrier.

18. A nucleic acid encoding the T cell receptor (TCR) of claim 1.

19. A vector comprising the nucleic acid of claim 18.

20. The vector of claim 19, wherein (a) the vector is a γ-retroviral vector, or (b) the vector is a plasmid comprising a backbone comprising fewer than about 500 nucleotides.

21. A lipid nanoparticle comprising the nucleic acid of claim 18.

22. A method for producing a cell that binds to a TP53 peptide that comprises a R175 mutation, comprising introducing into the cell the nucleic acid of claim 18.

23. A method of treating and/or preventing a tumor or a premalignant condition associated with TP53 in a subject, comprising administering to the subject an effective amount of the cell of claim 12 or a composition thereof.

24. The method of claim 23, wherein the TP53 mutation is a R175H mutation.

25. The method of claim 23, wherein the tumor is selected from the group consisting of brain tumors, soft tissue and bone sarcomas, adrenocortical carcinoma, rhabdomyosarcoma, Wilms tumor, pancreatic cancer, breast cancer, endometrial cancer, cervical cancer, anal cancer, bladder cancer, colorectal cancer, cholangiocarcinoma/bile duct cancer, lung cancer, ovarian cancer, esophageal cancer, gastric cancer, head and neck squamous cell carcinoma, nonmelanoma skin cancer, salivary gland cancer, melanoma, multiple myeloma, prostate cancer, T-lymphoblastic leukemia/lymphoma, acute myeloid leukemia, B-cell leukemia, myeloproliferative neoplasm, and histiocytosis.

26. The method of claim 23, wherein the premalignant condition is clonal hematopoiesis.

27. The method of claim 23, wherein the subject is a human.

28. The method of claim 27, wherein the subject comprises an HLA-A.

29. The method of claim 28, wherein the HLA-A is an HLA-A*02 superfamily member selected from the group comprising HLA-A*02, HLA-A*02:01, HLA-A*02:02, HLA-A*02:03, HLA-A*02:04, HLA-A*02:05, HLA-A*02:06, HLA-A*02:07, HLA-A*02:09, HLA-A*02:11, HLA-A*02:12, HLA-A*02:13, HLA-A*02:0215HLA-A*02:14, HLA-A*02:17, HLA-A*02:71, HLA-A*02:77, HLA-A*68:02, and HLA-A*69:01.

30. The method of claim 29, wherein the HLA-A*02 superfamily member is HLA-A*02:01.
